(19) United States
(12) Patent Application Publication
(10) Pub. No.: US 2025/0131982 A1
(43) Pub. Date: Apr. 24, 2025

(54) SINGLE MOLECULE GENOME-WIDE MUTATION AND FRAGMENTATION PROFILES OF CELL-FREE DNA

(71) Applicant: The Johns Hopkins University, Baltimore, MD (US)

(72) Inventors: Victor E. Velculescu, Glenwood, MD (US); Robert B. Scharpf, Baltimore, MD (US); Daniel C. Bruhm, Baltimore, MD (US)

(21) Appl. No.: 18/720,276

(22) PCT Filed: Dec. 15, 2022
(86) PCT No.: PCT/US2022/053052

§ 371 Date: Jun. 14, 2024

Related U.S. Application Data
(60) Provisional application No. 63/290,017, filed on Dec. 15, 2021.

(51) Int. Cl. G16B 20/20; C12Q 1/6886; G16B 30/00; G16B 40/20; G16H 50/20

(52) U.S. Cl. CPC G16B 20/20; C12Q 1/6886; G16B 30/00; G16B 40/20; G16H 50/20; C12Q 2600/106

(57) ABSTRACT

Methods for non-invasive cancer detection use a combination of genome-wide mutation and fragmentation features of cfDNA that facilitate cancer screening.

### CROSS REFERENCE TO RELATED APPLICATIONS

This Application claims the benefit of U.S. Provisional Application 63/290,017 filed on Dec. 15, 2021. The entire contents of this application are incorporated herein by reference in its entirety.

### STATEMENT REGARDING FEDERALLY SPONSORED RESEARCH

This invention was made with government support under grants CA006973, CA121113 and CA233259 awarded by the National Institutes of Health. The government has certain rights in the invention.

### FIELD

Embodiments are directed to methods for determining the frequency of somatic mutations in a subject and in particular the diagnosis and treatment of cancer.

### BACKGROUND

Most of the mortality of human cancer is a consequence of late diagnosis when therapies are less effective¹. Early screening for cancer has demonstrated clinical benefit in multiple cancer types, but implementation of screening approaches remains a challenge². For example, screening for lung cancer using low-dose computed tomography (LDCT) is currently recommended in the United States for adults 50-80 years in age who have smoked at least 20 pack years and currently smoke or have quit within the last 15 years³ Although screening with LDCT has been shown to reduce mortality^{4,5} adherence to this test is low (<6%) among high-risk individuals⁶, in part due to concerns about potential harms from its low specificity, radiation exposure, and unnecessary diagnostic procedures. Liquid biopsies may overcome some of these challenges and provide an attractive approach for non-invasive detection of lung cancer and other malignancies.

### SUMMARY

Provided herein is a non-invasive and an ultrasensitive analysis of single cell-free DNA (cfDNA) molecules to detect the frequency of somatic mutations across the genome. Patients with cancer were found to have altered mutational profiles associated with chromatin organization compared to healthy individuals.

Accordingly, in certain aspects, a method of determining the frequency of somatic mutations in a subject, comprises extracting cell-free DNA (cfDNA) from a subject's biological sample; generating genomic libraries from the extracted cfDNA; sequencing individual cfDNA molecules to obtain mutation profiles; determining multiregional differences in mutation profiles; and determining the frequency of somatic mutations in the subject.

In certain embodiments, the determination of genome-wide mutation and fragmentation profiles comprises identifying mutations in sequences of individual cfDNA molecules and changes in fragment lengths.

In certain embodiments, the mutation profiles comprise mutation frequency and type of mutation across the subject's genome.

In certain embodiments, the mutation profiles across the subject's genome are determined using non-overlapping bins ranging in size from at least about one thousand bases to at least about twenty million bases.

In certain embodiments, the mutation profiles across the subject's genome are determined using non-overlapping bins ranging in size from at least about one thousand bases to at least about ten million bases.

In certain embodiments, the mutation profiles across the subject's genome are determined using non-overlapping bins ranging in size from at least about one thousand bases to at least about five million bases.

In certain embodiments, the mutations for each sequenced molecule are determined after removing common germline variants, and unevaluable regions.

In certain embodiments, the frequency of single molecule somatic mutations and type of mutation across the subject's genome is diagnostic of cancer as compared to the frequency of single molecule somatic mutations and type of mutation across a normal subject's genome.

In certain aspects, a method of treating cancer in a subject, the method comprises extracting cell-free DNA (cfDNA) from a subject's biological sample; generating genomic libraries from the extracted cfDNA; sequencing individual cfDNA molecules to obtain mutation profiles; determining multiregional differences in mutation profiles and determining the frequency of somatic mutations in the subject; and on the basis thereof administering a cancer treatment to the subject.

In certain embodiments, the cancer treatment comprises: surgery, adjuvant chemotherapy, neoadjuvant chemotherapy, radiation therapy, hormone therapy, cytotoxic therapy, immunotherapy, adoptive T cell therapy, targeted therapy, and combinations thereof.

In certain embodiments, the cancer comprises colorectal cancer, lung cancer, breast cancer, gastric cancers, pancreatic cancers, bile duct cancers, brain cancer or ovarian cancer.

In certain embodiments, the lung cancer is small cell lung cancer (SCLC).

In certain embodiments, the lung cancer is non-small cell lung cancer (NSCLC).

In certain embodiments, the subjects with cancer comprise altered mutational profiles associated with chromatin organization as compared to healthy individuals.

In certain embodiments, the genome-wide mutation and fragmentation profiles comprises identifying mutations in sequences of individual cfDNA molecules and changes in fragment lengths.

In certain embodiments, the mutation profiles comprise mutation frequency and type of mutation across the subject's genome.

In certain embodiments, the mutation profiles across the subject's genome are determined using non-overlapping bins ranging in size from at least about one thousand bases to at least about twenty million bases.

In certain embodiments, the mutation profiles across the subject's genome are determined using non-overlapping bins ranging in size from at least about one thousand bases to at least about ten million bases.

In certain embodiments, the mutation profiles across the subject's genome are determined using non-overlapping bins ranging in size from at least about one thousand bases to at least about five million bases.

In certain embodiments, the genome-wide mutations for each sequenced molecule are determined after removing common germline variants, and unevaluable regions.

In certain embodiments, a method of determining regional frequency of mutations across a genome including sequencing of individual cfDNA molecules isolated from a subject, estimating mutation frequencies and types of mutations across the genome; determining the mutation types and frequencies in genomic regions altered in cancer to mutation profiles and regions mutated in normal cfDNA to determine multiregional differences in mutation profiles; thereby, determining regional frequency of mutations across a genome. In certain embodiments, the estimation of mutation frequencies and types of mutations across the genome comprise using non-overlapping bins ranging in size from thousands to millions of bases. In certain embodiments, tumor specific changes are quantified by one or more assays. In certain embodiments, the one or more assays comprise in silico dilution assays and/or downsampling assays. In certain embodiments, each sequenced molecule is scanned for single nucleotide changes after removing common germline variants and/or unevaluable regions. In certain embodiments, the genomic regions are characterized by late replication timing, low gene expression, B compartmentalization, high H3K9me3 abundance, low GC content, or a combination thereof. In certain embodiments, the frequency of putative mutations is defined as the number of variants per million evaluated positions across all the DNA molecules sequenced. In certain embodiments, the method further comprises combining mutational profiles and genome-wide fragmentation profiles. In certain embodiments, the method further comprises executing a machine learning model for determining changes in genome-wide mutational profiles that classifies or excludes the subject as having or at risk of having cancer based on the genome-wide mutational profile identified for the subject.

In certain embodiments, a method of determining whether a subject is responding to treatment, comprises any one or more of the methods embodied herein. In certain embodiments, the treatment is selected from surgery, adjuvant chemotherapy, neoadjuvant chemotherapy, radiation therapy, hormone therapy, cytotoxic therapy, immunotherapy, adoptive T cell therapy, targeted therapy, and combinations thereof.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “aligned”, “alignment”, “mapped” or “aligning”, “mapping” refer to one or more sequences that are identified as a match in terms of the order of their nucleic acid molecules to a known sequence from a reference genome. Such alignment can be done manually or by a computer algorithm, examples including the Efficient Local Alignment of Nucleotide Data (ELAND) computer program distributed as part of the Illumina Genomics Analysts pipeline. The matching of a sequence read in aligning can be a 100% sequence match or less than 100% (non-perfect match).

The term “cancer” as used herein is meant, a disease, condition, trait, genotype or phenotype characterized by unregulated cell growth or replication as is known in the art; including lung cancer (including non-small cell lung carcinoma), gastric cancer, colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, bladder cancer, prostate cancer, pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells. Additionally, cfDNA may come from other sources such as viruses, fetuses, etc.

The term “circulating tumor DNA” or “ctDNA” refers to nucleic acid fragments that originate from tumor cells or other types of cancer cells, which may be released into an individual's bloodstream as result of biological processes such as apoptosis or necrosis of dying cells or actively released by viable tumor cells.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

An “effective amount” as used herein, means an amount which provides a therapeutic or prophylactic benefit.

As used herein, the terms “fragmentation profile,” “position dependent differences in fragmentation patterns,” and “differences in fragment size and coverage in a position dependent manner across the genome” are equivalent and can be used interchangeably. In some embodiments, determining a cfDNA fragmentation profile in a mammal can be used for identifying a mammal as having cancer. For example, cfDNA fragments obtained from a mammal (e.g., from a sample obtained from a mammal) can be subjected to low coverage whole-genome sequencing, and the sequenced fragments can be mapped to the genome (e.g., in non-overlapping windows) and assessed to determine a cfDNA fragmentation profile. As described herein, a cfDNA fragmentation profile of a mammal having cancer is more heterogeneous (e.g., in fragment lengths) than a cfDNA fragmentation profile of a healthy mammal (e.g., a mammal not having cancer). As such, this disclosure also provides methods and materials for assessing, monitoring, and/or treating mammals (e.g., humans) having, or suspected of having, cancer. In some embodiments, this document provides methods and materials for identifying a mammal as having cancer. For example, a sample (e.g., a blood sample) obtained from a mammal can be assessed to determine the presence and, optionally, the tissue of origin of the cancer in the mammal based, at least in part, on the cfDNA fragmentation profile of the mammal. In some embodiments, methods and materials for monitoring a mammal as having cancer are provided. For example, a sample (e.g., a blood sample) obtained from a mammal can be assessed to determine the presence of the cancer in the mammal based, at least in part, on the cfDNA fragmentation profile of the mammal. In some embodiments, methods and materials for identifying a mammal as having cancer and administering one or more cancer treatments to the mammal to treat the mammal are provided. For example, a sample (e.g., a blood sample) obtained from a mammal can be assessed to determine if the mammal has cancer based, at least in part, on the cfDNA fragmentation profile of the mammal, and one or more cancer treatments can be administered to the mammal.

As used herein, the “frequency” of mutations is defined as the number of variants per million evaluated positions across all the DNA molecules sequenced.

The term “genomic nucleic acid,” or “genomic DNA,” refers to nucleic acid including chromosomal DNA that originates from one or more healthy (e.g., non-tumor) cells. In various embodiments, genomic DNA can be extracted from a cell derived from a blood cell lineage, such as a white blood cell (WBC).

As used herein, the term “mutational profile” refers to the mutation type and frequency as observed in bins across the genome. Comparison of mutation profiles between genomic regions more commonly altered in cancer and mutation profiles from regions more frequently mutated in normal cfDNA can be used to determine multiregional differences.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some embodiments, the methods of the invention find use in experimental animals, in veterinary application, and in the development of animal models for disease, including, but not limited to, rodents including mice, rats, and hamsters, and primates.

The term “reference genome” as used herein may refer to a digital or previously identified nucleic acid sequence database, assembled as a representative example of a species or subject. Reference genomes may be assembled from the nucleic acid sequences from multiple subjects, sample or organisms and does not necessarily represent the nucleic acid makeup of a single person. Reference genomes may be used to for mapping of sequencing reads from a sample to chromosomal positions. For example, a reference genome used for human subjects as well as many other organisms is found at the National Center for Biotechnology Information at ncbi.nlm.nih.gov.

The term “read segment” or “read” refers to any nucleotide sequences including sequence reads obtained from an individual and/or nucleotide sequences derived from the initial sequence read from a sample obtained from an individual.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient, or a patient with lung cancer. In certain embodiments, a sample that is “provided” can be obtained by the person (or machine) conducting the assay, or it can have been obtained by another, and transferred to the person (or machine) carrying out the assay. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiment, a sample comprises cerebrospinal fluid. In a specific embodiment, a sample comprises a blood sample. In another embodiment, a sample comprises a plasma sample. In yet another embodiment, a serum sample is used. The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The term “sequence reads” refers to nucleotide sequences read from a sample obtained from an individual. Sequence reads can be obtained through various methods known in the art.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

FIG. 1 is a schematic of an overall approach for cancer detection using single molecule cfDNA sequencing. Blood is collected from a population of individuals, some of whom have cancer. cfDNA is extracted from plasma and subject to single molecule sequencing using massively parallel sequencing approaches. Sequence alterations are used to obtain genome-wide mutation profiles and regional differences in cancer and non-cancer mutation frequencies and are identified using machine learning to distinguish individuals with and without cancer.

FIGS. 2A-2J are a series of graphs and plots showing the single molecule mutation analyses of lung cancers from the PCAWG consortium and normal samples. FIG. 2A: Number of mutations detected in PCAWG lung cancer samples of smoking individuals when downsampled across a range of sequencing coverage amounts and tumor fractions. FIG. 2B: Fraction of PCAWG lung cancer mutations observed in single DNA molecules at the different sequence coverage and tumor fractions indicated. FIG. 2C: Frequency of single molecule somatic and background C>A changes in lung cancer and blood derived matched normal samples without quality or germline filters. FIG. 2D: Frequency of single molecule somatic and background C>A changes in lung cancer and blood derived matched normal samples with quality and germline filters including filtering of 8-oxo-dG related sequence changes. FIG. 2E: Frequency of single molecule somatic and background C>A changes spanning a 50 Mb region of chromosome 1 in patient DO25320. The C>A frequency was computed in a sliding 2.5 Mb window with a step size of 100 kb. The red and black dashed lines represent the mutation frequencies of the top decile of bins most enriched in C>A changes in lung cancers and matched blood derived normal samples. FIG. 2F: Background C>A frequency of the top decile of bins most enriched in C>A changes in lung cancer and matched blood derived normal samples obtained after removal of the known PCAWG somatic mutations. For each sample, the background C>A frequencies are similar between these regions as can be seen with the solid identity line. FIG. 2G: Number of molecules with each background C>A change in lung cancer and blood derived normal samples. Most background changes are only observed in a single molecule, even at >30× coverage. FIG. 2H: Difference in regional C>A frequencies in normal or tumor samples after subtraction of the C>A frequency in the top decile of bins enriched in normal samples from the top decile of bins enriched in mutations in tumor samples using the GEMINI approach. The difference in regional C>A frequencies preferentially removes background changes, thereby enriching the frequency of the observed somatic mutations. FIG. 2I: Association between the regional difference in single molecule C>A frequency and the frequency of high-confidence somatic C>A changes reported in these samples by the PCAWG consortium. FIG. 2J: Receiver operating characteristic (ROC) curve for distinguishing lung cancer from normal samples using the GEMINI approach with the testing set downsampled to 1× coverage compared to using overall single molecule C>A frequencies after quality and germline filtering. The ROC for GEMINI approach without filtering 8-oxo-dG related changes results in an AUC of 0.47, highlighting the importance of removing these artifacts for identification of tumor-specific alterations.

FIGS. 3A-3B are a series of plots and graphs demonstrating the genome-wide mutation profiles of tissue and plasma samples were associated with replication timing. FIG. 3A: Somatic mutation frequencies PCAWG lung cancers from smoking individuals (n=65) were computed in sliding 2.5 Mb windows with a step size of 100 kb across the genome and represented as the average across individuals. FIG. 3B: Association of mutation frequencies across tissue-specific replication timing strata in tissue and cfDNA from patients with NSCLC, melanoma, BNHL, or no cancer. Replication timing was obtained as the wavelet-smoothed transform of the six fraction profile representing different time points during replication in 1 kb bins from IMR90, NHEK and GM12878 cell lines^{47,48} for analyses of NSCLC, melanoma, and BNHL respectively. The weighted average of the replication timing values was computed in 2.5 Mb bins, followed by grouping of bins into 5 equal bin sets containing bins with the earliest to latest replication timing. In each bin set, we computed the mutation frequency in tissue at different replication strata using the number of somatic mutations reported by the PCAWG Consortium per Mb of genome and compared this to the single molecule mutation frequency in plasma using a Pearson correlation. To control for potential systematic variability in measured genome-wide mutational frequencies, we subtracted from both cancer and non-cancer cfDNA samples the single molecule mutation frequency in each bin set in a separate panel of 20 non-cancer cfDNA samples. Mutation frequencies were then scaled within each sample and mutation type to have a minimum value of zero.

FIGS. 4A-4I are a series of plots and ROC curves demonstrating the detection of lung cancer using GEMINI and combined GEMINI/DELFI approach. FIG. 4A: GEMINI scores in high-risk individuals, age 50-80 with a ≥20 pack per year smoking history with or without lung cancer, with the number of individuals indicated at each stage or histology. Importantly, non-cancer individuals with and without benign nodules had similar GEMINI scores, and individuals with cancer had higher GEMINI scores. FIG. 4B: GEMINI scores of high-risk individuals without lung cancer as well as individuals without lung cancer as determined by imaging at baseline but who later developed lung cancer. FIG. 4C: GEMINI scores in the validation cohort in current or former smokers aged 50-80 with and without cancer. The validation cohort was enriched for early stage disease (stage I=25, stage II=2, stage III=2, stage IV=2, and 1 individual with unknown stage). FIG. 4D: ROC curve for detection of lung cancer in high-risk individuals in the LUCAS cohort (n=89 with lung cancer, n=74 without cancer) shows high performance using the GEMINI or GEMINI and DELFI approaches. FIG. 4E: ROC curve for detection of lung cancer in subset of high-risk individuals in the LUCAS cohort with at least 40 pack years (n=63 with lung cancer, n=46 without cancer) shows increased performance of GEMINI with higher smoking history. FIG. 4F: ROC curve for detection of high-risk individuals from LUCAS cohort who were diagnosed with stage I lung cancer (n=13 with lung cancer, n=74 without cancer). FIG. 4G: ROC curve for detection of stage I lung cancer among individuals in the validation cohort (n=25 with lung cancer, n=14 without cancer). FIG. 4H: ROC curve for detection of high-risk individuals from LUCAS cohort and a ≥40 pack year smoking history who were diagnosed with stage I lung cancer (n=9 with lung cancer, n=46 without cancer). FIG. 4I: ROC curve for detection of stage I lung cancer and a ≥40 pack year smoking history among individuals in the validation cohort (n=13 with lung cancer, n=5 without cancer).

FIGS. 5A-5F are a series of graphs and an ROC curve demonstrating the GEMINI approach for noninvasive detection across multiple cancer types. FIG. 5A: GEMINI scores in SCLC patients and high-risk individuals without cancer in the LUCAS and the validation cohorts show high performance for detecting cancer (Supplementary Table 4). FIG. 5B: Regional differences in single molecule C>A frequency in the LUCAS and validation cohorts demonstrates that the GEMINI approach can be used to identify bins most altered between SCLC and NSCLC. FIG. 5C: ROC curves for detection of SCLC (n=13) compared to non-cancer controls (n=88) (orange) as well as for distinguishing SCLC (n=13) from NSCLC (n=99) (purple) in the combined LUCAS and validation cohorts. FIG. 5D: Cross-validated regional differences in single molecule mutation frequencies in cfDNA in the liver cancer cohort, median-centered within each mutation type, show a high level of T>C mutations in patients with HCC. P-values were generated using the Wilcoxon rank sum test and were corrected for multiple comparisons using the Benjamini-Hochberg method. The horizontal dashed line indicates a p-value of 0.05. FIG. 5E: GEMINI scores in the liver cancer cohort with the number of individuals indicated at each stage demonstrate high sensitivity for detection of liver cancer across all stages. FIG. 5F: Principal coordinate analysis of the Euclidean distance matrix reflecting cross-validated pairwise differences in regional mutation frequencies between NSCLC, SCLC, and HCC. The first two principal coordinates are shown with contours indicating kernel density estimations for 0.7 and 0.95 probability for each cancer type. The composition of cancer types in clusters derived from K-means clustering with k=3 is indicated to the right.

FIG. 6 is a schematic showing an overview of cohorts analyzed. Each box represents a cohort analyzed and indicates whether the GEMINI approach was evaluated with either cross-validation or validated using a fixed model. Dashed lines indicate analyses of cohort subsets for evaluation of individual tumor types or comparison of cancer subtypes.

FIG. 7 is a series of plots showing the genomic mutation profiles in common cancers. Average somatic mutation frequencies computed in sliding 2.5 Mb windows with a step size of 100 kb across chromosome 1 obtained were obtained from an analysis of 2,511 PCAWG samples across 25 common cancer types.

FIG. 8 is a schematic of dilution and downsampling experiment. In this example, we consider a tumor sample that contains N somatic mutations at genomic positions 1, 2, . . . , N. We begin with 30 non-tumor derived observations and 10 tumor derived mutations (25% tumor purity). During the dilution step, non-tumor observations are spiked in until the desired tumor fraction is achieved. After dilution, fragments are randomly sampled from the set of all fragments to achieve the desired average coverage across genomic positions. The resulting number of observed mutations is counted, and lastly, the proportion of observed mutations that are only observed in a single fragment is computed. In this example, there are 3 observed mutations and one of them is only observed in a single molecule.

FIGS. 9A, 9B is a plot and a graph demonstrating the identification of background changes in single molecule sequencing related to 8-oxo-dG damage. FIG. 9A: Ratio of the frequency of each type of single base change in 62 tissue samples from PCAWG (31 lung cancer and 31 blood derived matched normal samples) when prior to mutation purines guanine or adenine (pu) are on read 1 (R1) or pyrimidines cytosine or thymine (py) are on read 2 (R2) to when the pyrimidine is on read 1 and the purine is on read 2 for both background changes and known germline variants. Background changes reflect sequence changes identified through single molecule analyses that were not reported as somatic variants by PCAWG. Here, germline variants reported by PCAWG were also removed from the background variants to enrich for likely artifactual changes. FIG. 9B: Ratio of known somatic mutations to background changes identified through single molecule analyses before removing likely 8-oxo-dG related sequence changes (R1_{pu or py}, R2_{pu or py}), and after filtering these changes where only bases with cytosine on R1 and guanine on read 2 are considered (R1_{py}, R2ₚᵤ).

FIGS. 10A, 10B are plots demonstrating the analyses of single molecule sequence changes in PCAWG lung cancer and normal samples. FIG. 10A: Single molecule mutation frequencies in PCAWG lung cancers (n=31) and blood derived matched normal samples (n=31). P-values were corrected for multiple comparisons using the Benjamini-Hochberg method. The horizontal dashed line indicates a p-value of 0.05. FIG. 10B: Cross-validated regional differences in single molecule mutation frequencies in PCAWG lung cancers (n=31) and blood derived matched normal samples (n=31), median-centered within each mutation type. P-values were generated using the Wilcoxon rank sum test and were corrected for multiple comparisons using the Benjamini-Hochberg method. The horizontal dashed line indicates a p-value of 0.05.

FIG. 11 is a graph demonstrating the analysis of somatic and background changes across mutation types in PCAWG lung cancers. Ratio of somatic to background changes identified through single molecule analyses after removal of potential 8-oxo-dG related artifacts for each mutation type analyzed. Somatic changes reflect sequence changes identified through single molecule analyses that were also reported as somatic mutations by PCAWG, whereas background changes were identified through single molecule analyses but not reported as somatic mutations by PCAWG. Overall, C:G>A:T changes represented the highest fraction of somatic changes.

FIG. 12 is a series of plots demonstrating the analysis of single molecule sequence changes across sequencing lanes in PCAWG lung cancer and normal samples. Single molecule mutation frequencies in PCAWG lung cancer and blood derived normal samples across sequencing lanes. For each sample, sequencing reads were split into separate Binary Alignment Map (BAM) files based on their associated read group, which indicates the sequencing reads from one lane of an NGS experiment. The resulting BAM files contained a median of 464 million reads (range: 6-738 million). Approximately 1 million reads were randomly sampled 5 times with replacement from each sequencing lane (a maximum of 6 lanes is shown per sample). Single molecule mutation frequencies varied widely within an individual sample depending on the analyzed lane and the type of sequence alteration.

FIG. 13 is a series of plots demonstrating the genome-wide somatic single molecule C>A mutation profiles in lung cancers. Single molecule C>A somatic mutation frequencies computed in sliding 2.5 Mb windows with a step size of 100 kb across the autosomes obtained from an aggregated analysis of the 31 PCAWG lung cancer samples showed widespread differences in mutation frequencies depending on genomic location.

FIG. 14 is a series of plots demonstrating somatic single molecule C>A mutation profiles across chromosome 4 in PCAWG lung cancers. Single molecule C>A somatic mutation frequencies computed in a sliding 2.5 Mb window with a step size of 100 kb across chromosome 4 from PCAWG lung cancer samples revealed similar mutation profiles among different lung cancers.

FIG. 15 is a schematic of a GEMINI regional mutation frequency analysis. The genome is divided into 1,144 non-overlapping 2.5 Mb bins (20 bins are depicted here) and the single molecule mutation frequency is computed in each bin as the number of sequence changes per million evaluable bases, defined as the number of positions in fragments in which each sequence change could be detected after quality and germline filtering. Samples in the training set are used to identify the bins that are most differentially mutated between cancer and non-cancer samples. In the training set, sequence data from all cancer samples and all non-cancer samples are combined, and the cancer and non-cancer single molecule mutation frequencies are computed in each bin. Next, the difference in single molecule mutation frequency is computed between cancer and non-cancer samples in each bin, and the 10% of bins most mutated in cancer samples relative to non-cancer samples, as well as the 10% of bins most mutated in non-cancer samples relative to cancer samples, are identified (indicated by triangles and circles respectively). In the testing set, the difference in single molecule mutation frequency is computed between these two sets of bins in a new sample not included in the training set, generating a regional difference in mutation frequency that can be used to classify the sample into being derived from a healthy individual or an individual with cancer. By taking the difference in single molecule mutation frequency between two sets of regions in the genome within an individual sample, this approach controls for the overall number of sequence changes in that sample that may result from technical variability in sequencing runs.

FIG. 16 is a graph demonstrating the effect of matched WBC filtering in PCAWG lung cancers on enrichment of somatic alterations by single molecule sequencing. Single molecule C>A frequency in PCAWG lung cancers (n=31) after removal of any sequence changes identified in matched blood derived normal samples at >30× coverage. The analysis revealed that subtraction of mutations observed in the matched normal sample was not effective in removing background changes because such alterations typically were observed once and were not present in both tumor and matched non-cancer samples.

FIGS. 17A-17C are a series of plots demonstrating the association of single molecule genome-wide mutation profiles of tissue and plasma samples with genomic features. The figures show the genome-wide mutation frequencies across strata of tissue-specific gene expression, A/B compartmentalization, and H3K9me3 abundance, respectively, in tissue and cfDNA from patients with NSCLC, melanoma, BNHL, or without cancer. The weighted average of each feature value was computed in 2.5 Mb bins, followed by grouping of bins into 5 equal bin sets ordered by feature value. In each bin set, we computed the mutation frequency in tissue at different strata using the number of somatic mutations reported by the PCAWG Consortium per Mb of genome and compared this to the single molecule mutation frequency in plasma using a Pearson correlation. To account for difference in the overall frequency of each mutation type in each bin in cfDNA, the single molecule mutation frequency in each bin set in a panel of non-cancer samples (n=20) was subtracted from the single molecule mutation frequency in each bin set in cancer and non-cancer cfDNA samples and the resulting values were scaled to have a minimum value of zero for each mutation type and sample type. FIG. 17A: Gene expression was computed as the sum of the transcripts per million (TPM) overlapping each 2.5 Mb bin weighted by the length of the transcript averaged across TCGA NSCLC, melanoma, and BNHL samples. FIG. 17B: A/B compartmentalization, largely representing open and closed regions of the genome, respectively, was measured as the first eigenvector of the correlation matrix of average methylation beta values in 100 kb bins across TCGA NSCLC samples for NSCLC analyses and was averaged across 12 TCGA cancer types for melanoma analyses. The first eigenvector for the genome contact matrix from Hi-C analyses of lymphoblastoid cells (GM12878 cell line) was used for BNHL analyses³³. FIG. 17C: The abundance of H3K9me3, a known marker of heterochromatin, was obtained from ChIP-seq of A549 cells (three pooled replicates), GM23248, and Karpas 422 cells (two pooled replicates) for NSCLC, melanoma, and BNHL analyses respectively as the fold change of coverage in enriched samples compared to control samples⁴⁸.

FIG. 18 is a plot demonstrating the regional differences in single molecule mutation frequencies in the high-risk LUCAS cohort. Cross-validated regional differences in single molecule mutation frequencies in cfDNA in individuals with lung cancer (n=89) and individuals without cancer (n=74), median-centered within each mutation type. Regional C>A mutation frequencies were preferentially altered between lung cancer and non-cancer samples, but not when randomly permuting class labels (p=0.36, Wilcoxon rank sum test). P-values were generated using the Wilcoxon rank sum test and were corrected for multiple comparisons using the Benjamini-Hochberg method. The horizontal dashed line indicates a p-value of 0.05.

FIG. 19 is a plot showing the analyses of C>A sequence changes by flow cell and sequencing lane in non-cancer individuals. Single molecule C>A frequencies and regional differences in single molecule C>A frequencies across flow cells and sequencing lanes for all non-cancer individuals from the LUCAS cohort (n=158). Although sequencing background mutation rates differed by lane resulting in multiple samples within a sequencing lane having similar single molecule C>A frequencies, explaining 99% of their variance (p<0.0001, F-test), this association was eliminated using regional differences in single molecule C>A frequencies obtained with the GEMINI approach (p=0.17, F-test).

FIGS. 20A-20K is a series of plots and schematics showing the genome-wide fixed bins utilized for analysis of single molecule mutation frequencies and detection of lung cancer in cfDNA. FIG. 20A: Percent similarity of bins identified as being enriched for mutations in lung cancer and non-cancer samples in each training fold compared to the sets of bins utilized in the fixed model that were identified from analyses of all samples. A high similarity across training folds indicated that bin selection was not driven by individual samples. FIG. 20B: Chromosomal location of bins enriched in mutations in cfDNA of patients with lung cancer and bins enriched in mutations in cfDNA of individuals without cancer. FIG. 20C: Compared to samples from individuals without cancer, samples from those with lung cancer had more C>A changes per genomic bin across samples in bins enriched in lung cancer and fewer of these changes in bins enriched in non-cancer. FIGS. 20D-20E: The average number of evaluable bases and copy number per genomic bin was similar in non-cancer individuals and individuals with lung cancer in bins enriched in lung cancer and bins enriched in non-cancer. Copy number was estimated using ichorCNA. FIGS. 20F-20K: Bins in the fixed model were associated with replication timing, gene expression, A/B compartmentalization, and H3K9me3 abundance, GC content, but not sequence mappability. FIG. 20F: Replication timing was obtained as the wavelet-smoothed transform of the six fraction profile representing different time points during replication in 1 kb bins from IMR90 cells^{47,49} and then computing the weighted average in each 2.5 Mb bin with higher values indicating earlier replication timing. FIG. 20G: Gene expression was computed as the sum of the transcripts per million (TPM) overlapping each 2.5 Mb bin weighted by the length of the transcript averaged across TCGA NSCLC samples and log transformed as log₁₀(TPM). FIG. 20H: A/B compartmentalization, largely representing open and closed regions of the genome, respectively, was measured as the first eigenvector of the correlation matrix of average methylation beta values in 100 kb bins across TCGA lung cancer samples³³. FIG. 20I: The abundance of H3K9me3, a known marker of heterochromatin, was obtained from ChIP-seq of A549 cells⁴⁸ and displayed as the fold change of coverage in enriched samples compared to control samples from three pooled replicates. FIG. 20J: GC content in each genomic bin was obtained from the hg19 reference genome. Bins enriched in lung cancer tend to be AT rich (GC poor) compared to bins enriched in non-cancer, which may be explained through our previous results that later replicating regions which are enriched in mutations in lung cancer have lower GC content (Spearman's rho=0.83, p<0.0001). FIG. 20K: Mappability, reflecting how uniquely 100-mer sequences align to a region of the genome, was computed as the weighted average in 2.5 Mb bins.

FIGS. 21A-21F are a series of plots showing an analyses of doublet base substitutions in tissue and plasma samples of lung cancer patients. FIG. 21A: Number of somatic doublet base substitutions identified by the PCAWG Consortium in lung cancer tissue samples from smoking individuals (n=65) revealed a high number of CC>AA changes compared to other doublet mutations. Solid horizontal lines indicate the median number of each mutation type across individuals. FIG. 21B: Ratio of single molecule CC>AA frequencies when CC or CC>AA is in read 1 and GG or GG>TT is in read 2 (R1_{CC}, R2_{GG}) relative to when GG or GG>TT is in read 1 and CC or CC>AA is in read 2 (R1_{GG}, R2_{CC}) aggregated across samples in the high-risk LUCAS cohort. Background CC>AA changes represent those alterations that were only observed in single cfDNA fragments in individuals without cancer, whereas likely somatic changes represent those alterations that are private to an individual sample from a patient with lung cancer and are observed in multiple cfDNA fragments. Within the high-risk LUCAS cohort, there were 67 private CC>AA changes that were observed in two or more fragments across 89 individuals with lung cancer, and only one such changes observed across 74 individuals without cancer, indicating that most of these alterations were likely somatic in origin. Bars represent 95% bootstrap confidence intervals for the ratios. Background CC>AA changes were more often detected as R1_{CC}, R2_{GG}, but no imbalance in likely somatic CC>AA changes was observed, indicating an enrichment of likely artifactual background CC>AA changes detected as R1_{CC}, R2_{GG}. FIG. 21C: Sequence context surrounding CC>AA changes (+/−5 bp) in the high-risk LUCAS cohort, where the number of mutations is indicated for each group, and the total height of the letters at each position indicates the information content of the position measured in bits. FIG. 21D: Single molecule CC>AA frequencies were elevated in individuals with lung cancer compared to non-cancer individuals with a larger separation observed after filtering CC>AA changes detected as R1_{GG}, R2_{CC}. FIGS. 21E-21F: Single molecule CC>AA frequencies were positively correlated with regional differences in single molecule C>A frequencies in cfDNA (FIG. 21E) and lung tumors (FIG. 21F) after filtering CC>AA changes detected as Rlcc, R2_{GG}.

FIGS. 22A-22F are a series of plots demonstrating the effect of clinical characteristics on GEMINI scores in non-cancer individuals in the LUCAS cohort. GEMINI scores are shown for FIG. 22A, males (n=87) and females (n=71), FIG. 22B, individuals with (n=43) or without (n=115) autoimmune disease, FIG. 22C, individuals with (n=28) or without (n=130) COPD, FIG. 22D, individuals of different ages, and FIGS. 22E-22F, compared to CRP (mg/L), and IL-6 levels (pg/mL).

FIGS. 23A, 23B are a series of plots demonstrating that GEMINI scores reflect tumor DNA content in cfDNA. FIG. 23A: GEMINI scores in the high-risk LUCAS cohort in individuals without cancer and individuals with lung cancer at different levels of ctDNA. A score >0.55 reflects a positive test for detection of lung cancer at 80% specificity. b, GEMINI scores in the liver cancer cohort in individuals with cirrhosis and individuals with liver cancer that have <3% or ≥3% ctDNA. A score >0.86 reflects a positive test for detection of liver cancer at 80% specificity. The percentage of ctDNA in each sample was estimated using ichorCNA.

FIGS. 24A, 24B are a series of ROC curves demonstrating the performance of GEMINI or the combined GEMINI/DELFI approach for detection of lung cancer. FIG. 24A: ROC curves for detection of lung cancer in the high-risk LUCAS cohort using GEMINI or the combined GEMINI/DELFI approach in patients with stages II-IV disease and in the subset of these patients that smoked ≥40 pack years. FIG. 24B: ROC curves for detection of lung cancer in the high-risk LUCAS cohort using GEMINI or the combined GEMINI/DELFI approach in patients with adenocarcinoma, squamous cell carcinoma, or small cell lung cancer and in the subset of these patients that smoked ≥40 pack years. Performance for Stage I disease is shown in FIGS. 4F, 4H.

FIG. 25 is a graph demonstrating GEMINI and DELFI scores as well as their combined performance for detecting cancer in the LUCAS cohort. GEMINI and DELFI scores are shown for each patient in the high-risk LUCAS cohort (n=163). Vertical and horizontal dashed lines indicate the threshold for a positive GEMINI and DELFI test respectively at 80% specificity, while filled circles indicate a positive test by the combined approach at the same specificity. Several individuals with cancer are detected by one approach but not the other, and the combined score detected more individuals with lung cancer compared to either approach in isolation.

FIG. 26 is a graph demonstrating the GEMINI/DELFI score and clinical outcome in lung cancer patients. Patients with lung cancer in the high-risk LUCAS cohort (n=89) were stratified in two groups based on the median GEMINI/DELFI score among lung cancer patients of 0.84. Patients with a GEMINI/DELFI score ≥0.84 (yellow) had a significantly worse overall survival compared to patients with a GEMINI/DELFI score <0.84 (blue) (p=0.004, Log-rank test).

FIGS. 27A-27D are a series of graphs and plots showing a comparison of cfDNA characteristics across non-cancer patients in LUCAS, DECAMP, and AHN cohorts. FIG. 27A: Average genome-wide coverage in non-cancer samples across cohorts. The horizontal dashed lines represent the median coverage of samples in each cohort. FIG. 27B: Regional differences in single molecule C>A frequencies are similar between cohorts (p=0.17, Kruskal-Wallis test). Solid horizontal lines represent the median in each group. FIG. 27C: For each non-cancer sample, the ratio of short (100-150 bp) to long (151-220 bp) fragments were computed in 473 non-overlapping 5 Mb bins and mean-centered. Median fragmentation profiles represent the median of these values across samples in each bin and are highly correlated between cohorts (Pearson correlation coefficient >0.97 for each pairwise comparison). FIG. 27D: Chromosomal arm-level Z-scores in non-cancer samples are similar between cohorts (p>0.05 for each chromosomal arm, Kruskal-Wallis test with Bonferroni correction).

FIGS. 28A-28C are a series of graphs demonstrating GEMINI scores and smoking exposure in lung cancer patients. FIG. 28A: Single molecule C>A frequencies were similar in never smokers with lung cancer (n=3) or without lung cancer (n=34) in the LUCAS cohort. In current or former smokers in the high-risk group, with a ≥20 pack year smoking history and age 50-80, the single molecule C>A frequencies were slightly higher in individuals with lung cancer (n=89) compared to individuals without lung cancer (n=74). FIG. 28B: GEMINI scores were similar in never smokers with lung cancer (n=3) or without lung cancer (n=34). In the high-risk group, GEMINI scores were higher in individuals with lung cancer (n=89) compared to those without lung cancer (n=74). Similarly, for individuals with a ≥40 pack year smoking history and age 50-80, the GEMINI scores were higher in those with lung cancer (n=63) compared to those without lung cancer (n=46). FIG. 28C: GEMINI scores were higher in individuals with lung cancer in the validation cohort in current/former smokers age 50-80 with (n=32) and without lung cancer (n=14) and in the subset with a ≥40 pack year smoking history with (n=18) and without lung cancer (n=5).

FIG. 29 is a graph showing a principal coordinate analysis in patients with cancer after excluding the most frequent mutation types. The regional difference in single molecule mutation frequency was computed between NSCLC, SCLC, and HCC using a leave-one-out procedure for C>G, C>T, T>A and T>G mutations, yielding 12 feature values. A Euclidean distance matrix reflecting pairwise differences between samples was generated from these 12 feature values. A principal coordinate analysis of the Euclidean distance matrix revealed a reduced separation of samples by cancer type compared to when C>A and T>C mutations were also analyzed (FIG. 5F).

FIG. 30 is a series of graphs showing GEMINI scores and MAF levels during therapy. Individuals with a smoking history as well as availability of targeted deep sequencing¹¹ and low coverage whole-genome sequencing data¹³ were analyzed before and during treatment with tyrosine kinase inhibitors (arrows indicate initiation of treatment). GEMINI scores were associated with the maximum mutant allele fraction at each timepoint (Spearman's rho=0.50, p=0.03).

### DETAILED DESCRIPTION

Somatic mutations are a hallmark of tumorigenesis and may be useful for non-invasive diagnosis of cancer. However, the detection of somatic alterations in the circulation has been challenging due to the limited number of tumor derived molecules in cell-free DNA (cfDNA). An ultrasensitive analysis of single cfDNA molecules was developed herein, to detect the frequency of somatic mutations across the genome and found that patients with cancer had altered mutational profiles associated with chromatin organization compared to healthy individuals. Combining genome-wide cfDNA mutational profiles and fragmentation features followed by CT imaging detected 95% of patients with cancer across stages and subtypes, including 95% of stage I and II patients, with a 90% combined specificity. The model was independently validated in a separate screening cohort of high-risk individuals with early stage lung cancer. Genome-wide mutational profiles distinguished individuals with small cell lung cancer from those with non-small cell lung cancer and could identify lung cancers earlier than standard approaches. This approach lays the groundwork for non-invasive cancer detection using a combination of genome-wide mutation and fragmentation features of cfDNA that may facilitate cancer screening.

### Gemini

Sequence alterations are abundant in cancer genomes but the proportion of fragments in cell-free DNA (cfDNA) that harbor tumor-specific (somatic) mutations is often low^{7,8}, making it difficult to detect bonafide variants amongst the background noise due to sequence changes introduced in library construction, gene selection, PCR amplification and sequencing. Extensive efforts have been ma.de to detect mutations that are present at low frequencies in cfDNA. However, these methods typically rely on deep sequencing and have been restricted to examining specific genes comprising a small subset of the genome⁹⁻¹¹. Due to the low number of genome equivalents derived from cancer cells in cfDNA, such approaches have limited efficacy for detecting the presence of cancer especially in early stage disease¹²⁻¹⁴. Additionally, sequence alterations in cfDNA may arise from white blood cells (WBCs), confounding the use of sequence mutations to detect patients with cancer^{7,15,16}.

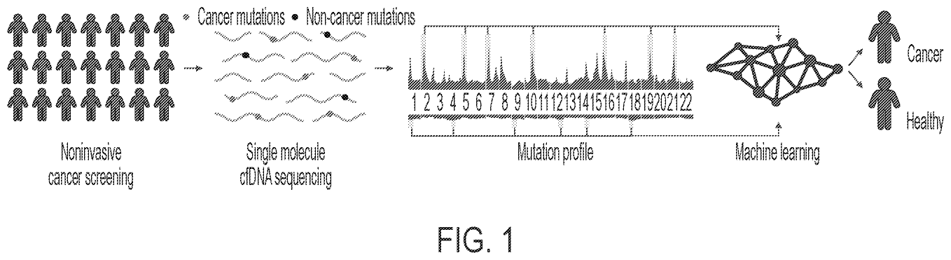

The method disclosed herein and termed genome-wide mutational incidence for noninvasive detection of cancer (GEMINI), identified a much larger number of tumor-derived alterations in cfDNA for cancer detection (FIG. 1). The method is based on sequencing individual cfDNA molecules to estimate the mutation frequency and type of alteration across the genome using non-overlapping bins ranging in size from thousands to millions of bases. For each individual, the mutational profile in genomic regions more commonly altered in cancer is compared to the profile from regions more frequently mutated in normal cfDNA to determine multiregional differences in mutation profiles. In this way, the GEMINI approach enriches for likely somatic mutations while taking into account individual variability in overall mutation number.

Accordingly, in certain embodiments, a method of determining the frequency of somatic mutations in a subject comprises extracting cell-free DNA (cfDNA) from a subject's biological sample; generating genomic libraries from the extracted cfDNA; sequencing individual cfDNA molecules to obtain mutation profiles; determining multiregional differences in mutation profiles; and, determining the frequency of somatic mutations in the subject.

The generation of genome-wide mutation profiles included identifying mutations in sequences of individual cfDNA molecules. The mutation profiles across the subject's genome are determined using non-overlapping bins ranging in size from at least about one hundred bases to at least about twenty million bases. In certain embodiments, the mutation profiles across the subject's genome are determined using non-overlapping bins ranging in size from at least about 500 bases to at least about fifteen million bases. In certain embodiments, the mutation profiles across the subject's genome are determined using non-overlapping bins ranging in size from at least about 750 bases to at least about ten million bases. In certain embodiments, the mutation profiles across the subject's genome are determined using non-overlapping bins ranging in size from at least about 900 bases to at least about ten million bases. In certain embodiments, the mutation profiles across the subject's genome determined using non-overlapping bins ranging in size from at least about one thousand bases to at least about five million bases.

In certain embodiments, the frequency of single molecule somatic mutations and type of mutation across the subject's genome is diagnostic of cancer as compared to the frequency of single molecule somatic mutations and type of mutation across a normal subject's genome.

In certain embodiments, the frequency of a somatic mutation at various loci is indicative of cancer. In certain embodiments, the type of mutation is indicative of cancer.

cfDNA Fragmentation Profiles: A cfDNA fragmentation profile can include one or more cfDNA fragmentation patterns. A cfDNA fragmentation pattern can include any appropriate cfDNA fragmentation pattern. Examples of cfDNA fragmentation patterns include, without limitation, median fragment size, fragment size distribution, ratio of small cfDNA fragments to large cfDNA fragments, and the coverage of cfDNA fragments. In some embodiments, a cfDNA fragmentation pattern includes two or more (e.g., two, three, or four) of median fragment size, fragment size distribution, ratio of small cfDNA fragments to large cfDNA fragments, and the coverage of cfDNA fragments. In some embodiments, cfDNA fragmentation profile can be a genome-wide cfDNA profile (e.g., a genome-wide cfDNA profile in windows across the genome). In some embodiments, cfDNA fragmentation profile can be a targeted region profile. A targeted region can be any appropriate portion of the genome (e.g., a chromosomal region). Examples of chromosomal regions for which a cfDNA fragmentation profile can be determined as described herein include, without limitation, a portion of a chromosome (e.g., a portion of 2q, 4p, 5p, 6q, 7p, 8q, 9q, 10q, 11q, 12q, and/or 14q) and a chromosomal arm (e.g., a chromosomal mm of 8q, 13q, 11q, and/or 3p). In some embodiments, a cfDNA fragmentation profile can include two or more targeted region profiles.

In some embodiments, a cfDNA fragmentation profile can be used to identify changes (e.g., alterations) in cfDNA fragment lengths. An alteration can be a genome-wide alteration or an alteration in one or more targeted regions/loci. A target region can be any region containing one or more cancer-specific alterations. In some embodiments, a cfDNA fragmentation profile can be used to identify (e.g., simultaneously identify) from about 10 alterations to about 500 alterations (e.g., from about 25 to about 500, from about 50 to about 500, from about 100 to about 500, from about 200 to about 500, from about 300 to about 500, from about 10 to about 400, from about 10 to about 300, from about 10 to about 200, from about 10 to about 100, from about 10 to about 50, from about 20 to about 400, from about 30 to about 300, from about 40 to about 200, from about 50 to about 100, from about 20 to about 100, from about 25 to about 75, from about 50 to about 250, or from about 100 to about 200, alterations).

A cfDNA fragmentation profile can be obtained using any appropriate method. In some embodiments, cfDNA from a mammal (e.g., a mammal having, or suspected of having, cancer) can be processed into sequencing libraries which can be subjected to whole genome sequencing (e.g., low-coverage whole genome sequencing), mapped to the genome, and analyzed to determine cfDNA fragment lengths. Mapped sequences can be analyzed in non-overlapping windows covering the genome. Windows can be any appropriate size. For example, windows can be from thousands to millions of bases in length. As one non-limiting example, a window can be about 5 megabases (Mb) long. Any appropriate number of windows can be mapped. For example, tens to thousands of windows can be mapped in the genome. For example, hundreds to thousands of windows can be mapped in the genome. A cfDNA fragmentation profile can be determined within each window.

In some embodiments, methods and materials described herein also can include machine learning. For example, machine learning can be used for identifying mutation frequencies, altered fragmentation profile (e.g., using coverage of cfDNA fragments, fragment size of cfDNA fragments, coverage of chromosomes, and mtDNA).

### Methods of Treatment

The methods embodied herein, include identifying a mammal as having cancer. The methods include, extracting cell-free DNA (cfDNA) from a subject's biological sample; generating genomic libraries from the extracted cfDNA; sequencing individual cfDNA molecules to obtain mutation profiles; determining multiregional differences in mutation profiles and determining frequency of somatic mutations in the subject; and administering a cancer treatment to the subject.

In certain embodiments, a subject is diagnosed as having cancer, e.g. early stage cancer. In certain embodiments, the type of cancer is identified, and the cancer is treated by various therapeutics, including therapeutics specific for the type of cancer. In certain embodiments, the cancer comprises colorectal cancer, lung cancer, breast cancer, gastric cancers, pancreatic cancers, bile duct cancers, brain cancer or ovarian cancer. In certain embodiments, the lung cancer is small cell lung cancer (SCLC). In certain embodiments, the lung cancer is non-small cell lung cancer (NSCLC).

The cancer treatment can be surgery, adjuvant chemotherapy, neoadjuvant chemotherapy, radiation therapy, hormone therapy, cytotoxic therapy, immunotherapy, adoptive T cell therapy, targeted therapy, or any combinations thereof. The method also can include administering to the mammal a cancer treatment (e.g., surgery, adjuvant chemotherapy, neoadjuvant chemotherapy, radiation therapy, hormone therapy, cytotoxic therapy, immunotherapy, adoptive T cell therapy, targeted therapy, or any combinations thereof). The mammal can be monitored for the presence of cancer after administration of the cancer treatment.

Cancer therapies in general also include a variety of combination therapies with both chemical and radiation-based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodornbicin, streptonigrin, streptozocin, tubercidin, ubenimex, zinostatin, zombicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as rnitotane, trilostane; folic acid replenisher such as frolinic acid; aceglatone; aldophosphamide glycoside; arninolevulinic acid; eniluracil; arnsacrine; bestrabucil; bisantrene; edatraxate; defofarnine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RPS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy, or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by γ-1FN-producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CDS effector memory T cells are characterized by having the expression of CD44^{high} CD62L^{low}.

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that has an abnormal structure due to mutation can act as a cancer antigen. In principle, cancer antigens can be products of mutated Oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHIC molecules, whereas cancer cells will display the mutant version. Some viral proteins are implicated in forming cancer, and some viral antigens are also cancer antigens. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Exemplary antigens of this type are CTAG1 B and MAGEA1 as well as Rindopepimut, a 14-mer intradermal injectable peptide vaccine targeted against epidermal growth factor receptor vlll (EGFRvlll; deletion of exons 2-7) variant. Rindopepimut is particularly suitable for treating glioblastoma when used in combination with an inhibitor of the CD95/CD95L signaling system as described herein. Also, proteins that are normally produced in very low quantities, but whose production is dramatically increased in cancer cells, may trigger an immune response. An example of such a protein is the enzyme tyrosinase, which is required for melanin production. Normally tyrosinase is produced in minute quantities but its levels are very much elevated in melanoma cells. Oncofetal antigens are another important class of cancer antigens. Examples are alphafetoprotein (AFP) and carcinoembryonic antigen (CEA). These proteins are normally produced in the early stages of embryonic development and disappear by the time the immune system is fully developed. Thus, self-tolerance does not develop against these antigens. Abnormal proteins are also produced by cells infected with oncoviruses, e.g. EBV and HPV. Cells infected by these viruses contain latent viral DNA which is transcribed, and the resulting protein produces an immune response. A cancer vaccine may include a peptide cancer vaccine, which in some embodiments is a personalized peptide vaccine. In some embodiments, the peptide cancer vaccine is a multivalent long peptide vaccine, a multi-peptide vaccine, a peptide cocktail vaccine, a hybrid peptide vaccine, or a peptide-pulsed dendritic cell vaccine

The immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In some aspects, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcinoembryonic antigen, Prostate-specific membrane antigen, Mucin-1, CD30, CD33, or CD40.

Examples of monoclonal antibodies include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); lbritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (MAb17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PDl mAb); antibody to GITR; GC1008 (anti-TGF-β antibody); metelimumab/CAT-192 (anti-TGF-β antibody); lerdelimumab/CAT-152 (anti-TGF-β antibody); ID 11 (anti-TGF-β antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fe); Abatacept (Extracellular domain of CTLA-4 fused to Fe); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-α4 integrin); Vedolizumab (anti-α4 β7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

### Systems

In some examples, the present disclosure provides systems, methods, or kits that can include data analysis realized in measurement devices (e.g., laboratory instruments, such as a sequencing machine), software code that executes on computing hardware. The software can be stored in memory and execute on one or more hardware processors. The software can be organized into routines or packages that can communicate with each other. A module can comprise one or more devices/computers, and potentially one or more software routines/packages that execute on the one or more devices/computers. For example, an analysis application or system can include at least a data receiving module, a data pre-processing module, a data analysis module (which can operate on one or more types of genomic data), a data interpretation module, or a data visualization module.

The data receiving module can connect laboratory hardware or instrumentation with computer systems that process laboratory data. The data pre-processing module can perform operations on the data in preparation for analysis. Examples of operations that can be applied to the data in the pre-processing module include affine transformations, denoising operations, data cleaning, reformatting, or subsampling. The data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. The data interpretation module can use analysis methods, for example, drawn from statistics, mathematics, or biology, to support understanding of the relation between the identified abnormal patterns and health conditions, functional states, prognoses, or risks. The data analysis module and/or the data interpretation module can include one or more machine learning models, which can be implemented in hardware, e.g., which executes software that embodies a machine learning model. The data visualization module can use methods of mathematical modeling, computer graphics, or rendering to create visual representations of data that can facilitate the understanding or interpretation of results. The present disclosure provides computer systems that are programmed to implement methods of the disclosure.

In some embodiments, the methods disclosed herein can include computational analysis on nucleic acid sequencing data of samples from an individual or from a plurality of individuals. An analysis can identify a variant inferred from sequence data to identify sequence variants based on probabilistic modeling, statistical modeling, mechanistic modeling, network modeling, or statistical inferences. Non-limiting examples of analysis methods include principal component analysis, autoencoders, singular value decomposition, Fourier bases, wavelets, discriminant analysis, regression, support vector machines, tree-based methods, networks, matrix factorization, and clustering. Non-limiting examples of variants include a germline variation or a somatic mutation. In some examples, a variant can refer to an already-known variant. The already-known variant can be scientifically confirmed or reported in literature. In some examples, a variant can refer to a putative variant associated with a biological change. A biological change can be known or unknown. In some examples, a putative variant can be reported in literature, but not yet biologically confirmed. Alternatively, a putative variant is never reported in literature, but can be inferred based on a computational analysis disclosed herein. In some examples, germline variants can refer to nucleic acids that induce natural or normal variations.

In certain embodiments, the computer system includes a central processing unit (CPU, also “processor” and “computer processor” herein), which can be a single core or multi core processor, or a plurality of processors for parallel processing; memory (e.g., cache, random-access memory, read-only memory, flash memory, or other memory); electronic storage unit (e.g., hard disk), communication interface (e.g., network adapter) for communicating with one or more other systems; and peripheral devices, such as adapters for cache, other memory, data storage and/or electronic display. The memory, storage unit, interface and peripheral devices may be in communication with the CPU through a communication bus (solid lines), such as a motherboard. The storage unit can be a data storage unit (or data repository) for storing data. One or more analyte feature inputs can be entered from the one or more measurement devices. Example analytes and measurement devices are described herein.

The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing over the network (“the cloud”) to perform various aspects of analysis, calculation, and generation of the present disclosure, such as, for example, activation of a valve or pump to transfer a reagent or sample from one chamber to another or application of heat to a sample (e.g., during an amplification reaction), other aspects of processing and/or assaying a sample, performing sequencing analysis, measuring sets of values representative of classes of molecules, identifying sets of features and feature vectors from assay data, processing feature vectors using a machine learning model to obtain output classifications, and training a machine learning model (e.g., iteratively searching for optimal values of parameters of the machine learning model). Such cloud computing may be provided by cloud computing platforms such as, for example, Amazon Web Services (AWS), Microsoft Azure, Google Cloud Platform, and IBM cloud. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions can be stored in a memory location, such as the memory. The instructions can be directed to the CPU, which can subsequently program or otherwise configure the CPU to implement methods of the present disclosure. The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system such as, for example, on the memory or electronic storage unit. The machine executable or machine-readable code can be provided in the form of software. During use, the code can be executed by the CPU. In some cases, the code can be retrieved from the storage unit and stored on the memory for ready access by the CPU. In some situations, the electronic storage unit can be precluded, and machine-executable instructions are stored on memory.

The code can be pre-compiled and configured for use with a machine having a processer adapted to execute the code or can be compiled during runtime. The code can be supplied in a programming language that can be selected to enable the code to execute in a pre-compiled or as compiled fashion.

Aspects of the systems and methods provided herein, such as the computer system can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium, or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system.

Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media can be involved in carrying one or more sequences of one or more instructions to a processor for execution.

The computer system can include or be in communication with an electronic display that comprises a user interface (UI) for providing, for example, a current stage of processing or assaying of a sample (e.g., a particular step, such as a lysis step, or sequencing step that is being performed). Inputs are received by the computer system from one or more measurement. Examples of UIs include, without limitation, a graphical user interface (GUI) and web-based user interface. The algorithm can, for example, process and/or assay a sample, perform sequencing analysis, measure sets of values representative of classes of molecules, identify sets of features and feature vectors from assay data, process feature vectors using a machine learning model to obtain output classifications, and train a machine learning model (e.g., iteratively search for optimal values of parameters of the machine learning model).

In some embodiments, systems capable of executing one or more algorithms, e.g., laptops, desktops, iPads, mobile devices etc., for determining changes in cfDNA mutation profiles, frequency of mutations and/or fragmentation profiles classifies the subject as a cancer patient based on the cfDNA mutation profiles, frequency of mutations and/or fragmentation for the subject. These systems further execute machine learning algorithms that can be used to generate models such as, for example, high-risk populations and low-risk general populations (a penalized logistic regression with the Mathios et al. (Mathios D, Johansen J S, Cristiano S, Medina J E, Phallen J, Larsen K R, et al. Detection and characterization of lung cancer using cell-free DNA fragmentomes. Nat Commun 2021; 12(1):5060) features as well as coverage from transcription factor binding sites. These models can be trained on the subject cohort with 5-fold cross validation with 10 repeats, and scores for each sample ae calculated by the mean across repeats and evaluated using AUC-ROC. For example, the first model used the high-risk non-cancer and HCC patients while the second used the non-cancer individuals without liver pathology. The locked high-risk model trained on the cohort was applied to a second and different cohort to generate cancer predictions on an external validation set. A “class label” can be applied to each sample indicating the classification of the sample for any number of input features. For example, the class labels for the set of cohorts could indicate the identity of cfDNA mutation profiles, frequency of mutations and/or fragmentation profiles based on genomic location etc. The resulting training sets are provided to machine learning unit, such as a neural network or a support vector machine. Using the training set, the machine learning unit may generate a model to classify the sample according to the cfDNA mutation profiles, frequency of mutations and/or fragmentation profile.

In some embodiments, a method is provided for creating a trained classifier, comprising the steps of: (a) providing a plurality of different classes, wherein each class represents a set of subjects with a shared characteristic (e.g. from one or more cohorts); (b) providing a multi-parametric model representative of the cell-free DNA molecules from each of a plurality of samples belonging to each of the classes, thereby providing a training data set; and (c) training a learning algorithm on the training data set to create one or more trained classifiers, wherein each trained classifier classifies a test sample into one or more of the plurality of classes.

As an example, a trained classifier may use a learning algorithm selected from the group consisting of: a random forest, a neural network, a support vector machine, and a linear classifier. Each of the plurality of different classes may be selected from the group consisting of healthy, breast cancer, colon cancer, lung cancer, pancreatic cancer, prostate cancer, ovarian cancer, melanoma, and liver cancer.

A trained classifier may be applied to a method of classifying a sample from a subject. This method of classifying may comprise: (a) providing a multi-parametric model representative of the cell-free DNA molecules from a test sample from the subject; and (b) classifying the test sample using a trained classifier. After the test sample is classified into one or more classes, a therapeutic intervention on the subject can be performed based on the classification of the sample.

In some embodiments, training sets are provided to a machine learning unit, such as a neural network or a support vector machine. Using the training set, the machine learning unit may generate a model to classify the sample according to a treatment response to one or more therapeutic inventions. This is also referred to as “calling”. The model developed may employ information from any part of a test vector.

In general, machine learning can be used to reduce a set of data generated from all (primary sample/analytes/test) combinations into an optimal predictive set of features, e.g., which satisfy specified criteria. In various examples statistical learning, and/or regression analysis can be applied. Simple to complex and small to large models making a variety of modeling assumptions can be applied to the data in a cross-validation paradigm. Simple to complex includes considerations of linearity to non-linearity and non-hierarchical to hierarchical representations of the features. Small to large models includes considerations of the size of basis vector space to project the data onto as well as the number of interactions between features that are included in the modelling process.

Machine learning techniques can be used to assess the commercial testing modalities most optimal for cost/performance/commercial reach as defined in the initial question. A threshold check can be performed: If the method applied to a hold-out dataset that was not used in cross validation surpasses the initialized constraints, then the assay is locked, and production initiated. For example, a threshold for assay performance may include a desired minimum accuracy, positive predictive value (PPV), negative predictive value (NPV), clinical sensitivity, clinical specificity, area under the curve (AUC), or a combination thereof. For example, a desired minimum accuracy, PPV, NPV, clinical sensitivity, clinical specificity, or combination thereof may be at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. As another example, a desired minimum AUC may be at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. A subset of assays may be selected from a set of assays to be performed on a given sample based on the total cost of performing the subset of assays, subject to the threshold for assay performance, such as desired minimum accuracy, positive predictive value (PPV), negative predictive value (NPV), clinical sensitivity, clinical specificity, area under the curve (AUC), and a combination thereof. If the thresholds are not met, then the assay engineering procedure can loop back to either the constraint setting for possible relaxation or to the wet lab to change the parameters in which data was acquired. Given the clinical question, biological constraints, budget, lab machines, etc., can constrain the problem.

In certain embodiments, the computer processing of a machine learning technique can include method(s) of statistics, mathematics, biology, or any combination thereof. In various examples, any one of the computer processing methods can include a dimension reduction method, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, statistical testing and neural network.

In certain embodiments, the computer processing of a machine learning technique can include logistic regression, multiple linear regression (MLR), dimension reduction, partial least squares (PLS) regression, principal component regression, autoencoders, variational autoencoders, singular value decomposition, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, neural networks (shallow and deep), artificial neural networks, Pearson product-moment correlation coefficient, Spearman's rank correlation coefficient, Kendall tau rank correlation coefficient, or any combination thereof. In some examples, the computer processing method is a supervised machine learning method including, for example, a regression, support vector machine, tree-based method, and neural network. In some examples, the computer processing method is an unsupervised machine learning method including, for example, clustering, network, principal component analysis, and matrix factorization.

For supervised learning, training samples (e.g., in thousands) can include measured data (e.g., of various analytes) and known labels, which may be determined via other time-consuming processes, such as imaging of the subject and analysis by a trained practitioner. Example labels can include classification of a subject, e.g., discrete classification of whether a subject has cancer or not or continuous classifications providing a probability (e.g., a risk or a score) of a discrete value. A learning module can optimize parameters of a model such that a quality metric (e.g., accuracy of prediction to known label) is achieved with one or more specified criteria. Determining a quality metric can be implemented for any arbitrary function including the set of all risk, loss, utility, and decision functions. A gradient can be used in conjunction with a learning step (e.g., a measure of how much the parameters of the model should be updated for a given time step of the optimization process).

As described above, examples can be used for a variety of purposes. For example, plasma (or other sample) can be collected from subjects symptomatic with a condition (e.g., known to have the condition) and healthy subjects. Genetic data (e.g., cfDNA) can be acquired analyzed to obtain a variety of different features, which can include features based on a genome wide analysis. These features can form a feature space that is searched, stretched, rotated, translated, and linearly or non-linearly transformed to generate an accurate machine learning model, which can differentiate between healthy subjects and subjects with the condition (e.g., identify a disease or non-disease status of a subject). Output derived from this data and model (which may include probabilities of the condition, stages (levels) of the condition, or other values), can be used to generate another model that can be used to recommend further procedures, e.g., recommend a biopsy or keep monitoring the subject condition.

In some embodiments, DNA from a population of several individuals can be analyzed by a set of multiplexed arrays. The data for each multiplexed array may be self-normalized using the information contained in that specific array. This normalization algorithm may adjust for nominal intensity variations observed in the two-color channels, background differences between the channels, and possible crosstalk between the dyes. The behavior of each base position may then be modeled using a clustering algorithm that incorporates several biological heuristics on mutation profiles, frequency of mutations and/or fragmentation profiles. In cases where few cfDNA fragments are observed (e.g., due to low minor-allele frequency), locations and shapes of the missing sequences may be estimated using neural networks. Depending on the profiles and percent sequence identity, a statistical score may be devised (a Training score). A score such as GenCall Score is designed to mimic evaluations made by a human expert's visual and cognitive systems. In addition, it has been evolved using the genotyping data from top and bottom strands. This score may be combined with several penalty terms (e.g., low intensity, mismatch between existing and predicted cfDNA fragments) in order to make up the Training score. The Training score is saved for use by the calling algorithm.

To call a therapeutic response, a calling algorithm may take the genetic information and treatment responses of a plurality of individuals having a disease or condition. The data may first be normalized (using the same procedure as for the clustering algorithm). The calling operation (classification) may be performed using, for example, a Bayesian model. The score for each call's Call Score can be the product of a Training Score and a data-to-model fit score. After scoring all the treatment responses, the application may compute a composite score.

In some embodiments, a training dataset comprises clinical data selected from the group consisting of cancer stage, type of surgical procedure, age, tumor grading, depth of tumor infiltration, occurrence of post-operative complications, and the presence of venous invasion. In some embodiments, the training dataset is pre-processed, comprising transforming the provided data into class-conditional probabilities.

Another embodiment uses machine learning techniques to train a statistical classifier, specifically a support vector machine, for each cancer stage category based on word occurrences in a corpus of histology reports for each patient. New reports can then be classified according to the most likely stage, facilitating the collection and analysis of population staging data.

In some embodiments, a machine learning algorithm is selected from the group consisting of: a supervised or unsupervised learning algorithm selected from support vector machine, random forest, nearest neighbor analysis, linear regression, binary decision tree, discriminant analyses, logistic classifier, and cluster analysis.

In general, a system can comprise a report generator for reporting on cancer test results and treatment options. The report generator system can be a central data processing system configured to establish communications directly with: a remote data site or laboratory, a medical practice/healthcare provider (treating professional) and/or a patient/subject through communication links. The laboratory can be medical laboratory, diagnostic laboratory, medical facility, medical practice, point-of-care testing device, or any other remote data site capable of generating subject clinical information. Subject clinical information includes but it is not limited to laboratory test data, X-ray data, examination and diagnosis. The healthcare provider or practice 26 includes medical services providers, such as doctors, nurses, home health aides, technicians and physician's assistants, and the practice is any medical care facility staffed with healthcare providers. In certain instances, the healthcare provider/practice is also a remote data site. In a cancer treatment embodiment, the subject may be afflicted with cancer, among others.

Other clinical information for a cancer subject includes the results of laboratory tests, imaging or medical procedure directed towards the specific cancer that one of ordinary skill in the art can readily identify. The list of appropriate sources of clinical information for cancer includes but it is not limited to: CT scan, MRI scan, ultrasound scan, bone scan, PET Scan, bone marrow test, barium X-ray, endoscopy, lymphangiogram, IVU (Intravenous urogram) or IVP (IV pyelogram), lumbar puncture, cystoscopy, immunological tests (anti-malignin antibody screen), and cancer marker tests.

The subject clinical information may be obtained from the laboratory manually or automatically. For simplicity of the system the information is obtained automatically at predetermined or regular time intervals. A regular time interval refers to a time interval at which the collection of the laboratory data is carried out automatically by the methods and systems described herein based on a measurement of time such as hours, days, weeks, months, years etc. In one embodiment of the invention, the collection of data and processing is carried out at least once a day. In one embodiment, the transfer and collection of data is carried out once every month, biweekly, or once a week, or once every couple of days. Alternatively, the retrieval of information may be carried out at predetermined but not regular time intervals. For instance, a first retrieval step may occur after one week and a second retrieval step may occur after one month. The transfer and collection of data can be customized according to the nature of the disorder that is being managed and the frequency of required testing and medical examinations of the subjects.

In certain embodiments, a genetic report is generated from a subject's sample, e.g. cfDNA. The polynucleotides in a sample can be sequenced, e.g., whole genome sequencing, NGS sequencing, producing a plurality of sequence reads. In some embodiments, genetic information comprises variables defining the genomic organization of cancer cells or the genomic organization of single disseminated cancer cells. In some embodiments, the genetic information comprises sequence or abundance data from one or more genetic loci in cell-free DNA from the individuals.

cfDNA genetic information is processed (72). Genetic variants can also be identified. Genetic variants include sequence variants, copy number variants and nucleotide modification variants. A sequence variant is a variation in a genetic nucleotide sequence. A copy number variant is a deviation from wild type in the number of copies of a portion of a genome. Genetic variants include, for example, single nucleotide variations (SNPs), insertions, deletions, inversions, transversions, translocations, gene fusions, chromosome fusions, gene truncations, copy number variations (e.g., aneuploidy, partial aneuploidy, polyploidy, gene amplification), abnormal changes in nucleic acid chemical modifications, abnormal changes in epigenetic patterns and abnormal changes in nucleic acid methylation. The process then determines the frequency of genetic variants in the sample containing the genetic material. Since this process is noisy, the process separates information from noise (73). The sensitivity of detecting genetic variants can be increased by increasing read depth of polynucleotides (e.g., by sequencing to a greater read depth at in a sample from a subject at two or more time points).

To increase the diagnosis confidence, a plurality of measurements can be taken. Or alternatively using measurements at a plurality of time points (e.g., 2, 3, 4, 5, 6, 7, 8, 9, 10, or more time points) to determine whether cancer is advancing, in remission or stabilized. The diagnostic confidence can be used to identify disease states. For example, cell free polynucleotides taken from a subject can include polynucleotides derived from normal cells, as well as polynucleotides derived from diseased cells, such as cancer cells. Polynucleotides from cancer cells may bear genetic variants, such as somatic cell mutations and copy number variants. When cell free polynucleotides from a sample from a subject are sequenced, and cfDNA mutation profiles, frequency of mutations and/or fragmentation profiles can be produced as described in the examples section which follows.

Numerous cancers may be detected using the methods and systems described herein. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as mutations. This phenomenon may be used to detect the presence or absence of cancers individuals using the methods and systems described herein.

In the early detection of cancers, any of the systems or methods herein described, including mutation detection or copy number variation detection may be utilized to detect cancers. These system and methods may be used to detect any number of genetic aberrations that may cause or result from cancers. These may include but are not limited to cfDNA mutation profiles, frequency of mutations, cfDNA fragmentation profiles, mutations, mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, abnormal changes in nucleic acid methylation infection and cancer.

Additionally, the systems and methods described herein may also be used to help characterize certain cancers. Genetic data produced from the system and methods of this disclosure may allow practitioners to help better characterize a specific form of cancer. Often times, cancers are heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer.

The systems and methods provided herein may be used to monitor already known cancers, or other diseases in a particular subject. This may allow either a subject or practitioner to adapt treatment options in accord with the progress of the disease. In this example, the systems and methods described herein may be used to construct genetic cfDNA mutation profiles, frequency of mutations and/or fragmentation profiles of a particular subject of the course of the disease. In some instances, cancers can progress, becoming more aggressive and genetically unstable. In other examples, cancers may remain benign, inactive or dormant. The system and methods of this disclosure may be useful in determining disease progression.

Further, the systems and methods described herein may be useful in determining the efficacy of a particular treatment option. In one example, certain treatment options may be correlated with genetic cfDNA mutation profiles, frequency of mutations and/or fragmentation profiles of cancers over time. This correlation may be useful in selecting a therapy. Additionally, if a cancer is observed to be in remission after treatment, the systems and methods described herein may be useful in monitoring residual disease or recurrence of disease.

Further, the methods of the disclosure may be used to characterize the heterogeneity of an abnormal condition in a subject, the method comprising generating a cfDNA mutation profile, frequency of mutations and/or fragmentation profile of extracellular polynucleotides in the subject, wherein the cfDNA mutation profile comprises a plurality of data resulting from profile variation and mutation analyses. In some cases, including but not limited to cancer, a disease may be heterogeneous. Disease cells may not be identical. In the example of cancer, some tumors are known to comprise different types of tumor cells, some cells in different stages of the cancer. In other examples, heterogeneity may comprise multiple foci of disease. Again, in the example of cancer, there may be multiple tumor foci, perhaps where one or more foci are the result of metastases that have spread from a primary site (also known as distant metastases).

The methods of this disclosure may be used to generate a profile, fingerprint, or set of data that is a summation of genetic information derived from different cells in a heterogeneous disease. This set of data may comprise copy number variation and mutation analyses alone or in combination.

Further, these reports are submitted and accessed electronically via the internet. Analysis of data occurs at a site other than the location of the subject. The report is generated and transmitted to the subject's location. Via an internet enabled computer, the subject accesses the reports reflecting his tumor burden.

The annotated information can be used by a health care provider to select other drug treatment options and/or provide information about drug treatment options to an insurance company. The method can include annotating the drug treatment options for a condition in, for example, the NCCN Clinical Practice Guidelines in Oncology™ or the American Society of Clinical Oncology (ASCO) clinical practice guidelines.

Reports are generated, mapping genome positions and cfDNA mutation profile variation for the subject with cancer. These reports, in comparison to other profiles of subjects with known outcomes, can indicate that a particular cancer is aggressive and resistant to treatment. The subject is monitored for a period and retested. If at the end of the period, the cfDNA mutation profiles, frequency of mutations and/or fragmentation variation profile does not vary, this may indicate that the current treatment is not working. A comparison is done with cfDNA mutation profiles of other subjects. For example, if it is determined that a change in cfDNA mutation variation indicates that the cancer is advancing, then the original treatment regimen as prescribed is no longer treating the cancer and a new treatment is prescribed.

In certain embodiments, the system receives genetic information from a DNA sequencer. The process then determines specific cfDNA alterations and frequencies thereof. These reports are submitted and accessed electronically via the internet. Analysis of data occurs at a site other than the location of the subject. The report is generated and transmitted to the subject's location. Via an internet enabled computer, the subject accesses the reports reflecting his tumor burden.

While temporal information can be used to enhance the information for cfDNA mutation profiles and frequency of mutations, other consensus methods can be applied. In other embodiments, the historical comparison can be used in conjunction with other consensus cfDNA mutation profiles, frequency of mutations and/or fragmentation profiles. Consensus cfDNA mutation profiles and frequency of mutations can be normalized against control samples. Measures of molecules mapping to reference sequences can also be compared across a genome to identify areas in the genome in which cfDNA mutation profiles and frequency of mutations varies, or remains the same. Consensus methods include, for example, linear or non-linear methods of building consensus cfDNA mutation profiles and frequency of mutations (such as voting, averaging, statistical, maximum a posteriori or maximum likelihood detection, dynamic programming, Bayesian, hidden Markov or support vector machine methods, etc.) derived from digital communication theory, information theory, or bioinformatics. After the sequence read coverage has been determined, a stochastic modeling algorithm is applied to convert the normalized nucleic acid sequence read coverage for each window region to the discrete copy number states. In some cases, this algorithm may comprise one or more of the following: Hidden Markov Model, dynamic programming, support vector machine, Bayesian network, trellis decoding, Viterbi decoding, expectation maximization, Kalman filtering methodologies and neural networks.

Artificial neural networks (NNets) mimic networks of “neurons” based on the neural structure of the brain. They process records one at a time, or in a batch mode, and “learn” by comparing their classification of the record (which, at the outset, is largely arbitrary) with the known actual classification of the record. In MLP-NNets, the errors from the initial classification of the first record is fed back into the network, and are used to modify the network's algorithm the second time around, and so on for many iterations. The neural networks use an iterative learning process in which data cases (rows) are presented to the network one at a time, and the weights associated with the input values are adjusted each time.

After all cases are presented, the process often starts over again. During this learning phase, the network learns by adjusting the weights so as to be able to predict the correct class label of input samples. Neural network learning is also referred to as “connectionist learning,” due to connections between the units. Advantages of neural networks include their high tolerance to noisy data, as well as their ability to classify patterns on which they have not been trained. One neural network algorithm is back-propagation algorithm, such as Levenberg-Marquadt. Once a network has been structured for a particular application, that network is ready to be trained. To start this process, the initial weights are chosen randomly. Then the training, or learning, begins.

The network processes the records in the training data one at a time, using the weights and functions in the hidden layers, then compares the resulting outputs against the desired outputs. Errors are then propagated back through the system, causing the system to adjust the weights for application to the next record to be processed. This process occurs over and over as the weights are continually tweaked. During the training of a network the same set of data is processed many times as the connection weights are continually refined.

In an embodiment, the training step of the machine learning unit on the training data set may generate one or more classification models for applying to a test sample. These classification models may be applied to a test sample to predict the response of a subject to a therapeutic intervention.

Comparison of sequence coverage to a control sample or reference sequence may aid in normalization across windows. In this embodiment, cell free DNAs are extracted and isolated from a readily accessible bodily fluid such as blood. For example, cell free DNAs can be extracted using a variety of methods known in the art, including but not limited to isopropanol precipitation and/or silica based purification. Cell free DNAs may be extracted from any number of subjects, such as subjects without cancer, subjects at risk for cancer, or subjects known to have cancer (e.g. through other means).

Following the isolation/extraction step, any of a number of different sequencing operations may be performed on the cell free polynucleotide sample. Samples may be processed before sequencing with one or more reagents (e.g., enzymes, unique identifiers (e.g., barcodes), probes, etc.). In some cases, if the sample is processed with a unique identifier such as a barcode, the samples or fragments of samples may be tagged individually or in subgroups with the unique identifier. The tagged sample may then be used in a downstream application such as a sequencing reaction by which individual molecules may be tracked to parent molecules.

The cell free polynucleotides can be tagged or tracked in order to permit subsequent identification and origin of the particular polynucleotide. The assignment of an identifier (e.g., a barcode) to individual or subgroups of polynucleotides may allow for a unique identity to be assigned to individual sequences or fragments of sequences. This may allow acquisition of data from individual samples and is not limited to averages of samples. In some examples, nucleic acids or other molecules derived from a single strand may share a common tag or identifier and therefore may be later identified as being derived from that strand. Similarly, all of the fragments from a single strand of nucleic acid may be tagged with the same identifier or tag, thereby permitting subsequent identification of fragments from the parent strand. In other cases, gene expression products (e.g., mRNA) may be tagged in order to quantify expression, by which the barcode, or the barcode in combination with sequence to which it is attached can be counted. In still other cases, the systems and methods can be used as a PCR amplification control. In such cases, multiple amplification products from a PCR reaction can be tagged with the same tag or identifier. If the products are later sequenced and demonstrate sequence differences, differences among products with the same identifier can then be attributed to PCR error. Additionally, individual sequences may be identified based upon characteristics of sequence data for the read themselves. For example, the detection of unique sequence data at the beginning (start) and end (stop) portions of individual sequencing reads may be used, alone or in combination, with the length, or number of base pairs of each sequence read unique sequence to assign unique identities to individual molecules. Fragments from a single strand of nucleic acid, having been assigned a unique identity, may thereby permit subsequent identification of fragments from the parent strand. This can be used in conjunction with bottlenecking the initial starting genetic material to limit diversity.

Generally, the methods and systems provided herein are useful for preparation of cell free polynucleotide sequences to a down-stream application sequencing reaction. Often, a sequencing method is next generation sequencing (NGS), classic Sanger sequencing, whole-genome bisulfite sequencing (WGSB), small-RNA sequencing, low-coverage Whole-Genome Sequencing (lcWGS), etc.

As used herein, the term “sequencing” refers to any of a number of technologies used to determine the sequence of a biomolecule, e.g., a nucleic acid such as DNA or RNA. Exemplary sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina or Applied Biosystems. In some embodiments, the sequencing method can be massively parallel sequencing, that is, simultaneously (or in rapid succession) sequencing any of at least 100, 1000, 10,000, 100,000, 1 million, 10 million, 100 million, or 1 billion polynucleotide molecules.

After sequencing, reads are assigned a quality score. A quality score may be a representation of reads that indicates whether those reads may be useful in subsequent analysis based on a threshold. In some cases, some reads are not of sufficient quality or length to perform the subsequent mapping step. Sequencing reads with a quality score at least 90%, 95%, 99%, 99.9%, 99.99% or 99.999% may be filtered out of the data set. In other cases, sequencing reads assigned a quality scored at least 90%, 95%, 99%, 99.9%, 99.99% or 99.999% may be filtered out of the data set. The genomic fragment reads that meet a specified quality score threshold are mapped to a reference genome, or a reference sequence that is known not to contain mutations. After mapping alignment, sequence reads are assigned a mapping score. A mapping score may be a representation or reads mapped back to the reference sequence indicating whether each position is or is not uniquely mappable. In instances, reads may be sequences unrelated to mutation analysis. For example, some sequence reads may originate from contaminant polynucleotides. Sequencing reads with a mapping score at least 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set. For each mappable base, bases that do not meet the minimum threshold for mappability, or low quality bases, may be replaced by the corresponding bases as found in the reference sequence.

Numerous cancers may be detected using the methods and systems described herein. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as mutations. This phenomenon may be used to detect the presence or absence of cancers individuals using the methods and systems described herein.

The types and number of cancers that may be detected may include but are not limited to blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like.

Additionally, the systems and methods described herein may also be used to help characterize certain cancers. Genetic data produced from the system and methods of this disclosure may allow practitioners to help better characterize a specific form of cancer. Often times, cancers are heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer.

The systems and methods provided herein may be used to monitor already known cancers, or other diseases in a particular subject. This may allow either a subject or practitioner to adapt treatment options in accord with the progress of the disease. In this example, the systems and methods described herein may be used to construct genetic profiles of a particular subject of the course of the disease. In some instances, cancers can progress, becoming more aggressive and genetically unstable. In other examples, cancers may remain benign, inactive or dormant. The system and methods of this disclosure may be useful in determining disease progression.

Further, the systems and methods described herein may be useful in determining the efficacy of a particular treatment option. In one example, successful treatment options may actually increase the amount of copy number variation or mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the systems and methods described herein may be useful in monitoring residual disease or recurrence of disease.

The data is sent over a direct connection or over the internet to a computer for processing. The data processing aspects of the system can be implemented in digital electronic circuitry, or in computer hardware, firmware, software, or in combinations of them. Data processing apparatus of the invention can be implemented in a computer program product tangibly embodied in a machine-readable storage device for execution by a programmable processor; and data processing method steps of the invention can be performed by a programmable processor executing a program of instructions to perform functions of the invention by operating on input data and generating output. The data processing aspects of the invention can be implemented advantageously in one or more computer programs that are executable on a programmable system including at least one programmable processor coupled to receive data and instructions from and to transmit data and instructions to a data storage system, at least one input device, and at least one output device. Each computer program can be implemented in a high-level procedural or object-oriented programming language, or in assembly or machine language, if desired; and, in any case, the language can be a compiled or interpreted language. Suitable processors include, by way of example, both general and special purpose microprocessors. Generally, a processor will receive instructions and data from a read-only memory and/or a random access memory. Storage devices suitable for tangibly embodying computer program instructions and data include all forms of nonvolatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

To provide for interaction with a user, the methods can be implemented using a computer system having a display device such as a monitor or LCD (liquid crystal display) screen for displaying information to the user and input devices by which the user can provide input to the computer system such as a keyboard, a two-dimensional pointing device such as a mouse or a trackball, or a three-dimensional pointing device such as a data glove or a gyroscopic mouse. The computer system can be programmed to provide a graphical user interface through which computer programs interact with users. The computer system can be programmed to provide a virtual reality, three-dimensional display interface.

### EXAMPLES

### Example 1: Single Molecule Genome-Wide Mutation And Fragmentation Profiles of Cell-Free DNA For Noninvasive Detection of Lung Cancer

It was considered whether identifying somatic sequence changes across the genome could allow for detection of an increased number of tumor derived cfDNA changes and increase the ability to detect early stage disease. A tumor genome contains thousands of somatic changes^{19,20} and knowledge of such alterations from tumor tissue have been used to guide targeted analyses in the circulation during therapy^{21,22}. In principle, if such genome-wide changes could be identified directly in cfDNA without knowledge of the alterations in the tumor, they could be useful for early cancer detection. However, such an approach would require the ability to efficiently detect somatic changes and to distinguish these from the multitude of other non-tumor derived alterations.

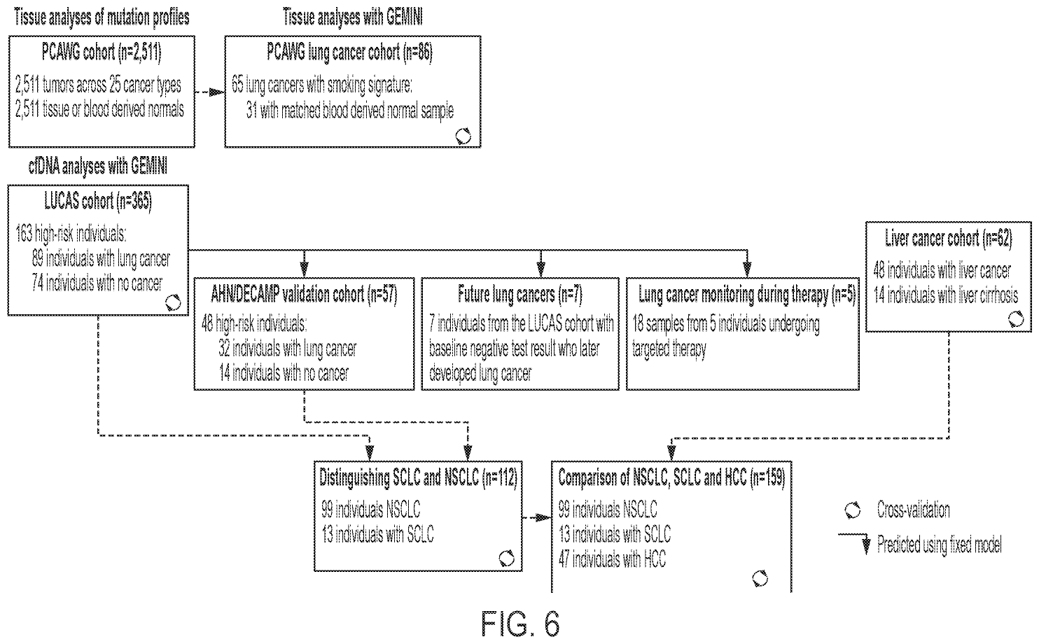

To address these challenges, an approach was developed herein, called GEnome-wide Mutational Incidence for NonInvasive detection of cancer (GEMINI), that could identify a much larger number of tumor-derived alterations in cfDNA for cancer detection (FIG. 1). This method was applied to analyze tissue and cfDNA samples from multiple patient cohorts (FIG. 6). The method is based on sequencing individual cfDNA molecules to estimate the mutation frequency and type of alteration across the genome using non-overlapping bins ranging in size from thousands to millions of bases. For each individual, the mutation type and frequency in genomic regions more commonly altered in cancer is compared to the profile from regions more frequently mutated in normal cfDNA to determine multiregional differences in mutation profiles. In this way, the GEMINI approach enriches for likely somatic mutations while taking into account individual variability in overall background changes.

### Results

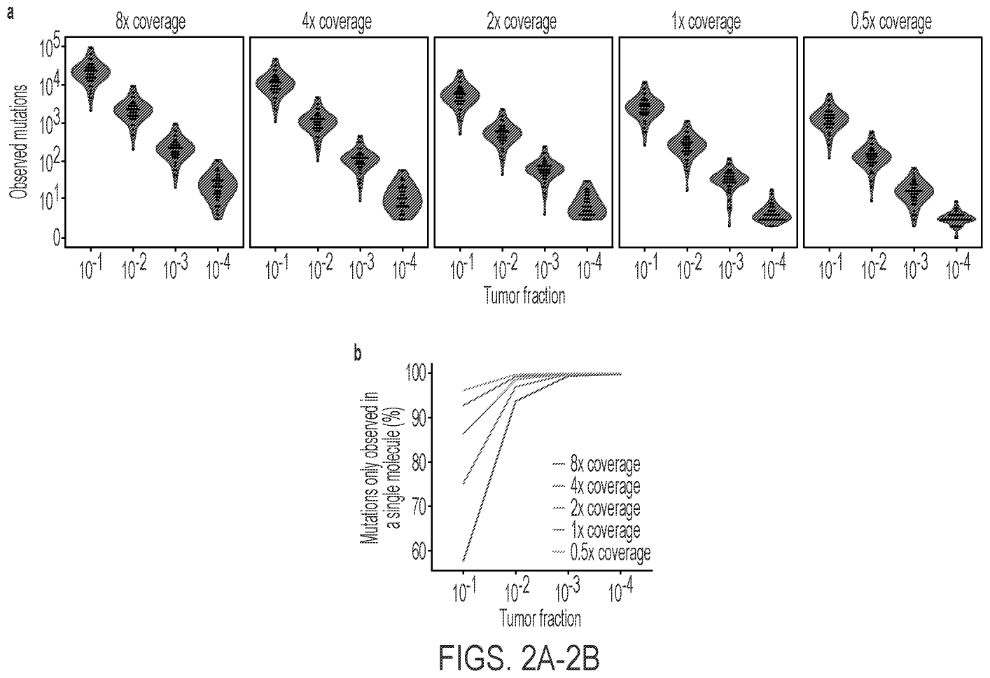

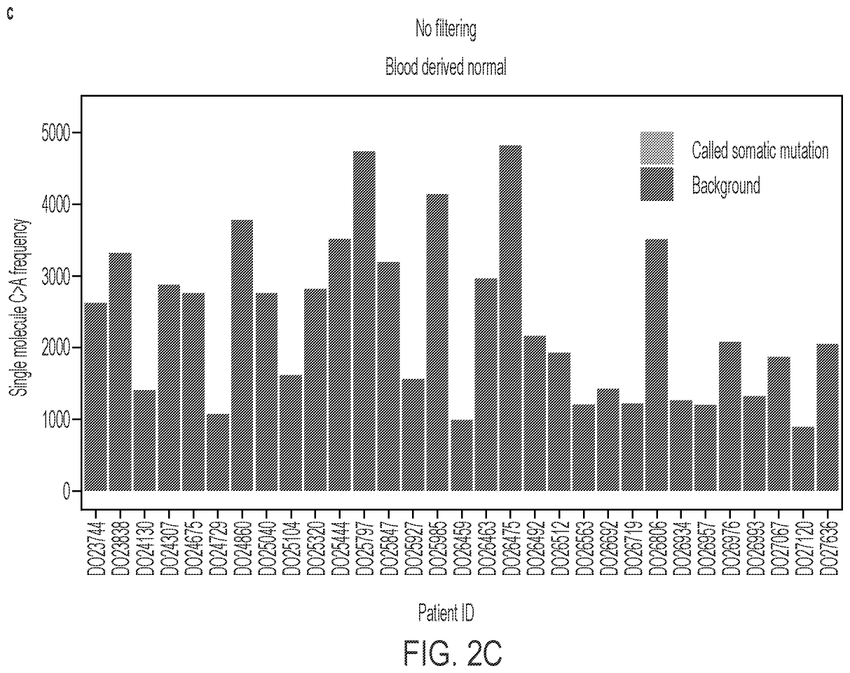

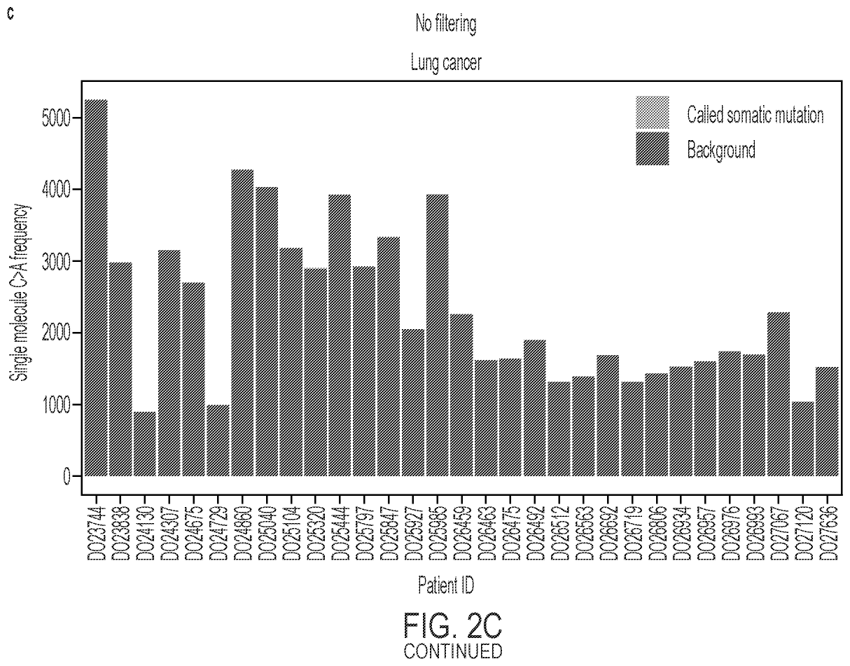

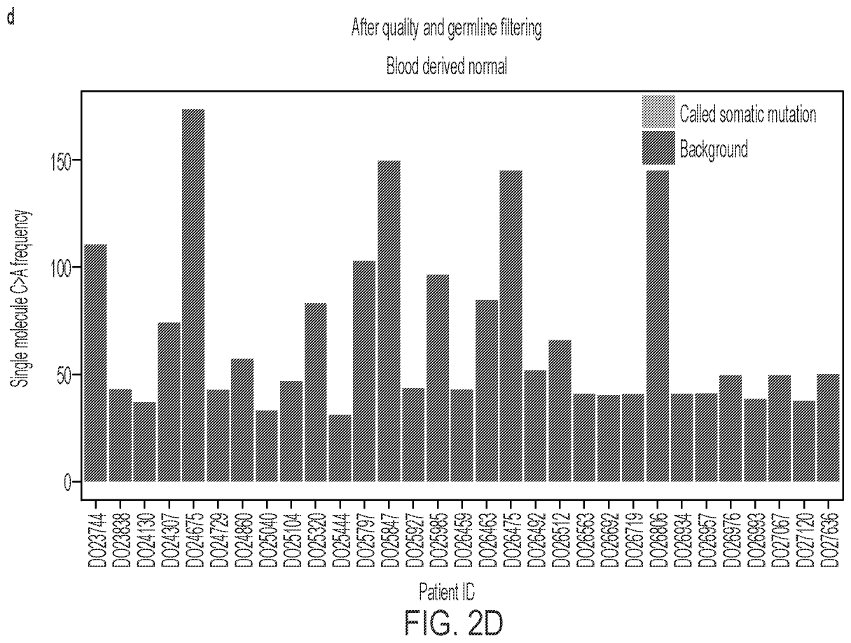

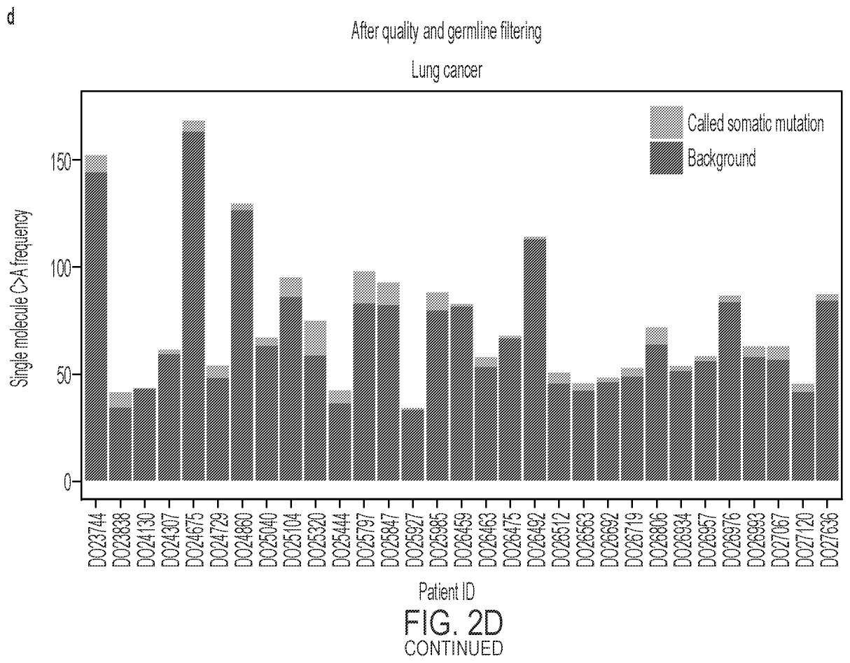

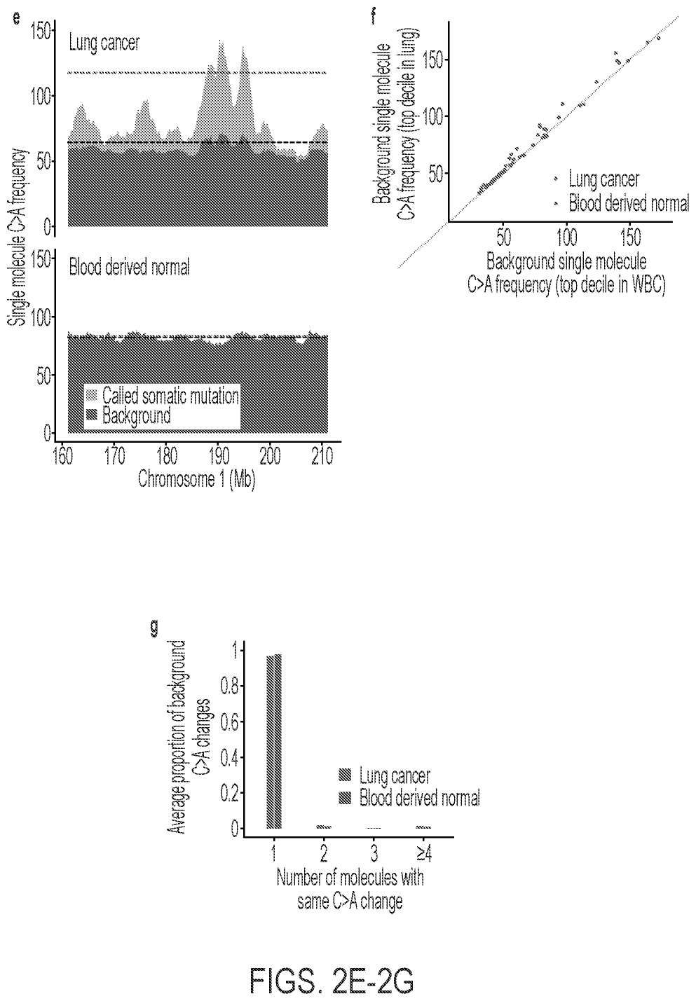

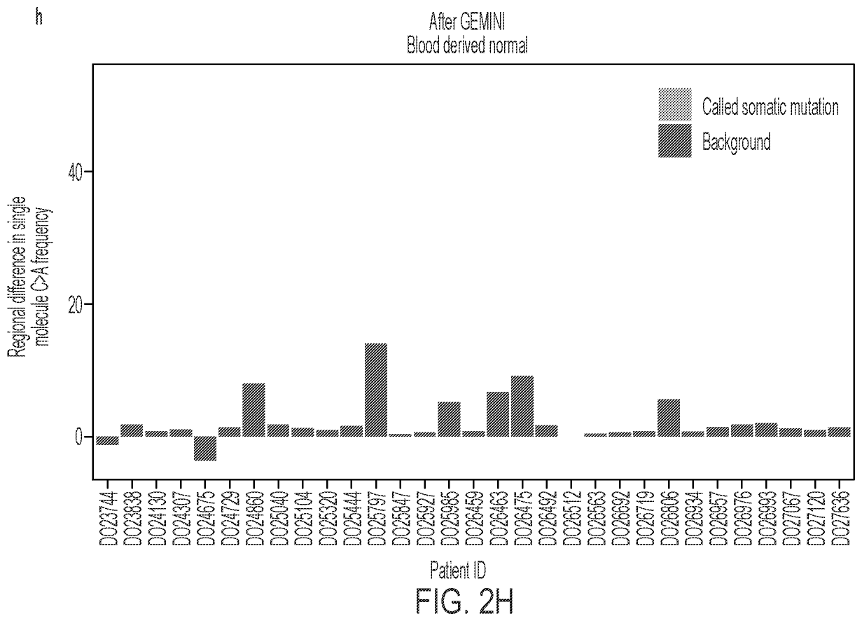

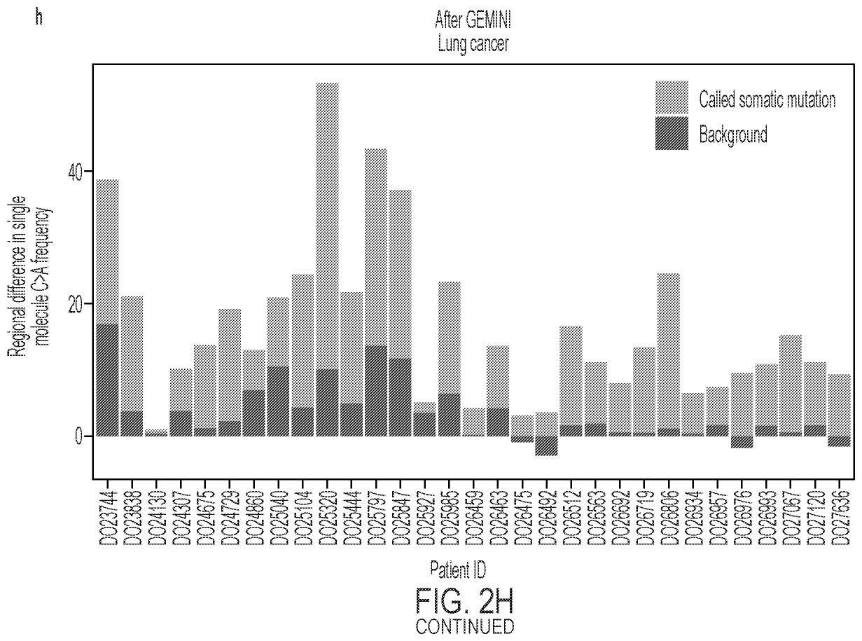

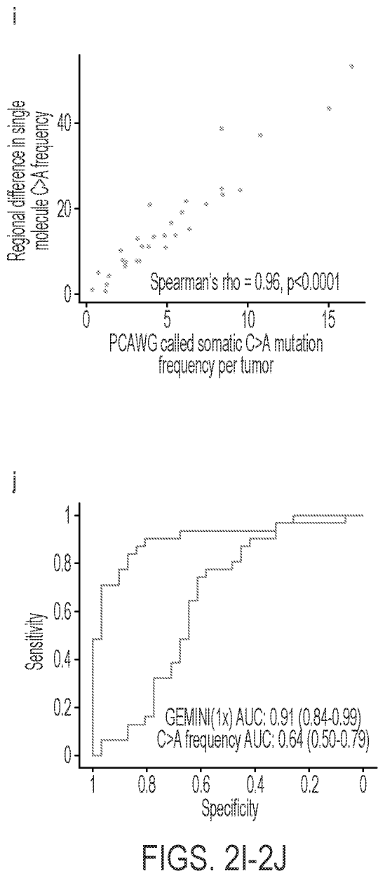

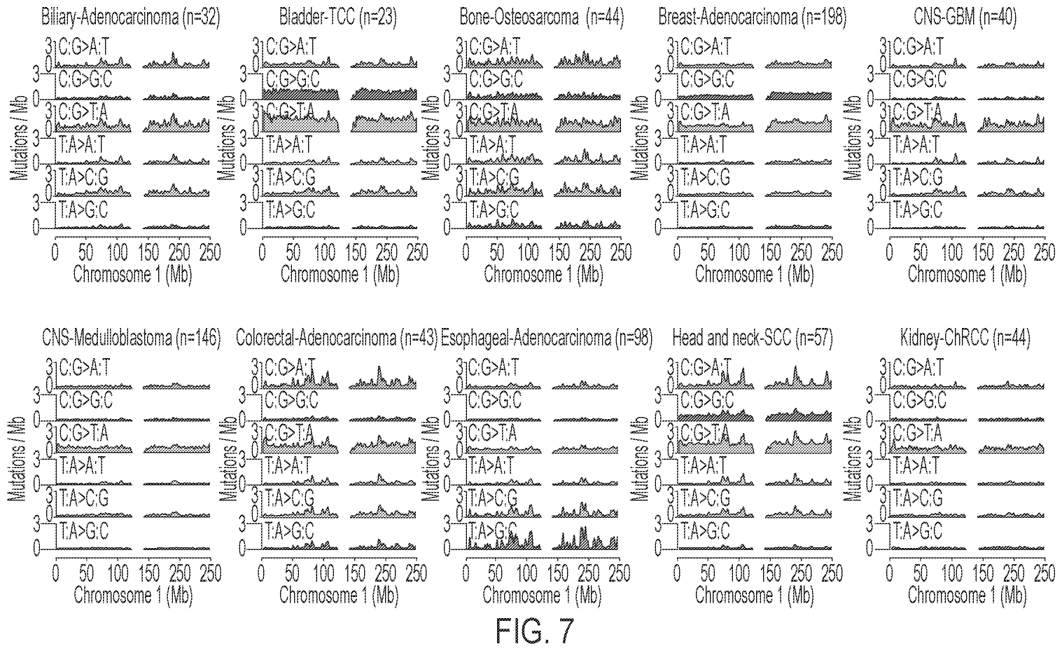

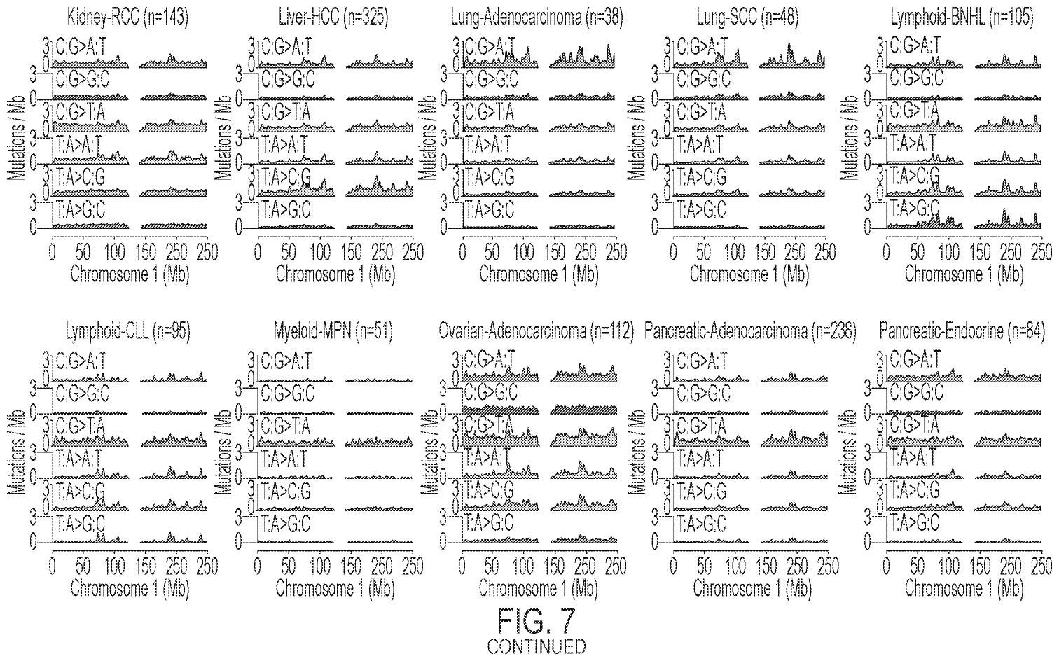

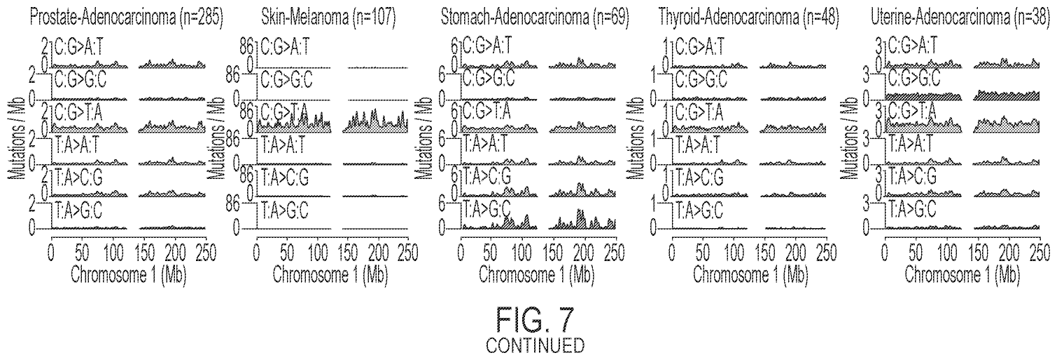

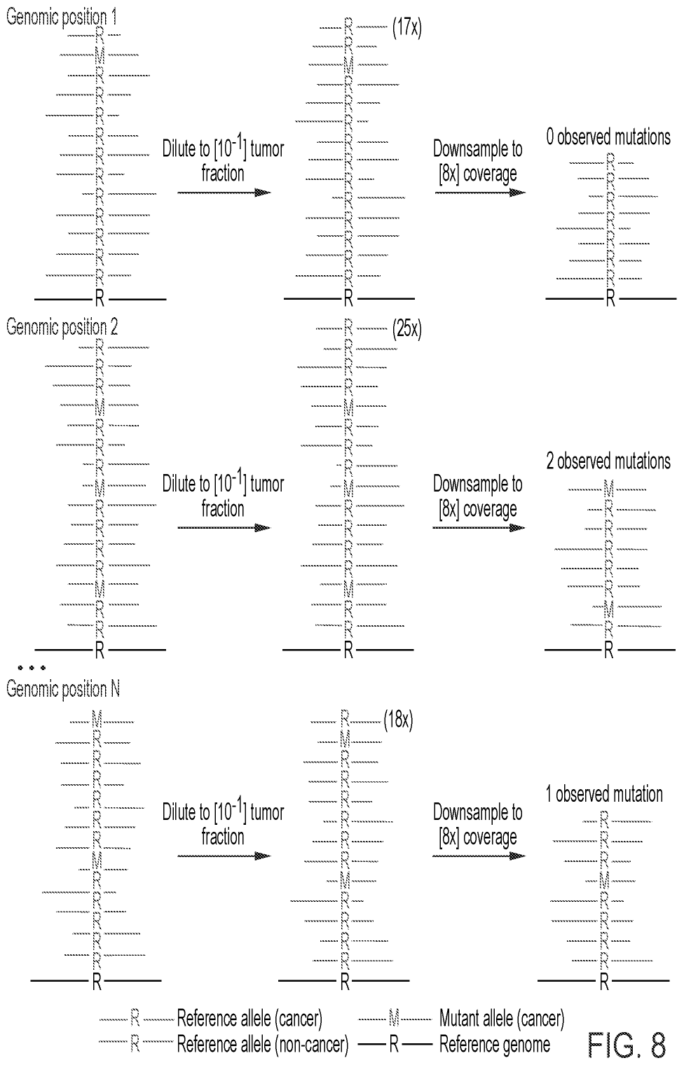

To develop this method, whole genome sequences of cancers were examined from 2,511 individuals across 25 different cancers from the Pan-Cancer Analysis of Whole Genomes (PCAWG) study^{25,26}, identifying distinct frequencies of somatic mutations across the genome in different tumor types (FIG. 7; Supplementary Table1). As an example, analysis of lung tumor and matched normal tissue genomes from 65 individuals with smoking exposure revealed that the cancers had an average of 52,209 (range 6,031 to 193,539) bonafide somatic mutations per genome. To quantify how many tumor-specific changes would be expected to see in the plasma in these individuals, in silico dilution and downsampling experiments were performed (FIG. 8). In these simulations, it was found that all patients would theoretically have a subset of detectable somatic mutations even at a tumor fraction as low as 1:10,000 when analyzed using whole genome sequencing at a coverage of 1× (FIG. 2A).

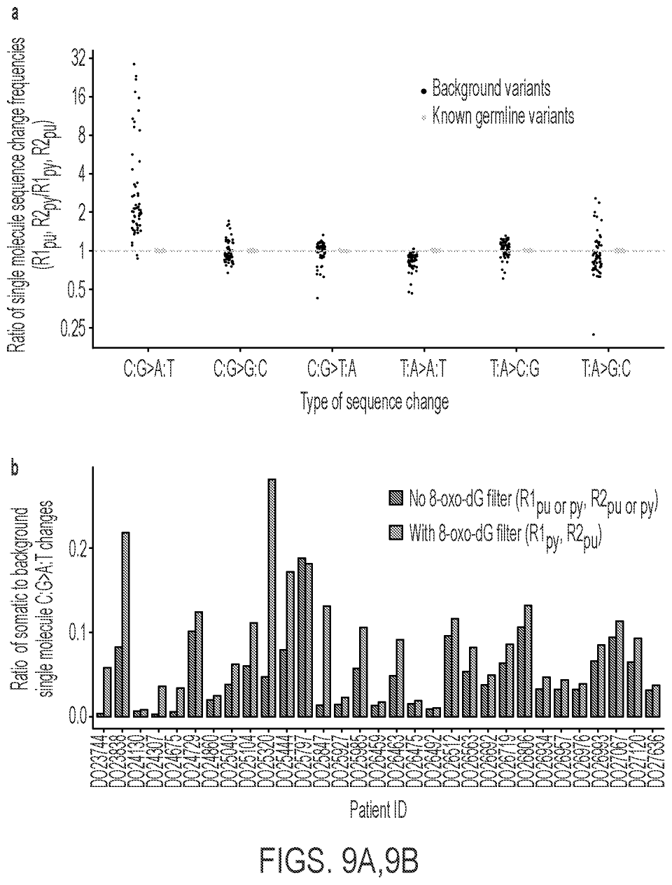

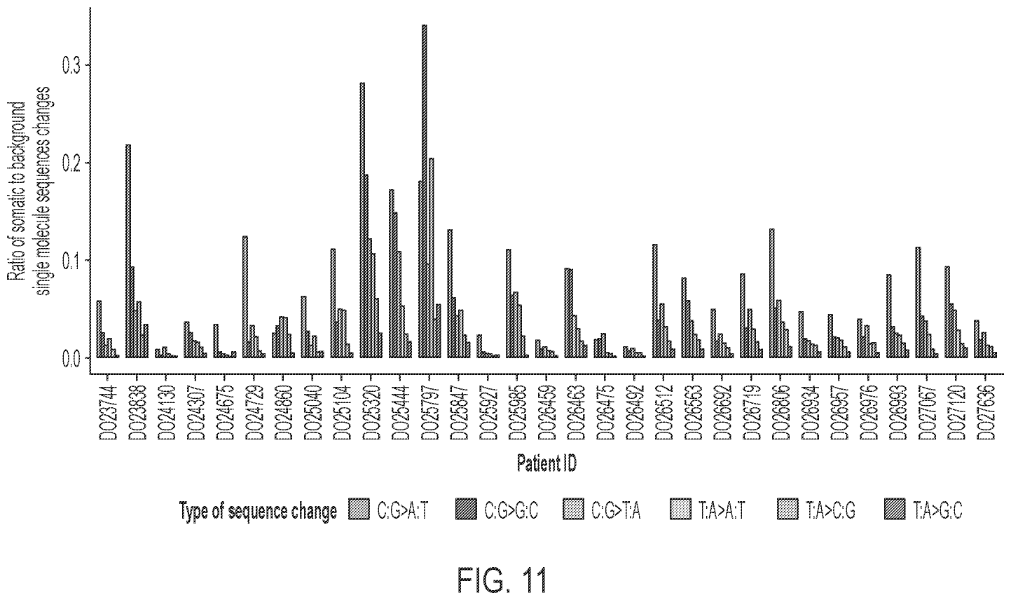

Because the majority of mutations detected at low whole genome coverage would be expected to be observed in single DNA molecules (FIG. 2B), rigorous methods were developed to examine the frequency of single molecule somatic mutations in a mixture of germline variants, WBC alterations, and experimental and sequencing artifacts (all considered background changes). Each sequenced molecule was scanned for single nucleotide changes, and after removing common germline variants and unevaluable regions, computed the frequency of putative mutations in high quality reads, defined as the number of variants per million evaluated positions across all the DNA molecules sequenced (Methods). As specific transversions likely related to accumulation of 7,8-dihydro-8-oxoguanine (8-oxo-dG)²⁷ were more highly represented among PCAWG single molecule alterations than other changes and higher than one would expect from analyses of similar transversions at sites of known polymorphisms, these changes were filtered from further consideration when they occurred in certain read combinations (FIGS. 9A, 9B, Methods). These changes were examined in the set of PCAWG lung tumors samples with matched normal blood cells (n=31), as blood cells represent the largest source of cfDNA in non-cancer individuals²⁸. The analyses were focused on the remaining C:G>A:T mutations (hereafter C>A) given their high abundance in tumors of current and former smokers²⁹. As expected, given the high and variable frequency of overall background changes, it was found that C>A mutation frequencies were similar in the tumor compared to normal samples (FIG. 2C), and were only slightly higher even after the filtering steps above and removal of germline variants, where only a small fraction of the tumor alterations was somatic in origin (average=7.5%, range 0.8%-22%) (FIGS. 2D, 10A, 11).

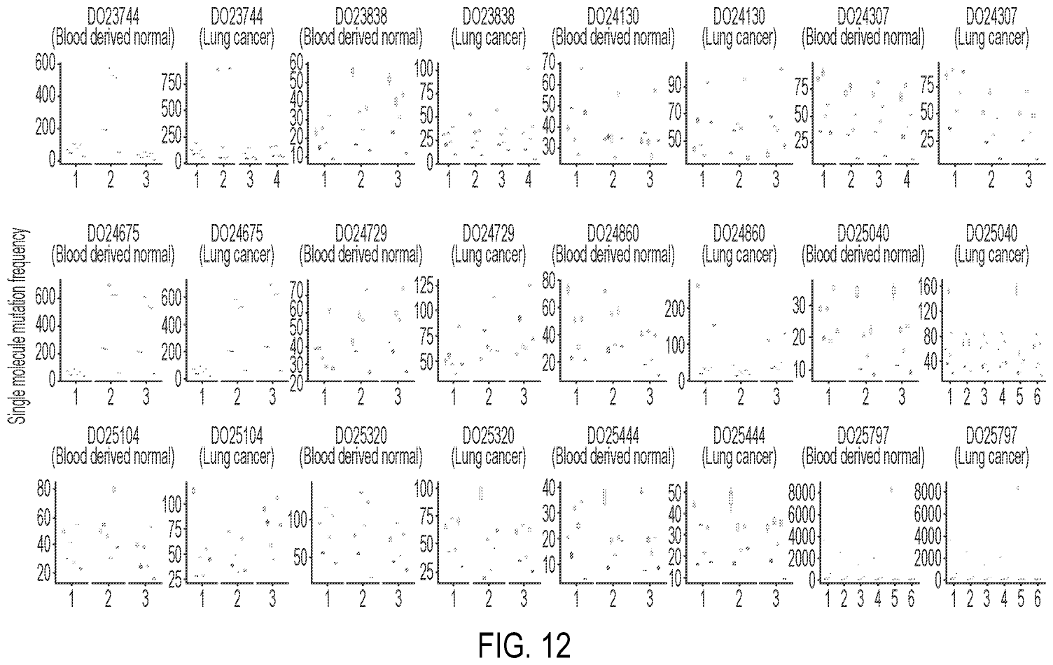

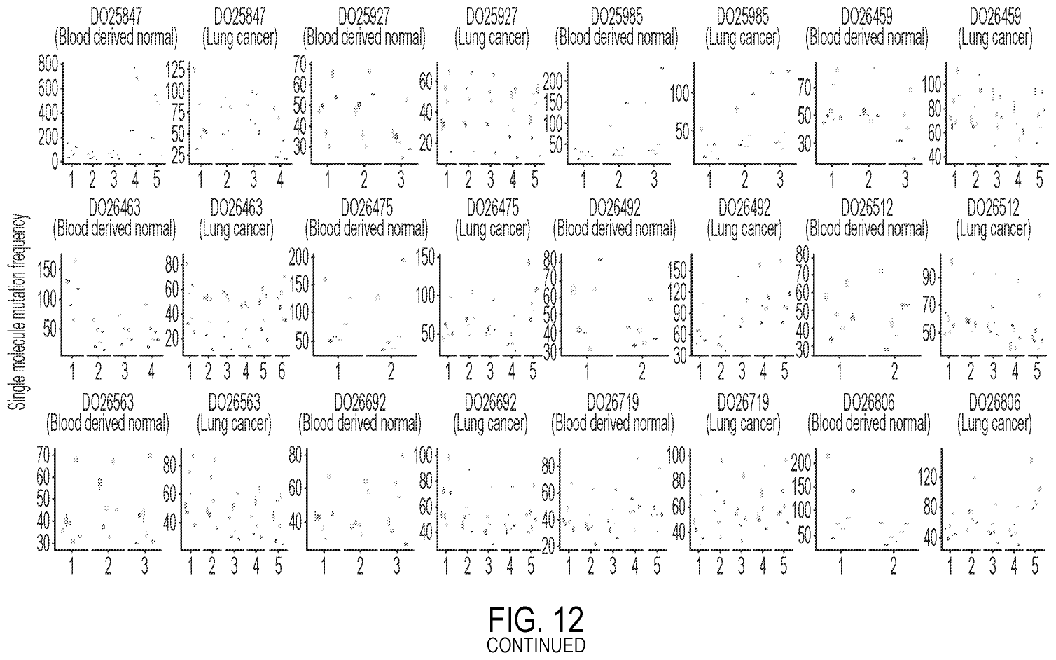

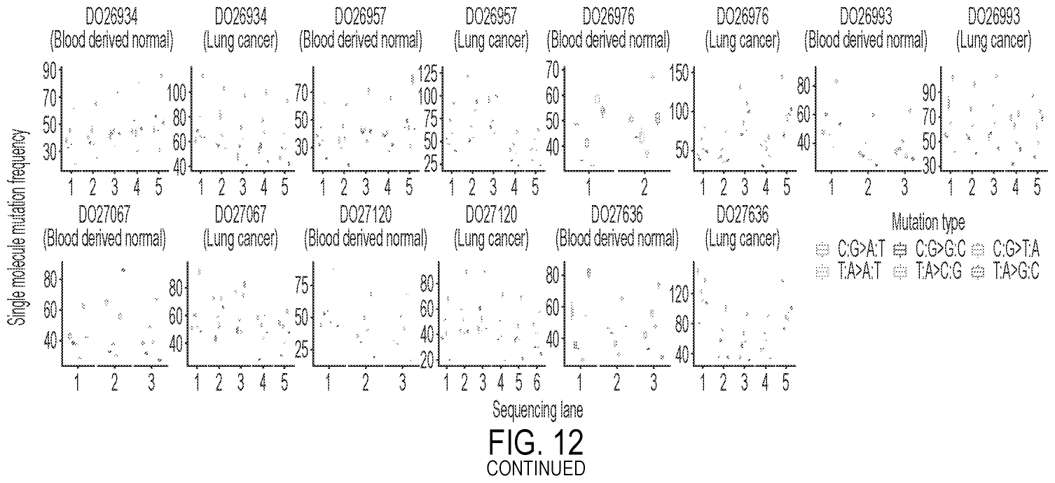

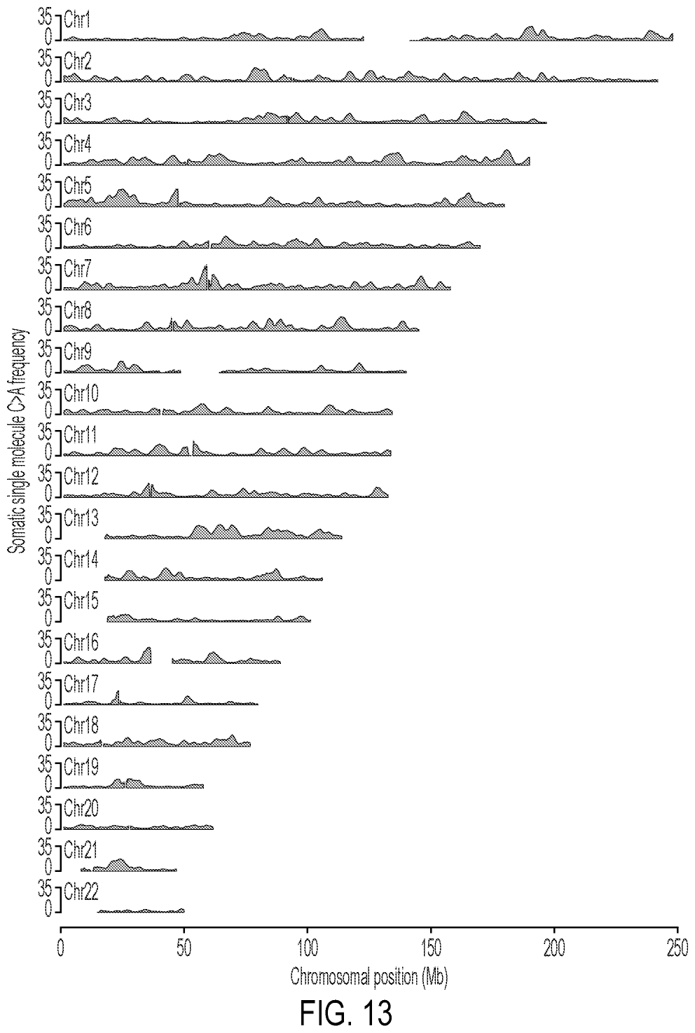

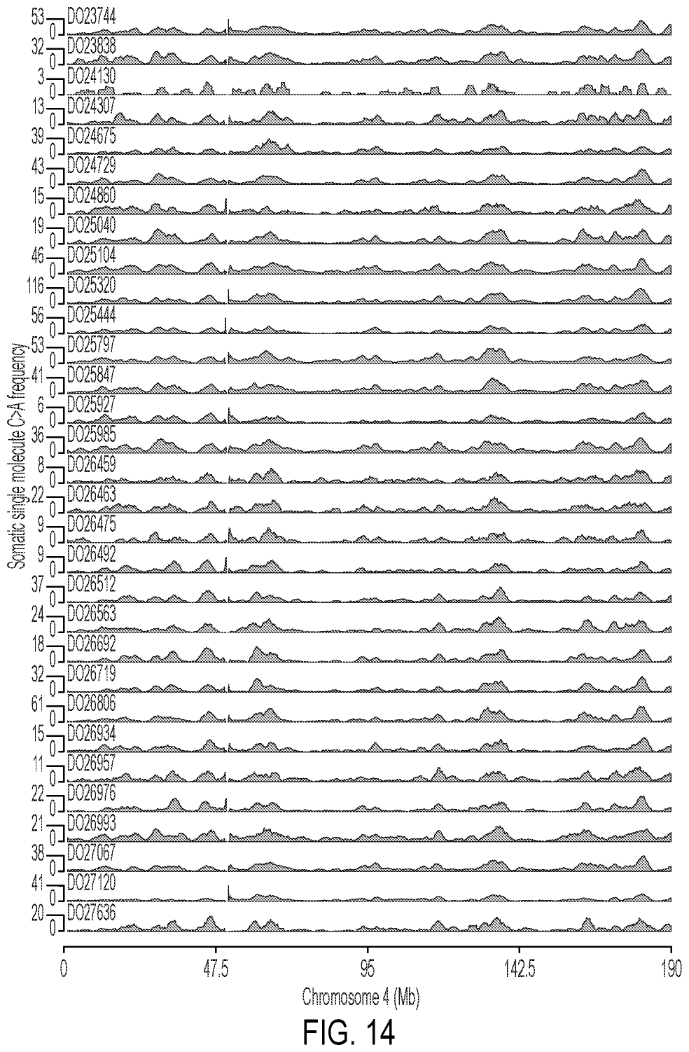

The high amount and variability of total background changes were investigated among samples, and it was found that these were largely related to sequencing lane- and run-specific artifacts (FIG. 12). It was hypothesized that controlling for overall background rates in a sample-specific manner could improve detection of tumor-derived changes. Previous analyses have shown that mutation rates differ across individual cancer genomes, with regions associated with euchromatin, including expressed genes and early-replicating regions, having a lower mutation rate compared to heterochromatin regions representing unexpressed genes and late-replicating regions^{30,31}. To examine the variation in mutation frequency across the genome, the 31 PCAWG paired samples were analyzed by binning the sequence data containing 3,076,901 mutations into 1,144 non-overlapping 2.5 megabase (Mb) bins and found regions throughout the genome with increased mutation frequencies shared by many tumors (FIGS. 13, 14).

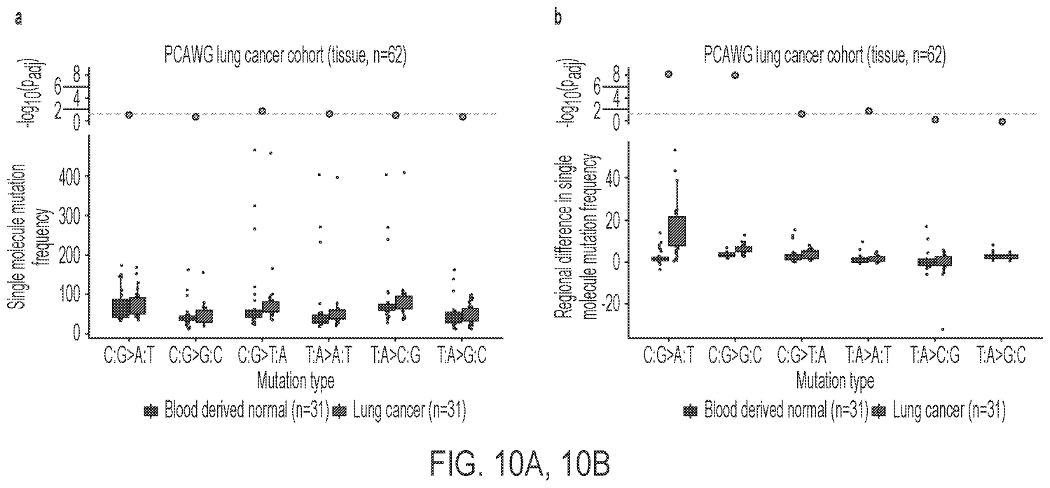

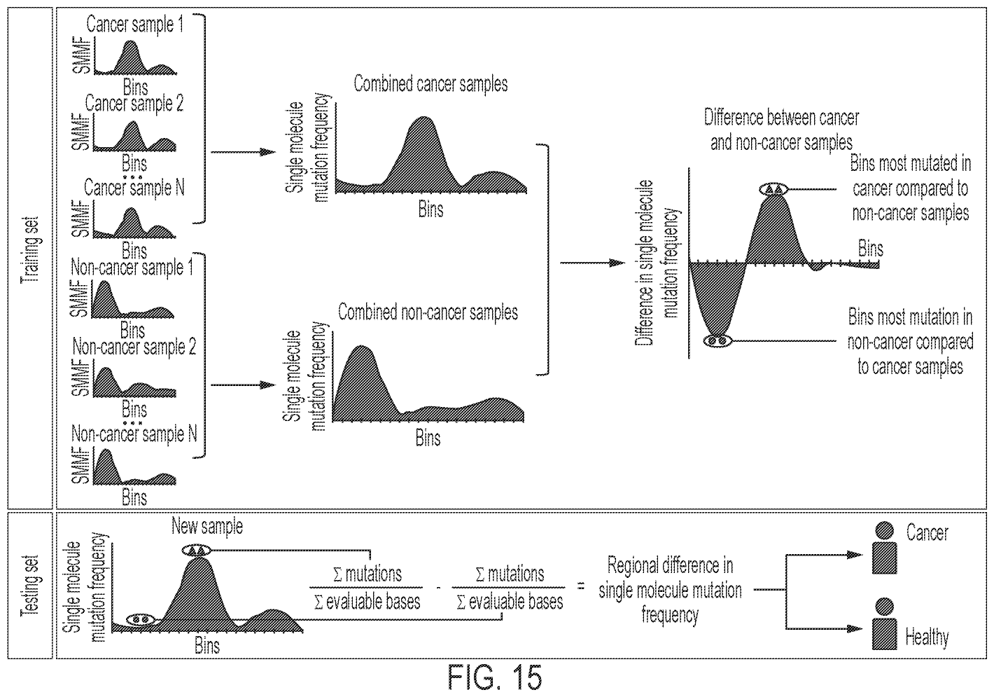

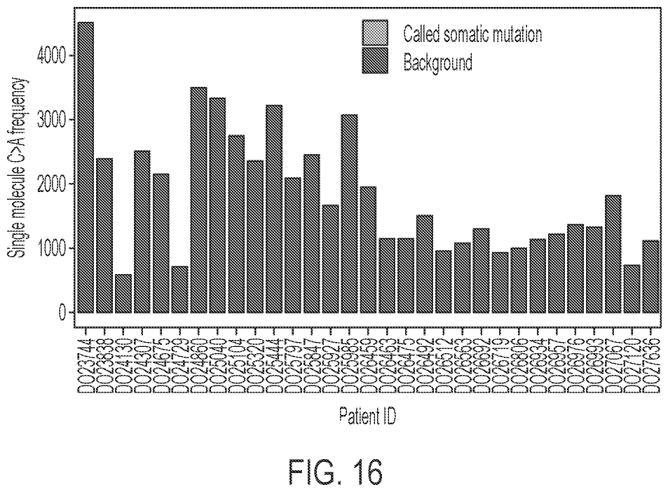

To evaluate the GEMINI approach for detection of tumor-derived DNA, we identified genomic regions with the highest C>A changes in a training set of cancers and controls and computed the average C>A difference at these regions for patients not represented in the training set (FIG. 15, Methods). Regions enriched for C>A changes were identified in the 31 PCAWG cancers but not normal samples (FIG. 2E) and it was found that background changes were highly correlated in cancer and control regions for each patient sample (Pearson correlation coefficient=0.99, p<0.0001) (FIG. 2F), suggesting that subtraction of alteration frequencies between cancer and control regions within a given patient sample would be useful for removing background mutations. In contrast, subtraction of specific mutations observed in the matched normal sample observed in the single-molecule sequencing data was not effective in removing background changes (FIG. 16) because such alterations typically occurred de novo and were seen once (FIG. 2G). After background subtraction, the remaining regional mutation frequencies were appreciably higher in tumors compared to normal samples (median of 13.4 compared to 1.3, respectively, p<0.0001, Wilcoxon rank sum test), with a high fraction of changes resulting from somatic mutations (average=80%, range=31%-100%) (FIG. 2H), and were highly correlated to the frequency of high-confidence somatic C>A changes reported in these samples by the PCAWG consortium (Pearson correlation coefficient=0.96, p<0.0001) (FIG. 2I). The GEMINI approach using C>A regional frequencies was able to distinguish PCAWG cancer from non-cancer samples with high accuracy (AUC=0.91, 95% CI=0.84-0.99) compared to mutation frequencies alone (AUC=0.64, 95% CI=0.50-0.79) using low coverage whole genome sequencing (FIGS. 2J, 10A, 10B). The overall approach for filtering background changes using variant quality filters, germline variant removal, and subtraction of regional mutation frequencies from single molecule sequencing resulted in a 1,903-fold enrichment in somatic mutations in these samples.

To determine if the GEMINI method could be used to noninvasively detect alterations in cfDNA from patients with cancer, the ability of the approach to detect sequence alterations was evaluated in individuals from a prospective lung cancer diagnostic cohort (LUCAS)¹⁸. Low coverage plasma whole genome sequence data (˜2× coverage) from the 365 individuals examined in this trial was analyzed, the majority of whom were at high-risk for lung cancer (age 50-80 and smoking history ≥20 pack years; Supplementary Table 2) and where blood samples were collected prior to clinical diagnosis. Given the short length of cfDNA fragments¹³ in addition to the filtering steps above and removal of 8-oxo-dG-related changes, the additional filter of restricting analyses to regions with identical sequence calls in overlapping reads in the paired-end library was employed. Requiring a Phred quality score ε 30 in both reads would theoretically reduce the error rate of a mutation due to sequencing errors and benefits from a higher degree of overlap for shorter tumor derived cfDNA sequences³², thereby potentially enriching detection of circulating tumor DNA (ctDNA) alterations. Supplementary Table 5 is a summary of cfDNA samples and the genomic analyses of lung cancer patients undergoing targeted therapy.

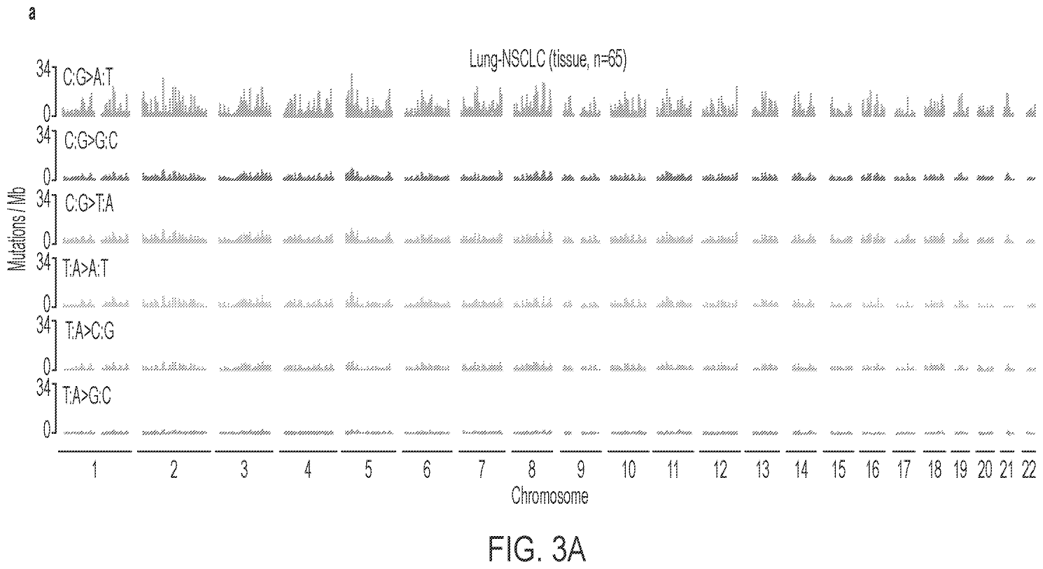

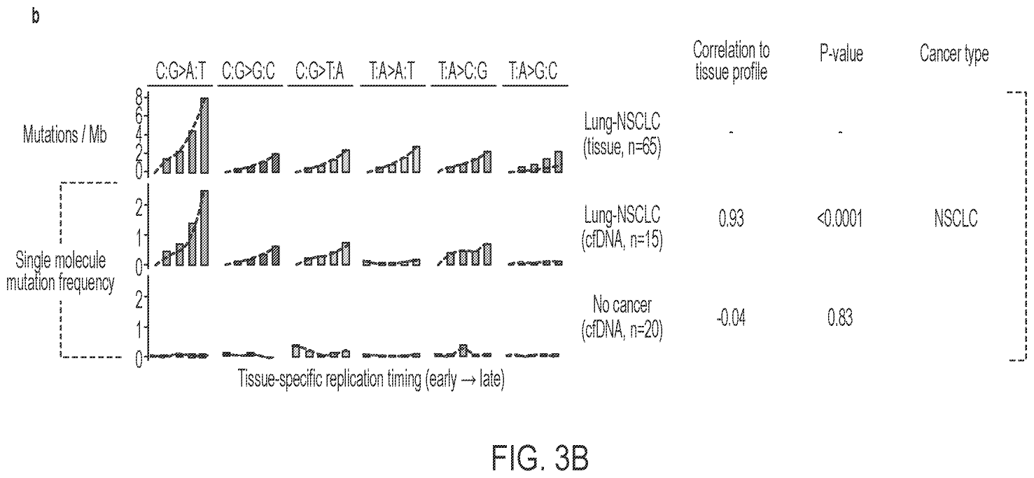

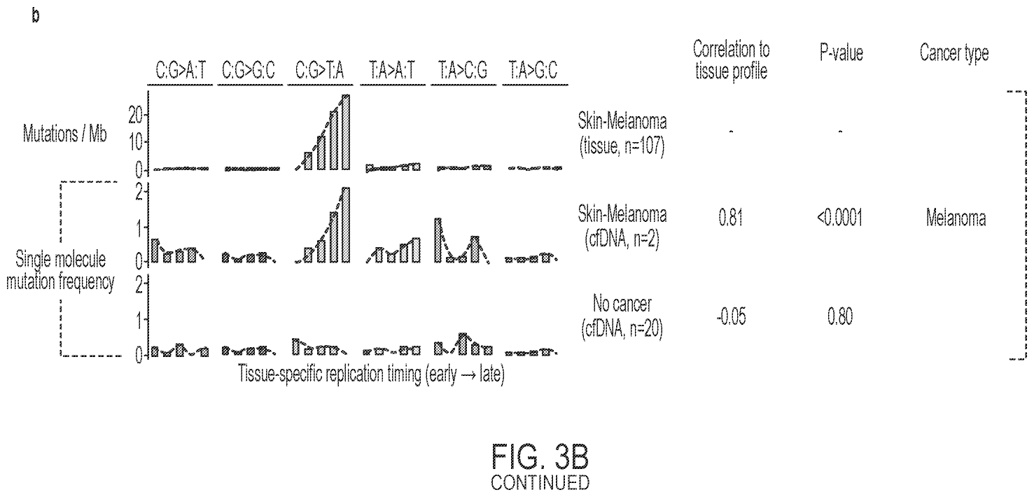

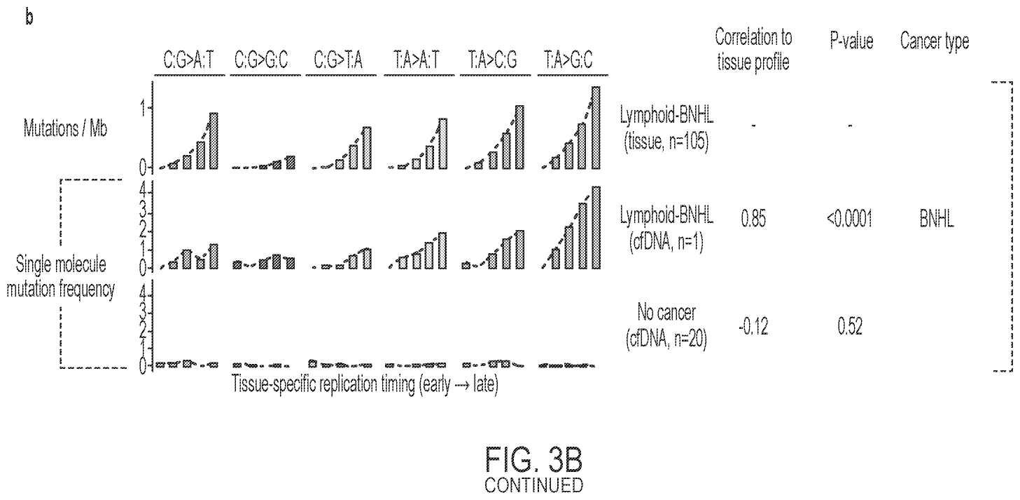

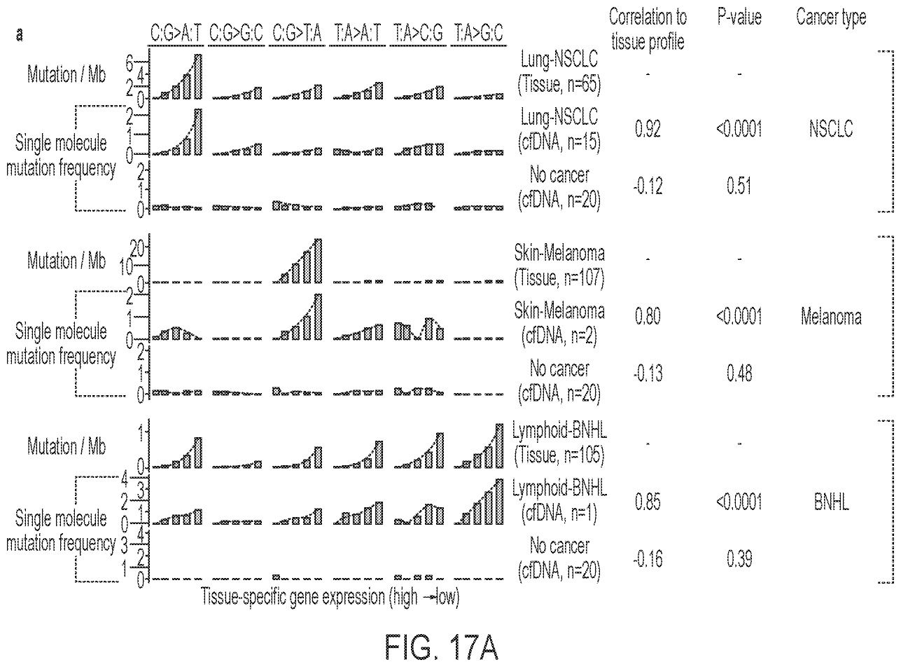

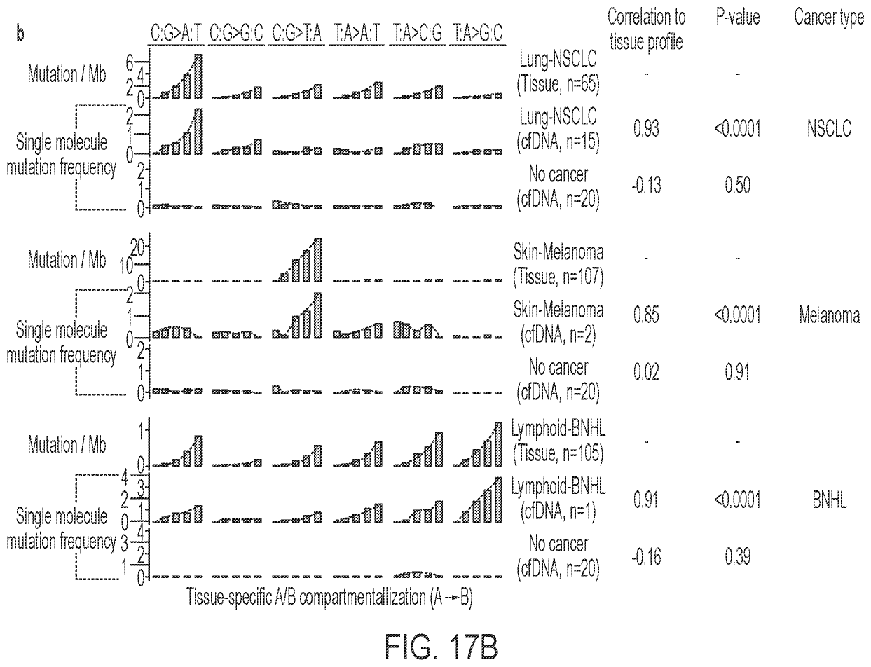

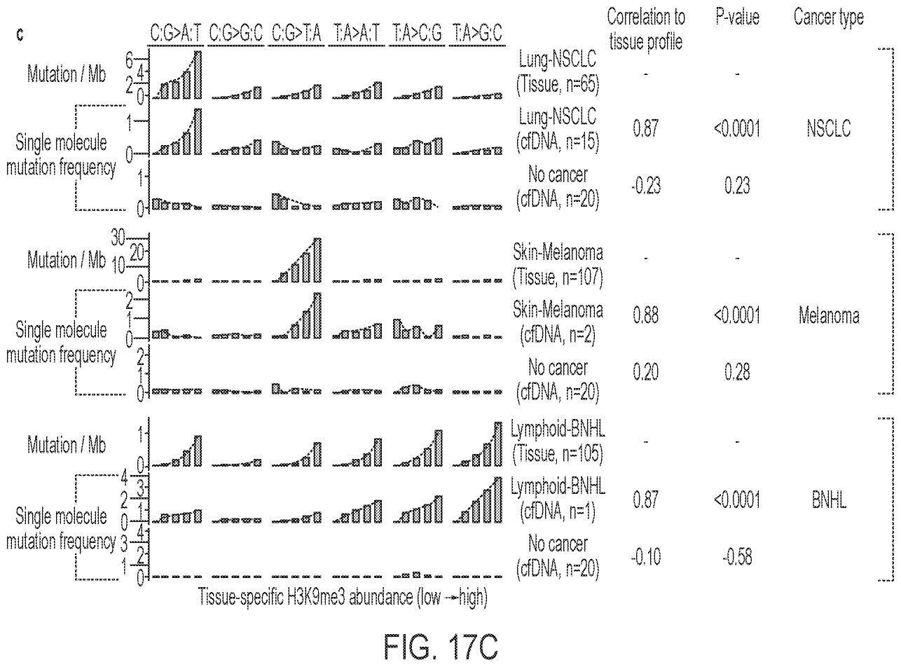

Comparison of single molecule sequencing of cfDNA from a subset of the LUCAS cohort and PCAWG tumor tissue samples revealed that the genome regions with increased frequency of cancer-type specific mutations were largely similar between tumor tissue and cfDNA of patients with lung cancer, as well as between tissue and cfDNA of patients with melanoma, and B-cell non-Hodgkin lymphoma (BNHL) (Pearson correlation >0.80, p<0.001 in all cases) and were located in areas of the genome associated with tissue-specific late replication timing (FIGS. 3A, 3B). Different mutation types among the tumors analyzed contributed to the increased mutation frequencies, including C>A changes in lung cancer, C>T in melanoma, and T>G in lymphoma. It was also found that tumor- and mutation type-specific regional mutation frequencies were related to gene expression³⁰, genome compartmentalization as measured by eigenvector analyses of methylation³³, as well histone 3 lysine 9 tri-methylation (H3K9me3), a known mark of heterochromatin³⁴, and were consistent between tumor and cfDNA analyses (Pearson correlation >0.80, p<0.001 in all cases) (FIGS. 17A-17C). Individuals without cancer or mutation types or regions not enriched in cancer did not have or were weakly correlated to these characteristics (FIGS. 3B, 17A-17C). Overall, these results suggest that mutation rate variability across the genome in cfDNA is related to chromatin organization and can be leveraged by the GEMINI approach to detect tumor-derived sequence changes in the circulation.

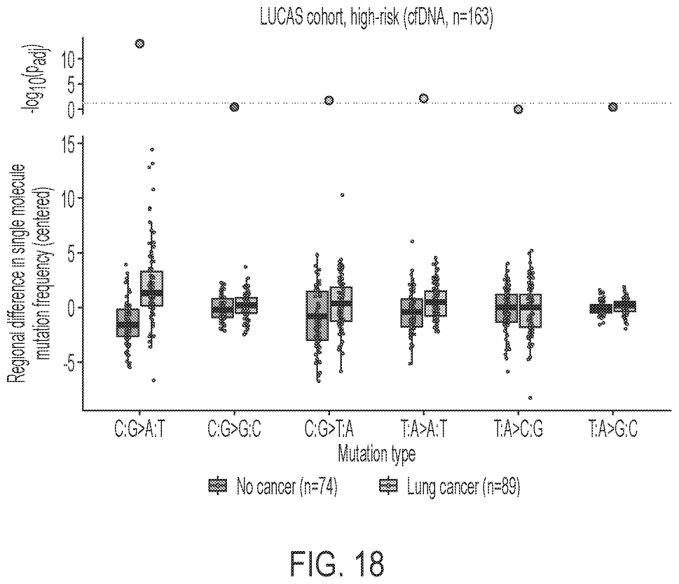

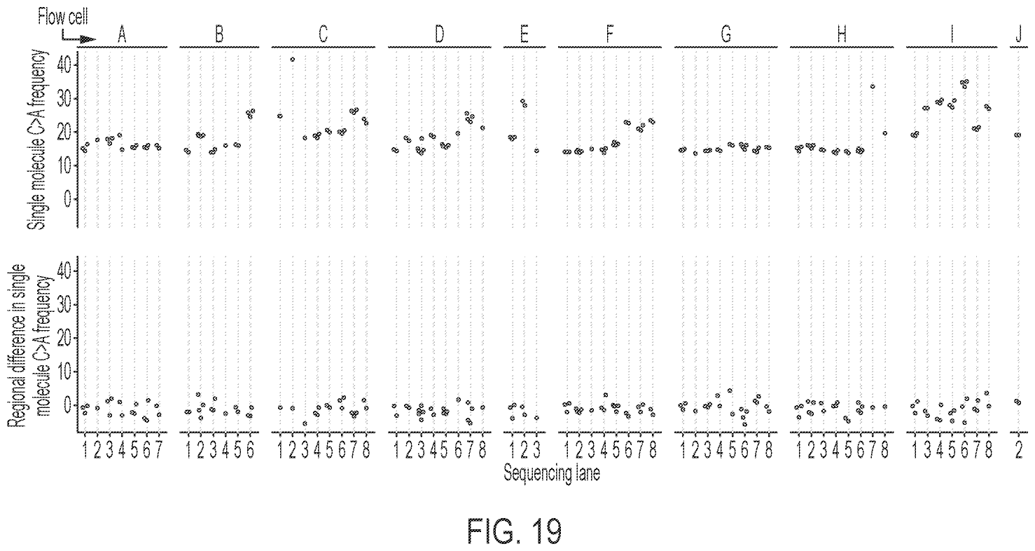

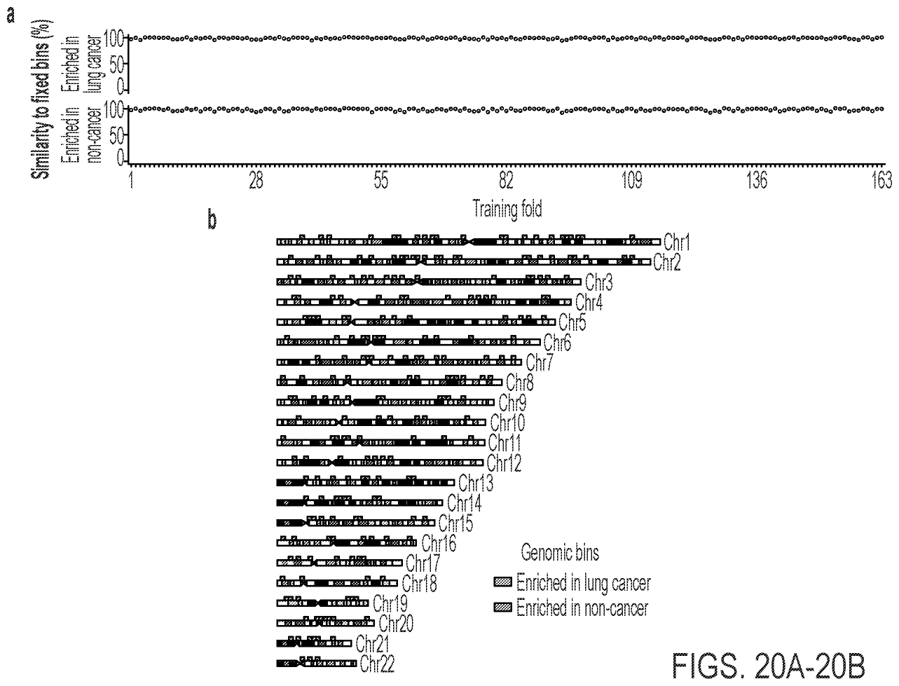

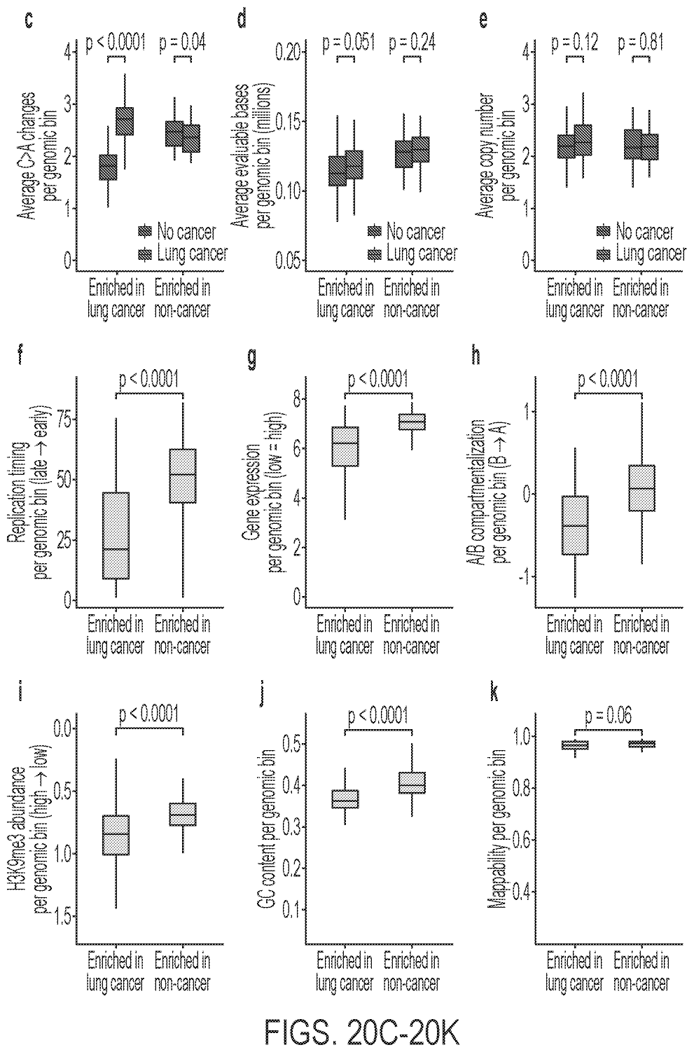

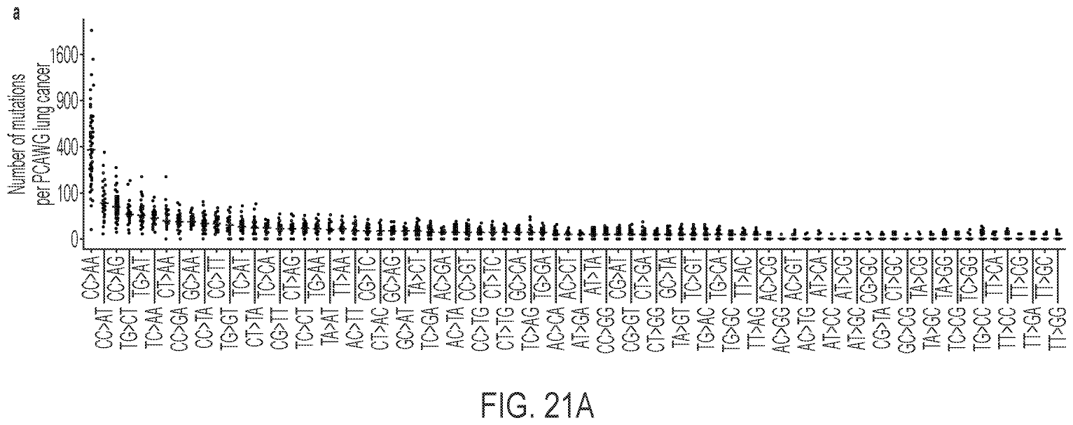

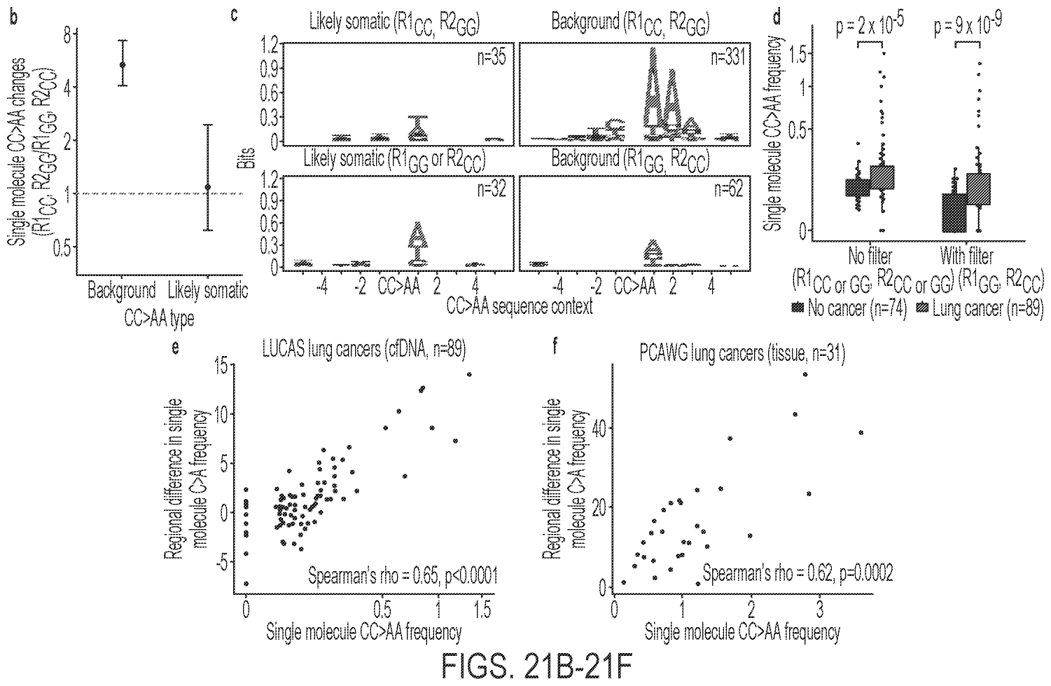

Using the GEMINI approach, cross-validated regional differences were identified in single molecule mutation frequencies for individuals in the LUCAS cohort. Similar to analyses in PCAWG lung cancers, regional C>A mutation frequencies were preferentially altered in individuals with lung cancer compared to non-cancer individuals (p<0.0001, Wilcox rank sum test) (FIG. 18) and in contrast to overall C>A frequencies were stable across sequencing lanes (FIG. 19). The regions identified were largely consistent across cross-validation folds and comprised high quality sequences with similar evaluable bases, copy number levels, and mappability but were located at genomic positions associated with different mutational frequencies between individuals with and without lung cancer reflecting the epigenomic characteristics described above (FIGS. 20A-20K, Supplementary Table 6). The identified regional differences in C>A mutation frequencies to CC>AA doublet mutations were further compared because these doublet mutations are enriched in lung cancers of smoking individuals²⁶ and have a very low likelihood of occurring by chance given the requirement of two identical changes occurring in adjacent positions (FIGS. 21A-21F). It was found that the frequency of high quality CC>AA changes was highly correlated with the regional difference in single molecule C>A frequency in both tissue (Spearman's rho=0.62, p=0.0002) and cfDNA samples (Spearman's rho=0.65, p<0.0001) (FIGS. 21E, 21F). These data strongly support the notion that GEMINI mutational frequencies reflect tumor-derived sequence changes in the circulation.

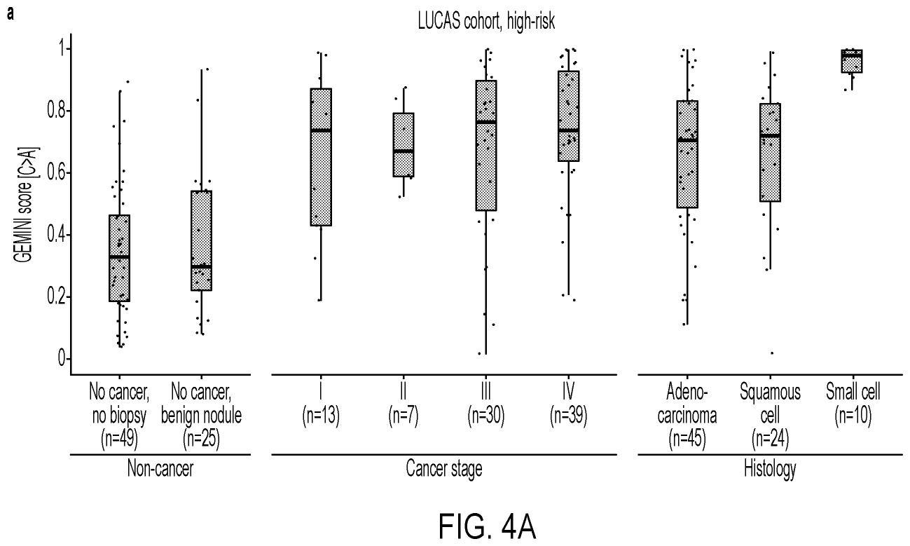

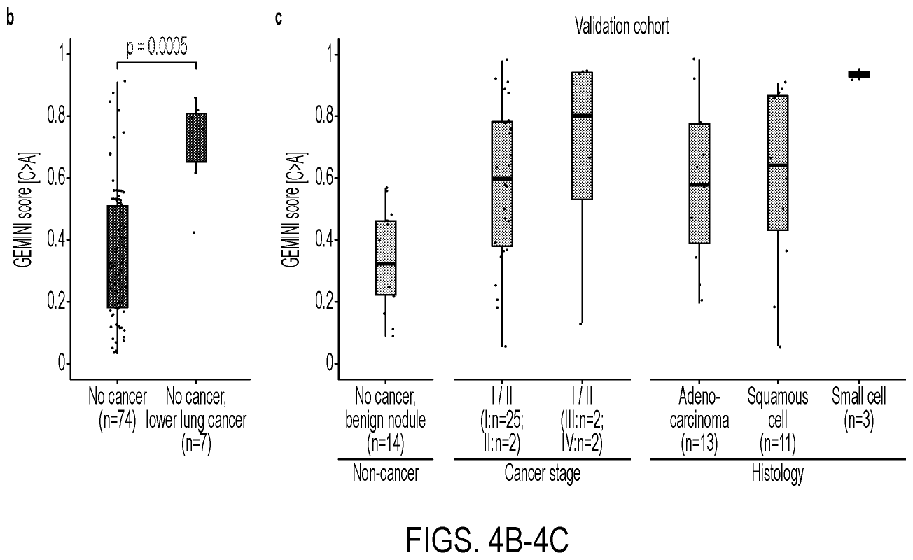

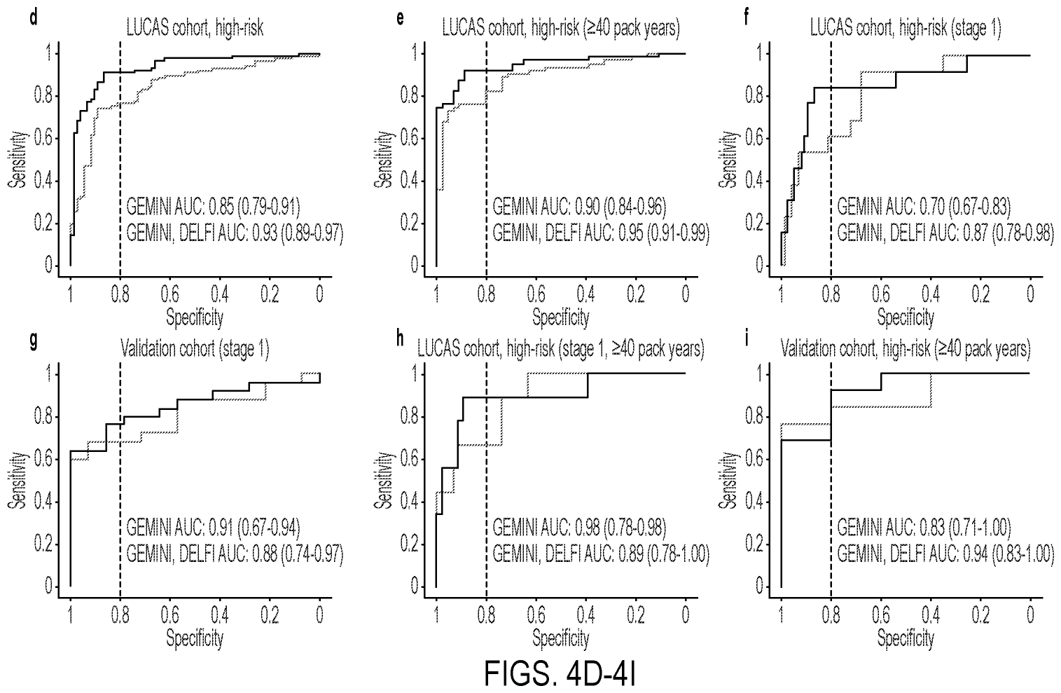

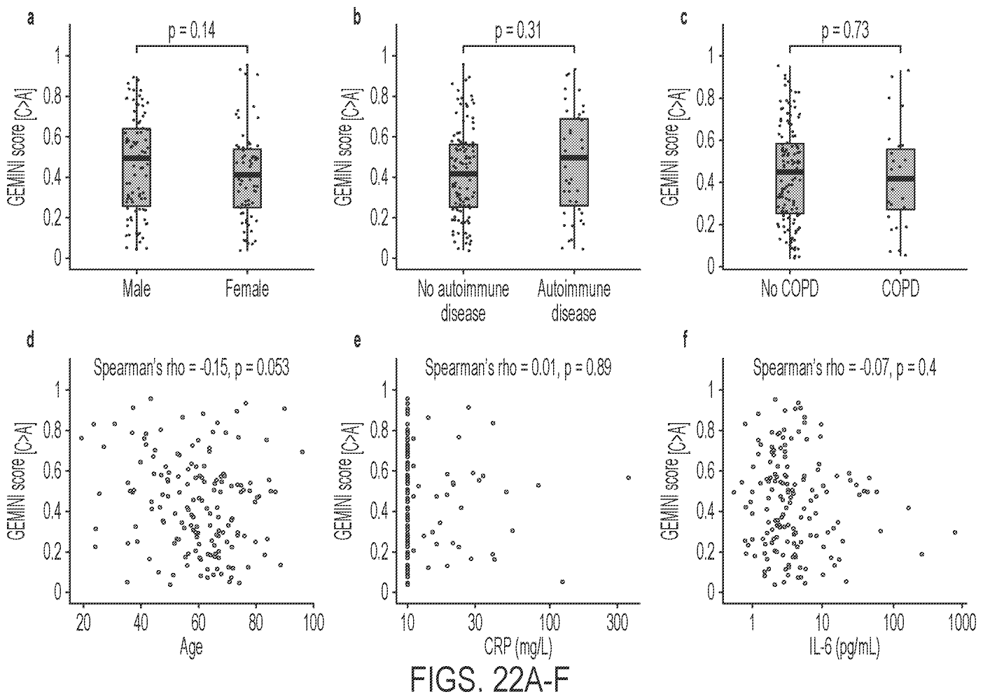

The regional differences were calibrated in single molecule C>A frequencies to GEMINI scores, reflecting an individual's probability of cancer (Methods). To evaluate whether clinical characteristics may affect genome-wide cfDNA mutation profiles, it was investigated whether non-malignant nodules, sex, age, or the presence of chronic obstructive pulmonary disease (COPD) or autoimmune diseases were associated with GEMINI scores. No difference in the GEMINI scores was observed when comparing non-cancer individuals with and without benign lesions (median GEMINI score 0.30 vs 0.33, p=0.94, Wilcoxon rank sum test) (FIG. 4A), or between men and women (p=0.14) (FIGS. 22A-22F). A correlation of the GEMINI score with age (rho=−0.15, p=0.053) was not observed, nor was it observed with the levels of the inflammatory markers CRP (rho=0.01, p=0.89) or IL-6 (rho=−0.07, p=0.40) (FIGS. 22A-22F). Similarly, changes were not observed in the GEMINI score between individuals with or without COPD (p=0.73) or autoimmune disease (p=0.31) (FIGS. 22A-22F). Together, these analyses show that single molecule mutation frequencies in cfDNA are not considerably affected by demographic characteristics or the presence of acute or chronic inflammatory conditions.

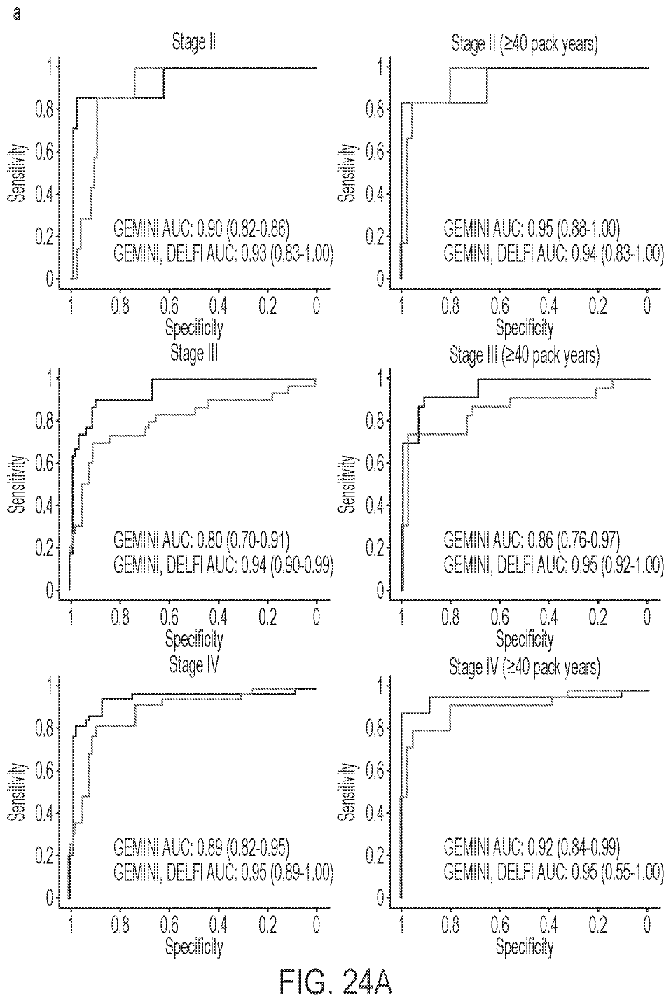

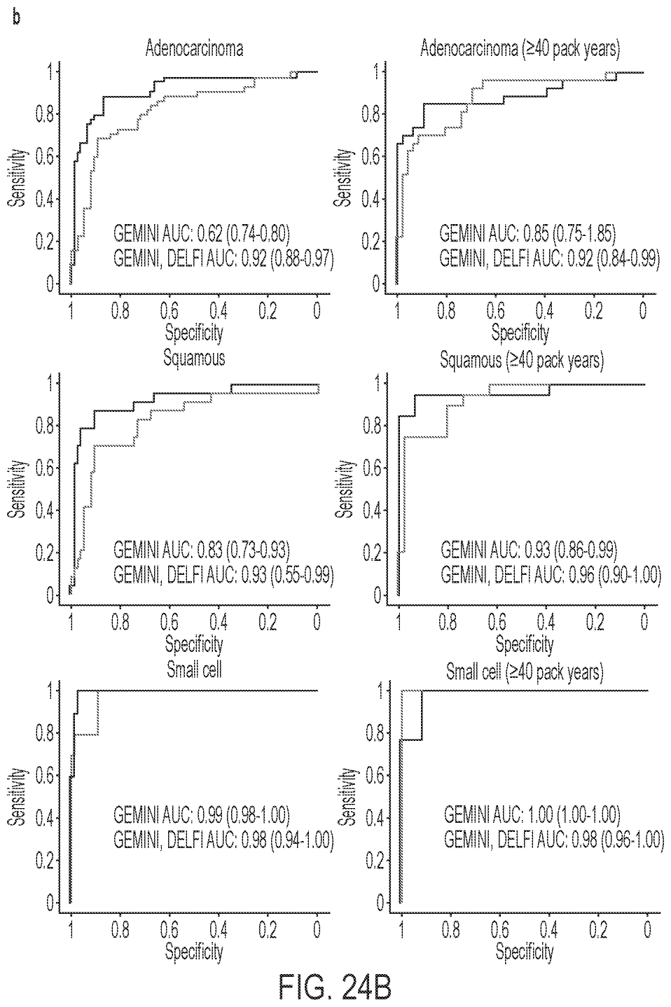

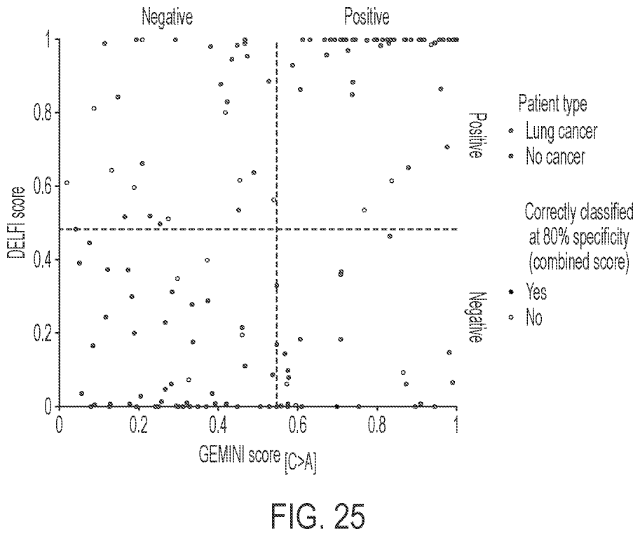

Next, the relationship between GEMINI scores and cancer stage and histology was evaluated. While the GEMINI score for non-cancer individuals was low (median scores of 0.30 and 0.33 for those with or without benign lesions, respectively), patients with cancer had significantly higher median scores across stages (stage I=0.74, stage II=0.67, stage III=0.76, and stage IV=0.74)(p<0.001 for stages I, II, III, or IV, Wilcoxon rank sum test) (FIG. 4a) and histologic subtypes (adenocarcinoma=0.71, squamous cell carcinoma=0.72, small cell lung cancer (SCLC)=0.98) (p<0.0001 for all subtypes, Wilcoxon rank sum test) (FIG. 4A). As expected, GEMINI scores were generally related to ctDNA levels, increasing with the tumor fraction estimated by ichorCNA³⁵ (p<0.0001, Wilcoxon rank sum test) (FIG. 18A). Higher GEMINI scores in SCL C patients likely reflect the known higher UtDNA fractions in this tumor type³⁶ and patients with lower GEMINI scores (e.g. below 0.5) were more likely to have a NSCLC histology, Supplementary Table7, (p=0.03, Fisher's exact test) and lower ichorCNA tumor fraction estimates (p=0.003, Wilcoxon rank sum test). A receiver operating characteristic (ROC) curve representing the sensitivity and specificity of the GEMINI approach to identify cancer patients revealed an overall area under the curve (AUC) of 0.85 (95% CI=0.79-0.91) (FIG. 4D), with high levels of detection across stages and subtypes (FIGS. 4F, 24A, 24B).

The fixed GEMINI model was used to evaluate samples from seven patients who did not have cancer at the time of blood collection but were subsequently diagnosed with lung cancer. These individuals had median GEMINI scores of 0.78, significantly higher than those of non-cancer individuals (p=0.0005, Wilcoxon rank sum test) (FIG. 4B). Six of seven of these individuals had a score above the threshold at an 80% specificity, with the time to lung cancer diagnosis ranging from 231 to 1868 days, providing evidence that abnormalities in cfDNA mutational profiles would be useful for cancer detection years earlier than standard diagnoses. Of these patients, 5 were diagnosed with NSCLC (two patients had stage I disease, one patient had stage III disease, and stage information was not available for the other two patients), one patient was diagnosed with SCLC (unknown stage), and the other patient for which we do not have stage or histology information died within a few months of their diagnosis. The patient who was not detected by GEMINI had the longest time from blood draw to diagnosis (1,954 days). Interestingly, at the time of the initial blood draw, cancer was not suspected for four of these patients based on CT imaging and no biopsy was performed. For the remaining three patients, there was suspicion of cancer based on CT imaging and the patients underwent biopsy, however their pathology report indicated a benign lung nodule, highlighting limitations of current diagnostic approaches.

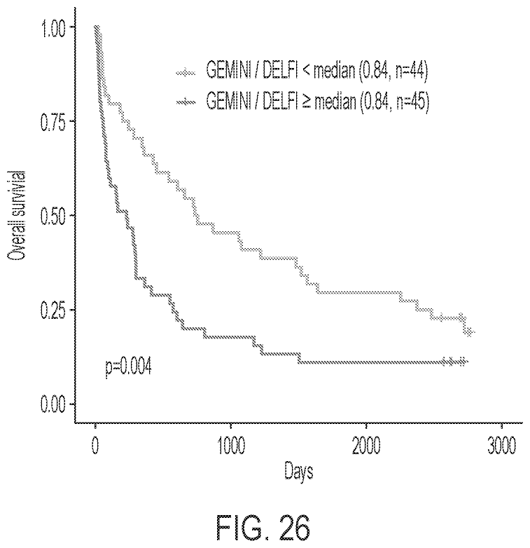

It was next examined whether GEMINI mutational profiles could be combined with genome-wide fragmentation features used by the DELFI approach as it was hypothesized that these methods measured complementary cfDNA characteristics and could be used to improve the ability to detect individuals with early stage lung cancer. The GEMINI and DELFI scores were integrated into a combined score to evaluate the predictive accuracy relative to these features used in isolation (Methods). While GEMINI and DELFI scores were positively correlated (Spearman's rho=0.50, p<0.0001), several samples missed by either approach in isolation were detected using the combined approach, resulting in a reduction in false negatives for example of 56% at 80% specificity (FIG. 25). The combined approach led to an increase of the overall performance, with an overall AUC of 0.93 (95% CI=0.89-0.97) (p<0.05 when compared to GEMINI or DELFI alone) (FIG. 4D). For stage I patients (n=13) the DELFI fragmentation or GEMINI analyses alone achieved AUCs of 0.73 (95% CI=0.59-0.88) and 0.80 (95% CI=0.67-0.93), respectively, and the combined approach resulted in an AUC of 0.87 (95% CI=0.76-0.98) (p<0.05 when compared to DELFI or GEMINI alone) (FIG. 4f). The performance of the combined GEMINI and fragmentation approach provided an overall sensitivity of 91% at a specificity of 80% (GEMINI/DELFI score >0.38) (Table 1). When considering this approach as a pre-screen to LDCT, the sensitivity of the combined approaches with LDCT would be >95% at a combined specificity of 85% (Table 1). Importantly, individuals with lower GEMINI/DELFI scores had better prognosis compared to individuals with higher scores (p=0.004, Log-rank test) (FIG. 26), reducing the concern of false negatives with this approach, as individuals with lower scores would have better prognosis and could be detected in subsequent screens.

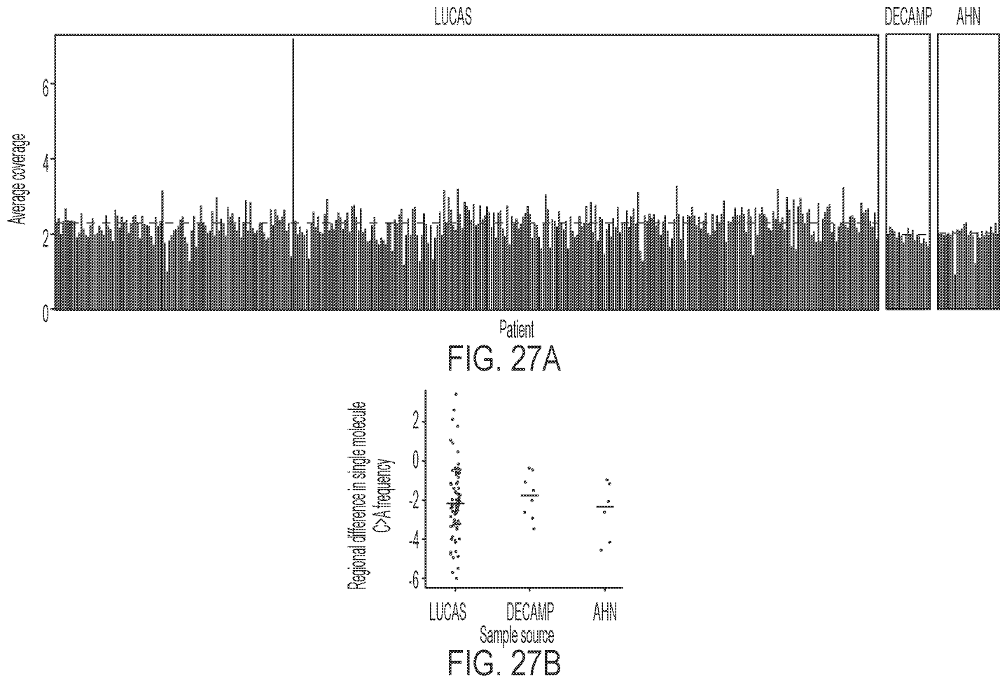

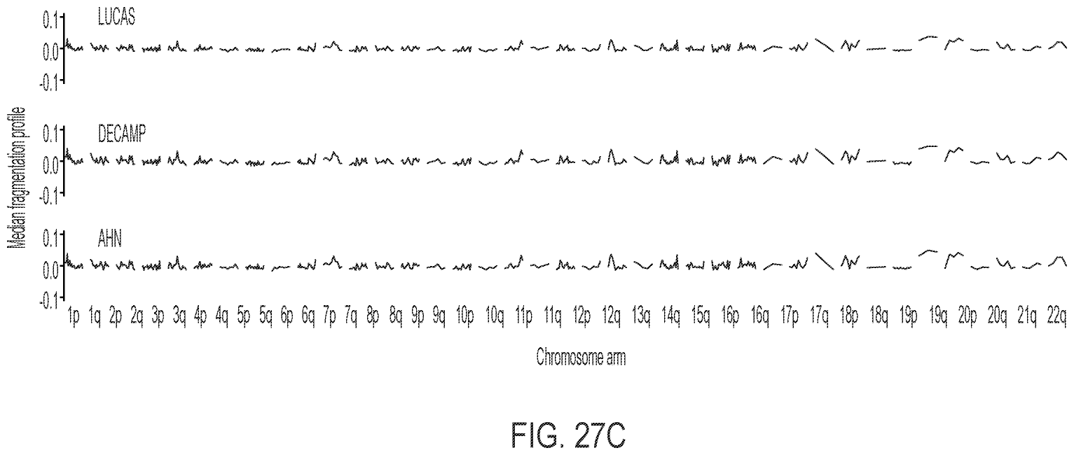

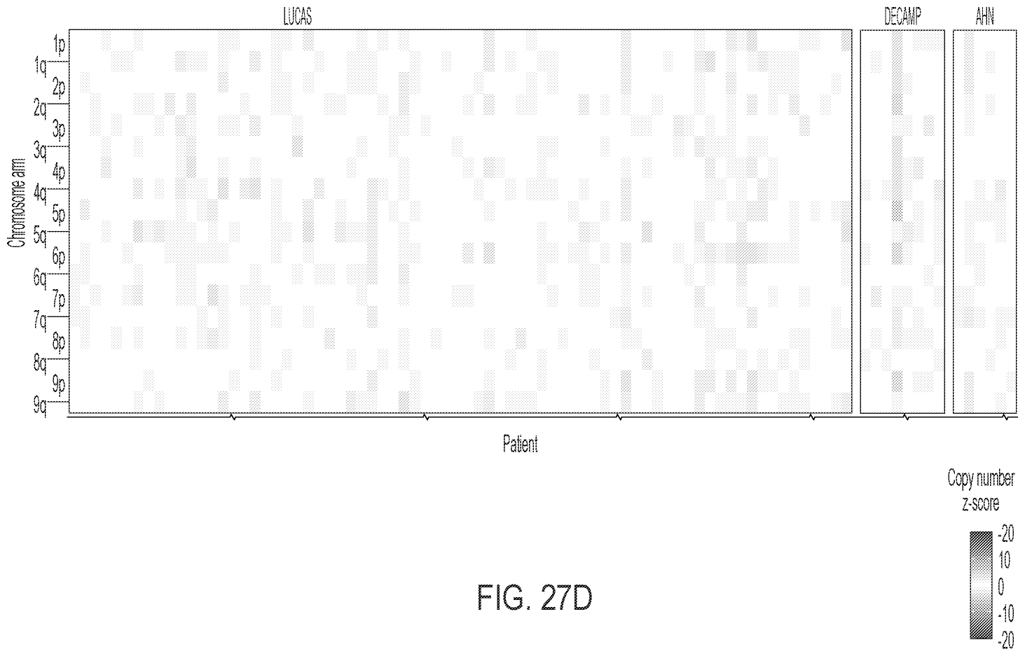

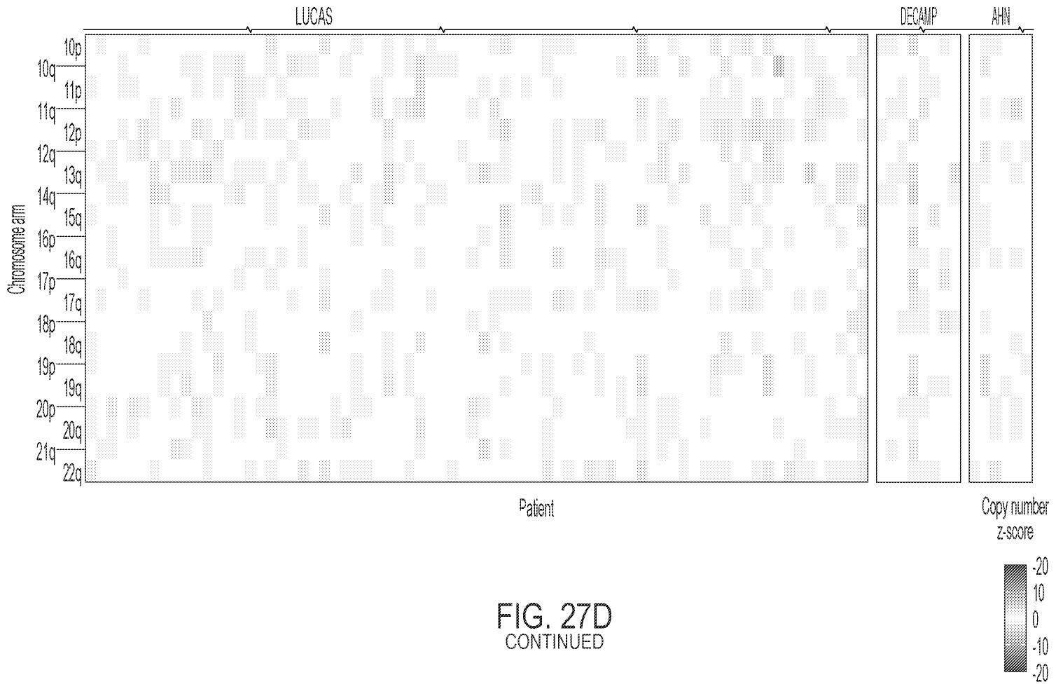

To externally validate the individual GEMINI method as well as the combined GEMINI/DELFI approach, an additional cohort of individuals from lung cancer screening programs were evaluated (n=57, Supplementary Table 3). This cohort included asymptomatic high-risk individuals with predominantly early stage cancers (stage I=32, II=4, III=3, IV=2 and unknown=1) where samples were collected prior to clinical diagnosis as well as individuals ultimately determined not to have cancer (n=15). Twenty one of 42 individuals with lung cancer (50%) were diagnosed with stage IA disease, similar to the proportion detected by LDCT in the National Lung Screening Trials. cfDNA was isolated from plasma of these individuals and performed low coverage whole genome sequencing with coverage and feature metrics similar to the LUCAS cohort (FIGS. 27A-27D). These samples were analyzed using the fixed GEMINI and fragmentation machine learning models from the LUCAS cohort analyses. Consistent with the initial studies, it was observed that GEMINI scores were higher in high-risk individuals (age 50-80 with smoking history) with cancer compared to those without cancer (p=0.001, Wilcoxon rank sum test) (FIG. 4C). Across the validation and LUCAS cohorts, GEMINI scores of later stage lung cancer patients (stage III/IV, median GEMINI score=0.74) were significantly higher than those of early stage patients (stage I/II, median GEMINI score=0.64)(p=:0.03, Wilcoxon rank sum test). The performance of the GEMINI approach for detecting stage I disease among individuals in this cohort was high, with an overall AUC of 0.81 (95% CI=0.67-0.94) and 0.86 (95% CI=0.74-0.97) when combined with fragmentation features (FIG. 4G). Overall, these analyses suggest that genome-wide mutational profiling is broadly generalizable for detection of early stage lung cancer in high-risk populations.

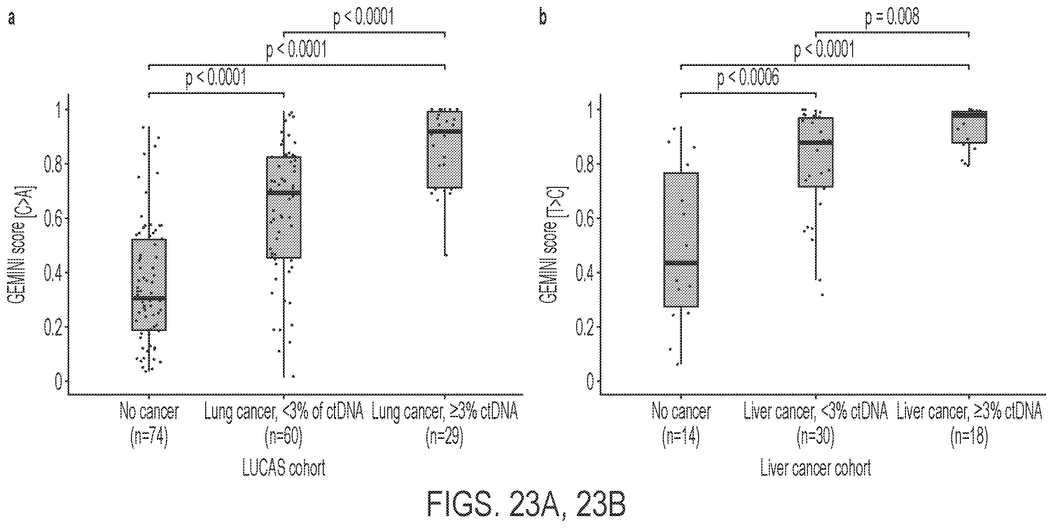

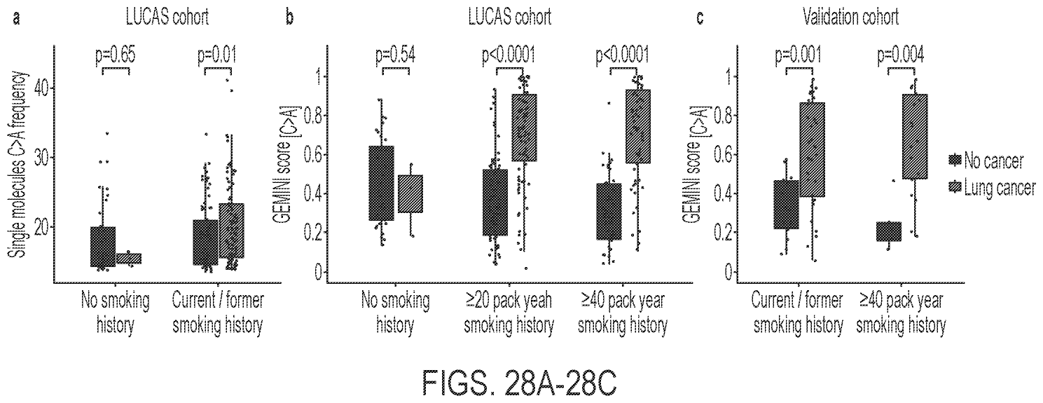

As somatic changes in lung cancer are related to smoking, it was hypothesized that there would be a relationship between the cfDNA mutational profiles and smoking history. Although overall cfDNA C>A mutation frequencies were similar among nonsmokers with and without lung cancer in the LUCAS cohort (p=0.65, Wilcoxon rank sum test), smokers with lung cancer had higher overall mutation frequencies than smokers without cancer (p=0.01, Wilcoxon rank sum test) and dramatically higher GEMINI scores (p<0.0001, Wilcoxon rank sum test) (FIGS. 23A, 23B). The GEMINI score was positively associated with years of smoking among cancer patients (rho=0.24, p=0.01). Interestingly, in individuals without cancer, the GEMINI score was negatively correlated with smoking exposure (rho=−0.25, p=0.002), potentially reflecting smoking-related DNA damage in non-cancer tissues³⁷ that may contribute to alterations of cfDNA. Analyses of patients in the LUCAS and validation cohorts suggested that the GEMINI approach may have higher performance in detecting individuals with greater smoking history (FIGS. 4E, 4H, 4I, 28A-28C), including an increase in GEMINI performance in the LUCAS cohort to an AUC of 0.90 and 0.95 with the combined GEMINI/DELFI approach (p<0.05, DeLong's test compared to GEMINI or DELFI alone which had AUCs of 0.90 and 0.88 respectively). A positive GEMINI test at a specificity of 80% was associated with a 13.5 fold increase in the odds of cancer among ≥20 pack year smokers (95% CI for odds ratio: 6.7-30.7, p<0.0001), and with a 20.1-fold increase among ≥40 pack-year smokers (95% CI for odds ratio: 7.7-54.6, p<0.0001). These observations are consistent with the notion that smoking exposure results in sequence alterations in both ctDNA and non-tumor cfDNA, affecting distinct genomic regions that may be helpful for improved cancer detection using the GEMINI approach.

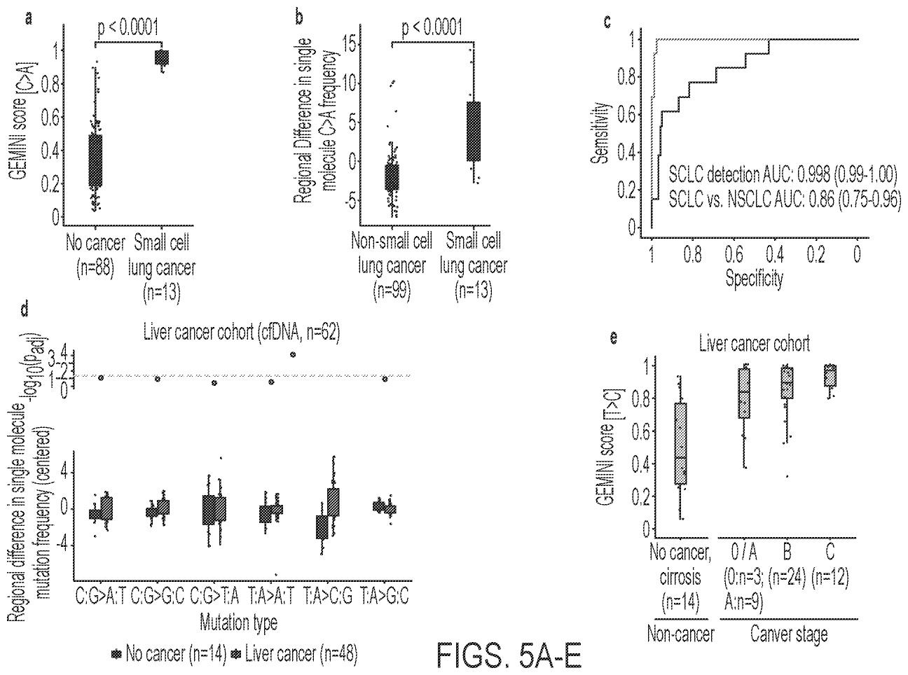

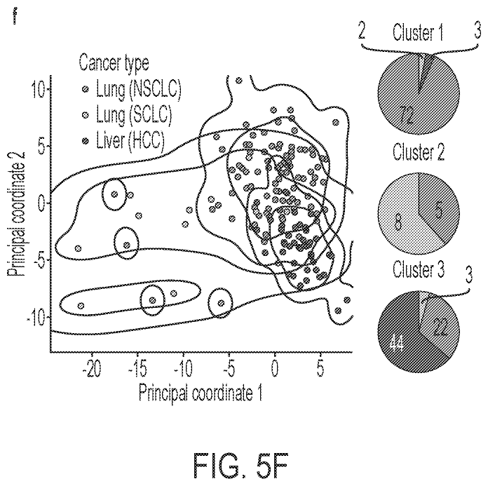

Given the important differences between biologic features and clinical management of SCLC and non-small cell lung cancer (NSCLC), we examined whether genome-wide mutational profiles could be used to detect SCLC and to non-invasively distinguish this cancer from other cancer types. The GEMINI score was extremely high in SCLC patients (n=13) compared to non-cancer individuals (n=88) (p<0.0001, Wilcoxon rank sum test) (FIG. 5A, Supplementary Tables 2, 3), and could distinguish among these with an AUC of >0.99 (95% CI=0.99-1.00) (FIG. 5C). The GEMINI approach was used to assess regional mutation differences in cfDNA of patients with SCLC compared to those with NSCLC (n=99) and found that mutation frequencies obtained in this way were higher in SCLC (p<0.0001, Wilcoxon rank sum test) (FIG. 5B, Supplementary Table 4), and could be used distinguish this cancer type from NSCLC (AUC=0.86, 95 CI=0.75-0.96) (FIG. 5C). These findings suggest that genome-wide mutation profiles may be useful for providing a non-invasive approach for detecting SCLC and distinguishing lung cancers with different histologic subtypes.

To explore the generalizability of the GEMINI approach to detect other cancers, the method was applied to evaluate a prospective cohort of individuals with or without liver cancer (n=62). Cross-validated regional differences in mutation frequencies identified a significant difference in genome-wide T>C mutation profiles (FIG. 5D) in individuals with liver cancer. The derived GEMINI scores were higher in individuals with liver cancer across all stages (0-A, B, and C) compared to those with cirrhosis (p<0.01 for each comparison) (FIG. 5E). Similar to analyses of lung cancer patients, GEMINI scores from patients with liver cancer were generally related to ctDNA levels, increasing with the tumor fraction estimated by ichorCNA³⁵ (p=0.008, Wilcoxon rank sum test) (FIG. 23B, Supplementary Table 8).

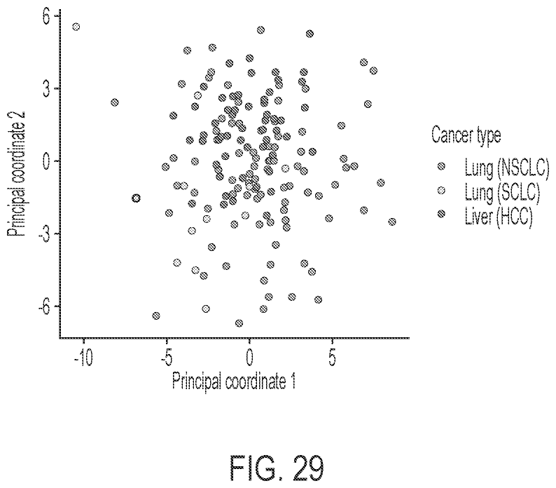

As cfDNA mutation profiles appeared cancer-type specific, we hypothesized that the GEMINI approach could be useful for distinguishing among different cancer types. Using GEMINI, differences in mutation profiles in cfDNA were compared between NSCLC, SCLC, and HCC (n=159) and it was found that profiles largely clustered into three groups with each cancer type comprising the majority of observations in a cluster (FIG. 5F) (Methods). Exclusion of the most common tumor-specific alterations (FIGS. 3A-3B, 7) prevented accurate grouping by cancer type (FIG. 29). Overall, these analyses suggested that mutation profiles may be a useful method for non-invasive determination of cancer origin.

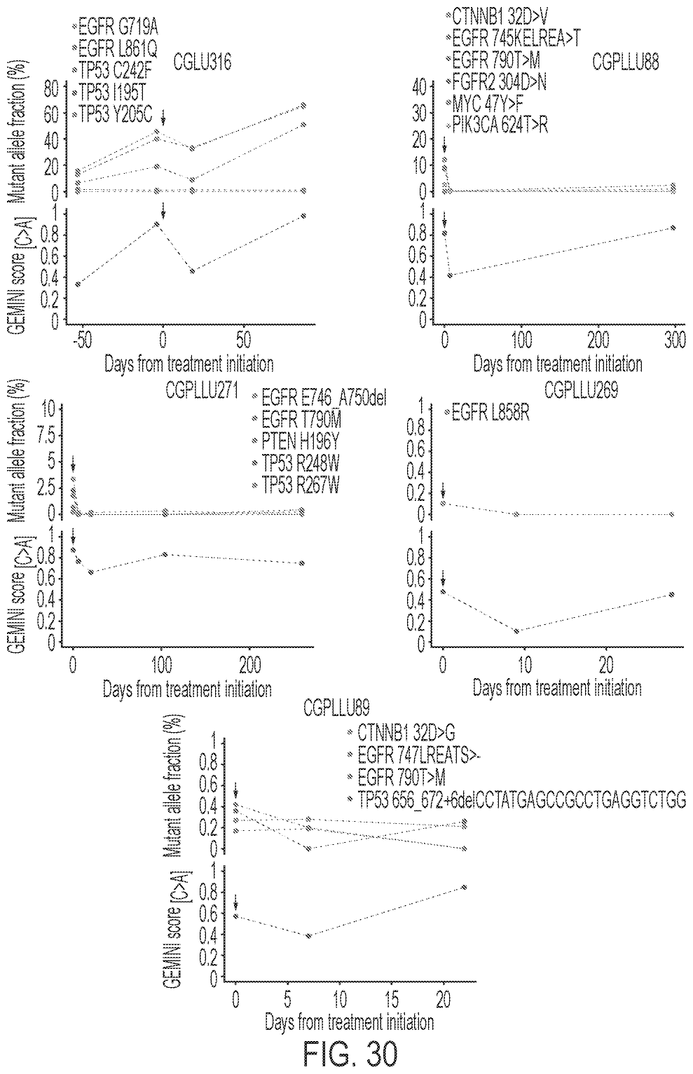

To explore whether the GEMINI approach could be used to monitor patients during therapy, we assessed serial blood samples from lung cancer patients undergoing treatment with EGFR or ERBB2 inhibitors with mutant allele fractions (MAFs) as low as 0.1%. Using the fixed model trained on the high-risk LUCAS cohort, it was found that after the initiation of therapy GEMINI scores decreased in all patients, consistent with an initial response to therapy, and over time GEMINI scores increased, consistent with the known progression of these individuals (FIG. 30). Comparison of GEMINI scores with mutant allele fractions from targeted sequencing of these patients revealed a significant correlation of ctDNA levels between the two methods (Spearman's correlation coefficient=0.50, p=0.03), indicating that GEMINI has high sensitivity to low MAF levels and reflects ctDNA burden during therapy.

In this study, it was shown that individuals with cancer can be detected non-invasively through single molecule mutation profiles obtained from low coverage whole genome sequencing of cfDNA. The altered tumor-type specific mutational landscapes were detectable in plasma for cancer patients and appear to be related to replication timing and other chromatin features of the genome where repair of DNA damage may be impaired³⁸. The method described here does not require deep sequencing of matched blood cells to filter hematopoietic alterations¹⁶, or tumor sequencing to identify tumor-specific mutations to evaluate in the plasma²², and therefore the approach is amenable for de novo detection and characterization of cancer. The combination of genome-wide sequence and fragmentation analyses of cfDNA provide an opportunity for cost-effective and scalable detection of cancer.

Although the majority of patients in the cohorts described represented individuals at risk for developing cancer, large-scale validation in a screening population for lung, liver and other cancers will be needed before clinical use. However, increasing the read length from 100 bp to 150 bp would increase the evaluable bases sequenced by both reads ˜4 fold. Although a variety of genome-wide tumor-specific mutation profiles were assessed, including for different lung cancer histologies, liver cancer, melanoma, and lymphoma, analysis of additional genome-wide mutational profiles using other sequence alterations will likely be more effective in other settings. As mutation rates vary substantially across the cancer genome³¹, the detection of altered regional mutational frequencies in cfDNA provides generalizable approach that may be useful for early cancer detection and monitoring.

### Example 2: Methods and Materials

### Study Populations Analyzed

Tissue samples from the PCAWG Consortium consisted of 2,778 tumors with somatic mutation calls³⁹. Hypermutated tumors, including those with putative polymerase epsilon or mismatch repair defects, as well as one tumor with temozolomide treatment were excluded from analysis (n=49), as well as cancer types with less than 20 samples (n=129 samples) and cancer types with an average of <250 mutations per sample (pilocytic astrocytoma, n=89 samples) resulting in 2,511 tumors across 25 common cancer types. Single molecule mutation analyses consisted of lung cancer and matched solid tissue or blood cells from 86 donors of who passed quality control metrics³⁹. This cohort consisted of 30 females and 56 males who were diagnosed with lung cancer between ages 41 and 83. Among these individuals, 38 had lung adenocarcinoma and 48 had lung squamous cell carcinoma, and 65 of them had mutations attributed to smoking related Signature 4. Of these 65 patients, 31 of them had both tumor tissue and blood derived normal sequencing data available. Additional information regarding these samples is available in at dcc.icgc.org/releases/PCAWG. See, also Supplementary Table 1.

As previously described¹⁸, the LUCAS cohort was a prospectively collected group of 365 patients that presented in the Department of Respiratory Medicine, Infiltrate Unite, Bispebjerg Hospital, Copenhagen with a positive imaging finding on a chest X-ray or a chest CT. Patients diagnosed with cancer with known active disease or who were under treatment at the time of enrollment were excluded. The study was conducted over 7 months from September 2012 to March 2013, and all patients had a clinical follow-up until death or April 2020. All patients provided written informed consent and the studies were performed according to the Declaration of Helsinki. The LUCAS study was approved by the Danish Regional Ethics Committee and the Danish Data Protection Agency. All patients had blood samples collected at their first clinic visit before the possible diagnosis of lung cancer was made. The analyzed cohort included 158 patients with no prior, baseline, or future cancers, 114 patients with baseline lung cancer, 15 patients with a lung metastasis, and 78 patients without lung cancer at the time of blood collection, but with either earlier or later lung cancers or another cancer type. The high-risk LUCAS cohort was defined as individuals at high risk for lung cancer (age 50-80, ≥20 pack year smoking history) and included individuals with primary lung cancer at baseline (n=89) and individuals without prior, baseline, or future cancer (n=74). (Supplementary Table 2). There was a median ˜4,451 (range: 392-2,111) haploid genome equivalents per ml of plasma analyzed.

The validation cohort was comprised of individuals from lung cancer screening programs (n=57) (Supplementary Table 3) including asymptomatic high-risk individuals with predominately early stage cancers or nodules determined to be benign. Individuals were enrolled either through the Detection of Early Lung Cancer Among Military Personnel (DECAMP) Consortium⁴⁰, or through screening efforts at the Allegheny Health Network (AHN). The DECAMP-1 protocol included current or former cigarette smokers with ≥20 pack-year exposure and radiological findings indicating an indeterminate pulmonary nodule of 0.7 to 3.0 cm in size identified within 12 months prior to enrollment with an additional CT scan within 3 months prior to enrollment. Individuals enrolled at the AHN were identified based on eligibility for high-risk screening for lung cancer using low-dose helical CT scanning or an indication for lung cancer screening based on other high-risk characteristics such as family history of lung cancer. All patients provided written informed consent to participate in these collections and the studies were performed according to the Declaration of Helsinki. All individuals had a liquid biopsy collected prior to a possible diagnosis of lung cancer.

The lung cancer monitoring cohort consisted of serial blood draws from a cohort of lung cancer patients undergoing treatment with EGFR or ERBB2 inhibitors¹¹. The study population included samples from serial blood draws (n=18) from patients with a smoking history (n=5) with both targeted and whole-genome sequencing available¹³. Patients were age 50-73, had stage II-IV lung adenocarcinoma (n=4) or mixed histology (n=1).

The liver cancer cohort consisted of 62 patients with either liver cancer (n=48) or cirrhosis (n=14). Samples were collected prospectively as part of the HCC biomarker registry at the Johns Hopkins University School of Medicine, under a protocol approved by the Johns Hopkins Institutional Review Board. Liver cancer was defined by appropriate imaging characteristics as defined by accepted guidelines. Tumor staging was determined by the Barcelona Clinic Liver Cancer staging system (BCLC). Detailed clinical data were extracted from electronic medical records (Supplementary Table 8).

### Blood Sample Collection and Preservation

The sample collection for the LUCAS cohort was performed at the time of the screening visit and executed as follows: venous peripheral blood was collected in one K2-EDTA tube. Within two hours from blood collection tubes were centrifuged at 2330 g at 4° C. for 10 minutes. After centrifugation, EDTA plasma were aliquoted and stored at −80° C.

For the validation cohort, venous peripheral blood for each individual was collected in one K2-EDTA tube (AHN) or one Streck tube (DECAMP). Tubes from the AHN and the DECAMP collections were centrifuged at low speed (800-1600 g) for 10 minutes. The plasma portion from the first spin was spun a second time for 10 minutes. After centrifugation plasma was aliquoted and stored at −80° C. for cfDNA analyses.

For the lung cancer monitoring cohort, whole blood was collected in EDTA tubes and processed immediately or within one day after storage at 4° C. or was collected in Streck tubes and processed within two days of collection as previously described¹³. Plasma and cellular components were separated by centrifugation at 800 g for 10 min at 4° C. Plasma was centrifuged a second time at 18,000 g at room temperature to remove any remaining cellular debris and stored at −80° C. until the time of DNA extraction.

For the liver cancer cohort, the sample collection was performed as follows: venous peripheral blood was collected in one K2-EDTA tube. Within two hours from blood collection tubes were centrifuged at 2330 g at 4° C. for 10 min, plasma was transferred to new tubes and the samples were spun at 14,000 rpm (18,000 rcf) for 10 minutes at room temperature to pellet any remaining cellular debris. After centrifugation EDTA plasma was aliquoted and stored at −80° C. for cfDNA analyses.

### Plasma Sequencing Library Preparation

For all plasma samples, circulating cell-free DNA was isolated from 2-4 ml of plasma using the Qiagen QIAamp Circulating Nucleic Acids Kit (Qiagen GmbH), eluted in 52 μl of RNase-free water containing 0.04% sodium azide (Qiagen GmbH), and stored in LoBind tubes (Eppendorf AG) at −20° C. Concentration and quality of cfDNA were assessed using the Bioanalyzer 2100 (Agilent Technologies).

Next-generation sequencing (NGS) cfDNA libraries from the LUCAS, validation, and liver cancer cohorts were prepared for whole genome sequencing using 15 ng of cfDNA when available, or the entire purified amount when less than 15 ng was available. In brief, genomic libraries were prepared using the NEBNext DNA Library Prep Kit for Illumina (New England Biolabs (NEB)) with four main modifications to the manufacturer's guidelines: (i) the library purification steps use the on-bead AMPure XP (Beckman Coulter) approach to minimize sample loss during elution and tube transfer steps; (ii) NEBNext End Repair, A-tailing and adaptor ligation enzyme and buffer volumes were adjusted as appropriate to accommodate on-bead AMPure XP purification; (iii) Illumina dual index adaptors were used in the ligation reaction; and (iv) cfDNA libraries were amplified with Phusion Hot Start Polymerase. All of these samples underwent a 4 cycle PCR amplification after the DNA ligation step. For the lung cancer monitoring cohort, next-generation sequencing (NGS) cfDNA libraries were prepared for WGS and targeted sequencing using 5-250 ng of cfDNA as previously described^{11,13}.

### Whole Genome Sequencing Datafrom PCAWG Samples

Somatic mutation calls, tumor purity, coverage statistics, as well as mutation signature abundances generated by SigProfiler²⁶ were downloaded from the International Cancer Genome Consortium (ICGC) Data Portal (https://dcc.icgc.org/releases/PCAWG). Bam files and germline variant calls were downloaded from the Bionimbus Protected Data Cloud (bionimbus.opensciencedatacloud.org). Barn files were indexed using SAMtools⁴¹.

Downsampling and Dilution of Somatic Mutations from PCAWG Lung Cancer Samples

The downsampling and dilution experiment methodology is shown FIG. 8. Specifically, somatic mutation calls (n=3,393,564 mutations) were obtained for individuals in PCAWG with a lung cancer with the presence of Signature 4 (n=65)²⁶. Mutations were excluded with a missing value for either the number of reference or mutant alleles observed (n=5,857) resulting in 3,387,707 somatic mutations across 65 individuals. For a given individual, each observation of the reference or mutant allele separately was considered. The number of sequenced observations were computed that were tumor derived as the total number of observations multiplied by the tumor purity of the sample. We then spiked in observations with the reference allele until 10⁻¹, 10⁻², 10⁻³, or 10⁻⁴ of the observations were of tumor origin. The average coverage of mutated positions following dilution was next computed, and the observations were randomly sampled to achieve a desired coverage of 8×, 4×, 2×, 1×, and 0.5×. For each known somatic mutation in an individual's cancer genome, we tallied the number of times that we observed the mutation for each combination of dilution amount and genome coverage and used this information to compute the percent of mutations observed in single DNA molecules.

### Whole Genome Sequencing of Plasma Samples

Libraries prepared from whole genomes of cancer patients and cancer-free individuals were sequenced at ˜2× coverage per sample using 100 bp paired-end runs (200 cycles) on the Illumina HiSeq 2000/2500 (LUCAS¹⁸, validation, and lung cancer monitoring cohorts¹³) and the NovaSeq 6000 (liver cancer cohort). To assess concordance between tissue and cfDNA mutation profiles in cancer types with few available samples, LUCAS samples were re-sequenced from patients with melanoma (n=2) and lymphoma (n=1) as well as well as 40 non-cancer controls and 15 individuals with largely advanced lung cancers to at median of 10× coverage on the Illumina NovaSeq 6000. Prior to alignment, adapter sequences were filtered from reads using fastp⁴². Sequence reads were aligned against the hg19 human reference genome using Bowtie2⁴³ and duplicate reads were removed using Sambamba⁴⁴. Sequencing data from each sample comprised >7.5 million fragments, >15 million reads, >10 million reads mapped to the reference genome, >85% of bases with a Phred quality score ε20 (Q20), and >80% of bases with a Phred quality score ε30 (Q30).

### Identification of Single and Doublet Base Changes in Single Molecules

The primary alignment of properly paired read pairs that mapped to autosomes in non-overlapping 100 kb bins were scanned and the base call, Phred score, and mapping quality (MAPQ) of each sequenced base using pysam was obtained. Only read pairs were considered with a MAPQ of at least 40 and only positions within each read with a Phred score of at least 30. To avoid counting multi-nucleotide variants in analyses of single base changes, positions were filtered where the two adjacent positions both contained the reference allele and had a Phred score of at least 30. A similar filter was used in analyses of doublet base changes to avoid counting larger multi-base variants. In addition, positions were removed that overlapped the Duke Excluded Regions track (bgdownload.cse.ucsc.edu/goldenpath/hg19/encodeDCC/wgEncodeMapability). In each 100 kb bin, the number of sequenced bases that were C:G or A:T in the reference genome were counted. Also counted were the number of times each type of single base change (C:G>A:T, C:G>G:C, C:G>T:A, T:A>A:T, T:A>C:G, and T:A>G:C) and CC:GG>AA:TT doublet base changes in 100 kb bins were observed. Observations were counted separately based on whether the purine or the pyrimidine of each base pair was in read 1 or read 2 of the paired-end sequencing data. To exclude potential germline variants, the gnomAD database (version 3.0) was used which contains genetic variants from >70,000 whole genomes⁴⁵. The gnomAD version 3.0 variant call format (VCF) file was downloaded that was available in hg38 coordinates from the gnomAD browser. First lifted was the position of each sequence change that was identified over from hg19 to hg38 using the R package rtracklayer. Sequence changes that did not lift over to hg38, that lifted over to hg38 but to multiple different locations, or that lifted over to hg38 but the reference genome sequence differed between the hg19 and hg38 genome builds were removed. Sequence changes that were identified with their population allele frequency as well as whether the variant passed gnomAD quality filters were annotated. Any candidate variants were subsequently removed if the variant was present in gnomAD but the variant did not pass gnomAD quality filters, or if the variant was present in gnomAD with an allele frequency >1/100,000. For PCAWG samples, the remaining variants were annotated in each sample indicating if they were called as a somatic or germline variant by the PCAWG consortium. For analyses of tissue samples, if any position in a fragment was sequenced by both read pairs, the position was kept from either read 1 or read 2 at random. For plasma samples, positions in fragments that were sequenced by both read 1 and read 2 of the read pair with the same base call were analyzed. To filter 8-oxo-dG-related sequence changes from single base analyses, any base where guanine or G>T was on read 1 and cytosine or C>A was on read 2 was excluded. To filter artifactual CC>AA changes, any bases where CC or CC>AA were on read 1 and GG or GG>TT were on read 2 were excluded. To account for potential differences in sequencing depth between samples, single molecule mutation frequencies were always computed as the number of each sequence change divided by the number of evaluable bases, defined as the number of positions in fragments in which each sequence change could be detected after quality and germline filtering.

Estimation of 8-oxo-dG Level

The 8-oxo-dG level was estimated for each sample as ratio of single molecule C>A frequencies when guanine or G>T was on read 1 and cytosine or C>A was on read 2 to when cytosine or C>A was on read 1 and guanine or G>T was on read 2.

### Generation of Regional Differences in Single Molecule Mutation Frequencies

The approach to compute the regional difference in single molecule mutation frequency for a given mutation type is shown in FIG. 15. Specifically, the 100 kb bins were first aggregated to 1144 non-overlapping 2.5 Mb bins. Let yᵢ⁰ and yᵢ¹ denote the number of sequence changes (e.g. C>A) at bin i for a non-cancer participant and a cancer participant, respectively. We denote the corresponding number of evaluable positions (e.g. number of C:G bases that pass quality filters) by xᵢ⁰ and xᵢ¹. The difference in the number of sequence changes at bin i relative to the number of evaluable bases comparing cancer participants to non-cancer participants for a training set comprised of n−1 samples with J cancer participants and K non-cancer participants (J+K=n−1) is given by

$${δ}_{i} = \frac{{\sum }_{j} {y}_{ij}^{1}}{{\sum }_{j} {x}_{ij}^{1}} - \frac{{\sum }_{k} {y}_{ik}^{0}}{{\sum }_{k} {x}_{ik}^{0}} for i = 1 , … , 1144.$$

Let δ₍ₛ₎ denote the s^{th} order statistic such that δ₍₁₎ is the bin most depleted for sequence changes in cancers relative to non-cancers and δ₍₁₁₄₄₎ the bin most enriched for sequence changes in cancers relative to non-cancers. Feature selection in the training set proceeds by identifying the bins at the bottom decile of δ (bins with values δ₍₁₎, . . . , δ₍₁₁₄₎) and the bins at the top decile (bins with values δ₍₁₁₄₄₎, . . . , δ₍₁₀₃₀₎). Denoting the bin sets for the top and bottom deciles by {A₋ₕ} and {B₋ₕ}, respectively, for a training set that excludes the h^{th}sample, the regional difference in single molecule mutation frequency for the test sample is given by

$$regional {difference}_{h} = \frac{{\sum }_{b ∈ \{ {B}_{- h} \}} {y}_{bh}}{{\sum }_{b ∈ \{ {B}_{- h} \}} {x}_{bh}} - \frac{{\sum }_{a ∈ \{ {A}_{- h} \}} {y}_{ah}}{{\sum }_{a ∈ \{ {A}_{- h} \}} {x}_{ah}}$$

Using leave-one-out cross-validation, this procedure was repeated such that every participant appears in the test set once and the regional differences in single molecule mutation frequency is obtained for all n participants.

### Downsampling the Regional Difference in Single Molecule C>A Frequency to 1× Coverage in PCAWG

For brevity, the alternative notation was used for the regional difference

$$\frac{{y}_{Bh}}{{x}_{Bh}} - \frac{{y}_{Ah}}{{x}_{Ah}}$$

where

y_{Bh}=Σ_{b∈{B}_{₋ₕ}_{}}y_{bh}. Denoting the down-sampled (*) regional difference by

$$regional {difference}_{h}^{*} = \frac{{y}_{Bh}^{*}}{{x}_{Bh}^{*}} - \frac{{y}_{Ah}^{*}}{{x}_{Ah}^{*}} ,$$

these quantities were derived first by determining the number of evaluable C:G positions in the hg19 reference genome, r_{A} and r_{B}. Next, we randomly sampled (without replacement) r_{A} indices from the set {1, . . . , x_{Ah}} and r_{B} indices from the set {1, . . . , x_{Bh}} to represent indices of evaluable positions in these bin sets. The number of indices in the two random samples that were less than or equal to y_{Ah} and y_{Bh} were used for y_{Ah}* and y_{Bh}*, respectively. The above procedure was repeated until all participants in the PCAWG had a down-sampled regional difference in the single molecule C>A frequency.
Association of the Single Molecule Mutation Frequencies with Tissue-Specific Genomic Features

Replication timing tracks generated by the UW ENCODE group computed by averaging the wavelet-smoothed transform of the six fraction profile representing different time points during replication in 1 kb bins were downloaded from the UCSC Genome Browser from IMR90, NHEK, and GM12878 cell lines. The weighted average was computed in each 2.5 Mb bin with higher values indicating earlier replication timing. Gene expression values were obtained as raw counts using recount3⁴⁶ and converted to transcripts per million (TPM) from lung adenocarcinoma (n=542), lung squamous cell carcinoma (n=504), melanoma (n=472), and N-cell non-Hodgkin lymphoma (n=48) generated by The Cancer Genome Atlas (TCGA). For each cancer type TPM values were first averaged for each gene across samples. The gene expression in each 2.5 Mb bin in each cancer type was computed as the sum of the TPM overlapping each bin weighted by the length of the transcript. These values were then averaged between lung adenocarcinoma and lung squamous cell carcinoma to obtain a single lung cancer gene expression estimate in each bin. A/B compartmentalization data generated at 100 kb resolution through eigenvector analysis of 450K methylation array data was obtained for 12 cancer types and through eigenvector analysis of Hi-C data for GM12878 cells³³. The weighted average of the eigenvectors in 100 kb bins were computed for each 2.5 Mb bin. The average of these values from lung adenocarcinoma and lung squamous cell carcinoma was used for lung cancer analyses, GM12878 was used for BNHL analyses, and the average across all 12 cancer types was used for melanoma analyses in absence of skin A/B compartmentalization data. ChIP-seq data for H3K9me3 of A549 cells (3 pooled replicates), GM23248 cells, and Karpas 422 cells (two pooled replicates) represented as the fold change of coverage in enriched samples with respect to control samples was downloaded from the ENCODE portal (accessions: ENCFF425LVX, ENCFF098PML, and ENCFF574RYG). The weighted average of the fold changes was computed in each 2.5 Mb bin for each cell type. GC content in each 2.5 Mb bin was obtained from the hg19 reference genome. Mappability, reflecting how uniquely 100-mer sequences align to a region of the genome, was downloaded (hgdownload.cse.ucsc. edu/goldenpath/hg19/encodeDCC/wgEncodeMapability/wgEncodeCrgMa pabilityAlign100mer.bigWig) and aggregated into 2.5 Mb bins as the weighted average of mappability scores overlapping each bin. Genome-wide copy number was estimated for each sample using ichorCNA. Average copy number per genomic bin was computed as the weighted average of the copy number in segments overlapping each bin.

### Generation of GEMINI Scores

To provide a calibrated score that captures the relationship between the regional difference in single molecule C>A frequency and the probability an individual has lung cancer in the high-risk LUCAS cohort, a logistic regression model was fitted for cancer status (lung GEMINI model) using the regional difference in single molecule C>A frequency as a covariate and extracted the fitted probability of cancer for each individual (lung GEMINI score). A lung GEMINI score >0.55 reflects a positive test for detection of lung cancer at 80% specificity. In addition, lung GEMINI scores were generated for the validation cohort, the cohort of patients with a baseline negative test that later developed lung cancer, the cohort of lung cancer patients that were monitored during therapy, as well as the remaining samples in the LUCAS cohort using the fixed bin sets and lung GEMINI model. For the liver cancer cohort, GEMINI scores were generated by fitting a logistic regression model for cancer status (liver GEMINI model) using the regional difference in single molecule T>C frequency as the covariate and extracting the fitted probability of cancer for each individual (liver GEMINI score). A liver GEMINI score >0.86 reflects a positive test for detection of liver cancer at 80% specificity (See, Supplementary Tables 1-8).

### Generation of the DELFI and Combined GEMINI DELFI Scores

To evaluate whether fragmentation features could further improve prediction of cancer status by GEMINI, the approach previously described¹⁸ was used on the same training sets used to generate cross-validated GEMINI scores. Briefly, the hg19 reference genome was tiled into non-overlapping 5 Mb bins. Bins with an average GC content <0.3 and an average mappability <0.9 were excluded, leaving 473 bins spanning approximately 2.4 GB of the genome. Fragment size analyses were conducted on fragments with a MAPQ of at least 30. Ratios of the number of short (100-150 bp) to long (151-220 bp) fragments across the 473 bins were normalized for GC-content and library size as previously described¹⁸. For each training set, a principal component analysis was performed on the fragmentation profiles and retained the minimum number of principal components needed to explain 90% of the variance between participants. Chromosomal arm copy number was summarized by computing a z-score for each arm using an expected coverage and standard deviation computed from an external reference set of 54 non-cancer controls (github.com/cancer-genomics/PlasmaToolsHiseq.hg19). The 39 z-scores and principal components were integrated as covariates in a logistic regression model with a LASSO penalty. To generate DELFI scores in the validation cohort, we used the model described previously¹⁸ that was trained on 158 non-cancers and 129 cancers. The combined GEMINI/DELFI scores was computed by averaging the individual GEMINI and DELFI scores for each patient.

Association of GEMINI Scores with the Fraction of Tumor DNA in Plasma

The percent of tumor DNA in plasma was estimated for samples in the LUCAS and liver cancer cohorts using ichorCNA³⁵.

Generation of the Regional Differences in Single Molecule C>A Frequencies between SCLC and NSCLC

The regional differences in single molecule C>A frequencies were computed as previously described where individuals with SCLC were compared with those with NSCLC. To maximize the number of samples used for identifying bin sets A and B, we combined samples from the high-risk LUCAS cohort (n=10 SCLC, n=75 NSCLC) with individuals who were smokers aged 50-80 from the validation cohort (n=3 SCLC, n=24 NSCLC).

### Analysis of Different Tumor Types

The regional difference in single molecule mutation frequency was computed as previously described by iteratively holding out each individual with either NSCLC, SCLC, or HCC (n=159) and identifying bin sets A and B using all other individuals. For each mutation type (C>A, C>G, C>T, T>A, T>C, and T>G), individuals with NSCLC were compared to those with SCLC, individuals with NSCLC were compared to those with HCC, and individuals with SCLC were compared to those with HCC, yielding 18 regional differences in mutation frequencies per individual. Principal coordinate analysis was performed on the similarity matrix generated from the Euclidean distance between pairwise samples using these 18 regional differences in mutation frequencies. K-means clustering was performed on the matrix of 18 regional differences in mutation frequencies with the number of clusters (k) set to 3. As a negative control, principal coordinate analysis was also performed on a similarity matrix generated from the Euclidean distance between pairwise samples after excluding C>A and T>C mutations that were most frequently observed in lung and liver cancers, resulting in 12 regional differences in mutation frequencies per individual.

### Statistics

The Wilcoxon rank sum test was used to generate p-values for two group comparisons. Correlation of continuous variables was performed using either the Pearson product-moment correlation coefficient or Spearman's rank correlation coefficient. All p-values were based on two-sided hypothesis tests. ROC curves were compared using DeLong's test. All confidence intervals for area under the ROC curve indicate a confidence level of 95% and were based on DeLong's method. Confidence intervals for coefficients in logistic regression models assume normality and were indicated at a 95% confidence level. An analysis of variance (ANOVA) was performed, and an F-test was used to assess whether the between sequencing lane variation of C>A frequencies or regional C>A frequencies were statistically significant. Analyses were performed with R ε 3.6.1 and Python 3.8.2. All boxplots represent the interquartile range with whiskers drawn to the highest value within the upper and lower fences (upper fence=0.75 quantile+1.5×interquartile range; lower fence=0.25 quantile ˜1.5 33 interquartile range). The solid middle line in the boxplot corresponds to the median value.

### Data Availability

Computer code, software versions, and the computing environment for reproducing results from this study will be provided as a GitHub repository (github.com/cancer-genomics/gemini_wflow). Sequence data and clinical variables from the LUCAS study are available from the European Genome-Phenome Archive (EGA) under accession code EGAS00001005340.

TABLE 1
Sensitivity of GEMINI and DELFI followed
by LDCT for lung cancer detection*

| | | GEMINI, | GEMINI, DELFI, |
|---|---|---|---|
| | GEMINI | DELFI | LDCT |
| Overall (n = 89) | 76% | 91% | 93% |
| Overall blended (n = 89) | 69% | 88% | 91% |
| Stage | | | |
| Stage I (n = 13) | 62% | 85% | 88% |
| Stage II (n = 7) | 86% | 86% | 95% |
| Stage III (n = 30) | 73% | 90% | 95% |
| Stage IV (n = 39) | 82% | 95% | 93% |
| Histology | | | |
| Adenocarcinoma (n = 45) | 73% | 89% | 93% |
| Squamous (n = 24) | 71% | 88% | 91% |
| Small cell (n = 10) | 100% | 100% | 95% |

*Sensitivities were determined at specificities of 80% for GEMINI or GEMINI, DELFI, or at a combined specificity of 85% for GEMINI, DELFI, LDCT with a GEMINI, DELFI pre-screen specificity of 62%. DELFI had overall sensitivity of 80% at 80% specificity. Based on the high-risk subset of patients analyzed from the LUCAS cohort¹⁸, LDCT in this setting had a specificity of 66% and an assumed sensitivity of 95% Overall blended sensitivity reflects the sensitivity expected in a screening population weighted by the proportion of lung cancers detected in the NLST population at each stage⁵.

### Other Embodiments

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

### REFERENCES

1. Sung, H. et al. Global Cancer Statistics 2020: GLOBOCAN Estimates of Incidence and Mortality Worldwide for 36 Cancers in 185 Countries. Ca Cancer J Clin 71, 209-249 (2021).
2. World Health Organization. Guide to Cancer Early Diagnosis. (2017).
3. Moyer, V. A. U.S. Preventive Services Task Force. Screening for lung cancer: U.S. Preventive Services Task Force recommendation statement. Annals of Internal Medicine 160, 330-8 (2014).
4. Koning, H. J. de et al. Reduced Lung-Cancer Mortality with Volume CT Screening in a Randomized Trial. New Engl J Med 382, 503-513 (2020).
5. National Lung Screening Trial Research Team. Reduced Lung-Cancer Mortality with Low-Dose Computed Tomographic Screening. New Engl J Medicine 365, 395-409 (2011).
6. Centers for Disease Control and Prevention, National Center for Health Statistics. Lung Cancer National Health Interview Survey. https://processreport.cancer.gov/detection/lung_cancer (2021).
7. American Cancer Society. American Cancer Society Guidelines for the Early Detection of Cancer. (2022).
8. Phallen, J. et al. Direct detection of early-stage cancers using circulating tumor DNA. Sci Transl Med 9, eaan2415 (2017).
9. Bettegowda, C. et al. Detection of Circulating Tumor DNA in Early- and Late-Stage Human Malignancies. Sci Transl Med 6, 224ra24-224ra24 (2014).
10. Cohen, J. D. et al. Detection and localization of surgically resectable cancers with a multi-analyte blood test. Science 359, 926-930 (2018).
11. Phallen, J. et al. Early Noninvasive Detection of Response to Targeted Therapy in Non-Small Cell Lung Cancer. Cancer Res 79, 1204-1213 (2019).
12. Newman, A. M. et al. Integrated digital error suppression for improved detection of circulating tumor DNA. Nat Biotechnol 34, 547-555 (2016).
13. Cristiano, S. et al. Genome-wide cell-free DNA fragmentation in patients with cancer. Nature 570, 385-389 (2019).
14. Shen, S. Y. et al. Sensitive tumour detection and classification using plasma cell-free DNA methylomes. Nature 563, 579-583 (2018).
15. Chabon, J. J. et al. Integrating genomic features for non-invasive early lung cancer detection. Nature 580, 245-251 (2020).
16. Leal, A. et al. White blood cell and cell-free DNA analyses for detection of residual disease in gastric cancer. Nat Commun 11, 525 (2019).
17. Razavi, P. et al. High-intensity sequencing reveals the sources of plasma circulating cell-free DNA variants. Nat Med 25, 1928-1937 (2019).
18. Mathios, D. et al. Detection and characterization of lung cancer using cell-free DNA fragmentomes. Nat Commun 12, 5060 (2021).
19. Siejka-Zielinska, P. et al. Cell-free DNA TAPS provides multimodal information for early cancer detection. Sci Adv 7, eabh0534 (2021).
20. Wang, T.-L. et al. Prevalence of somatic alterations in the colorectal cancer cell genome. Proc National Acad Sci 99, 3076-3080 (2002).
21. Sjöblom, T. et al. The Consensus Coding Sequences of Human Breast and Colorectal Cancers. Science 314, 268-274 (2006).
22. Zviran, A. et al. Genome-wide cell-free DNA mutational integration enables ultra-sensitive cancer monitoring. Nat Med 26, 1114-1124 (2020).
23. Leary, R. J. et al. Development of Personalized Tumor Biomarkers Using Massively Parallel Sequencing. Sci Transl Med 2, 20ra14-20ra14 (2010).
24. Wan, J. C. M. et al. Genome-wide mutational signatures in low-coverage whole genome sequencing of cell-free DNA. Nat Commun 13, 4953 (2022).
25. The ICGC/TCGA Pan-Cancer Analysis of Whole Genomes Consortium. Pan-cancer analysis of whole genomes. Nature 578, 82-93 (2020).
26. Alexandrov, L. B. et al. The repertoire of mutational signatures in human cancer. Nature 578, 94-101 (2020).
27. Chen, L., Liu, P., EvansJr., T. C. & Ettwiller, L. M. DNA damage is a pervasive cause of sequencing errors, directly confounding variant identification. Science 355, 752-756 (2017).
28. Moss, J. et al. Comprehensive human cell-type methylation atlas reveals origins of circulating cell-free DNA in health and disease. Nat Commun 9, 5068 (2018).
29. Alexandrov, L. B. et al. Mutational signatures associated with tobacco smoking in human cancer. Science 354, 618-622 (2016).
30. Lawrence, M. S. et al. Mutational heterogeneity in cancer and the search for new cancer genes. Nature 499, 214-218 (2013).
31. Gonzalez-Perez, A., Sabarinathan, R. & Lopez-Bigas, N. Local Determinants of the Mutational Landscape of the Human Genome. Cell 177, 101-114 (2019).
32. Mouliere, F. et al. Enhanced detection of circulating tumor DNA by fragment size analysis. Sci Transl Med 10, eaat4921 (2018).
33. Fortin, J.-P. & Hansen, K. D. Reconstructing A/B compartments as revealed by Hi-C using long-range correlations in epigenetic data. Genome Biol 16, 180 (2015).
34. Barski, A. et al. High-Resolution Profiling of Histone Methylations in the Human Genome. Cell 129, 823-837 (2007).
35. Adalsteinsson, V. A. et al. Scalable whole-exome sequencing of cell-free DNA reveals high concordance with metastatic tumors. Nat Commun 8, 1324 (2017).
36. Almodovar, K. et al. Longitudinal Cell-Free DNA Analysis in Patients with Small Cell Lung Cancer Reveals Dynamic Insights into Treatment Efficacy and Disease Relapse. J Thorac Oncol 13, 112-123 (2018).
37. Phillips, D. H. & Venitt, S. DNA and protein adducts in human tissues resulting from exposure to tobacco smoke. Int J Cancer 131, 2733-2753 (2012).
38. Supek, F. & Lehner, B. Differential DNA mismatch repair underlies mutation rate variation across the human genome. Nature 521, 81-84 (2015).
39. Consortium, T. I. P.-C. A. of W. G. Pan-cancer analysis of whole genomes. Nature 578, 82-93 (2020).
40. Billatos, E. et al. Detection of early lung cancer among military personnel (DECAMP) consortium: study protocols. Bmc Pulm Med 19, 59 (2019).
41. Li, H. et al. The Sequence Alignment/Map format and SAMtools. Bioinformatics 25, 2078-2079 (2009).
42. Chen, S., Zhou, Y., Chen, Y. & Gu, J. fastp: an ultra-fast all-in-one FASTQ preprocessor. Bioinformatics 34, i884-i890 (2018).
43. Langmead, B. & Salzberg, S. L. Fast gapped-read alignment with Bowtie 2. Nat Methods 9, 357-359 (2012).
44. Tarasov, A., Vilella, A. J., Cuppen, E., Nijman, I. J. & Prins, P. Sambamba: fast processing of NGS alignment formats. Bioinformatics 31, 2032-2034 (2015).
45. Karczewski, K. J. et al. The mutational constraint spectrum quantified from variation in 141,456 humans. Nature 581, 434-443 (2020).
46. Wilks, C. et al. recount3: summaries and queries for large-scale RNA-seq expression and splicing. Biorxiv 2021.05.21.445138 (2021) doi:10.1101/2021.05.21.445138.
47. Thurman, R. E., Day, N., Noble, W. S. & Stamatoyannopoulos, J. A. Identification of higher-order functional domains in the human ENCODE regions. Genome Res 17, 917-927 (2007).
48. The ENCODE Project Consortium. An Integrated Encyclopedia of DNA Elements in the Human Genome. Nature 489, 57-74 (2012).
49. Hansen, R. S. et al. Sequencing newly replicated DNA reveals widespread plasticity in human replication timing. Proc National Acad Sci 107, 139-144 (2010).

### Other Embodiments

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

SUPPLEMENTARY TABLE 1
Summary of PCAWG cohort and genomic analyses*

| | | | | | Whole | Whole | | Total | |
|---|---|---|---|---|---|---|---|---|---|
| | Lung | Matched | | | genome | genome | | somatic | |
| Patient | cancer | normal | Age at | | coverage | coverage | | point | |
| ID | subtype | specimen | diagnosis | Sex | (tumor) | (normal) | [symbol] | mutations | [symbol] |
| [symbol] | Squamous cell | Blood derived | 53 | Male | 32.1 | 35.5 | 0.31 | 25,120 | 0.37 |
| | carcinoma | | | | | | | | |
| [symbol] | Squamous cell | Solid tissue | 38 | Male | [symbol] | [symbol] | 0.24 | 43,005 | 0.52 |
| | carcinoma | | | | | | | | |
| [symbol] | Squamous cell | Blood derived | 61 | Male | 57.8 | 51.2 | 0.70 | 45,829 | 0.49 |
| | carcinoma | | | | | | | | |
| [symbol] | Squamous cell | Solid tissue | 62 | Male | 66.9 | 36.0 | 0.45 | 137,623 | 0.24 |
| | carcinoma | | | | | | | | |
| [symbol] | Squamous cell | Blood derived | 50 | Male | [symbol] | [symbol] | 0.56 | 24,266 | 0.21 |
| | carcinoma | | | | | | | | |
| [symbol] | Squamous cell | Solid tissue | 64 | Male | 51.5 | 57.7 | 0.63 | 36,861 | 0.42 |
| | carcinoma | | | | | | | | |
| [symbol] | Squamous cell | Blood derived | 73 | Male | 72.7 | 36.2 | 0.40 | 72,403 | 0.39 |
| | carcinoma | | | | | | | | |
| [symbol] | Squamous cell | Solid tissue | 70 | Male | 54.0 | 47.2 | 0.62 | 44,253 | 0.45 |
| | carcinoma | | | | | | | | |
| [symbol] | Squamous cell | Blood derived | 60 | Male | 70.7 | [symbol] | [symbol] | 51,403 | 0.45 |
| | carcinoma | | | | | | | | |
| [symbol] | Squamous cell | Solid tissue | 47 | Male | 67.0 | 55.5 | 0.45 | 44,254 | 0.31 |
| | carcinoma | | | | | | | | |
| [symbol] | Squamous cell | Solid tissue | 58 | Female | [symbol] | 42.0 | 0.71 | 51,403 | 0.43 |
| | carcinoma | | | | | | | | |
| [symbol] | Squamous cell | Blood derived | 73 | Male | 47.8 | 34.6 | 0.58 | [symbol] | 0.41 |
| | carcinoma | | | | | | | | |
| [symbol] | Squamous cell | Solid tissue | 77 | Male | 69.6 | 35.9 | 0.74 | [symbol] | 0.26 |
| | carcinoma | | | | | | | | |
| [symbol] | Squamous cell | Solid tissue | 52 | Male | 31.5 | 31.0 | 0.39 | 35,138 | 0.43 |
| | carcinoma | | | | | | | | |
| [symbol] | Squamous cell | Solid tissue | 54 | Male | 54.8 | 40.9 | 0.22 | 47,444 | 0.55 |
| | carcinoma | | | | | | | | |
| [symbol] | Squamous cell | Solid tissue | 77 | Male | 49.0 | 48.5 | 0.92 | 5,600 | 0 |
| | carcinoma | | | | | | | | |
| [symbol] | Squamous cell | Blood derived | 58 | Male | 56.0 | 49.1 | 0.53 | 24,701 | 0.40 |
| | carcinoma | | | | | | | | |
| [symbol] | Squamous cell | Solid tissue | 62 | Male | [symbol] | 35.8 | [symbol] | 82,212 | 0.54 |
| | carcinoma | | | | | | | | |
| [symbol] | Squamous cell | Blood derived | 70 | Male | [symbol] | 62.0 | [symbol] | 45,233 | 0.40 |
| | carcinoma | | | | | | | | |
| [symbol] | Squamous cell | Solid tissue | 75 | Male | 89.5 | 61.2 | 0.57 | [symbol] | 0.57 |
| | carcinoma | | | | | | | | |
| [symbol] | Squamous cell | Solid tissue | [symbol] | Male | 53.0 | 54.8 | [symbol] | 55,051 | 0.56 |
| | carcinoma | | | | | | | | |
| [symbol] | Squamous cell | Blood derived | 62 | Male | 56.6 | [symbol] | [symbol] | 54,735 | 0.48 |
| | carcinoma | | | | | | | | |
| [symbol] | Squamous cell | Blood derived | 53 | Male | 92.1 | 33.3 | 0.35 | 25,120 | 0.57 |
| | carcinoma | | | | | | | | |
| [symbol] | Squamous cell | Solid tissue | 55 | Male | 40.5 | 45.1 | [symbol] | [symbol] | 0.52 |
| | carcinoma | | | | | | | | |
| [symbol] | Squamous cell | Blood derived | 61 | Male | [symbol] | 31.2 | 0.70 | [symbol] | 0.49 |
| | carcinoma | | | | | | | | |
| [symbol] | Squamous cell | Solid tissue | 62 | Male | 66.5 | 36.0 | 0.43 | 137,822 | 0.24 |
| | carcinoma | | | | | | | | |
| [symbol] | Squamous cell | Blood derived | 50 | Male | 76.6 | [symbol] | 0.56 | [symbol] | 0.21 |
| | carcinoma | | | | | | | | |
| [symbol] | Squamous cell | Solid tissue | 52 | Male | [symbol] | 57.7 | 0.63 | [symbol] | 0.42 |
| | carcinoma | | | | | | | | |
| [symbol] | Squamous cell | Blood derived | 73 | Male | 72.7 | 36.2 | 0.40 | [symbol] | 0.39 |
| | carcinoma | | | | | | | | |
| [symbol] | Squamous cell | Solid tissue | 70 | Male | 54.0 | 47.2 | 0.62 | [symbol] | 0.46 |
| | carcinoma | | | | | | | | |
| [symbol] | Squamous cell | Blood derived | 60 | Female | 70.7 | 34.8 | 0.65 | [symbol] | 0.45 |
| | carcinoma | | | | | | | | |
| [symbol] | Squamous cell | Solid tissue | 47 | Male | 67.0 | 53.5 | 0.48 | 56,610 | 0.51 |
| | carcinoma | | | | | | | | |
| [symbol] | Squamous cell | Solid tissue | 54 | Female | [symbol] | 42.0 | 0.71 | [symbol] | 0.43 |
| | carcinoma | | | | | | | | |
| [symbol] | Squamous cell | Blood derived | 73 | Female | 47.8 | [symbol] | 0.62 | [symbol] | 0.41 |
| | carcinoma | | | | | | | | |
| [symbol] | Squamous cell | Solid tissue | 77 | Female | 69.6 | 35.9 | 0.74 | 13,202 | 0.25 |
| | carcinoma | | | | | | | | |
| [symbol] | Squamous cell | Solid tissue | 52 | Male | 51.3 | 51.0 | 0.59 | [symbol] | 0.48 |
| | carcinoma | | | | | | | | |
| [symbol] | Squamous cell | Solid tissue | 54 | Female | 54.2 | 40.9 | 0.22 | 47,444 | 0.55 |
| | carcinoma | | | | | | | | |
| [symbol] | Squamous cell | Solid tissue | 77 | Male | 49.0 | 48.5 | 0.92 | [symbol] | 0 |
| | carcinoma | | | | | | | | |
| [symbol] | Squamous cell | Blood derived | 56 | Male | 56.0 | 49.1 | 0.33 | [symbol] | 0.40 |
| | carcinoma | | | | | | | | |
| [symbol] | Squamous cell | Solid tissue | 68 | Male | 61.0 | [symbol] | 0.62 | 51,112 | 0.54 |
| | carcinoma | | | | | | | | |
| [symbol] | Squamous cell | Blood derived | 70 | Male | 63.6 | [symbol] | [symbol] | [symbol] | 0.40 |
| | carcinoma | | | | | | | | |
| [symbol] | Squamous cell | Solid tissue | 75 | Male | 89.6 | 61.2 | 0.57 | [symbol] | 0.57 |
| | carcinoma | | | | | | | | |
| [symbol] | Squamous cell | Solid tissue | 65 | Male | 53.0 | 54.8 | [symbol] | [symbol] | 0.36 |
| | carcinoma | | | | | | | | |
| [symbol] | Squamous cell | Blood derived | 68 | Male | 56.6 | 32.0 | [symbol] | [symbol] | 0.48 |
| | carcinoma | | | | | | | | |
| [symbol] | Squamous cell | Solid tissue | [symbol] | Male | 55.9 | 44.4 | 0.75 | [symbol] | 0 |
| | carcinoma | | | | | | | | |
| [symbol] | Squamous cell | Blood derived | 61 | Male | 71.0 | 67.2 | 0.41 | [symbol] | 0.33 |
| | carcinoma | | | | | | | | |
| [symbol] | Squamous cell | Blood derived | 58 | Male | [symbol] | 63.2 | 0.32 | [symbol] | 0.45 |
| | carcinoma | | | | | | | | |
| [symbol] | Squamous cell | Solid tissue | 65 | Male | [symbol] | [symbol] | 0.35 | [symbol] | 0.58 |
| | carcinoma | | | | | | | | |
| [symbol] | Squamous cell | Blood derived | 53 | Male | 66.6 | 57.6 | 0.63 | [symbol] | 0.36 |
| | carcinoma | | | | | | | | |
| [symbol] | Squamous cell | Solid tissue | 64 | Female | 50.0 | 54.5 | 0.62 | [symbol] | [symbol] |
| | carcinoma | | | | | | | | |
| [symbol] | Squamous cell | Blood derived | 66 | Male | 69.6 | 41.7 | 0.45 | [symbol] | 0.51 |
| | carcinoma | | | | | | | | |
| [symbol] | Squamous cell | Solid tissue | 71 | Male | 65.4 | 51.5 | 0.39 | [symbol] | 0.42 |
| | carcinoma | | | | | | | | |
| [symbol] | Squamous cell | Blood derived | 62 | Male | 69.4 | 49.1 | 0.56 | [symbol] | 0.71 |
| | carcinoma | | | | | | | | |
| [symbol] | Squamous cell | Solid tissue | 76 | Female | 40.1 | 66.9 | 0.34 | [symbol] | [symbol] |
| | carcinoma | | | | | | | | |
| [symbol] | Squamous cell | Solid tissue | 74 | Male | 67.3 | 45.1 | 0.40 | [symbol] | 0.17 |
| | carcinoma | | | | | | | | |
| [symbol] | Squamous cell | Blood derived | 66 | Female | 79.9 | [symbol] | 0.47 | [symbol] | 0.44 |
| | carcinoma | | | | | | | | |
| [symbol] | Squamous cell | Solid tissue | 68 | Male | 44.1 | 49.2 | 0.70 | [symbol] | 0.65 |
| | carcinoma | | | | | | | | |
| [symbol] | Squamous cell | Solid tissue | 73 | Female | 20.7 | 35.5 | 0.55 | [symbol] | 0.18 |
| | carcinoma | | | | | | | | |
| [symbol] | Squamous cell | Solid tissue | 75 | Male | 52.4 | [symbol] | 0.55 | [symbol] | 0.43 |
| | carcinoma | | | | | | | | |
| [symbol] | Squamous cell | Solid tissue | [symbol] | Male | 70.2 | [symbol] | 0.44 | [symbol] | 0.59 |
| | carcinoma | | | | | | | | |
| [symbol] | Squamous cell | Solid tissue | 66 | Male | [symbol] | [symbol] | 0.33 | [symbol] | 0.41 |
| | carcinoma | | | | | | | | |
| [symbol] | Squamous cell | Solid tissue | 62 | Male | 74.1 | [symbol] | [symbol] | [symbol] | 0.41 |
| | carcinoma | | | | | | | | |
| [symbol] | Squamous cell | Solid tissue | [symbol] | Female | 65.0 | 31.1 | [symbol] | [symbol] | 0.17 |
| | carcinoma | | | | | | | | |
| [symbol] | Squamous cell | Solid tissue | 56 | Male | 92.2 | 21.0 | 0.47 | [symbol] | 0.42 |
| | carcinoma | | | | | | | | |
| [symbol] | Squamous cell | Blood derive | 74 | Male | 55.4 | 30.6 | 0.70 | [symbol] | 0 |
| | carcinoma | | | | | | | | |
| [symbol] | Squamous cell | Blood derive | 56 | Male | 61.2 | 50.3 | 0.73 | [symbol] | 0.39 |
| | carcinoma | | | | | | | | |
| [symbol] | Squamous cell | Solid tissue | 54 | Male | 52.0 | 50.9 | 0.75 | [symbol] | 0.39 |
| | carcinoma | | | | | | | | |
| [symbol] | Adenocarcinoma | Solid tissue | 62 | Female | 47.0 | 43.1 | 0.02 | [symbol] | 0 |
| [symbol] | Adenocarcinoma | Blood derived | 59 | Female | 37.7 | 60.0 | 0.63 | [symbol] | 0 |

| | | Donor | | | | | | | | | | |
|---|---|---|---|---|---|---|---|---|---|---|---|---|
| | | used for | | | | | | | | | | |
| | | single | | | | | | | | | | |
| Patient | | molecule | | | | | | | | | | |
| ID | [symbol] | analyses | [symbol] | [symbol] | [symbol] | [symbol] | [symbol] | [symbol] | [symbol] | [symbol] | [symbol] | [symbol] |
| [symbol] | Yes | Yes | [symbol] | 1.95 | [symbol] | [symbol] | 4.24 | 3.57 | 601,701 | 42.67 | [symbol] | 1.07 |
| [symbol] | Yes | No | — | — | — | — | — | — | — | — | — | — |
| [symbol] | Yes | Yes | 2.64 | 1.84 | [symbol] | 57.63 | 13.55 | 14.53 | [symbol] | 24.64 | 6.73 | 7.20 |
| [symbol] | Yes | No | — | — | — | — | — | — | — | — | — | — |
| [symbol] | Yes | Yes | 1.45 | 1.10 | [symbol] | 67.59 | 2.20 | 2.16 | [symbol] | [symbol] | [symbol] | [symbol] |
| [symbol] | Yes | No | — | — | — | — | — | — | — | — | — | — |
| [symbol] | Yes | Yes | 1.34 | 1.97 | [symbol] | [symbol] | 0.68 | 1.20 | [symbol] | 31.75 | 1.71 | 2.29 |
| [symbol] | Yes | No | — | — | — | — | — | — | — | — | — | — |
| [symbol] | Yes | Yes | 1.39 | 1.38 | [symbol] | [symbol] | 16.61 | 16.74 | [symbol] | [symbol] | [symbol] | 0.51 |
| [symbol] | Yes | No | — | — | — | — | — | — | — | — | — | — |
| [symbol] | Yes | No | — | — | — | — | — | — | — | — | — | — |
| [symbol] | Yes | Yes | 1.93 | [symbol] | [symbol] | 15.34 | 11.14 | 12.79 | [symbol] | [symbol] | 0.44 | 0.54 |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | No | No | — | — | — | — | — | — | — | — | — | — |
| [symbol] | Yes | Yes | 1.39 | 1.91 | [symbol] | 47.98 | 7.96 | 9.61 | [symbol] | [symbol] | [symbol] | [symbol] |
| [symbol] | Yes | No | — | — | — | — | — | — | — | — | — | — |
| [symbol] | Yes | Yes | 1.84 | 2.01 | [symbol] | 52.45 | 13.45 | 13.54 | [symbol] | 40.44 | 0.78 | 1.17 |
| [symbol] | Yes | No | — | — | — | — | — | — | — | — | — | — |
| [symbol] | Yes | No | — | — | — | — | — | — | — | — | — | — |
| [symbol] | Yes | Yes | 1.44 | 0.87 | [symbol] | [symbol] | [symbol] | 15.25 | [symbol] | 144.75 | 5.60 | 5.82 |
| [symbol] | Yes | Yes | 1.60 | 1.95 | 2,513,169 | [symbol] | 4.14 | 3.57 | [symbol] | 42.67 | 0.80 | 1.07 |
| [symbol] | Yes | No | — | — | — | — | — | — | — | — | — | — |
| [symbol] | Yes | Yes | 2.84 | [symbol] | [symbol] | 57.63 | 15.63 | 14.55 | 1,400,173 | 24.61 | 6.73 | 7.20 |
| [symbol] | Yes | No | — | — | — | — | — | — | — | — | — | — |
| [symbol] | Yes | Yes | 1.45 | 1.10 | [symbol] | 67.59 | 1.10 | 2.16 | [symbol] | 144.32 | 9.09 | 9.24 |
| [symbol] | Yes | No | — | — | — | — | — | — | — | — | — | — |
| [symbol] | Yes | Yes | 1.34 | 1.97 | [symbol] | 115.74 | 0.63 | 1.20 | [symbol] | 31.75 | 1.71 | 2.29 |
| [symbol] | Yes | No | — | — | — | — | — | — | — | — | — | — |
| [symbol] | Yes | Yes | 1.59 | 1.38 | [symbol] | 30.27 | 16.61 | 18.74 | [symbol] | 63.32 | 0.02 | 0.51 |
| [symbol] | Yes | No | — | — | — | — | — | — | — | — | — | — |
| [symbol] | Yes | No | — | — | — | — | — | — | — | — | — | — |
| [symbol] | Yes | Yes | 1.98 | 1.96 | [symbol] | 45.24 | 11.11 | 12.79 | [symbol] | 40.78 | 0.41 | 0.54 |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | No | No | — | — | — | — | — | — | — | — | — | — |
| [symbol] | Yes | Yes | 1.59 | 1.94 | [symbol] | 47.96 | 7.96 | 9.54 | 751,518 | 40.10 | 0.56 | [symbol] |
| [symbol] | Yes | No | — | — | — | — | — | — | — | — | — | — |
| [symbol] | Yes | Yes | [symbol] | [symbol] | [symbol] | 32.48 | 13.43 | 13.34 | [symbol] | 40.46 | 0.75 | 1.17 |
| [symbol] | Yes | No | — | — | — | — | — | — | — | — | — | — |
| [symbol] | Yes | No | — | — | — | — | — | — | — | — | — | — |
| [symbol] | Yes | Yes | 1.14 | 0.87 | [symbol] | 71.50 | 24.60 | 23.25 | [symbol] | [symbol] | 5.60 | 3.82 |
| [symbol] | No | No | — | — | — | — | — | — | — | — | — | — |
| [symbol] | Yes | Yes | 1.23 | 2.20 | [symbol] | 33.30 | 6.42 | 6.13 | [symbol] | 40.79 | 0.85 | 0.27 |
| [symbol] | Yes | Yes | 1.55 | 2.12 | [symbol] | 57.99 | 7.43 | 8.41 | [symbol] | 40.95 | 1.42 | 1.77 |
| [symbol] | Yes | No | — | — | — | — | — | — | — | — | — | — |
| [symbol] | Yes | Yes | 1.41 | 1.76 | [symbol] | [symbol] | 7.76 | 6.93 | [symbol] | 49.43 | 1.80 | 2.18 |
| [symbol] | Yes | No | — | — | — | — | — | — | — | — | — | — |
| [symbol] | Yes | Yes | 1.54 | 1.76 | [symbol] | 16.41 | 7.76 | 6.93 | [symbol] | 49.45 | 1.50 | 2.10 |
| [symbol] | Yes | No | — | — | — | — | — | — | — | — | — | — |
| [symbol] | Yes | Yes | 1.36 | 1.92 | [symbol] | 52.53 | 15.25 | 15.22 | [symbol] | 49.51 | 1.17 | 1.31 |
| [symbol] | Yes | No | — | — | — | — | — | — | — | — | — | — |
| [symbol] | Yes | No | — | — | — | — | — | — | — | — | — | — |
| [symbol] | Yes | Yes | 1.79 | 1.34 | [symbol] | 43.10 | [symbol] | 10.95 | [symbol] | 37.40 | 0.93 | 0.27 |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | Yes | No | — | — | — | — | — | — | — | — | — | — |
| [symbol] | No | No | — | — | — | — | — | — | — | — | — | — |
| [symbol] | Yes | Yes | 1.37 | 2.00 | [symbol] | 27.01 | 7.72 | 7.69 | [symbol] | 49.90 | 1.35 | [symbol] |
| [symbol] | Yes | No | — | — | — | — | — | — | — | — | — | — |
| [symbol] | No | No | — | — | — | — | — | — | — | — | — | — |
| [symbol] | No | No | — | — | — | — | — | — | — | — | — | — |

[symbol]

indicates data missing or illegible when filed

SUPPLEMENTARY TABLE 2
Effect of variant filters in tissue and cfDNA samples*

| | PCAWG tumor samples (n = 31) | | | | | | |
|---|---|---|---|---|---|---|---|
| | | | | Somatic | Background | | Cumulative fold |
| | | | Total | single | single | Single | enrichment of |
| | | | single | molecule | molecule | molecule | frequency over |
| | Known | Background | molecule | matation | mutation | mutation | background |
| | somatic | sequence | sequence | frequency | frequency | frequency | mutation |
| Variant filter | mutations | changes | changes | (Muts/Mb) | (Muts/Mb) | (Muts/Mb) | frequency |
| No filter | 8,776 | 3,931,580 | 8,940,356 | 5.2 | 2333.8 | 2339.0 | — |
| [symbol] | 6,741 | 571,787 | 578,528 | 5.3 | 448.9 | 454.2 | 5.3 |
| MAPQ <40 | 6,713 | 524,475 | 531,187 | 5.5 | 427.6 | 433.1 | 5.8 |
| Blacklisted regions | 6,649 | 519,667 | 526,316 | 5.4 | 425.1 | 430.6 | 5.8 |
| Germline variants | 6,354 | 272,504 | 278,859 | 5.2 | 229.1 | 234.3 | 10.3 |
| 8-oxo-dG related sequence changes | 3,188 | 42,513 | 45,701 | 5.2 | 68.4 | 73.7 | 34.6 |
| Not in F and R reads (cfDNA only) | 3,188 | 42,513 | 45,701 | 5.2 | 68.4 | 73.7 | 34.6 |
| Regional mutation frequency | | | | 12.7 | 3.0 | 15.8 | 1,909.3 |
| difference^{†} | | | | | | | |

| | PCAWG blood | | | | LUCAS non-cancer | |
|---|---|---|---|---|---|---|
| | derived normals | | LUCAS cancer | | cfDNA (n = 74) | |
| | | Background | cfDNA (n = 89) | | | Background |
| | | single | | Single | Background | single |
| | | molecule | Single | molecule | single | molecule |
| | Background | mutation | molecule | mutation | molecule | mutation |
| | sequence | frequency | sequence | frequency | sequence | frequency |
| Variant filter | changes | (Muts/Mb) | changes | (Muts/Mb) | changes | (Muts/Mb) |
| No filter | [symbol] | 2355.3 | 2,801,880 | 1247.1 | 2,860,927 | 1244.6 |
| [symbol] | 540,299 | 420.8 | 944,435 | 467.7 | 958,189 | 462.6 |
| MAPQ <40 | 493,980 | 399.1 | 396,155 | 224.3 | 403,275 | 222.4 |
| Blacklisted regions | 489,178 | 396.6 | 387,596 | 220.1 | 394,657 | 218.3 |
| Germline variants | 241,459 | 198.7 | 63,172 | 36.1 | 63,769 | 35.4 |
| 8-oxo-dG related sequence changes | 40,468 | 67.3 | 31,178 | 35.7 | 31,468 | 35.0 |
| Not in F and R reads (cfDNA only) | 40,468 | 67.3 | 2,901 | 21.0 | 2,498 | 19.0 |
| Regional mutation frequency | | 2.1 | | 7.7 | | 0 |
| difference^{†} | | | | | | |

*Sequence data from tissue samples were randomly downsampled to [symbol] 2x coverage similar to cfDNA samples. Values reflect the average across samples.
^{†}Overall mutation frequencies in cfDNA samples were scaled such that non-cancer samples have mean values of zero. [symbol]
indicates data missing or illegible when filed

SUPPLEMENTARY TABLE 3
Summary of LUCAS cohort and genomic analyses⁺

| | | | | | | | | Autoimmune |
|---|---|---|---|---|---|---|---|---|
| | | | | | | | | disease |
| Patient | | Histological | Age at | | | | | (0 = No, |
| ID | Patient type | dagnosis | diagnosis | Sex | Stage | Smoking status | Pack years | 1 = Yes) |
| [symbol] | benign | Benign | 46 | Male | NA | smoker, stopped | 15 | 0 |
| | | | | | | over 6 months | | |
| [symbol] | no baseline | No baseline | 31 | Female | NA | smoker | 30 | 0 |
| | cancer | cancer | | | | | | |
| [symbol] | no lung cancer, | No baseline | 70 | Female | NA | never smoker | 0 | 0 |
| | other cancer, | cancer | | | | | | |
| | later lung cancer | | | | | | | |
| [symbol] | no lung cancer, | Benign | 50 | Male | NA | smoker, stopped | 10 | 0 |
| | other cancer | | | | | over 6 months | | |
| [symbol] | no baseline | No baseline | 73 | Male | NA | smoker | 100 | 1 |
| | cancer | cancer | | | | | | |
| [symbol] | benign | Benign | 23 | Male | NA | smoker, stopped | 1 | 0 |
| | | | | | | under 6 months | | |
| [symbol] | no baseline | No baseline | 59 | Male | NA | smoker | 43 | 0 |
| | cancer | cancer | | | | | | |
| [symbol] | no lung cancer, | Benign | 63 | Female | NA | smoker, stoppped | 15 | 1 |
| | other cancer | | | | | over 6 months | | |
| [symbol] | no lung cancer, | Benign | 71 | Male | NA | smoker | 60 | 0 |
| | other cancer | | | | | | | |
| [symbol] | lung cancer | Squamous | 60 | Female | [symbol] | smoker, stopped | 3 | 0 |
| | | | | | | over 6 months | | |
| [symbol] | no baseline | No baseline | 14 | Female | NA | never smoker | 0 | 1 |
| | cancer | cancer | | | | | | |
| [symbol] | lung cancer | Adenocarcinoma | 75 | Male | [symbol] | smoker, stopped | 50 | 0 |
| | | | | | | over 6 months | | |
| [symbol] | lung cancer | Adenocarcinoma | 62 | Male | IV | smmoker, stopped | 80 | 0 |
| | | | | | | over 6 months | | |
| [symbol] | no lung cancer, | No baseline | 69 | Female | NA | smoker, stopped | [symbol] | 1 |
| | other cancer | cancer | | | | ovver 6 months | | |
| [symbol] | no baseline | No baseline | 96 | Female | NA | never smoker | 0 | 1 |
| | cancer | cancer | | | | | | |
| [symbol] | no baseline | No baseline | 20 | Female | NA | smoker, stopped | 15 | 0 |
| | cancer | cancer | | | | over 6 months | | |
| [symbol] | no baseline | No baseline | 35 | Male | NA | smoker | 45 | 0 |
| | cancer | cancer | | | | | | |
| [symbol] | benign | Benign | 15 | Male | NA | smoker | 17 | 0 |
| [symbol] | lung cancer, | Adenocarcinoma | 71 | Female | [symbol] | smoker, stopped | 35 | 0 |
| | [symbol] lung cancer | | | | | over 6 months | | |
| [symbol] | no baseline | No baseline | 75 | Female | NA | smoker, stopped | 1 | 1 |
| | cancer | cancer | | | | over 6 months | | |
| [symbol] | no lung cancer, | No baseline | 30 | Female | NA | never smoker | 0 | 0 |
| | other cancer | cancer | | | | | | |
| [symbol] | no lung cancer, | No baseline | 70 | Female | NA | smoker | 35 | 1 |
| | other cancer | cancer | | | | | | |
| [symbol] | lung cancer | Adenocarcinoma | 79 | Female | IV | smoker | 30 | 0 |
| [symbol] | no baseline | No baseline | 37 | Male | NA | never smoker | 0 | 0 |
| | cancer | cancer | | | | | | |
| [symbol] | no lung cancer, | No baseline | 73 | Male | NA | smoker, stopped | 15 | 0 |
| | other cancer | cancer | | | | over 6 months | | |
| [symbol] | no baseline | No baseline | 65 | Female | NA | smoker, stopped | 40 | 0 |
| | cancer | cancer | | | | over 6 months | | |
| [symbol] | benign | Benign | 68 | Female | NA | smoker, stopped | 1 | 1 |
| | | | | | | over 6 months | | |
| [symbol] | no lung cancer, | Benign | 57 | Male | NA | smoker | 60 | 0 |
| | other cancer | | | | | | | |
| [symbol] | no baseline | No baseline | 84 | Female | NA | never smoker | 0 | 0 |
| | cancer | cancer | | | | | | |
| [symbol] | lung cancer | Adenocarcinoma | 60 | Male | [symbol] | smoker | 10 | 0 |
| [symbol] | lung cancer | Squamous | 53 | Female | [symbol] | smoker | 35 | 0 |
| [symbol] | no baseline | No baseline | 56 | Female | NA | smoker, stopped | 62 | 0 |
| | cancer | cancer | | | | under 6 months | | |
| [symbol] | no baseline | No baseline | 65 | Female | NA | smoker | 46 | 1 |
| | cancer | cancer | | | | | | |
| [symbol] | no baseline | No baseline | 73 | Male | NA | smoker, stopped | 34 | 0 |
| | cancer | cancer | | | | over 6 months | | |
| [symbol] | lung cancer | Adenocarcinoma | 56 | Male | IV | smoker, stopped | 25 | 0 |
| | | | | | | over 6 months | | |
| [symbol] | lung cancer | Adenocarcinoma | 63 | Male | [symbol] | smoker | 40 | 0 |
| [symbol] | benign | Benign | 44 | Male | NA | smoker | 30 | 0 |
| [symbol] | benign | Benign | 75 | Female | NA | smoker, stopped | 20 | 1 |
| | | | | | | over 6 months | | |
| [symbol] | no baseline | No baseline | 57 | Female | NA | smoker | 30 | 1 |
| | cancer | cancer | | | | | | |
| [symbol] | lung cancer | Adenocarcinoma | [symbol] | Male | IV | smoker, stopped | 25 | 0 |
| | | | | | | over 6 months | | |
| [symbol] | no lung cancer, | No baseline | 73 | Male | NA | smoker | 17 | 0 |
| | other cancer | cancer | | | | | | |
| [symbol] | lung cancer | Adenocarcinoma | 65 | Male | IV | never smoker | 0 | 0 |
| [symbol] | lung cancer, | Adenocarcinoma | 72 | Male | IV | smoker, stopped | 40 | 0 |
| | other cancer | | | | | over 6 months | | |
| [symbol] | no baseline | No baseline | [symbol] | Male | NA | smoker | 40 | 0 |
| | cancer | cancer | | | | | | |
| [symbol] | lung cancer | Squamous | 73 | Male | [symbol] | smoker | 50 | 0 |
| [symbol] | no baseline | No baseline | 59 | Male | NA | never smoker | 0 | 0 |
| | cancer | cancer | | | | | | |
| [symbol] | no lung cancer, | No baseline | 61 | Male | NA | smoker | 23 | 0 |
| | other cancer | cancer | | | | | | |
| [symbol] | no lung cancer, | No baseline | 63 | Male | NA | smoker, stopped | 30 | 0 |
| | other cancer | cancer | | | | over 6 months | | |
| [symbol] | lung cancer | Adenocarcinoma | 71 | Female | IV | smoker, stopped | 40 | 0 |
| | | | | | | over 6 months | | |
| [symbol] | lung cancer | Squamos | 65 | Female | [symbol] | unknown | 20 | 0 |
| [symbol] | benign | Benign | 47 | Female | NA | smoker, stopped | 3 | 0 |
| | | | | | | over 6 months | | |
| [symbol] | lung cancer, | Squamous | 63 | Female | [symbol] | smoker, stopped | 40 | 1 |
| | other cancer | | | | | over 6 months | | |
| [symbol] | no lung cancer, | No baseline | 79 | Female | NA | smoker | 10 | 1 |
| | other cancer | cancer | | | | | | |
| [symbol] | lung cancer | Adenocarcinoma | 55 | Female | [symbol] | smoker | 40 | 1 |
| [symbol] | no lung cancer, | No baseline | [symbol] | Female | NA | smoker, stopped | 4 | 0 |
| | other cancer | cancer | | | | over 6 months | | |
| [symbol] | lung cancer | SCLC | 56 | Female | [symbol] | smoker | 40 | 1 |
| [symbol] | no lung cancer, | No baseline | 92 | Female | NA | smoker, stopped | 15 | 0 |
| | other cancer | cancer | | | | over 6 months | | |
| [symbol] | no lung cancer, | No baseline | 77 | Male | NA | smoker, stopped | 35 | 0 |
| | other cancer | cancer | | | | under 6 months | | |
| [symbol] | no baseline | No baseline | 56 | Male | NA | smoker, stopped | 10 | 0 |
| | cancer | cancer | | | | over 6 months | | |
| [symbol] | lung cancer, | Adenosquamous | 72 | Female | [symbol] | smoker | 52 | 0 |
| | other cancer | | | | | | | |
| [symbol] | benign | Benign | 71 | Female | NA | smoker, stopped | 27 | 1 |
| | | | | | | over 6 months | | |
| [symbol] | lung cancer | Squamous | 53 | Female | NA | smoker, stopped | 45 | 1 |
| | | | | | | under 6 months | | |
| [symbol] | no baseline | No baseline | 53 | Male | NA | smoker | 50 | 0 |
| | cancer | cancer | | | | | | |
| [symbol] | lung cancer | Squamous | 61 | Male | IV | smoker, stopped | 45 | 0 |
| | | | | | | under 6 months | | |
| [symbol] | lung cancer | Adenocarcinoma | 59 | Female | IIIB | smoker | 55 | 1 |
| [symbol] | no lung cancer, | No baseline | 62 | Female | NA | never smoker | 0 | 1 |
| | other cancer | cancer | | | | | | |
| [symbol] | benign | Benign | 64 | Female | NA | never smoker | 0 | 1 |
| [symbol] | benign | Benign | 29 | Male | NA | never smoker | 0 | 0 |
| [symbol] | benign | Benign | 25 | Female | NA | never smoker | 0 | 0 |
| [symbol] | lung cancer | Squamous | 76 | Male | [symbol] | smoker | 40 | 0 |
| [symbol] | no lung cancer, | No baseline | 73 | Male | NA | smoker | 100 | 0 |
| | other cancer | cancer | | | | | | |
| [symbol] | lung cancer, | SCLC | 59 | Male | [symbol] | smoker, stopped | 45 | 0 |
| | other cancer | | | | | over 6 months | | |
| [symbol] | lung cancer | Squamous | 71 | Male | [symbol] | smoker, stopped | 35 | 1 |
| | | | | | | over 6 months | | |
| [symbol] | no lung cancer, | Benign | 72 | Male | NA | smopker | 110 | 0 |
| | other cancer | | | | | | | |
| [symbol] | no baseline | No baseline | [symbol] | Female | NA | smoker, stopped | 40 | 0 |
| | cancer | cancer | | | | under 6 months | | |
| [symbol] | [symbol] | [symbol] | 64 | Male | IV | smoker, stopped | 10 | 1 |
| | | | | | | over 6 months | | |
| [symbol] | lung cancer, | Adenocarcinoma | 71 | Female | [symbol] | smoker | 40 | 0 |
| | other cancer | | | | | | | |
| [symbol] | no lung cancer, | No baseline | 73 | Female | NA | smoker, stopped | 40 | 0 |
| | later lung cancer | cancer | | | | over 6 months | | |
| [symbol] | benign | Benign | 40 | Male | NA | never smoker | 0 | 1 |
| [symbol] | no baseline | No baseline | 41 | Male | NA | smoker, stopped | 21 | 0 |
| | cancer | cancer | | | | over 6 months | | |
| [symbol] | benign | Benign | 60 | Male | NA | smoker, stopped | 32 | 0 |
| | | | | | | over 6 months | | |
| [symbol] | no baseline | No baseline | 43 | Female | NA | smoker, stopped | 22 | 0 |
| | cancer | cancer | | | | over 6 months | | |
| [symbol] | met to lung, | Metastasis from | 76 | Male | IV | smoker, stopped | 1 | 0 |
| | other cancer | another primary | | | | over 6 months | | |
| | | cancer | | | | | | |
| [symbol] | no lung cancer, | Benign | 80 | Male | NA | smoker, stopped | 20 | 1 |
| | other cancer | | | | | over 6 months | | |
| [symbol] | no lung cancer, | No baseline | 52 | Female | NA | smoker, stopped | 12 | 1 |
| | other cancer | cancer | | | | over 6 months | | |
| [symbol] | benign | Benign | 56 | Female | NA | smoker | 50 | 0 |
| [symbol] | no baseline | No baseline | 55 | Female | NA | smoker | 40 | 0 |
| | cancer | cancer | | | | | | |
| [symbol] | benign | Benign | 36 | Male | NA | smoker | 36 | 1 |
| [symbol] | no baseline | No baseline | 39 | Female | NA | smoker, stopped | 38 | 1 |
| | cancer | cancer | | | | over 6 months | | |
| [symbol] | no baseline | No baseline | 71 | Female | NA | smoker, stopped | 20 | 0 |
| | cancer | cancer | | | | over 6 months | | |
| [symbol] | no lung cancer, | No baseline | 82 | Male | NA | smoker | 33 | 0 |
| | other cancer | cancer | | | | | | |
| [symbol] | benign | Benign | 41 | Male | NA | smoker | 25 | 0 |
| [symbol] | no lung cancer, | No baseline | 52 | Female | NA | smoker, stopped | 11 | 0 |
| | other cancer | cancer | | | | over 6 months | | |
| [symbol] | lung cancer, | Squamous | 24 | Male | IV | smoker, stopped | 70 | 0 |
| | prior lung cancer | | | | | over 6 months | | |
| [symbol] | no baseline | No baseline | 79 | Female | NA | smoker, stopped | 40 | 0 |
| | cancer | cancer | | | | over 6 months | | |
| [symbol] | no baseline | No baseline | 21 | Female | NA | smoker, stopped | 25 | 0 |
| | cancer | cancer | | | | under 6 months | | |
| [symbol] | lung cancer, | Adenocarcinoma | 55 | Female | IV | unknown | 35 | 0 |
| | other cancer | | | | | | | |
| [symbol] | no lung cancer, | No baseline | 59 | Female | NA | never smoker | 0 | 1 |
| | other cancer | cancer | | | | | | |
| [symbol] | no lung cancer, | No baseline | 75 | Female | NA | smoker | 40 | 0 |
| | other cancer | cancer | | | | | | |
| [symbol] | no baseline | No baseline | 63 | Female | NA | smoker, stopped | 23 | 0 |
| | cancer | Cancer | | | | under 6 months | | |
| [symbol] | lung cancer, | Squamous | 52 | Female | NA | smoker, stopped | 10 | 0 |
| | other cancer | | | | | over 6 months | | |
| [symbol] | benign | Benign | [symbol] | Female | NA | smoker | [symbol] | 0 |
| [symbol] | lung cancer | Adenocarcinoma | 50 | Male | IV | smoker, stopped | 20 | 0 |
| | | | | | | over 6 months | | |
| [symbol] | lung cancer, | Adenocarcinoma | 78 | Female | [symbol] | smoker, stopped | 22 | 0 |
| | other cancer | | | | | over 6 months | | |
| [symbol] | benign | Benign | 35 | Female | NA | smoker, stopped | 25 | 0 |
| | | | | | | over 6 months | | |
| [symbol] | lung cancer | Squamous | 58 | Male | IV | smoker, stopped | 24 | 1 |
| | | | | | | over 6 months | | |
| [symbol] | no baseline | No baseline | 59 | Male | NA | smoker, stopped | 35 | 0 |
| | cancer | cancer | | | | over 6 months | | |
| [symbol] | benign | Benign | 46 | Male | NA | smoker | 30 | 0 |
| [symbol] | no baseline | No baseline | 46 | Female | NA | smoker, stopped | 35 | 0 |
| | cancer | cancer | | | | under 6 months | | |
| [symbol] | benign | Benign | 53 | Male | NA | never smoker | 0 | 0 |
| [symbol] | met to lung | Metastasis from | 53 | Male | IV | never smoker | 0 | 0 |
| | | another primary | | | | | | |
| | | cancer | | | | | | |
| [symbol] | lung cancer | Adenocarcinoma | 56 | Female | IV | smoker | 52 | 1 |
| [symbol] | no baseline | No baseline | 35 | Female | NA | smoker, stopped | 14 | 0 |
| | cancer | cancer | | | | over 6 months | | |
| [symbol] | no baseline | No baseline | 61 | Male | NA | never smoker | 0 | 0 |
| | cancer | cancer | | | | | | |
| [symbol] | no baseline | No baseline | 52 | Female | NA | smoker, stopped | 23 | 0 |
| | cancer | cancer | | | | over 6 months | | |
| [symbol] | lung cancer | Squamous | 75 | Male | IV | smoker, stopped | 73 | 0 |
| | | | | | | over 6 months | | |
| [symbol] | no baseline | No baseline | 67 | Male | NA | smoker | 47 | 0 |
| | cancer | cancer | | | | | | |
| [symbol] | lung cancer, | Unknown | 74 | Male | IV | smoker, stopped | 50 | 1 |
| | prior lung cancer | | | | | over 6 months | | |
| [symbol] | benign | Benign | 56 | Male | NA | smoker, stopped | 5 | 0 |
| | | | | | | over 6 months | | |
| [symbol] | lung cancer | Squamous | 76 | Male | IV | unknown | NA | 0 |
| [symbol] | no baseline | No baseline | 53 | Female | NA | smoker, stopped | 10 | 0 |
| | cancer | cancer | | | | under 6 months | | |
| [symbol] | no baseline | No baseline | 34 | Male | NA | smoker, stopped | 3 | 0 |
| | cancer | cancer | | | | over 6 months | | |
| [symbol] | no baseline | No baseline | 64 | Female | NA | never smoker | 0 | 1 |
| | cancer | cancer | | | | | | |
| [symbol] | lung cancer, | SCLC | 57 | Female | [symbol] | smoker | 40 | 0 |
| | other cancer | | | | | | | |
| [symbol] | lung cancer | Adenocarcinoma | 62 | Male | IV | smoker, stopped | 40 | 0 |
| | | | | | | over 6 months | | |
| [symbol] | no lung cancer, | No baseline | 71 | Male | NA | smoker, stopped | 45 | 1 |
| | other cancer | cancer | | | | over 6 months | | |
| [symbol] | lung cancer | Adenocarcinoma | [symbol] | Female | IV | smoker, stopped | 17 | 0 |
| | | | | | | over 6 months | | |
| [symbol] | no baseline | No baseline | 52 | Male | NA | smoker | 30 | 1 |
| | cancer | cancer | | | | | | |
| [symbol] | benign | Benign | 46 | Male | NA | smoker | 80 | 0 |
| [symbol] | lung cancer | Adenocarcinoma | 39 | Female | [symbol] | smoker | 33 | 0 |
| [symbol] | lung cancer | SCLC | 73 | Female | IV | smoker | 40 | 1 |
| [symbol] | benign | Benign | 54 | Female | NA | smoker, stopped | 30 | 1 |
| | | | | | | under 6 months | | |
| [symbol] | no baseline | No baseline | 52 | Male | NA | smoker, stopped | 15 | 1 |
| | cancer | cancer | | | | over 6 months | | |
| [symbol] | no baseline | No baseline | 56 | Male | NA | smoker, stopped | 45 | 0 |
| | cancer | cancer | | | | over 6 months | | |
| [symbol] | lung cancer | Adenocarcinoma | 45 | Male | IV | smoker, stopped | 45 | 0 |
| | | | | | | over 6 months | | |
| [symbol] | lung cancer | Adenocarcinoma | [symbol] | Female | IV | never smoker | 0 | 0 |
| [symbol] | lung cancer, | Adenocarcinoma | [symbol] | Female | [symbol] | smoker | 40 | 0 |
| | other cancer | | | | | | | |
| [symbol] | no lung cancer, | No baseline | 63 | Female | NA | smoker, stopped | 4 | 1 |
| | other cancer | cancer | | | | over 6 months | | |
| [symbol] | lung cancer | [symbol] | 52 | Female | IV | smoker | 30 | 0 |
| [symbol] | benign | Benign | 56 | Female | NA | smoker | 40 | 0 |
| [symbol] | lung cancer | Adenocarcinoma | 71 | Male | [symbol] | smoker, stopped | 40 | 0 |
| | | | | | | over 6 months | | |
| [symbol] | lung cancer | Adenocarcinoma | 27 | Male | [symbol] | smoker, stopped | 13 | 2 |
| | | | | | | over 6 months | | |
| [symbol] | benign | Benign | 64 | Female | NA | smoker | 45 | 0 |
| [symbol] | no lung cancer, | No baseline | 54 | Male | NA | smoker, stopped | [symbol] | 0 |
| | other cancer | cancer | | | | over 6 months | | |
| [symbol] | lung cancer | Unknown | 62 | Female | [symbol] | smoker, stopped | 35 | 0 |
| | | | | | | under 6 months | | |
| [symbol] | benign | Benign | 43 | Male | NA | never smoker | 0 | 0 |
| [symbol] | benign | Benign | 62 | Male | NA | smoker, stopped | 40 | 1 |
| | | | | | | over 6 months | | |
| [symbol] | no baseline | No baseline | 63 | Female | NA | never smoker | 0 | 1 |
| | cancer | cancer | | | | | | |
| [symbol] | benign | Benign | 63 | Female | NA | smoker | 10 | 0 |
| [symbol] | lung cancer | [symbol] | 75 | Male | [symbol] | never smoker | 0 | 0 |
| [symbol] | lung cancer | Squamous | 59 | Female | [symbol] | smoker. stopped | 40 | 0 |
| | | | | | | over 6 months | | |
| [symbol] | no baseline | No baseline | 39 | Female | NA | smoker | 25 | 0 |
| | cancer | cancer | | | | | | |
| [symbol] | lung cancer | SCLC | 63 | Female | [symbol] | smoker, stopped | 25 | 0 |
| | | | | | | under 6 months | | |
| [symbol] | no lung cancer, | Benign | 76 | Female | NA | smoker | 55 | 0 |
| | prior lung cancer | | | | | | | |
| [symbol] | no baseline | No baseline | 33 | Male | NA | smoker, stopped | 47 | 1 |
| | cancer | cancer | | | | over 6 months | | |
| [symbol] | met to lung | Metastasis from | 23 | Female | IV | smoker, stopped | 1 | 0 |
| | | another primary | | | | over 6 months | | |
| | | cancer | | | | | | |
| [symbol] | lung cancer | [symbol] | 76 | Female | IV | smoker, stopped | 12 | 0 |
| | | | | | | over 6 months | | |
| [symbol] | met to lung, | Metastasis from | 59 | Female | IV | never smoker | 0 | 0 |
| | other cancer | another primary | | | | | | |
| | | cancer | | | | | | |
| [symbol] | no baseline | No baseline | 70 | Male | NA | smoker | 65 | 0 |
| | cancer | cancer | | | | | | |
| [symbol] | no lung cancer, | No baseline | 59 | Male | NA | smoker | 20 | 0 |
| | other cancer | cancer | | | | | | |
| [symbol] | no lung cancer, | No baseline | 77 | Male | NA | smoker, stopped | 30 | 0 |
| | other cancer | cancer | | | | over 6 months | | |
| [symbol] | lung cancer, | Adenocarcinoma | 67 | Female | [symbol] | smoker, stopped | 23 | 0 |
| | other cancer | | | | | over 6 months | | |
| [symbol] | benign | Benign | 76 | Male | NA | never smoker | 0 | 0 |
| [symbol] | lung cancer | Adenocarcinoma | 82 | Female | IV | smoker, stopped | 50 | 1 |
| | | | | | | over 6 months | | |
| [symbol] | no lung cancer, | Benign | 63 | Male | NA | smoker, stopped | 70 | 0 |
| | other cancer | | | | | over 6 months | | |
| [symbol] | lung cancer | Adenocarcinoma | 81 | Female | IV | smoker, stopped | 60 | 0 |
| | | | | | | over 6 months | | |
| [symbol] | no baseline | No baseline | 54 | Male | NA | smoker | 40 | 0 |
| | cancer | cancer | | | | | | |
| [symbol] | lung cancer | Adenocarcinoma | 64 | Female | IV | smoker, stopped | 30 | 0 |
| | | | | | | over 6 months | | |
| [symbol] | no baseline | No baseline | 62 | Female | NA | smoker | 35 | 0 |
| | cancer | cancer | | | | | | |
| [symbol] | lung cancer | Adenocarcinoma | 73 | Male | [symbol] | smoker | 37 | 0 |
| [symbol] | no lung cancer, | No baseline | 60 | Male | NA | smoker, stopped | 35 | 0 |
| | other cancer | cancer | | | | over 6 months | | |
| [symbol] | no baseline | No baseline | 60 | Male | NA | smoker | 50 | 0 |
| | cancer | cancer | | | | | | |
| [symbol] | benign | Benign | 60 | Male | NA | smoker | 42 | 0 |
| [symbol] | met to lung | Metastasis from | 55 | Male | IV | smoker, stopped | 15 | 0 |
| | | another primary | | | | over 6 months | | |
| | | cancer | | | | | | |
| [symbol] | no lung cancer, | Benign | 25 | Male | NA | smoker, stopped | 35 | 0 |
| | other cancer | | | | | over 6 months | | |
| [symbol] | lung cancer | Squamous | 80 | Female | [symbol] | smoker | 40 | 0 |
| [symbol] | no lung cancer, | No baseline | 74 | Male | NA | smoker, stopped | 30 | 0 |
| | other cancer | cancer | | | | over 6 months | | |
| [symbol] | benign | Benign | 37 | Male | NA | smoker | 12 | 0 |
| [symbol] | lung cancer | Adenocarcinoma | 64 | Female | IV | smoker,stopped | 29 | 0 |
| | | | | | | over 6 months | | |
| [symbol] | no baseline | No baseline | 36 | Male | NA | smoker, stopped | 40 | 0 |
| | cancer | cancer | | | | under 6 months | | |
| [symbol] | benign | Benign | 59 | Male | NA | smoker, stopped | 43 | 0 |
| | | | | | | over 6 months | | |
| [symbol] | no baseline | No baseline | 73 | Female | NA | smoker, stopped | 7 | 0 |
| | cancer | cancer | | | | over 6 months | | |
| [symbol] | lung cancer | Squamous | 50 | Male | [symbol] | smoker | 75 | 1 |
| [symbol] | lung cancer | SCLC | 76 | Female | [symbol] | smoker, stopped | 30 | 0 |
| | | | | | | over 6 months | | |
| [symbol] | lung cancer | SCLC | 39 | Male | [symbol] | smoker, stopped | 90 | 0 |
| | | | | | | over 6 months | | |
| [symbol] | benign | Benign | 74 | Male | NA | smoker, stopped | 5 | 0 |
| | | | | | | over 6 months | | |
| [symbol] | no baseline | No baseline | 61 | Male | NA | never smoker | 0 | 0 |
| | cancer | cancer | | | | | | |
| [symbol] | no baseline | No baseline | 74 | Male | NA | smoker, stopped | 10 | 0 |
| | cancer | cancer | | | | over 6 months | | |
| [symbol] | benign | Benign | 47 | Female | NA | never smoker | 0 | 0 |
| [symbol] | benign | Benign | 64 | Male | NA | never smoker | 0 | 0 |
| [symbol] | lung cancer | Adenocarcinoma | 42 | Male | IV | smoker | 30 | 1 |
| [symbol] | no lung cancer, | No baseline | 76 | Male | NA | smoker, stopped | 60 | 0 |
| | other cancer | cancer | | | | over 6 months | | |
| [symbol] | benign | Benign | 73 | Female | NA | smoker, stopped | 45 | 1 |
| | | | | | | over 6 months | | |
| [symbol] | lung cancer | Squamous | 64 | Female | [symbol] | smoker, stopped | 30 | 0 |
| | other cancer | | | | | over 6 months | | |
| [symbol] | lung cancer | Adenocarcinoma | 72 | Female | IV | smoker, stopped | 30 | 0 |
| | | | | | | over 6 months | | |
| [symbol] | lung cancer | Adenocarcinoma | 72 | Female | IV | smoker, stopped | 20 | 0 |
| | | | | | | over 6 months | | |
| [symbol] | no baseline | No baseline | 71 | Female | NA | smoker, stopped | 20 | 0 |
| | cancer | cancer | | | | over 6 months | | |
| [symbol] | no lung cancer, | No baseline | 70 | Male | NA | never smoker | 0 | 0 |
| | other cancer | cancer | | | | | | |
| [symbol] | benign | Benign | 51 | Female | NA | smoker | 40 | 0 |
| [symbol] | lung cancer | SCLC | 60 | Male | IV | smoker, stopped | 48 | 1 |
| | | | | | | over 6 months | | |
| [symbol] | no baseline | No baseline | 61 | Female | NA | smoker, stopped | 45 | 1 |
| | cancer | cancer | | | | under 6 months | | |
| [symbol] | no lung cancer, | No baseline | 57 | Male | NA | smoker, stopped | 30 | 1 |
| | prior lung cancer, | cancer | | | | over 6 months | | |
| | later lung cancer | | | | | | | |
| [symbol] | no baseline | No baseline | 44 | Male | NA | never smoker | 0 | 1 |
| | cancer | cancer | | | | | | |
| [symbol] | lung cancer | Adenocarcinoma | 35 | Female | IV | smoker | 30 | 1 |
| [symbol] | met to lung, | Metastasis from | 78 | Female | IV | smoker, stopped | 1 | 0 |
| | other cancer | another primary | | | | over 6 months | | |
| | | cancer | | | | | | |
| [symbol] | no baseline | No baseline | 49 | Female | NA | smoker | 30 | 0 |
| | cancer | cancer | | | | | | |
| [symbol] | lung cancer | Squamos | 71 | Male | IV | smoker, stopped | 120 | 0 |
| | | | | | | over 6 months | | |
| [symbol] | no baseline | No baseline | 74 | Male | NA | smoker | 63 | 1 |
| | cancer | cancer | | | | | | |
| [symbol] | met to lung, | Metastasis from | 20 | Female | IV | smoker, stopped | 25 | 0 |
| | other cancer | \|another primary | | | | over 6 months | | |
| | | cancer | | | | | | |
| [symbol] | lung cancer | [symbol] | 63 | Male | IV | smoker, stopped | 67 | 0 |
| | | | | | | under 6 months | | |
| [symbol] | met to lung | Metastasis from | 33 | Male | IV | never smoker | 0 | 0 |
| | other cancer | another primary | | | | | | |
| | | cancer | | | | | | |
| [symbol] | met to lung | Metastasis from | 65 | Female | IV | smoker | 5 | 1 |
| | other cancer | another primary | | | | | | |
| | | cancer | | | | | | |
| [symbol] | no lung cancer, | No baseline | 61 | Male | NA | smoker, stopped | 33 | 1 |
| | other cancer | cancer | | | | over 6 months | | |
| [symbol] | lung cancer, | Adenocarcinoma | 81 | Female | IV | smoker | 30 | 0 |
| | other cancer | | | | | | | |
| [symbol] | lung cancer | Adenocarcinoma | 55 | Female | [symbol] | smoker | 50 | 0 |
| [symbol] | no baseline | No baseline | 71 | Male | NA | smoker, stopped | 50 | 0 |
| | cancer | cancer | | | | under 6 months | | |
| [symbol] | no baseline | No baseline | 59 | Female | NA | smoker | 50 | 0 |
| | cancer | cancer | | | | | | |
| [symbol] | met to lung | Metastasis from | 56 | Female | IV | smoker | 21 | 0 |
| | | another primary | | | | | | |
| | | cancer | | | | | | |
| [symbol] | benign | Benign | 63 | Female | NA | smoker, stopped | 12 | 0 |
| | | | | | | over 6 months | | |
| [symbol] | no lung cancer, | No baseline | 75 | Male | NA | smoker, stopped | 50 | 0 |
| | other cancer | cancer | | | | over 6 months | | |
| [symbol] | no lung cancer, | No baseline | 62 | Female | NA | smoker | 40 | 0 |
| | other cancer, | cancer | | | | | | |
| | later lung cancer | | | | | | | |
| [symbol] | no baseline | No baseline | 57 | Male | NA | never smoker | 0 | 1 |
| | cancer | cancer | | | | | | |
| [symbol] | no baseline | No baseline | 70 | Male | NA | smoker, stopped | 100 | 1 |
| | cancer | cancer | | | | under 6 months | | |
| [symbol] | no baseline | No baseline | 77 | Female | NA | smoker, stopped | 20 | 0 |
| | cancer | cancer | | | | over 6 months | | |
| [symbol] | no lung cancer, | Benign | 50 | Female | NA | smoker, stopped | 30 | 0 |
| | other cancer | | | | | over 6 months | | |
| [symbol] | no baseline | No baseline | 79 | Male | NA | smoker, stopped | 22 | 0 |
| | cancer | cancer | | | | over 6 months | | |
| [symbol] | lung cancer, | Unknown | 75 | Female | IV | smoker | 25 | 0 |
| | other cancer | | | | | | | |
| [symbol] | lung cancer, | Adenocarcinoma | 76 | Female | IV | smoker | 27 | 0 |
| | other cancer | | | | | | | |
| [symbol] | no baseline | No baseline | 55 | Female | NA | smoker | 60 | 0 |
| | cancer | cancer | | | | | | |
| [symbol] | no lung cancer, | Benign | 63 | Male | NA | smoker, stopped | 15 | 1 |
| | other cancer | | | | | over 6 months | | |
| [symbol] | lung cancer | Adenocarcinoma | 58 | Female | NA | smoker | 24 | 0 |
| [symbol] | lung cancer | Adenocarcinoma | 57 | Female | IV | smoker | 43 | 1 |
| [symbol] | lung cancer, | Squamous | 76 | Male | NA | smoker, stopped | 70 | 0 |
| | other cancer | | | | | under 6 months | | |
| [symbol] | no lung cancer, | No baseline | 70 | Female | NA | never smoker | 0 | 1 |
| | other cancer | cancer | | | | | | |
| [symbol] | benign | Benign | 24 | Male | NA | smoker | 5 | 0 |
| [symbol] | no baseline | No baseline | 70 | Female | NA | smoker, stopped | 25 | 1 |
| | cancer | cancer | | | | over 6 months | | |
| [symbol] | lung cancer, | Adenocarcinoma | 65 | Female | [symbol] | smoker, stopped | 10 | 0 |
| | other cancer | | | | | over 6 months | | |
| [symbol] | no lung cancer, | No baseline | 51 | Female | NA | smoker, stopped | 10 | 1 |
| | other cancer | cancer | | | | over 6 months | | |
| [symbol] | no baseline | No baseline | 69 | Male | NA | never smoker | 0 | 0 |
| | cancer | cancer | | | | | | |
| [symbol] | no lung cancer, | No baseline | 52 | Female | NA | smoker, stopped | 40 | 1 |
| | other cancer | cancer | | | | over 6 months | | |
| [symbol] | no lung cancer, | No baseline | 71 | Male | NA | smoker | 70 | 0 |
| | other cancer | cancer | | | | | | |
| [symbol] | benign | Benign | 36 | Male | NA | smoker | 20 | 1 |
| [symbol] | lung cancer, | Adenocarcinoma | 56 | Female | IV | smoker | 15 | 0 |
| | other cancer | | | | | | | |
| [symbol] | no lung cancer, | Benign | 75 | Female | NA | smoker, stopped | 12 | 0 |
| | other cancer | | | | | over 6 months | | |
| [symbol] | lung cancer | Squamous | 68 | Female | [symbol] | smoker | 52 | 1 |
| [symbol] | benign | Benign | 56 | Male | NA | smoker, stopped | 30 | 1 |
| | | | | | | over 6 months | | |
| [symbol] | benign | Benign | 43 | Male | NA | never smoker | 0 | 0 |
| [symbol] | benign | Benign | 41 | Female | NA | smoker, stopped | 20 | 1 |
| | | | | | | under 6 months | | |
| [symbol] | no lung cancer, | No baseline | 37 | Female | NA | sssmoker, stopped | 20 | 1 |
| | other cancer | cancer | | | | over 6 months | | |
| [symbol] | benign | Benign | 17 | Male | NA | smoker, stopped | 12 | 0 |
| | | | | | | under 6 months | | |
| [symbol] | no lung cancer, | No baseline | 67 | Male | NA | smoker | 55 | 1 |
| | later lung cancer | cancer | | | | | | |
| [symbol] | benign | Benign | 37 | Male | NA | never smoker | 0 | 1 |
| [symbol] | lung cancer | [symbol] | 76 | Male | IV | smoker | 70 | 0 |
| [symbol] | no lung cancer, | Benign | 75 | Female | NA | smoker, stopped | 3 | 0 |
| | other cancer | | | | | over 6 months | | |
| [symbol] | no baseline | No baseline | 66 | Female | NA | never smoker | 0 | 0 |
| | cancer | cancer | | | | | | |
| [symbol] | met to lung | Metastasis from | 37 | Male | IV | smoker, stopped | 60 | 0 |
| | | another primary | | | | under 6 months | | |
| | | cancer | | | | | | |
| [symbol] | no lung cancer, | Benign | 74 | Male | NA | smoker | 60 | 0 |
| | later lung cancer | | | | | | | |
| [symbol] | lung cancer | SCLC | 77 | Male | IV | smoker, stopped | 50 | 0 |
| | | | | | | over 6 months | | |
| [symbol] | no baseline | No baseline | 50 | Female | NA | smoker, stopped | 40 | 0 |
| | cancer | cancer | | | | over 6 months | | |
| [symbol] | lung cancer, | Adenocarcinoma | 35 | Female | [symbol] | smoker | 40 | 0 |
| | later lung cancer | | | | | | | |
| [symbol] | benign | Benign | 49 | Female | NA | smoker, stopped | 35 | 0 |
| | | | | | | under 6 months | | |
| [symbol] | no lung cancer, | Benign | 63 | Male | NA | smoker, stopped | 43 | 1 |
| | other cancer | | | | | over 6 months | | |
| [symbol] | no lung cancer, | No baseline | 61 | Female | NA | never smoker | 0 | 0 |
| | other cancer | cancer | | | | | | |
| [symbol] | no baseline | No baseline | 65 | Female | NA | smoker, stopped | 15 | 1 |
| | cancer | cancer | | | | over 6 months | | |
| [symbol] | lung cancer, | Adenocarcinoma | 75 | Female | IV | smoker, stopped | 65 | 0 |
| | other cancer | | | | | under 6 months | | |
| [symbol] | benign | Benign | 77 | Male | NA | smoker, stopped | 20 | 0 |
| | | | | | | over 6 months | | |
| [symbol] | benign | Benign | 70 | Male | NA | smoker, stopped | 30 | 0 |
| | | | | | | over 6 months | | |
| [symbol] | no baseline | No baseline | 53 | Male | NA | smoker | 23 | 0 |
| | cancer | cancer | | | | | | |
| [symbol] | met to lung, | Metastasis from | 65 | Male | IV | smoker | 75 | 0 |
| | other cancer | another primary | | | | | | |
| | | cancer | | | | | | |
| [symbol] | lung cancer | Adenocarcinoma | 62 | Female | IV | smoker | 30 | 0 |
| [symbol] | lung cancer | Squamous | 60 | Female | NA | smoker | 60 | 0 |
| [symbol] | no ung cancer, | No baseline | 37 | Female | NA | smoker, stopped | 25 | 1 |
| | other cancer | cancer | | | | over 6 months | | |
| [symbol] | no lung cancer, | No baseline | 66 | Female | NA | smoker, stopped | 25 | 1 |
| | other cancer | cancer | | | | over 6 months | | |
| [symbol] | no baseline | No baseline | 56 | Female | NA | smoker | 35 | 0 |
| | cancer | cancer | | | | | | |
| [symbol] | no baseline | No baseline | 72 | Male | NA | smoker, stopped | 20 | 0 |
| | cancer | cancer | | | | over 6 months | | |
| [symbol] | no lung cancer, | No baseline | 65 | Male | NA | never smoker | 0 | 0 |
| | other cancer | cancer | | | | | | |
| [symbol] | lung cancer, | Adenocarcinoma | 72 | Male | IV | smoker | 100 | 0 |
| | other cancer | | | | | | | |
| [symbol] | no baseline | No baseline | 60 | Male | NA | smoker, stopped | 25 | 0 |
| | cancer | cancer | | | | over 6 months | | |
| [symbol] | no lung cancer, | Benign | 56 | Male | NA | smoker | 65 | 0 |
| | later lung cancer | | | | | | | |
| [symbol] | no lung cancer, | Benign | 69 | Male | NA | smoker | 55 | 1 |
| | other cancer | | | | | | | |
| [symbol] | no lung cancer, | Benign | 26 | Male | NA | never smoker | 0 | 1 |
| | other cancer | | | | | | | |
| [symbol] | no lung cancer, | No baseline | 30 | Female | NA | smoker, stopped | 60 | 0 |
| | other cancer | caancer | | | | under 6 months | | |
| [symbol] | no baseline | No baseline | 80 | Female | NA | never smoker | 0 | 0 |
| | cancer | cancer | | | | | | |
| [symbol] | no lung cancer, | No baseline | 79 | Male | NA | smoker, stopped | 19 | 1 |
| | other cancer | cancer | | | | over 6 months | | |
| [symbol] | lung cancer | Squamous | 67 | Male | [symbol] | smoker | 60 | 1 |
| [symbol] | lung cancer, | Adenocarcinoma | 71 | Female | IV | smoker | 150 | 1 |
| | prior lung cancer | | | | | | | |
| [symbol] | no baseline | No baseline | 35 | Male | NA | never smoker | 0 | 0 |
| | cancer | cancer | | | | | | |
| [symbol] | benign | Benign | 49 | Male | NA | smoker | 35 | 1 |
| [symbol] | no lung cancer, | Benign | 62 | Male | NA | smoker, stopped | 30 | 1 |
| | other cancer | | | | | over 6 months | | |
| [symbol] | no lung cancer, | No baseline | 36 | Female | NA | never smoker | 0 | 0 |
| | other cancer | cancer | | | | | | |
| [symbol] | no lung cancer, | No baseline | 65 | Female | NA | smoker, stopped | 15 | 0 |
| | other cancer | cancer | | | | over 6 months | | |
| [symbol] | lung cancer | Adenosquamous | 35 | Female | [symbol] | smoker, stopped | 40 | 0 |
| | | | | | | over 6 months | | |
| [symbol] | lung cancer | Squamous | 74 | Male | IV | smoker, stopped | 50 | 0 |
| | | | | | | over 6 months | | |
| [symbol] | no baseline | No baseline | 60 | Male | NA | smoker, stopped | 60 | 1 |
| | cancer | cancer | | | | over 6 months | | |
| [symbol] | benign | Benign | 47 | Female | NA | smoker | 30 | 0 |
| [symbol] | lung cancer | Squamous | 74 | Female | [symbol] | smoker, stopped | 50 | 1 |
| | | | | | | under 6 months | | |
| [symbol] | no lung cancer, | No baseline | 36 | Female | NA | smoker | 45 | 0 |
| | later lung cancer | cancer | | | | | | |
| [symbol] | lung cancer | Adenocarcinoma | 67 | Male | [symbol] | smoker, stopped | 75 | 0 |
| | | | | | | over 6 months | | |
| [symbol] | no baseline | No baseline | 37 | Female | NA | smoker, stopped | 2 | 1 |
| | cancer | cancer | | | | over 6 months | | |
| [symbol] | no baseline | No baseline | 58 | Male | NA | smoker | 40 | 0 |
| | cancer | cancer | | | | | | |
| [symbol] | benign | Benign | 49 | Female | NA | smoker | 25 | 0 |
| [symbol] | no lung cancer, | No baseline | 58 | Female | NA | smoker | 45 | 0 |
| | other cancer | cancer | | | | | | |
| [symbol] | no baseline | No baseline | 32 | Female | NA | smoker, stopped | 25 | 0 |
| | cancer | cancer | | | | over 6 months | | |
| [symbol] | no baseline | No baseline | 67 | Male | NA | never smoker | 0 | 0 |
| | cancer | cancer | | | | | | |
| [symbol] | lung cancer | Adenocarcinoma | 70 | Female | [symbol] | smoker, stopped | 50 | 0 |
| | | | | | | under 6 months | | |
| [symbol] | no baseline | No baseline | 65 | Male | NA | never smoker | 0 | 0 |
| | cancer | cancer | | | | | | |
| [symbol] | benign | Benign | 39 | Female | NA | smoker | 45 | 0 |
| [symbol] | benign | Benign | 43 | Male | NA | smoker, stopped | 10 | 0 |
| | | | | | | over 6 months | | |
| [symbol] | no baseline | No baseline | 30 | Male | NA | smoker, stopped | 10 | 1 |
| | cancer | cancer | | | | over 6 months | | |
| [symbol] | lung cancer | Adenocarcinoma | 64 | Male | [symbol] | smoker, stopped | 15 | 0 |
| | | | | | | over 6 months | | |
| [symbol] | lung cancer, | Adenocarcinoma | 79 | Female | [symbol] | smoker | 65 | 0 |
| | other cancer | | | | | | | |
| [symbol] | no baseline | No baseline | 73 | Female | NA | smoker, stopped | 10 | 1 |
| | cancer | cancer | | | | over 6 months | | |
| [symbol] | lung cancer | Adenocarcinoma | 79 | Male | IV | smoker, stopped | 35 | 0 |
| | | | | | | under 6 months | | |
| [symbol] | lung cancer, | SCLC | 31 | Female | IV | smoker, stopped | 35 | 0 |
| | other cancer | | | | | over 6 months | | |
| [symbol] | benign | Benign | 52 | Male | NA | smoker, stopped | 20 | 0 |
| | | | | | | under 6 months | | |
| [symbol] | benign | Benign | 42 | Female | NA | never smoker | 0 | 1 |
| [symbol] | met to lung, | Metastasis from | 93 | Female | IV | never smoler | 0 | 0 |
| | other cancer | another primary | | | | | | |
| | | cancer | | | | | | |
| [symbol] | benign | Benign | 67 | Male | NA | smoker, stopped | 40 | 1 |
| | | | | | | over 6 months | | |
| [symbol] | no baseline | No baseline | 66 | Male | NA | never smoker | 0 | 0 |
| | cancer | cancer | | | | | | |
| [symbol] | lung cancer | SCLC | [symbol] | Female | [symbol] | smoker | 20 | 0 |
| [symbol] | no baseline | No baseline | 57 | Female | NA | smoker, stopped | 48 | 1 |
| | cancer | cancer | | | | over 6 months | | |
| [symbol] | no baseline | No baseline | 51 | Male | NA | never smoker | 0 | 1 |
| | cancer | cancer | | | | | | |
| [symbol] | benign | Benign | 15 | Female | NA | smoker | 5 | 1 |
| [symbol] | benign | Benign | 56 | Female | NA | smoker | 25 | 0 |
| [symbol] | no lung cancer, | No baseline | 73 | Female | NA | smoker, stopped | 40 | 1 |
| | other cancer | cancer | | | | over 6 months | | |
| [symbol] | no lung cancer, | No baseline | 64 | Female | NA | smoker, stopped | 20 | 0 |
| | other cancer | cancer | | | | over 6 months | | |
| [symbol] | no baseline | No baseline | 75 | Male | NA | smoker, stopped | NA | 0 |
| | cancer | cancer | | | | over 6 months | | |
| [symbol] | no baseline | No baseline | 59 | Male | NA | smoekr, stopped | 30 | 0 |
| | cancer | cancer | | | | over 6 months | | |
| [symbol] | no lung cancer, | No baseline | 50 | Female | NA | smoker, stopped | NA | 1 |
| | other cancer | cancer | | | | over 6 months | | |
| [symbol] | benign | Benign | 67 | Male | NA | never smoker | 0 | 0 |
| [symbol] | lung cancer | Adenocarcinoma | 59 | Male | [symbol] | smoker | 30 | 0 |
| [symbol] | lung cancer, | Adenocarcinoma | 64 | Male | [symbol] | smoker | 150 | 0 |
| | prior lung cancer | | | | | | | |
| [symbol] | no baseline | No baseline | 60 | Male | NA | smoker | 53 | 1 |
| | cancer | cancer | | | | | | |
| [symbol] | benign | Benign | 68 | Male | NA | smoker, stopped | 30 | 0 |
| | | | | | | over 6 months | | |
| [symbol] | no baseline | No baseline | 68 | Male | NA | smoker, stopped | 40 | 0 |
| | center | center | | | | under 6 months | | |
| [symbol] | lung cancer, | Adenocarcinoma | 76 | Male | IV | smoker | 50 | 0 |
| | other cancer | | | | | | | |
| [symbol] | met to lung, | Metastasis from | 55 | Male | IV | smoker | 40 | 0 |
| | other cancer | another primary | | | | | | |
| | | cancer | | | | | | |
| [symbol] | nolung cancer, | No baseline | 55 | Male | NA | never smoker | 0 | 1 |
| | other cancer | cancer | | | | | | |
| [symbol] | no lung cancer, | No baseline | 58 | Female | NA | smoker, stopped | 20 | 0 |
| | other cancer | cancer | | | | over 6 months | | |
| [symbol] | benign | Benign | 78 | Female | NA | never smoker | 0 | 0 |
| [symbol] | lung cancer | Adenocarcinoma | 35 | Male | IV | smoker, stopped | 23 | 0 |
| | | | | | | over 6 months | | |
| [symbol] | no lung cancer, | No baseline | 75 | Male | NA | smoker, stopped | NA | 0 |
| | other cancer | cancer | | | | over 6 months | | |
| [symbol] | no lung cancer, | No baseline | 84 | Female | NA | smoker, stopped | 15 | 1 |
| | other cancer | cancer | | | | under 6 months | | |
| [symbol] | no lung cancer, | Benign | 52 | Female | NA | smoker, stopped | 15 | 0 |
| | later lung cancer | | | | | under 6 months | | |
| [symbol] | no lung cancer, | No baseline | 54 | Female | NA | smoker | 39 | 1 |
| | other cancer | cancer | | | | | | |
| [symbol] | no baseline | No baseline | 38 | Female | NA | smoker | 20 | 0 |
| | cancer | cancer | | | | | | |
| [symbol] | lung cancer, | Squamous | 58 | Male | [symbol] | smoker, stopped | 90 | 0 |
| | other cancer | | | | | over 6 months | | |
| [symbol] | benign | Benign | 63 | Male | NA | smoker, stopped | 41 | 0 |
| | | | | | | under 6 months | | |
| [symbol] | no baseline | No baseline | 58 | Male | NA | smoker, stopped | 1 | 0 |
| | cancer | cancer | | | | over 6 months | | |
| [symbol] | lung cancer | [symbol] | 65 | Male | IV | smoker | 50 | 0 |
| [symbol] | lung cancer | Squamous | 70 | Male | [symbol] | smoker | 100 | 0 |
| [symbol] | lung cancer | [symbol] | 74 | Male | [symbol] | smoker | 50 | 0 |
| [symbol] | lung cancer | Squamous | 85 | Female | IV | smoker, stopped | 40 | 0 |
| | | | | | | over 6 months | | |
| [symbol] | lung cancer, | Adenocarcinoma | 30 | Male | IV | smoker, stopped | 20 | 0 |
| | other cancer | | | | | over 6 months | | |
| [symbol] | benign | Benign | 39 | Male | NA | never smoker | 0 | 0 |
| [symbol] | benign | Benign | 59 | Male | NA | smoker | 100 | 0 |
| [symbol] | no lung cancer, | No baseline | [symbol] | Female | NA | smoker, stopped | 3 | 0 |
| | other cancer | cancer | | | | over 6 months | | |
| [symbol] | lung cancer | Adenocarcinoma | 50 | Male | [symbol] | smoker | 45 | 1 |
| [symbol] | benign | Benign | 37 | Male | NA | smoker | 15 | 1 |
| [symbol] | benign | Benign | 61 | Female | NA | smoker, stopped | 25 | 0 |
| | | | | | | under 6 months | | |
| [symbol] | lung cancer | Adenocarcinoma | 74 | Female | [symbol] | smoker, stopped | 25 | 0 |
| | | | | | | over 6 months | | |
| [symbol] | lung cancer | Squamous | 80 | Male | IV | smoker | 50 | 1 |
| [symbol] | lung cancer | Adenocarcinoma | 56 | Male | IV | smoker, stopped | 10 | 0 |
| | | | | | | under 6 months | | |
| [symbol] | lung cancer | Adenocarcinoma | 81 | Male | IV | smoker, stopped | 40 | 0 |
| | | | | | | over 6 months | | |

| | | | | | | Time from |
|---|---|---|---|---|---|---|
| | COPD | | | Prior | Future | blood draw |
| Patient | (0 = No, | [symbol] | IL-6 | cancer | cancer | to cancer |
| ID | 1 = Yes) | (mg/L) | (pg/mL) | type | type | diagnosis |
| [symbol] | 0 | 10 | 2.9 | NA | NA | NA |
| [symbol] | 0 | 10 | 7.9 | NA | NA | NA |
| [symbol] | 0 | 10 | 5.9 | [symbol] | uterine cancer, | [symbol] |
| | | | | | basal cell | |
| | | | | | carcinoma | |
| | | | | | NSCLC adeno | |
| | | | | | and NSCLC NEC | |
| [symbol] | 0 | 17 | 7.7 | NA | colorectal | 2471 |
| | | | | | cancer | |
| [symbol] | 1 | 10 | 11.9 | NA | NA | NA |
| [symbol] | 0 | 10 | 9.5 | NA | NA | NA |
| [symbol] | 1 | 10 | 4.1 | NA | NA | NA |
| [symbol] | 0 | 20 | 26 | [symbol] | NA | [symbol] |
| [symbol] | 1 | 15 | 3.6 | bladder cancer | NA | [symbol] |
| | | | | stage III | | |
| [symbol] | 0 | 22 | 8.1 | NA | NA | NA |
| [symbol] | 0 | 10 | 4.3 | NA | NA | NA |
| [symbol] | 0 | 10 | 2.3 | NA | NA | NA |
| [symbol] | 1 | 36 | [symbol] | NA | NA | NA |
| [symbol] | 1 | 30 | 1.2 | uterine cancer | NA | −9232 |
| | | | | in situ, | | |
| | | | | operated | | |
| [symbol] | 0 | 10 | 19 | NA | NA | NA |
| [symbol] | 0 | 10 | 5.5 | NA | NA | NA |
| [symbol] | 0 | 10 | 14.2 | NA | NA | NA |
| [symbol] | 0 | 10 | 1.1 | NA | NA | NA |
| [symbol] | 1 | 10 | 1.9 | [symbol] | NA | [symbol] |
| [symbol] | 0 | 10 | 1 | NA | NA | NA |
| [symbol] | 0 | 10 | 4.3 | [symbol] | NA | −147 |
| [symbol] | 0 | 10 | 3.4 | breast cancer | NA | −10995 |
| | | | | operated | | |
| [symbol] | 0 | 41 | 15.7 | NA | NA | NA |
| [symbol] | 0 | 10 | 2.8 | NA | NA | NA |
| [symbol] | 0 | 10 | 14.4 | NA | basal cell | 1875 |
| | | | | | carcinoma | |
| [symbol] | 0 | 10 | 1.1 | NA | NA | NA |
| [symbol] | 0 | 10 | NA | NA | NA | NA |
| [symbol] | 0 | 15 | [symbol] | NA | basal cell | [symbol] |
| | | | | | carcinoma, | |
| | | | | | hepatocellular | |
| | | | | | carcinoma | |
| [symbol] | 0 | 10 | 1.4 | NA | NA | NA |
| [symbol] | 0 | 10 | 4.4 | NA | NA | NA |
| [symbol] | 1 | 15 | 8.2 | NA | NA | NA |
| [symbol] | 0 | 10 | 5.3 | NA | NA | NA |
| [symbol] | 0 | 10 | 10.1 | NA | NA | NA |
| [symbol] | 0 | 10 | 2.9 | NA | NA | NA |
| [symbol] | 0 | 84 | 12.7 | NA | NA | NA |
| [symbol] | 0 | 11 | 22.4 | NA | NA | NA |
| [symbol] | 0 | 10 | 2.9 | NA | NA | NA |
| [symbol] | 1 | 10 | 4.5 | NA | NA | NA |
| [symbol] | 0 | 10 | 2.3 | NA | NA | NA |
| [symbol] | 1 | 13 | 8.3 | NA | NA | NA |
| [symbol] | 0 | 10 | 2.7 | NA | squamous cell | 634 |
| | | | | | carcinoma | |
| [symbol] | 0 | 27 | NA | NA | NA | NA |
| [symbol] | 0 | 11 | 4.8 | prostate cancer | NA | −522 |
| | | | | operated | | |
| [symbol] | 0 | 17 | 16.8 | NA | NA | NA |
| [symbol] | 0 | 18 | 5.2 | NA | NA | NA |
| [symbol] | 0 | 10 | 3.1 | NA | NA | NA |
| [symbol] | 0 | 144 | 21.3 | Superficial | [symbol] | −1434, 231 |
| | | | | spreading | | |
| | | | | malignant | | |
| | | | | melanoma, | | |
| | | | | operated | | |
| [symbol] | 0 | 10 | 11.8 | prostate cancer | NA | −1773 |
| | | | | operated | | |
| [symbol] | 1 | 10 | 3.3 | NA | basal cell | NA |
| | | | | | carcinoma | |
| [symbol] | 0 | 32 | 11.8 | NA | NA | NA |
| [symbol] | 0 | 10 | 7.1 | NA | NA | NA |
| [symbol] | 0 | 10 | 3.1 | uterine cancer | NA | −7579 |
| | | | | in situ, | | |
| | | | | operated | | |
| [symbol] | 1 | 10 | 3.9 | basal cell | NA | −3578 |
| | | | | carcinoma | | |
| [symbol] | 0 | 11 | 7.2 | NA | NA | NA |
| [symbol] | 0 | 10 | 1.7 | uterine cancer | NA | −10997 |
| | | | | in situ, | | |
| | | | | operated | | |
| [symbol] | 1 | 10 | 2.2 | NA | NA | NA |
| [symbol] | 0 | 19 | NA | basal cell | basal cell | [symbol] |
| | | | | carcinoma | carcinoma, | |
| | | | | | ovarian cancer | |
| | | | | | disseminated | |
| [symbol] | 1 | 15 | 2.2 | basal cell | NA | −1092 |
| | | | | carcinoma | | |
| [symbol] | 0 | 10 | 1.7 | NA | NA | NA |
| [symbol] | 1 | 10 | 1.4 | NA | breast cancer | 1246 |
| [symbol] | 0 | 23 | 10.3 | NA | NA | NA |
| [symbol] | 0 | 29 | 12.6 | NA | NA | NA |
| [symbol] | 1 | 40 | 265 | NA | NA | NA |
| [symbol] | 0 | 41 | 17.6 | NA | NA | NA |
| [symbol] | 0 | 10 | 4.2 | NA | NA | NA |
| [symbol] | 0 | 10 | 0.84 | squamous cell | NA | −253 |
| | | | | carcinoma | | |
| | | | | operated | | |
| [symbol] | 0 | 10 | 2.4 | NA | NA | NA |
| [symbol] | 0 | 10 | 2.7 | NA | NA | NA |
| [symbol] | 0 | 10 | 11.4 | NA | NA | NA |
| [symbol] | 0 | 14 | 16.7 | NA | NA | NA |
| [symbol] | 0 | 10 | 5.2 | NA | prostate cancer | [symbol] |
| [symbol] | 0 | 33 | 20.3 | bladder cancer | NA | −2328 |
| | | | | operated | | |
| [symbol] | 0 | 11 | 8.3 | NA | NA | NA |
| [symbol] | 0 | 25 | 44.7 | [symbol] | NA | −4201 |
| [symbol] | 1 | 10 | 3 | NA | NA | NA |
| [symbol] | 0 | 15 | 11.5 | Superficial | NA | −3503 |
| | | | | spreading | | |
| | | | | malignant | | |
| | | | | melanoma, | | |
| | | | | operated | | |
| [symbol] | 1 | 10 | 9.2 | breast cancer | NA | −2516 |
| | | | | operated | | |
| [symbol] | 1 | 10 | 3.5 | NA | NA | 1954 |
| [symbol] | 0 | 10 | 1.02 | NA | NA | NA |
| [symbol] | 0 | 10 | 2.3 | NA | NA | NA |
| [symbol] | 1 | 34 | 16.2 | NA | NA | NA |
| [symbol] | 0 | 10 | 2.1 | NA | NA | NA |
| [symbol] | 0 | 10 | 7.6 | rectal cancer | rectal cancer | [symbol] |
| | | | | operated | recurrent, | |
| | | | | | pancreatic | |
| | | | | | cancer | |
| [symbol] | 0 | 11 | 5.7 | squamous cell | basal cell | −1667, |
| | | | | carcinoma, | carcinoma | −720, 1633 |
| | | | | basal cell | | |
| | | | | carcinoma | | |
| | | | | operated | | |
| [symbol] | 0 | 10 | 4.1 | [symbol] | [symbol] | [symbol] |
| [symbol] | 0 | 10 | 1.1 | NA | NA | NA |
| [symbol] | 1 | 10 | 4.9 | NA | NA | NA |
| [symbol] | 0 | 10 | 19 | NA | NA | NA |
| [symbol] | 1 | 10 | 4.1 | NA | NA | NA |
| [symbol] | 1 | 10 | 6.5 | NA | NA | NA |
| [symbol] | 1 | 10 | 10.1 | NA | prostate cancer, | 171, 377, 189 |
| | | | | | malignant | |
| | | | | | melanoma, | |
| | | | | | colon cancer | |
| | | | | | adeno | |
| [symbol] | 0 | 11 | 5.5 | NA | NA | NA |
| [symbol] | 0 | 10 | 1.5 | hodgkins | basal cell | [symbol] |
| | | | | lymphoma, basal | carcinoma | |
| | | | | cell carcinoma | | |
| [symbol] | 0 | 66 | [symbol] | [symbol] | NA | [symbol] |
| [symbol] | 0 | 10 | 3.2 | NA | NA | NA |
| [symbol] | 1 | 15 | 7.9 | NA | NA | NA |
| [symbol] | 0 | 27 | 10 | uterine cancer | basal cell | −361 |
| | | | | operated | carcinoma | |
| [symbol] | 0 | 10 | 2.1 | NA | breast cancer | 130 |
| | | | | | operated | |
| [symbol] | 1 | 10 | 2.3 | basal cell | NA | −2901 |
| | | | | carcinoma | | |
| [symbol] | 0 | 10 | 2.3 | NA | NA | NA |
| [symbol] | 0 | [symbol] | 21 | breast cancer | NA | −5232 |
| | | | | operated | | |
| [symbol] | 1 | 10 | 4 | NA | NA | NA |
| [symbol] | 1 | 332 | 1076 | NA | NA | NA |
| [symbol] | 0 | 10 | 3.3 | breast cancer | [symbol] | [symbol] |
| | | | | operated, | | |
| | | | | malignant | | |
| | | | | melanoma | | |
| | | | | operated | | |
| [symbol] | 0 | 10 | [symbol] | NA | NA | NA |
| [symbol] | 0 | [symbol] | 23 | NA | NA | NA |
| [symbol] | 0 | 10 | 1.5 | NA | NA | NA |
| [symbol] | 0 | 10 | 1.5 | NA | NA | NA |
| [symbol] | 0 | 10 | 0.5 | NA | NA | NA |
| [symbol] | 0 | 20 | 1.99 | NA | NA | NA |
| [symbol] | 0 | 22 | 12.4 | NA | NA | NA |
| [symbol] | 0 | 10 | 9.2 | NA | NA | NA |
| [symbol] | 0 | 21 | 0.7 | NA | NA | NA |
| [symbol] | 0 | 10 | 1.2 | NA | NA | NA |
| [symbol] | 0 | 10 | 2 | NA | NA | NA |
| [symbol] | 1 | 144 | 20.3 | NA | NA | NA |
| [symbol] | 0 | 10 | 2.2 | NA | NA | NA |
| [symbol] | 0 | [symbol] | 72.3 | [symbol] | NA | −134 |
| [symbol] | 0 | 10 | 1.6 | NA | NA | NA |
| [symbol] | 0 | 25 | 9.7 | NA | NA | NA |
| [symbol] | 0 | 10 | 0.54 | NA | NA | NA |
| [symbol] | 0 | 32 | 25 | NA | NA | NA |
| [symbol] | 0 | 10 | 0.98 | NA | NA | NA |
| [symbol] | 0 | 10 | 1.58 | breast cancer | NA | −62 |
| | | | | operated | | |
| [symbol] | 0 | 10 | 1.31 | NA | NA | NA |
| [symbol] | 1 | 10 | 15.2 | NA | prostate cancer, | [symbol] |
| | | | | | bladder cancer | |
| [symbol] | 0 | 12 | 16.3 | NA | NA | NA |
| [symbol] | 0 | 10 | 4.9 | NA | NA | NA |
| [symbol] | 1 | 20 | [symbol] | NA | NA | NA |
| [symbol] | 1 | 14 | 17.8 | NA | NA | NA |
| [symbol] | 1 | 56 | 13.5 | NA | NA | NA |
| [symbol] | 0 | 10 | 2.7 | NA | NA | NA |
| [symbol] | 0 | 10 | 1.2 | NA | NA | NA |
| [symbol] | 0 | 10 | 2.6 | NA | NA | NA |
| [symbol] | 1 | 35 | 1 | NA | NA | NA |
| [symbol] | 0 | 10 | 1.1 | NA | NA | NA |
| [symbol] | 0 | 10 | 1.8 | NA | colon cancer | 1513 |
| [symbol] | 0 | 10 | 3.9 | NA | basal cell | [symbol] |
| | | | | | carcinoma | |
| [symbol] | 0 | 27 | 13.9 | NA | NA | NA |
| [symbol] | 0 | 29 | 15 | NA | NA | NA |
| [symbol] | 0 | 39 | 17.9 | NA | NA | NA |
| [symbol] | 0 | 20 | 2.3 | NA | NA | NA |
| [symbol] | 0 | 20 | 0.3 | NA | NA | NA |
| [symbol] | 0 | 20 | 2.7 | [symbol] | NA | −7 |
| [symbol] | 0 | 42 | 18.7 | NA | NA | NA |
| [symbol] | 0 | 20 | 1.8 | NA | NA | NA |
| [symbol] | 0 | 10 | 2.5 | NA | NA | NA |
| [symbol] | 0 | 10 | 2.5 | NA | NA | NA |
| [symbol] | 0 | 10 | 1.3 | NA | NA | NA |
| [symbol] | 0 | 10 | 0.5 | NA | NA | NA |
| [symbol] | 1 | 10 | 2.7 | NA | NA | NA |
| [symbol] | 0 | 10 | 3.2 | NA | NA | NA |
| [symbol] | 1 | 20 | 5.7 | NA | NA | NA |
| [symbol] | 0 | 10 | 4.0 | lung cancer, | NA | −1920 |
| | | | | NSCLC operated | | |
| | | | | adeno | | |
| [symbol] | 0 | 10 | 6 | NA | NA | NA |
| [symbol] | 0 | 13 | 8.7 | NA | NA | NA |
| [symbol] | 1 | 16 | 7.2 | NA | NA | NA |
| [symbol] | 0 | 36 | 9.5 | breast cancer | recurrent | −816 |
| | | | | operated | breast | |
| | | | | | cancer | |
| [symbol] | 0 | 14 | 4.3 | NA | NA | NA |
| [symbol] | 0 | 21 | 3.6 | [symbol] | [symbol] | [symbol] |
| [symbol] | 1 | 25 | 33.6 | basal cell | NA | −1946 |
| | | | | carcinoma | | |
| [symbol] | 0 | 10 | 0.93 | basal cell | NA | −2793 |
| | | | | carcinoma | | |
| [symbol] | 0 | 10 | 1.1 | NA | NA | NA |
| [symbol] | 1 | 10 | 5.3 | NA | NA | NA |
| [symbol] | 1 | 43 | 14.4 | [symbol] | NA | [symbol] |
| [symbol] | 0 | 10 | 2.5 | NA | NA | NA |
| [symbol] | 0 | 14 | 5.5 | NA | NA | NA |
| [symbol] | 0 | 10 | 6.4 | NA | NA | NA |
| [symbol] | 0 | 10 | 2.2 | NA | NA | NA |
| [symbol] | 0 | 10 | 5.4 | NA | NA | NA |
| [symbol] | 0 | 10 | 6.7 | NA | [symbol] | 1707 |
| [symbol] | 1 | 10 | 1.7 | NA | NA | NA |
| [symbol] | 0 | 10 | 1.5 | NA | NA | NA |
| [symbol] | 0 | 10 | 3.8 | NA | NA | NA |
| [symbol] | 1 | 35 | 7.2 | colon cancer | NA | −45 |
| | | | | operated adeno | | |
| [symbol] | 1 | 13 | 18.9 | NA | NA | NA |
| [symbol] | 0 | 10 | 4.1 | [symbol] | NA | −1611 |
| [symbol] | 0 | 10 | 3.2 | NA | NA | NA |
| [symbol] | 0 | 42 | 700 | NA | NA | NA |
| [symbol] | 0 | 10 | 1.5 | NA | NA | NA |
| [symbol] | 1 | 10 | 2.3 | NA | NA | NA |
| [symbol] | 0 | 10 | 2.3 | NA | NA | NA |
| [symbol] | 1 | 40 | 33.8 | NA | NA | NA |
| [symbol] | 0 | 18 | 11.9 | NA | NA | NA |
| [symbol] | 0 | 30 | 32.1 | NA | NA | NA |
| [symbol] | 0 | 84 | 30.5 | NA | NA | NA |
| [symbol] | 0 | 11 | 2.3 | NA | NA | NA |
| [symbol] | 1 | 23 | 3.4 | NA | NA | NA |
| [symbol] | 0 | 10 | 1.7 | NA | NA | NA |
| [symbol] | 0 | 10 | 4.6 | NA | NA | NA |
| [symbol] | 0 | 45 | 4.1 | NA | NA | NA |
| [symbol] | 0 | 10 | 5.6 | NA | basal cell | 1542 |
| | | | | | carcinoma | |
| [symbol] | 1 | 15 | 6.3 | NA | NA | NA |
| [symbol] | 1 | 15 | 15.9 | NA | bladder cancer | 695 |
| [symbol] | 1 | 24 | [symbol] | NA | NA | NA |
| [symbol] | 1 | 20 | 3.8 | NA | NA | NA |
| [symbol] | 1 | 10 | 4.5 | NA | NA | NA |
| [symbol] | 0 | 10 | 2.4 | [symbol] | basal cell | [symbol] |
| | | | | | carcinoma | |
| [symbol] | 0 | 10 | 3.2 | NA | NA | NA |
| [symbol] | 0 | 10 | 5.2 | NA | NA | NA |
| [symbol] | 0 | 10 | 6.5 | NA | NA | NA |
| [symbol] | 0 | 10 | 1.1 | [symbol] | [symbol] | [symbol] |
| [symbol] | 0 | 39 | 2.3 | NA | NA | NA |
| [symbol] | 1 | 151 | 27.5 | NA | NA | NA |
| [symbol] | 0 | 20 | 4.2 | breast cancer | NA | −392 |
| | | | | operated | | |
| [symbol] | 0 | 10 | 2.1 | NA | NA | NA |
| [symbol] | 0 | 10 | 10.4 | NA | NA | NA |
| [symbol] | 0 | 10 | 5.6 | NA | NA | NA |
| [symbol] | 0 | 40 | 8.4 | breast cancer | NA | −2202 |
| | | | | operated | | |
| [symbol] | 0 | 83 | 81.3 | NA | NA | NA |
| [symbol] | 0 | 10 | 1 | [symbol] | NA | −742 |
| [symbol] | 0 | 10 | 3.1 | [symbol] | NA | [symbol] |
| [symbol] | 1 | 10 | 3.9 | NA | prostate cancer | 27 |
| | | | | | operated | |
| [symbol] | 0 | 10 | 31.9 | breast cancer | NA | −2513 |
| | | | | operated | | |
| [symbol] | 1 | 11 | 3.9 | NA | NA | NA |
| [symbol] | 0 | 17 | 10.3 | NA | NA | NA |
| [symbol] | 0 | 11 | 2.1 | NA | NA | NA |
| [symbol] | 0 | 20 | 13.9 | NA | NA | NA |
| [symbol] | 0 | 10 | 3.5 | NA | NA | NA |
| [symbol] | 0 | 10 | 2.9 | bladder cancer | NA | −2655 |
| | | | | operated | | |
| [symbol] | 0 | 10 | 3.9 | breast cancer | [symbol] | [symbol] |
| | | | | operated | | |
| [symbol] | 0 | 10 | 8.9 | NA | NA | NA |
| [symbol] | 1 | 124 | 21.3 | NA | NA | NA |
| [symbol] | 1 | 10 | 3.2 | NA | NA | NA |
| [symbol] | 0 | 10 | 1.1 | NA | breast cancer | 661 |
| | | | | | operated | |
| [symbol] | 0 | 10 | 3.4 | NA | NA | NA |
| [symbol] | 0 | 71 | 78.7 | [symbol] | NA | [symbol] |
| [symbol] | 1 | 53 | 110 | NA | breast cancer | 1391 |
| [symbol] | 1 | 30 | 3.5 | NA | NA | NA |
| [symbol] | 0 | 32 | 22.3 | squamous cell | [symbol] | −1979, 602 |
| | | | | carcinoma | | |
| | | | | operated | | |
| [symbol] | 0 | 22 | 6.4 | NA | NA | NA |
| [symbol] | 0 | 35 | 19.3 | NA | NA | NA |
| [symbol] | 0 | 10 | 15 | squamous cell | NA | −1665 |
| | | | | carcinoma | | |
| | | | | operated | | |
| [symbol] | 0 | 10 | 3 | basal cell | basal cell | [symbol] |
| | | | | carcinoma | carcinoma | |
| [symbol] | 0 | 10 | 3.3 | NA | NA | NA |
| [symbol] | 0 | 10 | 3.2 | NA | NA | NA |
| [symbol] | 0 | 10 | 5.2 | basal cell | NA | −1517, −1295 |
| | | | | carcinoma | | |
| [symbol] | 0 | 10 | 5.4 | squamous cell | NA | −2228 |
| | | | | carcinoma | | |
| | | | | operated | | |
| [symbol] | 0 | 10 | 2.4 | NA | NA | NA |
| [symbol] | 0 | 10 | 2.6 | [symbol] | breast cancer | [symbol] |
| | | | | | operated | |
| [symbol] | 0 | 13 | 5.8 | renal cancer | [symbol] | [symbol] |
| | | | | operated | | |
| [symbol] | 0 | 10 | 1.3 | NA | NA | NA |
| [symbol] | 1 | 84 | 22.5 | hodgkins | NA | −13150, −325 |
| | | | | lymphoma, basal | | |
| | | | | cell carcinoma | | |
| [symbol] | 0 | 10 | 4.5 | thyroid cancer | [symbol] | [symbol] |
| | | | | operated, rectal | | |
| | | | | cancer operated | | |
| [symbol] | 0 | 84 | 19.7 | NA | NA | NA |
| [symbol] | 0 | 40 | 5.5 | NA | NA | NA |
| [symbol] | 0 | 10 | 0.95 | NA | NA | NA |
| [symbol] | 0 | 10 | 1.2 | NA | NA | NA |
| [symbol] | 1 | 10 | 1.3 | basal cell | Chronic | [symbol] |
| | | | | carcinoma | lymphocytic | |
| | | | | | leukemia | |
| [symbol] | 0 | 10 | 1.9 | NA | NA | NA |
| [symbol] | 0 | 12 | 5.7 | NA | NSCLC operated | 251 |
| | | | | | stage I | |
| | | | | | squamous | |
| [symbol] | 0 | 10 | 2.9 | NA | NA | NA |
| [symbol] | 0 | 39 | 12.3 | NA | NA | NA |
| [symbol] | 0 | 55 | 45.1 | NA | basal cell | 24 |
| | | | | | carcinoma | |
| [symbol] | 0 | 10 | 3.9 | NA | NA | NA |
| [symbol] | 1 | 295 | 45.6 | NA | NA | NA |
| [symbol] | 1 | 10 | 10.7 | NA | [symbol] | 1853 |
| [symbol] | 0 | 12 | 16.7 | NA | NA | NA |
| [symbol] | 0 | 10 | 2.1 | NA | NA | NA |
| [symbol] | 0 | 10 | 3.2 | NA | [symbol] | 1132 |
| [symbol] | 0 | 11 | 2.5 | NA | NA | NA |
| [symbol] | 0 | 10 | 5.2 | NA | basal cell | 1124 |
| | | | | | carcinoma | |
| [symbol] | 0 | 10 | 5.2 | coloncancer | NA | −24 |
| | | | | operated adeno | | |
| [symbol] | 1 | 10 | 5.7 | NA | NA | NA |
| [symbol] | 1 | 10 | 5 | uterine cancer | NA | −13005 |
| | | | | in situ, | | |
| | | | | operated | | |
| [symbol] | 0 | 10 | 3.3 | NA | NA | NA |
| [symbol] | 0 | 10 | 4.3 | NA | NA | NA |
| [symbol] | 0 | 10 | 5 | NA | NA | NA |
| [symbol] | 0 | 10 | 13.3 | [symbol] | NA | −165 |
| [symbol] | 0 | 37 | 18.3 | NA | NA | NA |
| [symbol] | 1 | 10 | 2.2 | NA | NA | NA |
| [symbol] | 0 | 16 | 5.4 | breast cancer | NA | −3105 |
| | | | | operated | | |
| [symbol] | 1 | 42 | 334 | [symbol] | NA | −2516 |
| [symbol] | 1 | 10 | 2.5 | NA | NA | NA |
| [symbol] | 0 | 10 | 5.7 | NA | NA | NA |
| [symbol] | 0 | 18 | 13 | NA | [symbol] | 2457 |
| [symbol] | 0 | 10 | 1.3 | squamous cell | NA | −252 |
| | | | | carcinoma | | |
| | | | | operated | | |
| [symbol] | 1 | 16 | 1.5 | NA | NA | NA |
| [symbol] | 0 | 12 | 115.2 | tonsil cancer, | [symbol] | [symbol] |
| | | | | operated | | |
| [symbol] | 0 | 19 | 5 | basal cell | NA | −3795 |
| | | | | carcinoma | | |
| [symbol] | 0 | 10 | 2.8 | testicular cancer | NA | −720 |
| | | | | operated | | |
| [symbol] | 1 | 10 | 5.5 | NA | uterine cancer | 350 |
| [symbol] | 0 | 20 | 3.3 | NA | NA | NA |
| [symbol] | 0 | 10 | 1 | [symbol] | NA | [symbol] |
| [symbol] | 1 | 10 | NA | NA | NA | NA |
| [symbol] | 0 | 10 | 6 | [symbol] | NA | [symbol] |
| [symbol] | 0 | 21 | 19 | NA | NA | NA |
| [symbol] | 0 | 29 | 24.1 | NA | NA | NA |
| [symbol] | 0 | 10 | 2.5 | prostate cancer | NA | [symbol] |
| | | | | adeno | | |
| [symbol] | 0 | 10 | 0.6 | hodgkins lymphoma | NA | −7287 |
| [symbol] | 1 | 10 | 20.1 | NA | squamous cell | 1185 |
| | | | | | carcinoma | |
| [symbol] | 0 | 30 | 14.9 | NA | NA | NA |
| [symbol] | 0 | 29 | 22.3 | NA | NA | NA |
| [symbol] | 0 | 10 | 15.2 | NA | NA | NA |
| [symbol] | 0 | 19 | 33.4 | NA | NA | NA |
| [symbol] | 0 | 10 | 1.3 | NA | NA | NA |
| [symbol] | 0 | 10 | 2.2 | NA | lung cancer, | 1616 |
| | | | | | SCLC | |
| [symbol] | 1 | 34 | 10 | NA | NA | NA |
| [symbol] | 0 | 27 | 4.3 | NA | NA | NA |
| [symbol] | 0 | 20 | 2.7 | NA | NA | NA |
| [symbol] | 1 | 10 | 1.6 | NA | NA | NA |
| [symbol] | 0 | 10 | 0.7 | [symbol] | NA | −49 |
| [symbol] | 0 | 10 | 2 | NA | NA | NA |
| [symbol] | 0 | 50 | 44.1 | NA | NA | NA |
| [symbol] | 0 | 13 | 4.3 | NA | NA | NA |
| [symbol] | 0 | 10 | 0.87 | NA | NA | NA |
| [symbol] | 0 | 10 | 2.18 | NA | NA | NA |
| [symbol] | 0 | 10 | 3 | NA | NA | NA |
| [symbol] | 0 | 10 | 0.78 | NA | NA | NA |
| [symbol] | 1 | 10 | [symbol] | NA | NA | NA |
| [symbol] | 0 | 10 | 5.3 | basal cell | NA | −751 |
| | | | | carcinoma | | |
| [symbol] | 0 | 21 | 4 | NA | NA | NA |
| [symbol] | 0 | 21 | 9.7 | NA | NA | NA |
| [symbol] | 0 | 10 | 7.7 | uterine cancer | NA | [symbol] |
| | | | | operated | | |
| [symbol] | 0 | 10 | [symbol] | NA | NA | NA |
| [symbol] | 0 | 20 | 1.6 | NA | NA | NA |
| [symbol] | 0 | 12 | 20.6 | [symbol] | NA | [symbol] |
| [symbol] | 0 | 10 | NA | NA | NA | NA |
| [symbol] | 0 | 10 | 0.81 | NA | NA | NA |
| [symbol] | 1 | 10 | 6.96 | NA | NA | NA |
| [symbol] | 0 | 10 | 2.7 | NA | NA | NA |
| [symbol] | 0 | 10 | 3.9 | NA | NA | NA |
| [symbol] | 0 | 10 | 3.8 | NA | NA | NA |
| [symbol] | 1 | 24 | 168 | NA | NA | NA |
| [symbol] | 1 | 10 | 3.9 | bladder cancer | NA | [symbol] |
| | | | | operated | | |
| [symbol] | 0 | 10 | 0.4 | breast cancer | basal cell | [symbol] |
| | | | | operated | carcinoma | |
| [symbol] | 0 | 10 | 1.9 | NA | NA | NA |
| [symbol] | 0 | 10 | 1.5 | NA | NA | NA |
| [symbol] | 0 | 10 | 3.7 | [symbol] | NA | −1791, −1112 |
| [symbol] | 0 | 10 | 4.1 | NA | NA | NA |
| [symbol] | 1 | 58 | 792 | NA | NA | NA |
| [symbol] | 0 | 10 | 1.1 | [symbol] | NA | [symbol] |
| [symbol] | 0 | 41 | 17.3 | NA | NA | NA |
| [symbol] | 1 | 15 | 766 | NA | NA | NA |
| [symbol] | 0 | 10 | 1.7 | NA | NA | NA |
| [symbol] | 0 | 22 | 15.3 | prostate cancer | NA | [symbol] |
| | | | | adeno | | |
| [symbol] | 0 | 29 | 12.4 | Superficial | NA | −525 |
| | | | | spreading | | |
| | | | | malignant | | |
| | | | | melanoma, | | |
| | | | | operated | | |
| [symbol] | 0 | 14 | 3.5 | NA | [symbol] | 53, 2343 |
| [symbol] | 0 | 10 | 2.6 | breast cancer | NA | −2124 |
| | | | | operated | | |
| [symbol] | 1 | 10 | 2.7 | NA | NA | NA |
| [symbol] | 1 | 13 | 1.1 | NA | NA | NA |
| [symbol] | 0 | 10 | 15.2 | NA | bladder cancer | 2355 |
| [symbol] | 0 | 10 | 1.4 | basal cell | [symbol] | [symbol] |
| | | | | carcinoma | | |
| [symbol] | 0 | 10 | NA | NA | [symbol] | 236 |
| [symbol] | 0 | 10 | 4.3 | breast cancer | NA | [symbol] |
| | | | | operated | | |
| [symbol] | 0 | 10 | 19.4 | NA | NA | NA |
| [symbol] | 0 | 13 | 4.1 | NA | [symbol] | 49, 278 |
| [symbol] | 0 | 10 | 3 | NA | NA | NA |
| [symbol] | 0 | 10 | 2.1 | NA | NA | NA |
| [symbol] | 1 | 31 | 9.3 | NA | NA | NA |
| [symbol] | 0 | 10 | 2.1 | NA | NA | NA |
| [symbol] | 1 | 195 | 605 | NA | NA | NA |
| [symbol] | 0 | 28 | 12.3 | NA | NA | NA |
| [symbol] | 0 | 25 | 145 | colon cancer | NA | [symbol] |
| | | | | operated adeno, | | |
| | | | | squamous cell | | |
| | | | | carcinoma, basal | | |
| | | | | cell carcinoma | | |
| [symbol] | 0 | 10 | 3.5 | NA | NA | NA |
| [symbol] | 1 | 10 | 5.5 | NA | NA | NA |
| [symbol] | 0 | 10 | 3.6 | colon cancer | NA | −22 |
| | | | | operated adeno | | |
| [symbol] | 0 | 75 | 6.7 | NA | NA | NA |
| [symbol] | 0 | 10 | 7.1 | NA | NA | NA |
| [symbol] | 0 | 363 | 46.9 | NA | NA | NA |
| [symbol] | 0 | 24 | 11.1 | NA | NA | NA |
| [symbol] | 0 | 29 | 14.8 | NA | NA | NA |
| [symbol] | 0 | 107 | 103.8 | NA | NA | NA |
| [symbol] | 1 | 66 | 13.6 | NA | NA | NA |

[symbol]

indicates data missing or illegible when filed

SUPPLEMENTARY TABLE 3
Summary of LUCAS cohort and genomic analyses* (CONTINUED)

| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|---|---|---|---|---|---|---|
| Alive | [symbol] | 1 | 14 | [symbol] | A | 1 | [symbol] | [symbol] | 100 | [symbol] | [symbol] | [symbol] | [symbol] | [symbol] | 2.3 | [symbol] | [symbol] | [symbol] | [symbol] | [symbol] | [symbol] | — | — |
| Alive | | 1 | | | F | 2 | | | 100 | | | | | | 2.4 | | | | | | | 0.01 | 0.20 |
| Alive | | 1 | 22 | | C | 5 | | | 100 | | | | | | 2.0 | | | | | | | — | — |
| Deceased | | 1 | 2 | | [symbol] | 4 | | | 100 | | | | | | 2.3 | | | | | | | — | — |
| Deceased | | 1 | 3 | | | 3 | | | 100 | | | | | | [symbol] | | | | | | | 0.00 | 0.15 |
| Alive | | 1 | 4 | | | 5 | | | 100 | | | | | | | | | | | | | — | — |
| Alive | | 1 | 5 | | | 2 | | | 100 | | | | | | | | | | | | | 0.00 | 0.15 |
| Deceased | | 1 | 5 | | | 2 | | | 100 | | | | | | | | | | | | | — | — |
| Deceased | | 1 | 4 | 2 | | 6 | | | 100 | | | | | | 2.3 | | | | | | | — | — |
| Deceased | | 1 | 1 | 2 | | 5 | | | 100 | | | | | | 1.9 | | | | | | | — | — |
| Alive | | 1 | 6 | 2 | | 3 | | | 100 | | | | | | 2.0 | | | | | | | — | — |
| Deceased | | 1 | 2 | 2 | G | 4 | | | 100 | | | | | | [symbol] | | | | | | | | |
| Deceased | | 2 | 3 | 3 | [symbol] | 1 | | | 100 | | | | | | | | | | | | | 1.00 | 1.00 |
| Alive | | 2 | 5 | 2 | G | | | | 100 | | | | | | 2.0 | | | | | | | — | — |
| Deceased | | 2 | 24 | 2 | J | 2 | | | 100 | | | | | | 1.9 | | | | | | | — | — |
| Deceased | | 2 | 23 | [symbol] | C | 2 | | | 100 | | | | | | 2.2 | | | | | | | — | — |
| Alive | | 2 | 16 | | D | 3 | | | 100 | | | | | | [symbol] | | | | | | | 0.01 | 0.15 |
| Alive | | 2 | 15 | | D | 7 | | | 100 | | | | | | 2.0 | | | | | | | — | — |
| Alive | | 2 | 4 | | [symbol] | 5 | | | 100 | | | | | | 2.0 | | | | | | | 0.20 | 0.35 |
| Alive | | 2 | 20 | 2 | | 2 | | | 100 | | | | | | 2.2 | | | | | | | — | — |
| Alive | | 2 | [symbol] | 2 | | 2 | | | 100 | | | | | | 2.1 | | | | | | | — | — |
| Deceased | | 2 | 11 | 1 | D | 2 | | | 100 | | | | | | 2.0 | 1.21 | | | | | 0.98 | — | — |
| Deceased | | 2 | 5 | 2 | G | 3 | | | 100 | | | | | | 2.5 | [symbol] | | | | | 0.78 | 1.00 | 0.90 |
| Alive | | 2 | 12 | 3 | H | 3 | | | 100 | | | | | | 2.2 | | | | | | 0.32 | — | — |
| Alive | | 3 | [symbol] | 2 | [symbol] | 2 | | | 100 | | | | | | 2.1 | | | | | | 0.29 | — | — |
| Alive | | 3 | 14 | 1 | A | 4 | | | 100 | | | | | | [symbol] | 1.32 | | | | | 0.13 | 0.30 | 0.22 |
| Alive | | 3 | 17 | 1 | D | 3 | | | 100 | | | | | | 2.7 | 1.57 | | | | | 0.09 | — | — |
| Deceased | | 3 | 18 | 1 | A | 7 | | | 100 | | | | | | 2.5 | 1.62 | | | | | [symbol] | — | — |
| Deceased | | 3 | 14 | [symbol] | A | [symbol] | | | 100 | | | | | | 2.2 | [symbol] | | | | | 0.26 | — | — |
| Alive | | 3 | 6 | 2 | G | 2 | | | 100 | | | | | | 2.5 | | | | | | 0.79 | — | — |
| Deceased | | 3 | 12 | 3 | H | 4 | | | 100 | | | | | | 2.3 | | | | | | 0.29 | 1.00 | 0.62 |
| Deceased | | 3 | 12 | [symbol] | H | [symbol] | | | 100 | | | | | | 2.4 | | | | | | [symbol] | 0.00 | 0.23 |
| Alive | | 3 | 1 | 2 | [symbol] | 4 | | | 100 | | | | | | 2.0 | 1.29 | | | | | 0.09 | 0.81 | 0.45 |
| Alive | | 3 | 2 | 2 | G | 4 | | | 100 | | | | | | 2.3 | [symbol] | | | | | [symbol] | 0.00 | 0.45 |
| Deceased | | 3 | 14 | 3 | A | 4 | | | 100 | | | | | | 2.5 | 1.43 | | | | | 0.78 | 0.12 | 0.44 |
| Deceased | | 3 | 11 | 3 | H | 3 | | | 100 | | | | | | 2.5 | 1.76 | | | | | 0.67 | 0.96 | 0.02 |
| Alive | | 4 | 5 | [symbol] | F | [symbol] | | | 100 | | | | | | 2.3 | 1.57 | | | | | 0.67 | — | — |
| Deceased | | 4 | 4 | 2 | [symbol] | | | | 100 | | | | | | 1.9 | 1.27 | | | | | 0.93 | 0.99 | 0.95 |
| Alive | | 5 | 5 | 2 | G | | | | 100 | | | | | | 2.5 | 1.71 | | | | | 0.33 | 0.28 | 0.33 |
| Deceased | | 5 | [symbol] | 3 | F | 3 | | | 100 | | | | | | 2.3 | [symbol] | | | | | [symbol] | — | — |
| Deceased | | 5 | | 3 | [symbol] | 3 | | | 100 | | | | | | 2.3 | | | | | | | — | — |
| Deceased | | 5 | 18 | 3 | A | 7 | | | 100 | | | | | | 2.2 | | | | | | 0.19 | — | — |
| Alive | | 5 | [symbol] | 3 | F | [symbol] | | | 100 | | | | | | 2.0 | 1.50 | | | | | 0.68 | 0.18 | 0.39 |
| Alive | | 5 | | 2 | [symbol] | 2 | | | 100 | | | | | | 1.7 | [symbol] | | | | | 0.35 | 0.00 | 0.17 |
| Deceased | | 5 | | 2 | | 3 | | | 100 | | | | | | [symbol] | 1.22 | | | | | [symbol] | 0.36 | 0.53 |
| Alive | | 5 | | 3 | | 7 | | | 100 | | | | | | 2.2 | [symbol] | | | | | 0.62 | — | — |
| Deceased | | 5 | 8 | 3 | D | 2 | | | 100 | | | | | | 2.3 | 1.40 | | | | | [symbol] | — | — |
| Deceased | | 5 | 21 | 3 | C | 5 | | | 100 | | | | | | 3.2 | 1.43 | | | | | | — | — |
| Deceased | | 5 | [symbol] | 2 | E | 2 | | | 100 | | | | | | [symbol] | [symbol] | | | | | | 0.71 | 0.84 |
| Deceased | | 5 | 24 | 2 | J | 2 | | | 100 | | | | | | 1.0 | | | | | | | 0.61 | 0.31 |
| Alive | | 6 | 11 | [symbol] | H | 6 | | | 100 | | | | | | 1.8 | 1.56 | | | | | | — | — |
| Alive | | 6 | 1 | 2 | [symbol] | 4 | | | 100 | | | | | | 2.0 | 1.36 | | | | | | 0.07 | 0.53 |
| Alive | | 6 | 13 | 3 | F | 3 | | | 100 | | | | | | 2.1 | 1.47 | | | | | | — | — |
| Deceased | | 6 | 1 | 2 | [symbol] | 4 | | | 100 | | | | | | 2.2 | 1.25 | | | | | | 0.92 | 0.90 |
| Alive | | 6 | 2 | 3 | F | 6 | | | 100 | | | | | | 2.2 | 1.41 | | | | | | — | — |
| Alive | | 6 | 2 | 2 | G | 4 | | | 100 | | | | | | 2.4 | 1.54 | | | | | | 0.00 | 0.47 |
| Alive | | 6 | 23 | 3 | C | 3 | | | 100 | | | | | | 2.3 | 1.22 | | | | | | — | — |
| Alive | | 6 | 8 | 3 | F | 6 | | | 100 | | | | | | 1.9 | 1.43 | | | | | | — | — |
| Alive | | 6 | 11 | 3 | H | 5 | | | 100 | | | | | | 1.8 | 1.22 | | | | | | — | — |
| Deceased | | 6 | 3 | 2 | G | 5 | | | 100 | | | | | | 1.3 | 1.55 | | | | | | 0.01 | 0.46 |
| Alive | | 6 | 16 | 1 | D | 5 | | | 100 | | | | | | 2.1 | 1.35 | | | | | | 0.32 | 0.37 |
| Alive | | 7 | [symbol] | 2 | [symbol] | 2 | | | 100 | | | | | | 2.5 | 1.29 | | | | | | 1.00 | |
| Deceased | | 7 | 10 | 3 | F | 2 | | | 100 | | | | | | 1.7 | 1.47 | | | | | | 0.52 | 0.39 |
| Deceased | | 7 | 5 | 2 | G | 2 | | | 100 | | | | | | 2.3 | 1.30 | | | | | | 1.02 | 0.92 |
| Deceased | | 7 | 6 | 2 | G | 6 | | | 100 | | | | | | 2.8 | 1.57 | | | | | | [symbol] | 0.64 |
| Alive | | 7 | 17 | 1 | D | 2 | | | 100 | | | | | | 2.3 | [symbol] | | | | | | — | — |
| Deceased | | 7 | 13 | 3 | F | 3 | | | 100 | | | | | | 2.2 | 1.27 | | | | | | — | — |
| Alive | | 7 | 15 | 1 | A | 2 | | | 100 | | | | | | 2.0 | 1.52 | | | | | | — | — |
| Alive | | 7 | 7 | 1 | D | 2 | | | 100 | | | | | | 2.1 | 1.32 | | | | | | — | — |
| Deceased | | 7 | 16 | 1 | A | 2 | | | 100 | | | | | | 2.6 | 1.21 | | | | | | | 0.63 |
| Deceased | | 7 | 2 | 2 | [symbol] | 7 | | | 100 | | | | | | 2.0 | 1.45 | | | | | | — | — |
| Deceased | | 7 | 23 | 3 | C | 4 | | | 100 | | | | | | 3.0 | 1.18 | | | | | | 1.00 | 1.00 |
| Deceased | | 7 | 21 | 3 | H | 2 | | | 100 | | | | | | 2.1 | 1.26 | | | | | | 1.00 | 0.91 |
| Alive | | 8 | 3 | 2 | G | 1 | | | 100 | | | | | | 2.4 | 1.53 | | | | | | — | — |
| Alive | | 8 | 7 | 3 | F | 2 | | | 100 | | | | | | 2.3 | 1.52 | | | | | | 0.01 | 0.21 |
| Deceased | | 8 | 14 | 1 | A | 4 | | | 100 | | | | | | 2.0 | 1.32 | | | | | | — | — |
| Alive | | 8 | 17 | 2 | [symbol] | 1 | | | 100 | | | | | | 2.7 | 1.27 | | | | | | 0.00 | 0.15 |
| Deceased | | 8 | 8 | [symbol] | F | [symbol] | | | 100 | | | | | | 2.3 | 1.26 | | | | | | 0.35 | 0.32 |
| Deceased | | 8 | [symbol] | 2 | [symbol] | | | | 100 | | | | | | 2.5 | 1.17 | | | | | | — | — |
| Alive | | 8 | 1 | 2 | | | | | 100 | | | | | | 2.3 | 1.23 | | | | | | — | — |
| Alive | | 8 | 2 | 2 | | 7 | | | 100 | | | | | | 2.1 | 1.47 | | | | | | — | — |
| Alive | | 8 | 3 | 2 | G | 2 | | | 100 | | | | | | 2.3 | 1.20 | | | | | | 0.02 | 0.33 |
| Alive | | 8 | 2 | 2 | G | 3 | | | 100 | | | | | | 1.9 | 1.19 | | | | | | — | — |
| Deceased | | 8 | 16 | 1 | D | 3 | | | 100 | | | | | | 2.3 | [symbol] | | | | | | — | — |
| Alive | | 8 | 5 | 2 | G | 2 | | | 100 | | | | | | 2.9 | | | | | | | — | — |
| Deceased | | 9 | 5 | 2 | [symbol] | 3 | | | 100 | | | | | | 2.1 | | | | | | | — | — |
| Alive | | 9 | 5 | 2 | G | 2 | | | 100 | | | | | | 2.9 | | | | | | | 0.09 | 0.51 |
| Alive | | 9 | 5 | 2 | [symbol] | 5 | | | 100 | | | | | | 2.2 | 1.23 | | | | | | 0.00 | 0.04 |
| Deceased | | 9 | 11 | 3 | H | 1 | | | 100 | | | | | | 2.0 | 1.36 | | | | | | — | — |
| Deceased | | 9 | 9 | 3 | F | 4 | | | 100 | | | | | | 2.2 | [symbol] | | | | | | — | — |
| Deceased | | 9 | 13 | 3 | F | 3 | | | 100 | | | | | | 2.3 | | | | | | | 0.00 | 0.18 |
| Deceased | | 9 | 7 | 2 | [symbol] | 3 | | | 100 | | | | | | 2.3 | 1.22 | | | | | | — | — |
| Alive | | 9 | 7 | 2 | | 1 | | | 100 | | | | | | 2.0 | 1.23 | | | | | | — | — |
| Alive | | 9 | 11 | 3 | H | 6 | | | 100 | | | | | | 1.9 | 1.42 | | | | | | — | — |
| Deceased | | 9 | 14 | 1 | A | [symbol] | | | 100 | | | | | | 1.9 | 1.47 | | | | | | — | — |
| Alive | | 10 | 16 | 1 | D | 3 | | | 100 | | | | | | 2.7 | 1.53 | | | | | | 0.50 | 0.37 |
| Deceased | | 10 | 9 | 3 | F | 2 | | | 100 | | | | | | 2.3 | 1.77 | | | | | | — | — |
| Deceased | | 10 | 16 | 1 | D | 2 | | | 100 | | | | | | 2.7 | 1.54 | | | | | | 0.64 | [symbol] |
| Alive | | 10 | 23 | 3 | C | 3 | | | 100 | | | | | | 2.3 | 1.57 | | | | | | — | — |
| Alive | | 10 | [symbol] | 2 | B | 3 | | | 100 | | | | | | [symbol] | [symbol] | | | | | | — | — |
| Deceased | | 10 | | 3 | H | 2 | | | 100 | | | | | | | 1.92 | | | | | | 0.00 | 0.35 |
| Deceased | | 10 | | 3 | C | 3 | | | 100 | | | | | | | [symbol] | | | | | | — | — |
| Deceased | | 10 | 15 | 1 | A | 3 | | | 100 | | | | | | 2.1 | 1.47 | | | | | | — | — |
| Deceased | 21 | 10 | 17 | 1 | D | 6 | | | 100 | | | | | | 2.3 | 1.28 | | | | | | 0.37 | [symbol] |
| Deceased | [symbol] | 10 | 1 | 2 | [symbol] | 2 | | | 100 | | | | | | 1.4 | 1.27 | | | | | | 1.00 | |
| Deceased | | 10 | 21 | 3 | C | 1 | | | 100 | | | | | | 7.2 | 1.42 | | | | | | — | — |
| Deceased | 52 | 10 | 2 | 2 | [symbol] | 7 | | | 100 | | | | | | 2.4 | 1.49 | | | | | | 0.00 | 0.35 |
| Alive | [symbol] | 11 | 11 | 3 | H | 4 | | | 100 | | | | | | 2.0 | 1.33 | | | | | | — | — |
| Alive | | 11 | 1 | 7 | [symbol] | 4 | | | 100 | | | | | | 2.2 | 1.26 | | | | | | — | — |
| Alive | | 11 | 2 | 2 | | 7 | | | 100 | | | | | | 2.2 | 1.27 | | | | | | — | — |
| Alive | | 11 | 3 | 2 | G | 1 | | | 100 | | | | | | 2.0 | 1.08 | | | | | | — | — |
| Alive | | 11 | 3 | 2 | G | 1 | | | 100 | | | | | | 2.1 | 1.33 | | | | | | — | — |
| Deceased | | 11 | 4 | 2 | [symbol] | 6 | | | 100 | | | | | | 1.4 | 1.10 | | | | | | [symbol] | [symbol] |
| Alive | | 11 | 4 | 2 | | 6 | | | 100 | | | | | | 2.3 | 1.13 | | | | | | — | — |
| Alive | | 11 | 5 | 2 | G | 7 | | | 100 | | | | | | [symbol] | 1.11 | | | | | | — | — |
| Alive | | 11 | [symbol] | 2 | [symbol] | 3 | | | 100 | | | | | | 2.2 | 1.14 | | | | | | | |
| Deceased | | 12 | 5 | 2 | G | 7 | | | 100 | | | | | | 2.5 | 1.20 | | | | | | 1.09 | 0.39 |
| Alive | | 12 | 18 | 1 | A | 7 | | | 100 | | | | | | 2.1 | 1.33 | | | | | | 0.03 | 0.12 |
| Deceased | | 12 | 14 | 1 | A | 4 | | | 100 | | | | | | 2.0 | 1.23 | | | | | | 1.00 | [symbol] |
| Alive | | 12 | 13 | 2 | B | 3 | | | 100 | | | | | | 2.3 | 1.22 | | | | | | — | — |
| Deceased | | 12 | 5 | 2 | G | 3 | | | 100 | | | | | | 2.9 | 1.17 | | | | | | — | — |
| Alive | | 12 | 20 | 2 | B | 6 | | | 100 | | | | | | 2.1 | 1.18 | | | | | | — | — |
| Alive | | 12 | 12 | 1 | B | 2 | | | 100 | | | | | | 2.3 | 1.18 | | | | | | — | — |
| Alive | | 12 | 9 | 3 | F | 4 | | | 100 | | | | | | 2.1 | 1.35 | | | | | | — | — |
| Deceased | | 12 | 15 | 1 | D | 7 | | | 100 | | | | | | 2.4 | 1.09 | | | | | | 0.05 | [symbol] |
| Deceased | | 12 | 22 | 3 | [symbol] | 6 | | | 100 | | | | | | 2.6 | 1.17 | | | | | | 1.00 | |
| Deceased | | 13 | 15 | 1 | A | 3 | | | 100 | | | | | | 2.3 | 1.40 | | | | | | — | — |
| Deceased | | 13 | 9 | 3 | F | 2 | | | 100 | | | | | | 2.2 | 1.70 | | | | | | — | — |
| Alive | | 13 | 9 | 1 | B | 3 | | | 100 | | | | | | 2.1 | 1.69 | | | | | | 0.29 | 0.33 |
| Alive | | 13 | 17 | 1 | B | 6 | | | 100 | | | | | | 2.6 | 1.72 | | | | | | — | — |
| Alive | | 13 | 13 | 3 | F | 3 | | | 100 | | | | | | 2.1 | 1.70 | | | | | | 1.00 | 0.92 |
| Deceased | | 13 | 22 | 3 | [symbol] | 6 | | | 100 | | | | | | 2.5 | 1.23 | | | | | | 1.00 | 1.00 |
| Alive | | 13 | 20 | 2 | F | 2 | | | 100 | | | | | | 2.3 | 1.34 | | | | | | 0.20 | 0.19 |
| Alive | | 13 | 12 | 3 | [symbol] | 3 | | | 100 | | | | | | 2.3 | 1.49 | | | | | | — | — |
| Alive | | 13 | 14 | 2 | B | 3 | | | 100 | | | | | | 2.1 | 1.43 | | | | | | 0.48 | 0.35 |
| Deceased | | 13 | 17 | 1 | B | 6 | | | 100 | | | | | | 2.7 | 1.25 | | | | | | — | — |
| Alive | | 13 | 19 | 1 | A | 6 | | | 100 | | | | | | 2.1 | 1.47 | | | | | | — | — |
| Deceased | | 13 | 15 | 1 | A | 5 | | | 100 | | | | | | 2.2 | 1.30 | | | | | | 0.00 | 0.30 |
| Alive | | 13 | 7 | 3 | F | 6 | | | 100 | | | | | | 1.8 | 1.57 | | | | | | — | — |
| Deceased | | 13 | 11 | 3 | C | 7 | | | 100 | | | | | | 1.9 | [symbol] | | | | | | 1.00 | [symbol] |
| Alive | | 13 | 1 | 2 | G | 6 | | | 100 | | | | | | 2.2 | 1.52 | | | | | | | |
| Deceased | | 13 | 23 | 2 | [symbol] | 5 | | | 100 | | | | | | 2.5 | 1.75 | | | | | | 1.00 | |
| Deceased | | 14 | 24 | 2 | | 2 | | | 100 | | | | | | 1.9 | | | | | | | — | — |
| Alive | | 14 | 2 | 2 | | 1 | | | 100 | | | | | | 1.7 | 1.75 | | | | | | 0.01 | 0.13 |
| Deceased | | 14 | 24 | 2 | | 2 | | | 100 | | | | | | 1.9 | 1.81 | | | | | | — | — |
| Deceased | | 14 | 2 | 2 | | 7 | | | 100 | | | | | | 1.9 | | | | | | | [symbol] | 0.30 |
| Alive | | 14 | 3 | 2 | G | 1 | | | 100 | | | | | | 1.7 | 2.23 | | | | | | — | — |
| Alive | | 14 | 4 | 2 | G | 3 | | | 100 | | | | | | 2.3 | 1.40 | | | | | | 0.17 | [symbol] |
| Alive | | 14 | 5 | 2 | G | 2 | | | 100 | | | | | | 2.3 | 1.30 | | | | | | — | — |
| Alive | | 14 | 5 | 2 | [symbol] | 1 | | | 100 | | | | | | 1.5 | 1.34 | | | | | | [symbol] | [symbol] |
| Alive | | 14 | 3 | 2 | G | 1 | | | 100 | | | | | | 2.4 | 1.43 | | | | | | — | — |
| Deceased | | 14 | 4 | 2 | G | 5 | | | 100 | | | | | | 2.3 | 1.45 | | | | | | 1.08 | [symbol] |
| Alive | | 14 | 13 | 3 | H | 2 | | | 100 | | | | | | 2.3 | 1.24 | | | | | | 0.08 | |
| Deceased | | 14 | 5 | 2 | G | 7 | | | 100 | | | | | | 2.7 | 1.53 | | | | | | 1.00 | |
| Deceased | | 14 | 12 | 3 | H | 3 | | | 100 | | | | | | 1.2 | 1.74 | | | | | | — | — |
| Deceased | | 14 | 24 | 2 | [symbol] | 2 | | | 100 | | | | | | 2.0 | 1.74 | | | | | | — | — |
| Alive | | 14 | 8 | 2 | | [symbol] | | | 100 | | | | | | [symbol] | 1.47 | | | | | 0.23 | — | — |
| Deceased | | 14 | 13 | 1 | | | | | 100 | | | | | | | [symbol] | | | | | 0.25 | — | — |
| Deceased | | 15 | 8 | 3 | | | | | 100 | | | | | | 2.7 | 1.24 | | | | | 0.72 | — | — |
| Deceased | | 15 | 23 | 2 | | | | | 100 | | | | | | 2.7 | [symbol] | | | | | 0.12 | 0.00 | 0.05 |
| Alive | | 15 | 2 | 2 | | | | | 100 | | | | | | 2.0 | | | | | | 0.32 | — | — |
| Alive | | 15 | 1 | 3 | | | | | 100 | | | | | | 1.3 | | | | | | 0.39 | — | — |
| Alive | | 15 | 20 | 3 | | | | | 100 | | | | | | 2.0 | | | | | | 0.92 | 0.15 | 0.35 |
| Alive | | 15 | 15 | 1 | | | | | 100 | | | | | | [symbol] | 1.78 | | | | | 0.18 | — | — |
| Deceased | | 15 | 23 | 3 | | | | | 100 | | | | | | | 1.42 | | | | | 0.31 | — | — |
| Deceased | | 15 | 3 | 2 | | | | | 100 | | | | | | 1.2 | 1.22 | | | | | [symbol] | — | — |
| Deceased | | 15 | 5 | 3 | | | | | 100 | | | | | | 2.2 | 1.24 | | | | | | — | — |
| Alive | | 15 | 22 | 3 | | | | | 100 | | | | | | 1.3 | 1.11 | | | | | | [symbol] | [symbol] |
| Deceased | | 15 | 10 | 3 | | | | | 100 | | | | | | [symbol] | 1.21 | | | | | 0.32 | | |
| Alive | | 15 | 9 | 1 | | | | | 100 | | | | | | 2.2 | 1.45 | | | | | 0.57 | | 0.31 |
| Alive | | 15 | 7 | 3 | | | | | 100 | | | | | | [symbol] | 1.28 | | | | | 0.58 | | 0.73 |
| Alive | | 15 | 4 | 2 | | | | | 100 | | | | | | 2.0 | 1.48 | | | | | 0.30 | — | — |
| Alive | | 15 | 32 | 3 | | | | | 100 | | | | | | 3.2 | 1.40 | | | | | 0.46 | 0.11 | 0.29 |
| Alive | | 15 | 19 | 1 | A | | | | 100 | | | | | | 2.3 | 1.42 | | | | | 0.08 | — | 0.12 |
| Alive | | 16 | 22 | 3 | [symbol] | | | | 100 | | | | | | 3.0 | 1.64 | | | | | 0.57 | — | — |
| Deceased | | 16 | 5 | 2 | | | | | 100 | | | | | | 2.7 | 1.74 | | | | | 0.55 | — | — |
| Deceased | | 16 | 11 | 3 | | | | | 100 | | | | | | 2.3 | 1.53 | | | | | 0.53 | [symbol] | 0.71 |
| Alive | | 16 | 6 | 2 | | | | | 100 | | | | | | 2.1 | 2.09 | | | | | 0.58 | — | — |
| Alive | | 16 | 11 | 3 | | | | | 100 | | | | | | 3.7 | [symbol] | | | | | 0.43 | — | — |
| Deceased | | 16 | 13 | 2 | | | | | 100 | | | | | | 2.5 | | | | | | 0.27 | 1.00 | 0.93 |
| Alive | | 16 | 18 | 1 | | | | | 100 | | | | | | 2.2 | | | | | | 0.33 | 0.00 | 0.10 |
| Alive | | 16 | 1 | 2 | | | | | 100 | | | | | | 2.5 | | | | | | 0.33 | 0.00 | 0.15 |
| Alive | 2702 | 16 | 2 | 2 | | | | | 100 | | | | | | 2.2 | 1.32 | | | | | | — | — |
| Deceased | [symbol] | 16 | 17 | 1 | | | | | 100 | | | | | | 2.6 | 1.47 | | | | | | 1.00 | [symbol] |
| Alive | 2701 | 16 | 15 | 1 | | | | | 100 | | | | | | 2.4 | 1.55 | | | | | | 1.00 | |
| Deceased | [symbol] | 16 | 9 | 1 | | | | | 100 | | | | | | 2.2 | 1.52 | | | | | | 1.00 | 1.00 |
| Alive | 2701 | 16 | 3 | 2 | | | | | 100 | | | | | | [symbol] | [symbol] | | | | | | — | — |
| Deceased | | 16 | 4 | 2 | | | | | 100 | | | | | | | 1.57 | | | | | | — | — |
| Alive | | 16 | 5 | 2 | | | | | 100 | | | | | | 2.8 | 2.20 | | | | | | 0.33 | 0.55 |
| Alive | | 16 | 6 | 2 | | | | | 100 | | | | | | 2.4 | 1.72 | | | | | | — | — |
| Alive | | 17 | 21 | 3 | | | | | 100 | | | | | | 2.5 | 1.30 | | | | | | — | — |
| Deceased | | 17 | 13 | 1 | | | | | 100 | | | | | | 2.2 | 1.30 | | | | | | — | — |
| Alive | | 17 | 14 | 2 | | | | | 100 | | | | | | 2.6 | 1.34 | | | | | | — | — |
| Deceased | | 17 | 11 | 3 | | | | | 100 | | | | | | 2.2 | 1.28 | | | | | | 0.31 | [symbol] |
| Alive | | 17 | 23 | 2 | | | | | 100 | | | | | | 2.6 | 1.51 | | | | | | 0.65 | |
| Deceased | | 17 | 12 | 3 | | | | | 100 | | | | | | 1.9 | 1.51 | | | | | | [symbol] | |
| Deceased | | 17 | [symbol] | 3 | | | | | 100 | | | | | | 2.8 | 1.27 | | | | | | 1.00 | |
| Alive | | 17 | 11 | 3 | | | | | 100 | | | | | | 2.6 | 1.58 | | | | | | [symbol] | |
| Alive | | 17 | 17 | 1 | | | | | 100 | | | | | | 2.2 | 1.29 | | | | | | — | — |
| Alive | | 17 | [symbol] | 1 | | | | | 100 | | | | | | 2.0 | 1.18 | | | | | | 0.00 | 0.04 |
| Deceased | | 17 | | 1 | | | | | 100 | | | | | | 2.8 | 1.31 | | | | | | 1.00 | 0.96 |
| Alive | | 17 | | 1 | | | | | 100 | | | | | | 1.7 | 1.31 | | | | | | 0.52 | 0.53 |
| Alive | | 17 | | 2 | | | | | 100 | | | | | | 2.5 | 1.15 | | | | | | — | — |
| Alive | | 17 | | 3 | | | | | 100 | | | | | | 2.3 | 1.34 | | | | | | — | — |
| Deceased | | 17 | | 2 | | 5 | | | 100 | | | | | | 2.4 | 1.37 | | | | | | 1.00 | 0.83 |
| Alive | | 17 | 8 | 3 | | 1 | | | 100 | | | | | | 2.2 | 1.32 | | | | | | — | — |
| Alive | | 18 | 18 | 1 | | 7 | | | 100 | | | | | | 2.3 | [symbol] | | | | | | — | — |
| Deceased | | 18 | 23 | 2 | | 5 | | | 100 | | | | | | 2.5 | 1.35 | | | | | | 1.00 | 0.73 |
| Alive | | 18 | 23 | 3 | C | 3 | | | 100 | | | | | | 2.6 | 1.35 | | | | | | [symbol] | [symbol] |
| Deceased | | 18 | [symbol] | 2 | [symbol] | 3 | | | 100 | | | | | | [symbol] | 1.34 | | | | | | — | — |
| Deceased | 6 | 18 | 11 | 3 | | 4 | | | 100 | | | | | | 2.3 | 1.19 | | | | | | 1.00 | 0.95 |
| Alive | [symbol] | 18 | 14 | 1 | | 1 | | | 100 | | | | | | 1.9 | 1.54 | | | | | | — | — |
| Deceased | | 18 | 11 | 3 | | 5 | | | 100 | | | | | | 2.3 | 1.36 | | | | | | — | — |
| Deceased | | 18 | 17 | 1 | | 6 | | | 100 | | | | | | 2.3 | 1.72 | | | | | | — | — |
| Deceased | | 18 | 20 | 2 | | 1 | | | 100 | | | | | | 1.9 | 1.38 | | | | | | — | — |
| Deceased | | 18 | 12 | 3 | | 3 | | | 100 | | | | | | 1.6 | [symbol] | | | | | | [symbol] | 0.79 |
| Alive | | 18 | 10 | 3 | | 8 | | | 100 | | | | | | 2.3 | | | | | | | 0.00 | 0.28 |
| Deceased | 2209 | 18 | 22 | 3 | | 4 | | | 100 | | | | | | 3.1 | 1.50 | | | | | | 1.00 | 0.50 |
| Deceased | [symbol] | 18 | 9 | 1 | | 1 | | | 100 | | | | | | 2.7 | 1.55 | | | | | | — | — |
| Alive | | 19 | 1 | 2 | | [symbol] | | | 100 | | | | | | 1.6 | 1.17 | | | | | | — | — |
| Alive | | 19 | 23 | 3 | | | | | 100 | | | | | | 2.4 | [symbol] | | | | | | — | — |
| Alive | | 19 | 12 | 3 | | | | | 100 | | | | | | 2.4 | 1.36 | | | | | | — | — |
| Alive | | 19 | 2 | 3 | | 1 | | | 100 | | | | | | 2.2 | 1.35 | | | | | | — | — |
| Deceased | 1100 | 19 | 3 | 2 | | 6 | | | 100 | | | | | | 2.6 | 1.11 | | | | | | 0.04 | 0.04 |
| Deceased | 313 | 19 | 4 | 2 | | 3 | | | 100 | | | | | | 2.3 | 1.25 | | | | | | 0.03 | 0.23 |
| Alive | [symbol] | 19 | 13 | 3 | | 3 | | | 100 | | | | | | 2.1 | 1.31 | | | | | | — | — |
| Alive | | 19 | 3 | 3 | | 7 | | | 100 | | | | | | 2.4 | 1.31 | | | | | | 0.00 | 0.27 |
| Deceased | | 19 | 14 | 1 | | 1 | | | 100 | | | | | | 2.6 | 1.30 | | | | | | 1.00 | 0.23 |
| Deceased | | 19 | 19 | 1 | | 7 | | | 100 | | | | | | 2.5 | [symbol] | | | | | | 1.00 | 0.26 |
| Alive | | 19 | 5 | 2 | | 3 | | | 100 | | | | | | 2.1 | 1.15 | | | | | | 0.01 | 0.03 |
| Deceased | | 19 | 7 | 3 | | [symbol] | | | 100 | | | | | | 1.9 | 1.19 | | | | | | — | — |
| Deceased | | 19 | [symbol] | 1 | | 4 | | | 100 | | | | | | 2.0 | 1.12 | | | | | | 1.00 | 0.97 |
| Deceased | 11 | 19 | | 3 | | 1 | | | 100 | | | | | | 2.5 | 1.12 | | | | | | 1.00 | 1.00 |
| Deceased | [symbol] | 19 | 17 | 2 | | 2 | | | 100 | | | | | | 2.2 | 1.20 | | | | | | 0.99 | 0.91 |
| Alive | | 19 | 13 | 3 | | 3 | | | 100 | | | | | | 2.3 | [symbol] | | | | | | — | — |
| Alive | | 20 | [symbol] | 2 | | 1 | | | 100 | | | | | | 2.3 | 1.88 | | | | | | — | — |
| Alive | | 20 | 7 | 2 | | 3 | | | 100 | | | | | | 2.3 | 1.65 | | | | | | 0.13 | [symbol] |
| Deceased | | 20 | 9 | 1 | | 1 | | | 100 | | | | | | 2.9 | 2.05 | | | | | | — | — |
| Deceased | | 20 | 1 | 2 | | 6 | | | 100 | | | | | | 2.3 | 1.58 | | | | | | — | — |
| Alive | | 20 | 27 | 3 | C | 8 | | | 100 | | | | | | 2.3 | 1.42 | | | | | | — | — |
| Alive | | 20 | 2 | 3 | F | 1 | | | 100 | | | | | | 1.8 | 2.09 | | | | | | — | — |
| Alive | | 20 | 3 | 2 | [symbol] | [symbol] | | | 100 | | | | | | 2.5 | 1.41 | | | | | | — | — |
| Alive | | 20 | 13 | 1 | | | | | 100 | | | | | | 2.4 | 1.78 | | | | | | — | — |
| Deceased | | 20 | 10 | 3 | H | | | | 100 | | | | | | 1.5 | 1.57 | | | | | | — | — |
| Alive | | 20 | 4 | 2 | G | | | | 100 | | | | | | 2.1 | 2.37 | | | | | | — | — |
| Deceased | 78 | 20 | 14 | 1 | D | | | | 100 | | | | | | 2.3 | 1.73 | | | | | | 1.00 | 0.23 |
| Alive | [symbol] | 20 | 15 | 1 | A | | | | 100 | | | | | | 1.9 | 1.93 | | | | | | 0.81 | 0.73 |
| Alive | | 20 | 16 | 1 | D | | | | 100 | | | | | | 2.4 | 1.97 | | | | | | — | — |
| Alive | | 20 | 14 | 1 | A | | | | 100 | | | | | | 2.2 | 2.27 | | | | | | — | — |
| Deceased | | 20 | 3 | 2 | G | | | | 100 | | | | | | 2.7 | 2.31 | | | | | | — | — |
| Alive | | 20 | 12 | 3 | H | | | | 100 | | | | | | 2.1 | 2.50 | | | | | | — | — |
| Deceased | | 21 | 6 | 2 | [symbol] | 3 | | | 100 | | | | | | 2.3 | 1.18 | | | | | | — | — |
| Alive | | 21 | 14 | 1 | | 7 | | | 100 | | | | | | 2.3 | 1.17 | | | | | | — | — |
| Deceased | | 21 | 12 | 1 | | 4 | | | 100 | | | | | | 2.6 | 1.26 | | | | | | 1.00 | 1.00 |
| Alive | | 21 | 9 | 3 | | 4 | | | 100 | | | | | | 2.2 | 1.37 | | | | | | — | — |
| Alive | | 21 | 13 | 1 | A | 3 | | | 100 | | | | | | 2.3 | 1.48 | | | | | | — | — |
| Deceased | | 21 | 15 | 1 | A | 8 | | | 100 | | | | | | 2.7 | 1.43 | | | | | | — | — |
| Deceased | | 21 | 16 | 1 | A | 8 | | | 100 | | | | | | 2.3 | 1.28 | | | | | | — | — |
| Deceased | 27 | 21 | 20 | 3 | [symbol] | 8 | | | 100 | | | | | | 3.3 | 1.23 | | | | | | 1.00 | 1.00 |
| Alive | [symbol] | 22 | 1 | 2 | | 6 | | | 100 | | | | | | 1.6 | 1.17 | | | | | | [symbol] | [symbol] |
| Deceased | | 22 | 24 | 2 | | 3 | | | 100 | | | | | | 1.3 | 1.31 | | | | | | | |
| Alive | | 12 | 13 | 1 | A | 2 | | | 100 | | | | | | 2.5 | 1.28 | | | | | | — | — |
| Alive | | 22 | 11 | 3 | H | 5 | | | 100 | | | | | | 2.3 | 2.37 | | | | | | — | — |
| Alive | | 22 | 21 | 3 | C | 7 | | | 100 | | | | | | 2.6 | 1.13 | | | | | | — | — |
| Alive | | 22 | 23 | 2 | B | 5 | | | 100 | | | | | | 2.4 | 1.29 | | | | | | — | — |
| Deceased | | 22 | 21 | 3 | C | 5 | | | 100 | | | | | | 2.3 | 1.20 | | | | | | 1.03 | 0.87 |
| Alive | | 22 | 17 | 2 | B | 2 | | | 100 | | | | | | 2.2 | 1.13 | | | | | | 0.01 | 0.07 |
| Alive | | 22 | [symbol] | 3 | F | 6 | | | 100 | | | | | | 2.4 | 1.17 | | | | | | 0.00 | 0.12 |
| Alive | | 22 | 10 | 1 | D | 3 | | | 100 | | | | | | 2.0 | 1.35 | | | | | | 0.01 | 0.29 |
| Deceased | | 22 | 15 | 1 | D | [symbol] | | | 100 | | | | | | 2.8 | 1.10 | | | | | | — | — |
| Deceased | | 22 | 13 | 1 | A | 2 | | | 100 | | | | | | 2.5 | 1.19 | | | | | | [symbol] | 0.97 |
| Alive | | 22 | 7 | 1 | D | 3 | | | 100 | | | | | | 2.1 | 1.34 | | | | | | 1.00 | [symbol] |
| Alive | | 22 | 22 | 3 | C | 3 | | | 100 | | | | | | 1.7 | 1.35 | | | | | | — | — |
| Deceased | | 22 | 19 | 1 | A | [symbol] | | | 100 | | | | | | 1.9 | 1.67 | | | | | | — | — |
| Alive | | 22 | 17 | 2 | B | 2 | | | 100 | | | | | | 2.1 | 1.35 | | | | | | 0.00 | 0.30 |
| Alive | | 23 | 20 | 2 | E | 1 | | | 100 | | | | | | 3.3 | 1.35 | | | | | | 0.37 | 0.25 |
| Alive | | 23 | 20 | 2 | E | 2 | | | 100 | | | | | | 2.5 | 1.17 | | | | | | — | — |
| Deceased | | 23 | 10 | 1 | D | 8 | | | 100 | | | | | | 1.3 | 1.33 | | | | | | 1.00 | 0.91 |
| Alive | | 23 | 19 | 1 | D | 3 | | | 100 | | | | | | 2.5 | 1.31 | | | | | | 0.00 | 0.12 |
| Deceased | | 23 | 19 | 1 | A | 3 | | | 100 | | | | | | 1.3 | 1.19 | | | | | | — | — |
| Deceased | | 23 | 18 | 1 | D | 8 | | | 100 | | | | | | 1.3 | 1.22 | | | | | | — | — |
| Alive | | 23 | 22 | 3 | C | [symbol] | | | 100 | | | | | | 2.5 | 1.12 | | | | | | — | — |
| Alive | | 23 | 1 | 2 | G | | | | 100 | | | | | | 2.7 | 1.33 | | | | | | — | — |
| Alive | | 23 | 7 | 3 | H | 1 | | | 100 | | | | | | 2.3 | 1.23 | | | | | | — | — |
| Deceased | | 23 | 2 | 3 | F | 1 | | | 100 | | | | | | 2.2 | 1.29 | | | | | | — | — |
| Deceased | | 23 | 12 | 3 | H | 2 | | | 100 | | | | | | 2.5 | 1.35 | | | | | | 1.00 | [symbol] |
| Deceased | | 23 | 10 | 3 | H | 7 | | | 100 | | | | | | 2.2 | 1.11 | | | | | | 0.55 | 0.43 |
| Alive | | 23 | 13 | 1 | A | 5 | | | 100 | | | | | | 2.4 | 1.54 | | | | | | — | — |
| Deceased | | 23 | 8 | 5 | F | 5 | | | 100 | | | | | | 2.8 | [symbol] | | | | | | — | — |
| Alive | | 23 | 3 | 2 | [symbol] | 8 | | | 100 | | | | | | 2.0 | | | | | | | — | — |
| Alive | | 23 | 4 | 2 | G | 3 | | | 100 | | | | | | 2.0 | 1.27 | | | | | | — | — |
| Alive | | 24 | 5 | 2 | G | 2 | | | 100 | | | | | | 2.5 | 1.44 | | | | | | — | — |
| Deceased | | 24 | [symbol] | 2 | A | 8 | | | 100 | | | | | | 2.1 | 1.32 | | | | | | 0.84 | 0.49 |
| Deceased | | 24 | | 3 | A | 5 | | | 100 | | | | | | 2.3 | 1.23 | | | | | | 1.00 | 0.31 |
| Alive | | 24 | | 2 | B | 5 | | | 100 | | | | | | 2.3 | 1.20 | | | | | | 0.37 | 0.27 |
| Alive | | 24 | 22 | 3 | C | 4 | | | 100 | | | | | | 2.3 | 1.33 | | | | | | — | — |
| Alive | | 24 | 18 | 2 | B | 1 | | | 100 | | | | | | 2.9 | 1.25 | | | | | | 1.00 | 0.37 |
| Alive | | 24 | 5 | 2 | [symbol] | 3 | | | 100 | | | | | | 1.8 | 1.04 | | | | | | — | — |
| Deceased | | 24 | 18 | 3 | D | 3 | | | 100 | | | | | | 2.4 | 1.35 | | | | | | 0.38 | 0.44 |
| Alive | | 24 | 17 | 2 | B | 2 | | | 100 | | | | | | 2.3 | 1.61 | | | | | | — | — |
| Alive | | 24 | 21 | 3 | C | 6 | | | 100 | | | | | | 2.3 | 1.32 | | | | | | 0.22 | 0.34 |
| Alive | | 24 | 1 | 2 | C | 4 | | | 100 | | | | | | 2.5 | 1.35 | | | | | | — | — |
| Alive | | 24 | 18 | 1 | A | 7 | | | 100 | | | | | | 2.5 | 1.32 | | | | | | — | — |
| Alive | | 24 | 21 | 5 | C | 7 | | | 100 | | | | | | 2.5 | 1.31 | | | | | | 0.05 | 0.16 |
| Alive | | 24 | 30 | 5 | H | 7 | | | 100 | | | | | | 2.7 | 1.15 | | | | | | — | — |
| Deceased | | 24 | 21 | 5 | C | 7 | | | 100 | | | | | | 2.5 | 1.20 | | | | | | 0.97 | 0.05 |
| Alive | | 25 | 20 | 2 | B | 4 | | | 100 | | | | | | 2.1 | 1.32 | | | | | | — | — |
| Alive | | 25 | 21 | 3 | C | 7 | | | 100 | | | | | | 2.7 | 1.27 | | | | | | 0.31 | 0.39 |
| Alive | | 25 | 10 | 3 | H | 6 | | | 100 | | | | | | 2.5 | 1.26 | | | | | | — | — |
| Alive | | 25 | 19 | 2 | B | 3 | | | 100 | | | | | | 1.4 | 1.44 | | | | | | — | — |
| Alive | | 25 | 21 | 3 | C | 7 | | | 100 | | | | | | 2.7 | 1.22 | | | | | | — | — |
| Deceased | | 25 | 17 | 2 | B | 2 | | | 100 | | | | | | 3.0 | 1.34 | | | | | | 0.93 | 0.55 |
| Alive | | 25 | 7 | 3 | F | 7 | | | 100 | | | | | | 2.6 | 1.34 | | | | | | — | — |
| Deceased | 20 | 25 | 21 | 3 | C | 2 | | | 100 | | | | | | 2.7 | 1.15 | | | | | | 0.27 | 0.91 |
| Deceased | 24 | 25 | 9 | 1 | D | 2 | | | 100 | | | | | | 2.9 | 1.45 | | | | | | — | — |
| Alive | [symbol] | 25 | 3 | 3 | [symbol] | 5 | | | 100 | | | | | | 2.4 | 1.35 | | | | | | 0.05 | 0.15 |
| Alive | | 25 | 21 | 3 | C | 7 | | | 100 | | | | | | 2.2 | 1.35 | | | | | | — | — |
| Deceased | | 25 | 23 | 2 | B | 1 | | | 100 | | | | | | 2.1 | 1.12 | | | | | | — | — |
| Alive | | 25 | 12 | 3 | H | 2 | | | 100 | | | | | | 2.8 | 1.43 | | | | | | 0.06 | 0.17 |
| Alive | | 25 | 19 | 2 | B | [symbol] | | | 100 | | | | | | 2.4 | 1.24 | | | | | | — | — |
| Deceased | | 25 | 22 | 3 | C | 5 | | | 100 | | | | | | 3.2 | 1.30 | | | | | | 1.03 | 1.00 |
| Alive | | 26 | 17 | 2 | B | 2 | | | 100 | | | | | | 2.3 | 1.29 | | | | | | 0.04 | 0.21 |
| Alive | | 26 | 12 | 3 | H | 2 | | | 100 | | | | | | 2.9 | 1.31 | | | | | | — | — |
| Alive | | 26 | 14 | 1 | A | 2 | | | 100 | | | | | | 2.1 | 1.30 | | | | | | — | — |
| Deceased | | 26 | 18 | 1 | D | 1 | | | 100 | | | | | | 3.0 | 1.32 | | | | | | 0.50 | 0.61 |
| Deceased | | 26 | 19 | 1 | A | 6 | | | 100 | | | | | | 2.7 | 1.30 | | | | | | — | — |
| Alive | | 26 | 15 | 2 | [symbol] | 2 | | | 100 | | | | | | 1.7 | 1.19 | | | | | | — | — |
| Alive | | 26 | 20 | 2 | | 2 | | | 100 | | | | | | 2.5 | 1.18 | | | | | | — | — |
| Alive | | 26 | 7 | 3 | | 7 | | | 100 | | | | | | 1.6 | 1.31 | | | | | | 0.00 | 0.15 |
| Deceased | | 26 | 14 | 2 | B | 6 | | | 100 | | | | | | 2.7 | 1.35 | | | | | | — | — |
| Alive | | 26 | 9 | 1 | B | 1 | | | 100 | | | | | | 3.0 | 1.22 | | | | | | — | — |
| Deceased | | 26 | 12 | 5 | H | 3 | | | 100 | | | | | | 2.8 | 1.20 | | | | | | 0.54 | 0.29 |
| Deceased | | 26 | 22 | 3 | C | 5 | | | 100 | | | | | | 2.3 | 1.19 | | | | | | 0.47 | 0.63 |
| Alive | | 26 | 3 | 3 | [symbol] | 5 | | | 100 | | | | | | 2.6 | 1.31 | | | | | | 0.32 | 0.34 |
| Deceased | | 26 | 23 | 2 | B | 3 | | | 100 | | | | | | 2.7 | 1.83 | | | | | | 0.00 | 0.15 |
| Alive | | 26 | 5 | 3 | [symbol] | 5 | | | 100 | | | | | | 2.0 | 1.24 | | | | | | 0.00 | 0.27 |
| Deceased | | 26 | 12 | 3 | H | 2 | | | 100 | | | | | | 2.1 | 1.13 | | | | | | 0.00 | 0.09 |
| Deceased | | 27 | 13 | 2 | [symbol] | 1 | | | 100 | | | | | | 1.8 | 1.45 | | | | | | — | — |
| Alive | | 27 | 9 | 1 | D | 2 | | | 100 | | | | | | 2.8 | 1.50 | | | | | | — | — |
| Alive | | 27 | 23 | 3 | C | 3 | | | 100 | | | | | | 1.8 | 1.29 | | | | | | — | — |
| Alive | | 27 | 3 | 3 | H | 6 | | | 100 | | | | | | 2.4 | 1.37 | | | | | | — | — |
| Deceased | | 27 | 17 | 2 | B | 1 | | | 100 | | | | | | 2.8 | 1.54 | | | | | | 1.00 | 0.92 |
| Alive | | 27 | 18 | 1 | D | 5 | | | 100 | | | | | | 2.7 | 1.69 | | | | | | — | — |
| Alive | | 27 | 20 | 2 | [symbol] | 2 | | | 100 | | | | | | 3.8 | 1.25 | | | | | | — | — |
| Alive | | 27 | 9 | 1 | D | 1 | | | 100 | | | | | | 3.1 | 1.76 | | | | | | — | — |
| Alive | | 27 | 18 | 1 | D | 2 | | | 100 | | | | | | 2.3 | 1.51 | | | | | | — | — |
| Alive | | 27 | 22 | 3 | C | 4 | | | 100 | | | | | | 1.8 | 1.27 | | | | | | — | — |
| Alive | | 27 | 19 | 2 | [symbol] | 6 | | | 100 | | | | | | 2.2 | 1.22 | | | | | | 1.00 | 0.37 |
| Alive | | 27 | 15 | 1 | A | 6 | | | 100 | | | | | | 2.3 | 1.55 | | | | | | 0.24 | 0.13 |
| Alive | | 27 | 20 | 2 | [symbol] | 1 | | | 100 | | | | | | 3.3 | 1.65 | | | | | | — | — |
| Deceased | | 27 | 7 | 5 | F | 7 | | | 100 | | | | | | 2.3 | 1.39 | | | | | | 1.00 | 0.99 |
| Deceased | | 27 | 9 | 5 | H | 8 | | | 100 | | | | | | 2.2 | 1.73 | | | | | | 0.07 | 0.20 |
| Deceased | | 27 | 8 | 3 | F | 3 | | | 100 | | | | | | 2.3 | 1.43 | | | | | | 0.95 | 0.72 |
| Deceased | | 28 | 11 | 3 | H | 4 | | | 100 | | | | | | 2.3 | 1.31 | | | | | | — | — |
| Deceased | | 28 | 7 | 3 | F | 7 | | | 100 | | | | | | 2.4 | 1.45 | | | | | | — | — |
| Alive | | 28 | 13 | 1 | A | 5 | | | 100 | | | | | | 2.2 | 1.65 | | | | | | — | — |
| Deceased | | 28 | 8 | 3 | F | 5 | | | 100 | | | | | | 2.1 | 1.73 | | | | | | 0.00 | 0.29 |
| Deceased | | 28 | 19 | 1 | A | 2 | | | 100 | | | | | | 2.8 | 1.52 | | | | | | — | — |
| Alive | | 28 | 13 | 2 | B | 3 | | | 100 | | | | | | 2.6 | 1.85 | | | | | | 0.06 | 0.32 |
| Alive | | 28 | 16 | 1 | D | 8 | | | 100 | | | | | | 2.6 | 1.33 | | | | | | — | — |
| Deceased | | 28 | 11 | 3 | H | 4 | | | 100 | | | | | | 2.7 | 1.45 | | | | | | 0.14 | 0.35 |
| Deceased | | 28 | 15 | 1 | A | 8 | | | 100 | | | | | | 2.4 | 1.20 | | | | | | 1.00 | 0.84 |
| Deceased | | 28 | 15 | 2 | A | 5 | | | 100 | | | | | | 2.2 | 1.68 | | | | | | 0.96 | 0.71 |
| Deceased | | 28 | 7 | 3 | F | 7 | | | 100 | | | | | | 2.6 | 1.27 | | | | | | — | — |
| Deceased | | 28 | 22 | 3 | C | 4 | | | 100 | | | | | | 1.9 | 1.73 | | | | | | — | — |

[symbol]

indicates data missing or illegible when filed

SUPPLEMENTARY TABLE 4
Summary of high coverage cfDNA samples and genomic analyses

| | | | | | Number of |
|---|---|---|---|---|---|
| | | Sequencing | | Read | sequenced |
| Patient ID | Patient type | approach | Read type | length | fragments |
| CGPLLU276P | No cancer | WGS | Paired-end | 100 | 111,957,591 |
| CGPLLU278P | No cancer | WGS | Paired-end | 100 | 114,037,7 [symbol] |
| CGPLLU294P | Lung-Adenocarcinoma | WGS | Paired-end | 100 | 214,120,371 |
| CGPLLU304P | No cancer | WGS | Paired-end | 100 | 232,622,449 |
| CGPLLU306P | Lung-Adenocarcinoma | WGS | Paired-end | 100 | 259,238,568 |
| CGPLLU315P | No cancer | WGS | Paired-end | 100 | 180,302,298 |
| CGPLLU332P | No cancer | WGS | Paired-end | 100 | 482,245,711 |
| CGPLLU335P | Lung-SCC | WGS | Paired-end | 100 | 235,844,337 |
| CGPLLU344P | Lung-SCC | WGS | Paired-end | 100 | 197,441,771 |
| CGPLLU346P | No cancer | WGS | Paired-end | 100 | 268,592,163 |
| CGPLLU347P | Skin-Melanoma | WGS | Paired-end | 100 | 264,0 [symbol] 1,871 |
| CGPLLU353P | No cancer | WGS | Paired-end | 100 | 129,620,634 |
| CGPLLU358P | No cancer | WGS | Paired-end | 100 | 2 [symbol] 8,914,035 |
| CGPLLU362P | No cancer | WGS | Paired-end | 100 | 1 [symbol] 8,851,762 |
| CGPLLU367P | No cancer | WGS | Paired-end | 100 | 129,530,151 |
| CGPLLU372P | No cancer | WGS | Paired-end | 100 | 262,644,051 |
| CGPLLU387P | No cancer | WGS | Paired-end | 100 | 165,537,916 |
| CGPLLU401P | No cancer | WGS | Paired-end | 100 | 233,104,285 |
| CGPLLU403P | Lung-Adenocarcinoma | WGS | Paired-end | 100 | 273,329,638 |
| CGPLLU405P | No cancer | WGS | Paired-end | 100 | 125,590,262 |
| CGPLLU407P | No cancer | WGS | Paired-end | 100 | 111,635,066 |
| CGPLLU413P | No cancer | WGS | Paired-end | 100 | 166,704,737 |
| CGPLLU414P | Lung-Adenocarcinoma | WGS | Paired-end | 100 | 271,065,301 |
| CGPLLU424P | Lung-SCC | WGS | Paired-end | 100 | 211,646,960 |
| CGPLLU432P | No cancer | WGS | Paired-end | 100 | 184,677,106 |
| CGPLLU442P | No cancer | WGS | Paired-end | 100 | 536,917,638 |
| CGPLLU446P | No cancer | WGS | Paired-end | 100 | 213,821,280 |
| CGPLLU452P | Lung-Adenocarcinoma | WGS | Paired-end | 100 | 233,859,246 |
| CGPLLU454P | No cancer | WGS | Paired-end | 100 | 547,702,301 |
| CGPLLU456P | Lung-SCC | WGS | Paired-end | 100 | 201,69 [symbol] ,325 |
| CGPLLU469P | Lung-Adenocarcinoma | WGS | Paired-end | 100 | 255,511,074 |
| CGPLLU474P | No cancer | WGS | Paired-end | 100 | 129,875,218 |
| CGPLLU480P | Lung-SCC | WGS | Paired-end | 100 | 258,320,701 |
| CGPLLU489P | No cancer | WGS | Peired-end | 100 | 180,8 [symbol] 1,885 |
| CGPLLU490P | No cancer | WGS | Paired-end | 100 | 110,862,313 |
| CGPLLU496P | No cancer | WGS | Paired-end | 100 | 212,160,007 |
| CGPLLU497P | No cancer | WGS | Paired-end | 100 | 262,108,163 |
| CGPLLU499P | No cancer | WGS | Paired-end | 100 | 403,173,954 |
| CGPLLU501P | Lung-Adenocarcinoma | WGS | Paired-end | 100 | 245,442,915 |
| CGPLLU504P | Lung-Adenocarcinoma | WGS | Paired-end | 100 | 237,335,015 |
| CGPLLU505P | Lung-Adenocarcinoma | WGS | Paired-end | 100 | 270,435,688 |
| CGPLLU509P | No cancer | WGS | Paired-end | 100 | 225,868,024 |
| CGPLLU519P | No cancer | WGS | Paired-end | 100 | 146,724,698 |
| CGPLLU529P | Lymphoid-BNHL | WGS | Paired-end | 100 | 308,682,852 |
| OGPLLU539P | No cancer | WGS | Paired-end | 100 | 222,479,680 |
| CGPLLU541P | No cancer | WGS | Paired-end | 100 | 157,557,222 |
| CGPLLU543P | Lung-Adenocarcinoma | WGS | Paired-end | 100 | 203,760,829 |
| CGPLLU551P | No cancer | WGS | Paired-end | 100 | 136,798,991 |
| OSPLLU557P | Skin-Melanoma | WGS | Paired-end | 100 | 211,095,500 |
| CGPLLU567P | No cancer | WGS | Paired-end | 100 | 137,537,985 |
| CGPLLU573P | No cancer | WGS | Paired-end | 100 | 161,756,112 |
| CGPLLU581P | No cancer | WGS | Paired-end | 100 | 141,782,605 |
| CGPLLU589P | No cancer | WGS | Paired-end | 100 | 184,549,262 |
| CGPLLU592P | No cancer | WGS | Paired-end | 100 | 260,993,299 |
| CGPLLU596P | No cancer | WGS | Paired-end | 100 | 270,677,726 |
| CGPLLU599P | No cancer | WGS | Paired-end | 100 | 133,952,771 |
| CGPLLU610P | No cancer | WGS | Paired-end | 100 | 169,086,140 |
| CGPLLU635P | No cancer | WGS | Paired-end | 100 | 273,761,253 |

| | Number of | Percent | Percent | Reads | Whole | Estimated |
|---|---|---|---|---|---|---|
| | sequenced | of Q20 | of Q30 | mapped to | genome | 8-oxo-dG |
| Patient ID | reads | bases | bases | genome | coverage | level |
| CGPLLU276P | 223,915,182 | 97.87% | 94.09% | 167,211,701 | 5.4 | 1.21 |
| CGPLLU278P | 228,075,596 | 97.09% | 92.75% | 166,779,614 | 5.4 | 1.29 |
| CGPLLU294P | 428,240,742 | 97.15% | 92.91% | 313,975,177 | 10.1 | 1.14 |
| CGPLLU304P | 465,244,898 | 97.75% | 93.99% | 333,577,133 | 10.8 | 1.37 |
| CGPLLU306P | 518,477,136 | 96.95% | 92.24% | 352,085,707 | 11.7 | 1.59 |
| CGPLLU315P | 360,604,596 | 95.08% | 88.75% | 219,144,799 | 7.1 | 1.90 |
| CGPLLU332P | 964,491,422 | 96.35% | 91.65% | 598,985,989 | 19.3 | 1.51 |
| CGPLLU335P | 471, [symbol] 88,674 | 96.96% | 92.39% | 346,124,316 | 11.2 | 1.48 |
| CGPLLU344P | 394,883,542 | 96.34% | 90.57% | 257,601,478 | 8.3 | 1.22 |
| CGPLLU346P | 537,184,326 | 97.39% | 93.06% | 401,904,050 | 13.0 | 1.99 |
| CGPLLU347P | 528,123,742 | 96.99% | 92.33% | 360,59 [symbol] ,585 | 11.6 | 1.18 |
| CGPLLU353P | 259,241,268 | 97.52% | 93.80% | 196,325,407 | 6.3 | 1.08 |
| CGPLLU358P | 577,828,070 | 97.02% | 93.05% | 417,887,11 [symbol] | 13.5 | 1.87 |
| CGPLLU362P | 337,703,524 | 97.07% | 92.92% | 246,293,720 | 8.0 | 2.03 |
| CGPLLU367P | 259,060,302 | 97.17% | 92.91% | 190,012, [symbol] | 6.1 | 2.25 |
| CGPLLU372P | 525,288,102 | 97.28% | 92.91% | 381,928,987 | 12.3 | 1.78 |
| CGPLLU387P | 331,075,832 | 96.55% | 91.79% | 228,174,881 | 7.4 | 1.27 |
| CGPLLU401P | 466,208,570 | 97.00% | 92.81% | 335,659,502 | 10.8 | 1.58 |
| CGPLLU403P | 54 [symbol] , [symbol] 59,276 | 97.46% | 93.65% | 400,084,110 | 12.9 | 1.49 |
| CGPLLU405P | 251,180,524 | 97.33% | 92.65% | 1 [symbol] 4,167,308 | 5.9 | 1.92 |
| CGPLLU407P | 223,270,132 | 96.80% | 91.87% | 1 [symbol] 5,486,613 | 5.3 | 1.40 |
| CGPLLU413P | 333,409,474 | 97.11% | 92.39% | 230,890, [symbol] 06 | 7.5 | 1.67 |
| CGPLLU414P | 542,130,602 | 96.45% | 91.47% | 389,889,382 | 12.5 | 2.34 |
| CGPLLU424P | 423,293,920 | 97.44% | 93.06% | 306,827,015 | 9.9 | 1.67 |
| CGPLLU432P | 369,354,212 | 95.99% | 90.95% | 261,760,610 | 8.5 | 1.57 |
| CGPLLU442P | 1,073,835,276 | 97.20% | 92.77% | 769,204,282 | 24.8 | 1.66 |
| CGPLLU446P | 427,542,560 | 96.60% | 92.16% | 308,565,122 | 10.0 | 1.61 |
| CGPLLU452P | 467,718,492 | 96.48% | 91.23% | 296,332,349 | 9.6 | 1.79 |
| CGPLLU454P | 1,095,404,602 | 97.49% | 93.39% | 802,168,883 | 25.9 | 1.75 |
| CGPLLU456P | 403,392,650 | 96.54% | 92.27% | 279,876,358 | 9.0 | 1.57 |
| CGPLLU469P | 511,022,148 | 95.67% | 89.09% | 317,945,299 | 10.3 | 1.40 |
| CGPLLU474P | 259,750,436 | 96.45% | 91.12% | 191,316,533 | 6.2 | 1.42 |
| CGPLLU480P | 516,641,402 | 96.19% | 90.59% | 333,009,780 | 10.8 | 1.39 |
| CGPLLU489P | 361,723,770 | 95.63% | 90.00% | 248,609,427 | 8.0 | 1.63 |
| CGPLLU490P | 221,724,626 | 97.42% | 93.38% | 168,954,669 | 5.5 | 1.67 |
| CGPLLU496P | 424,320,014 | 97.75% | 93.81% | 309,103,628 | 10.0 | 0.89 |
| CGPLLU497P | 524,216,326 | 97.60% | 93.71% | 389,815,872 | 12.6 | 1.39 |
| CGPLLU499P | 806,347,908 | 97.32% | 93.06% | 580,407,122 | 18.7 | 1.58 |
| CGPLLU501P | 490,885,830 | 97.54% | 93.49% | 353,156,858 | 11.4 | 1.02 |
| CGPLLU504P | 474,670,030 | 97.33% | 93.06% | 332,274,133 | 10.7 | 1.31 |
| CGPLLU505P | 540,871,376 | 96.08% | 89.98% | 327,655,828 | 10.6 | 0.97 |
| CGPLLU509P | 451,736,048 | 97.47% | 93.39% | 332,857,454 | 10.8 | 2.53 |
| CGPLLU519P | 293,449,396 | 97.44% | 93.21% | 214,290,983 | 6.9 | 1.80 |
| CGPLLU529P | 617,365,704 | 97.31% | 93.36% | 445,142,405 | 14.4 | 1.43 |
| OGPLLU539P | 444,959,360 | 97.08% | 92.25% | 315,954,289 | 10.2 | 1.20 |
| CGPLLU541P | 315,114,444 | 97.47% | 93.41% | 235,286,862 | 7.6 | 1.08 |
| CGPLLU543P | 407,521,658 | 97.17% | 93.03% | 309,428,859 | 10.0 | 1.21 |
| CGPLLU551P | 273,597,982 | 97.19% | 92.92% | 199,419,299 | 6.4 | 1.22 |
| OSPLLU557P | 422,191,000 | 97.62% | 93.67% | 310,392,660 | 10.0 | 1.09 |
| CGPLLU567P | 275,075,970 | 97.03% | 93.00% | 199,358,378 | 6.4 | 1.03 |
| CGPLLU573P | 323,512,224 | 95.35% | 89.94% | 213,019,380 | 6.9 | 1.22 |
| CGPLLU581P | 283,565,210 | 97.31% | 92.84% | 201,297,726 | 6.5 | 1.28 |
| CGPLLU589P | 369,098,524 | 97.20% | 92.75% | 276,227,570 | 8.9 | 1.01 |
| CGPLLU592P | 521,986,598 | 96.13% | 90.96% | 369,209,914 | 11.9 | 1.31 |
| CGPLLU596P | 541,355,452 | 96.11% | 91.08% | 372,038,938 | 12.0 | 1.25 |
| CGPLLU599P | 267,905,542 | 97.31% | 93.04% | 197,341,456 | 6.4 | 1.27 |
| CGPLLU610P | 338,172,280 | 96.99% | 92.64% | 241,149,969 | 7.8 | 1.00 |
| CGPLLU635P | 547,522,506 | 96.50% | 91.54% | 402,442,860 | 13.0 | 1.25 |

[symbol]

indicates data missing or illegible when filed

SUPPLEMENTARY TABLE 5
Summary of cfDNA samples and genomic analyses of lung cancer patients undergoing targeted therapy*

| | | | | | | | | Days to |
|---|---|---|---|---|---|---|---|---|
| | Histological | | | | | Pack | | initial RECIST |
| Patient ID | diagnosis | Age | Sex | Stage | Smoking status | years | Treatment | assessment |
| CGLU316 | Mixed | 50 | Female | IIIA | Former smoker | 15 | [symbol] | 42 |
| CGPLLU269 | Adenocarcinoma | 52 | Female | IV | Former smoker | 8 | Mavelertinib | 25 |
| CGPLLU271 | Adenocarcinoma | 73 | Male | IV | Former smoker | NA | Mavelertinib | 36 |
| CGPLLU88 | Adenocarcinoma | 59 | Male | IIA | Former smoker | NA | [symbol] | 35 |
| CGPLLU89 | Adenocarcinoma | 54 | Female | IV | Former smoker | NA | [symbol] | 37 |

| | RECIST | | | | | |
|---|---|---|---|---|---|---|
| | percent | | | | | |
| | change in | | Days to | | | Status |
| | target | Initial radiographic | RECIST | Days on | Overall response | at last |
| Patient ID | lesions | response type | progression | Treatment | at last follow-up | follow-up |
| CGLU316 | NA | Progressive Disease | 42 | 73 | Progressive disease | Alive |
| CGPLLU269 | −5.9 | Stable Disease | NA | 47 | Progressive disease | Deceased |
| CGPLLU271 | −0.2 | Stable Disease | 246 | [symbol] | Progressive disease | Deceased |
| CGPLLU88 | −40.7 | Partial Response | 541 | 548 | Progressive disease | Deceased |
| CGPLLU89 | −41.2 | Partial Response | 200 | 427 | Progressive disease | Deceased |

| Timepoint | | | | Number of | Number of | |
|---|---|---|---|---|---|---|
| (days relative to | Sequencing | | Read | sequenced | sequenced | Percent of |
| treatment initiation) | approach | Read type | length | fragments | reads | Q20 bases |
| Pre-treatment, Day −53 | WGS | Paired-end | 100 | 55,769,867 | 111,539,734 | 92.65% |
| Pre-treatment, Day −4 | WGS | Paired-end | 100 | 42,612,384 | 85,224,768 | 92.51% |
| Post-treatment, Day 18 | WGS | Paired-end | 100 | 43,916,255 | 87,832,510 | 92.78% |
| Post-treatment, Day 87 | WGS | Paired-end | 100 | 50,967,948 | 101,935,896 | 92.57% |
| Pre-treatment, Day 0 | WGS | Paired-end | 100 | 50,086,599 | 100,173,198 | 89.27% |
| Post-treament, Day 9 | WGS | Paired-end | 100 | 48,924,695 | 97,849,390 | 87.28% |
| Post-trestrant, Day 28 | WGS | Paired-end | 100 | 45,999,749 | 91,999,498 | 89.44% |
| Pre-treatment, Day 0 | WGS | Paired-end | 100 | 68,496,676 | 136,993,352 | 94.05% |
| Post-treatment, Day 6 | WGS | Paired-end | 100 | 49,313,763 | 98,627,526 | 89.14% |
| Post-treatment, Day 20 | WGS | Paired-end | 100 | 47,121,118 | 94,242,236 | 88.82% |
| Post-treatment, Day 104 | WGS | Paired-end | 100 | 49,089,804 | 98,179,608 | 88.54% |
| Post-treatment, Day 259 | WGS | Paired-end | 100 | 44,030,080 | 88,060,160 | 90.99% |
| Pre-tremoment, Day 0 | WGS | Paired-end | 100 | 43,827,775 | 87,655,550 | 93.01% |
| Post-treatment, Day 7 | WGS | Paired-end | 100 | 44,858,615 | 89,717,230 | 93.05% |
| Post-treatment, Day 297 | WGS | Paired-end | 100 | 50,655,434 | 101,310,868 | 92.17% |
| Pre-treatment, Day 0 | WGS | Paired-end | 100 | 43,230,041 | 86,460,082 | 91.82% |
| Post-treatment, Day 7 | WGS | Paired-end | 100 | 40,433,167 | 80,866,334 | 91.36% |
| Post-treatment, Day 22 | WGS | Paired-end | 100 | 34,781,256 | 69,562,512 | 93.28% |

| | | | | | | Maximum |
|---|---|---|---|---|---|---|
| | | | | | | mutant allele |
| Timepoint | | | Whole | Estimated | | fraction from |
| (days relative to | Percent of | Reads mapped | genome | 8-oxo-dG | GEMINI | targeted |
| treatment initiation) | Q30 bases | to genome | coverage | level | score [symbol] | sequencing |
| Pre-treatment, Day −53 | 87.39% | 10 [symbol] ,327,297 | 3.3 | 3.40 | 0.33 | 15.72% |
| Pre-treatment, Day −4 | 87.21% | 78,807,227 | 2.5 | 3.09 | 0.90 | 45.67% |
| Post-treatment, Day 18 | 87.59% | 81,368,204 | 2.6 | 3.01 | 0.46 | 33.38% |
| Post-treatment, Day 87 | 87.28% | 94,105,646 | 3.0 | 2.58 | 0.98 | 66.01% |
| Pre-treatment, Day 0 | 83.33% | 88,595,949 | 2.9 | 3.58 | 0.48 | 0.10% |
| Post-treament, Day 9 | 80.96% | 82,978,903 | 2.7 | 3.54 | 0.10 | 0% |
| Post-trestrant, Day 28 | 83.56% | 81,7 [symbol] 7,487 | 2.6 | 3.30 | 0.45 | 0% |
| Pre-treatment, Day 0 | 90.52% | 124,970,639 | 4.0 | 3.27 | 0.88 | 3.36% |
| Post-treatment, Day 6 | 83.16% | 87,597,839 | 2.8 | 3.47 | 0.77 | 0.17% |
| Post-treatment, Day 20 | 82.90% | 81,619, [symbol] 70 | 2.6 | 1.53 | 0.66 | 0.18% |
| Post-treatment, Day 104 | 81.90% | 83,8 [symbol] 0,903 | 2.7 | 1.50 | 0.83 | 0.33% |
| Post-treatment, Day 259 | 85.34% | 79,978,020 | 2.6 | 1.52 | 0.75 | 0.44% |
| Pre-tremoment, Day 0 | 87.93% | 81,537,455 | 2.6 | 1.33 | 0.82 | 12.17% |
| Post-treatment, Day 7 | 88.07% | 82,955,860 | 2.7 | 1.22 | 0.42 | 0.24% |
| Post-treatment, Day 297 | 86.99% | 92,297,948 | 3.0 | 1.36 | 0.87 | 2.42% |
| Pre-treatment, Day 0 | 86.03% | 79,669,676 | 2.6 | 1.44 | 0.57 | 0.42% |
| Post-treatment, Day 7 | 85.57% | 73,240,688 | 2.4 | 1.35 | 0.38 | 0.28% |
| Post-treatment, Day 22 | 88.58% | 63,632,0 [symbol] 3 | 2.1 | 1.28 | 0.85 | 0.26% |

*Clinical, coverage, and mutant allele fraction information for patients undergoing targeted therapy were obtained from Phallen et al [symbol] and Cristiano et al [symbol] [symbol]
indicates data missing or illegible when filed

SUPPLEMENTARY TABLE 6
Summary of validation cohort and genomic analyses

| | Collection | | | | | | | |
|---|---|---|---|---|---|---|---|---|
| | center/ | | Histological | Age at | | | | Pack |
| Patient ID | Consort [symbol] | Patient type | diagnosis | [symbol] | Sex | Stage | Smoking status | years |
| [symbol] | [symbol] | Benign | Benign | [symbol] | Male | NA | Current smoker | [symbol] |
| [symbol] | [symbol] | Lung cancer | SCLC | [symbol] | Male | Unknown | Current smoker | 50 |
| [symbol] | [symbol] | Lung cancer | Squamous cell | 70 | Male | IA | Current smoker | 100 |
| | | | carcinoma | | | | | |
| [symbol] | [symbol] | Lung cancer | Adenocarcinoma | [symbol] | Female | IA | Former smoker | 4 [symbol] |
| [symbol] | [symbol] | Lung cancer | Squamous cell | 66 | Male | IA | Former smoker | 7 [symbol] |
| | | | carcinoma | | | | | |
| [symbol] | [symbol] | Lung cancer | Squamous cell | [symbol] | Male | [symbol] | Former smoker | 40 |
| | | | carcinoma | | | | | |
| [symbol] | [symbol] | Lung cancer | Squamous cell | [symbol] | Male | IA | Former smoker | [symbol] |
| | | | carcinoma | | | | | |
| [symbol] | [symbol] | Lung cancer | Unknown | 69 | Male | IA | Former smoker | [symbol] |
| [symbol] | [symbol] | Benign | Benign | 74 | Male | NA | Former smoker | 45 |
| [symbol] | [symbol] | Lung cancer | SCLC | 75 | Male | IV | Current smoker | 13 [symbol] |
| [symbol] | [symbol] | Lung cancer | Adenocarcinoma | 74 | Female | IA | Former smoker | 4 [symbol] |
| [symbol] | [symbol] | Benign | Benign | 70 | Female | NA | Current smoker | 45 |
| [symbol] | [symbol] | Benign | Benign | [symbol] | Male | NA | Former smoker | 5 [symbol] |
| [symbol] | [symbol] | Benign | Benign | 54 | Male | NA | Current smoker | 30 |
| [symbol] | [symbol] | Benign | Benign | 65 | Male | NA | Former smoker | [symbol] |
| [symbol] | [symbol] | Lung cancer | Unknown | [symbol] | Male | IA | Former smoker | [symbol] |
| [symbol] | [symbol] | Benign | Benign | [symbol] | Male | NA | Current smoker | 22 |
| [symbol] | [symbol] | Lung cancer | Squamous cell | [symbol] | Male | [symbol] | Current smoker | 52 |
| | | | carcinoma | | | | | |
| [symbol] | [symbol] | Benign | Benign | [symbol] | Male | NA | Current smoker | [symbol] |
| [symbol] | [symbol] | Benign | Benign | [symbol] | Female | NA | Current smoker | 100 |
| [symbol] | [symbol] | Lung cancer | SCLC | 79 | Female | IV | Former smoker | 50 |
| [symbol] | [symbol] | Benign | Benign | 57 | Male | NA | Former smoker | NA |
| [symbol] | [symbol] | Lung cancer | [symbol] | 55 | Male | IA | Former smoker | 25 |
| [symbol] | [symbol] | Lung cancer | [symbol] | 47 | Female | IA | Former smoker | [symbol] |
| [symbol] | [symbol] | Lung cancer | Adenocarcinoma | 61 | Female | IA | Never smoker | 0 |
| [symbol] | [symbol] | Lung cancer | [symbol] | 60 | Male | [symbol] | Former smoker | [symbol] |
| [symbol] | [symbol] | Lung cancer | Squamous cell | [symbol] | Female | [symbol] | Former smoker | NA |
| | | | carcinoma | | | | | |
| [symbol] | [symbol] | Lung cancer | Adenocarcinoma | [symbol] | Male | [symbol] | Former smoker | NA |
| [symbol] | [symbol] | Lung cancer | Squamous cell | 77 | Male | IA | Former smoker | 60 |
| | | | carcinoma | | | | | |
| [symbol] | [symbol] | Lung cancer | Adenocarcinoma | [symbol] | Female | [symbol] | Never smoker | 0 |
| [symbol] | [symbol] | Lung cancer | Adenocarcinoma | 65 | Male | IA | Former smoker | 68 |
| [symbol] | [symbol] | Lung cancer | Adenocarcinoma | 84 | Female | [symbol] | Former smoker | 30 |
| [symbol] | [symbol] | Benign | Benign | [symbol] | Male | NA | Former smoker | [symbol] |
| [symbol] | [symbol] | Lung cancer | Adenocarcinoma | 71 | Female | IA | Former smoker | 30 |
| [symbol] | [symbol] | Lung cancer | Squamous cell | 72 | Female | IA | Former smoker | 60 |
| | | | carcinoma | | | | | |
| [symbol] | [symbol] | Lung cancer | Squamous cell | 73 | Female | IA | Former smoker | 75 |
| | | | carcinoma | | | | | |
| [symbol] | [symbol] | Lung cancer | Adenocarcinoma | 75 | Female | IA | Former smoker | 22 |
| [symbol] | [symbol] | Lung cancer | Adenocarcinoma | 65 | Male | [symbol] | Former smoker | 50 |
| [symbol] | [symbol] | Lung cancer | Adenocarcinoma | 63 | Male | [symbol] | Never smoker | 0 |
| [symbol] | [symbol] | Lung cancer | Adenocarcinoma | [symbol] | Male | IA | Former smoker | 25 |
| [symbol] | [symbol] | Lung cancer | [symbol] | 73 | Female | IA | Former smoker | 2.5 |
| [symbol] | [symbol] | Lung cancer | [symbol] | 69 | Female | [symbol] | Never smoker | 0 |
| [symbol] | [symbol] | Lung cancer | Adenocarcinoma | 62 | Female | [symbol] | Former smoker | 48 |
| [symbol] | [symbol] | Lung cancer | Adenocarcinoma | 69 | Male | IA | Former smoker | NA |
| [symbol] | [symbol] | Lung cancer | Squamous cell | 71 | Female | IA | Current smoker | 100 |
| | | | carcinoma | | | | | |
| [symbol] | [symbol] | Benign | Benign | 74 | Female | NA | Former smoker | 40 |
| [symbol] | [symbol] | Lung cnacer | Adenocarcinoma | 73 | Male | [symbol] | Never smoker | 0 |

| | | | | | Number of | Number of | |
|---|---|---|---|---|---|---|---|
| | | Sequencing | | Read | sequenced | sequenced | Percent of |
| Patient ID | [symbol] | approach | Read type | length | fragments | reads | Q20 bases |
| [symbol] | 0 | WGS | Paired-end | 100 | [symbol] | [symbol] | [symbol] |
| [symbol] | 0 | WGS | Paired-end | 100 | [symbol] | [symbol] | [symbol] |
| [symbol] | 0 | WGS | Paired-end | 100 | [symbol] | [symbol] | [symbol] |
| [symbol] | NA | WGS | Paired-end | 100 | [symbol] | [symbol] | [symbol] |
| [symbol] | NA | WGS | Paired-end | 100 | [symbol] | [symbol] | [symbol] |
| [symbol] | NA | WGS | Paired-end | 100 | [symbol] | [symbol] | [symbol] |
| [symbol] | NA | WGS | Paired-end | 100 | [symbol] | [symbol] | [symbol] |
| [symbol] | NA | WGS | Paired-end | 100 | [symbol] | [symbol] | [symbol] |
| [symbol] | NA | WGS | Paired-end | 100 | [symbol] | [symbol] | [symbol] |
| [symbol] | 0 | WGS | Paired-end | 100 | [symbol] | [symbol] | [symbol] |
| [symbol] | NA | WGS | Paired-end | 100 | [symbol] | [symbol] | [symbol] |
| [symbol] | 0 | WGS | Paired-end | 100 | [symbol] | [symbol] | [symbol] |
| [symbol] | NA | WGS | Paired-end | 100 | [symbol] | [symbol] | [symbol] |
| [symbol] | 0 | WGS | Paired-end | 100 | [symbol] | [symbol] | [symbol] |
| [symbol] | NA | WGS | Paired-end | 100 | [symbol] | [symbol] | [symbol] |
| [symbol] | NA | WGS | Paired-end | 100 | [symbol] | [symbol] | [symbol] |
| [symbol] | 0 | WGS | Paired-end | 100 | [symbol] | [symbol] | [symbol] |
| [symbol] | 0 | WGS | Paired-end | 100 | [symbol] | [symbol] | [symbol] |
| [symbol] | 0 | WGS | Paired-end | 100 | [symbol] | [symbol] | [symbol] |
| [symbol] | 0 | WGS | Paired-end | 100 | [symbol] | [symbol] | [symbol] |
| [symbol] | NA | WGS | Paired-end | 100 | [symbol] | [symbol] | [symbol] |
| [symbol] | 27 | WGS | Paired-end | 100 | [symbol] | [symbol] | [symbol] |
| [symbol] | 37 | WGS | Paired-end | 100 | [symbol] | [symbol] | [symbol] |
| [symbol] | 21 | WGS | Paired-end | 100 | [symbol] | [symbol] | [symbol] |
| [symbol] | NA | WGS | Paired-end | 100 | [symbol] | [symbol] | [symbol] |
| [symbol] | 25 | WGS | Paired-end | 100 | [symbol] | [symbol] | [symbol] |
| [symbol] | 12 | WGS | Paired-end | 100 | [symbol] | [symbol] | [symbol] |
| [symbol] | 45 | WGS | Paired-end | 100 | [symbol] | [symbol] | [symbol] |
| [symbol] | 31 | WGS | Paired-end | 100 | [symbol] | [symbol] | [symbol] |
| [symbol] | NA | WGS | Paired-end | 100 | [symbol] | [symbol] | [symbol] |
| [symbol] | 15 | WGS | Paired-end | 100 | [symbol] | [symbol] | [symbol] |
| [symbol] | [symbol] 1 | WGS | Paired-end | 100 | [symbol] | [symbol] | [symbol] |
| [symbol] | 25 | WGS | Paired-end | 100 | [symbol] | [symbol] | [symbol] |
| [symbol] | 20 | WGS | Paired-end | 100 | [symbol] | [symbol] | [symbol] |
| [symbol] | 15 | WGS | Paired-end | 100 | [symbol] | [symbol] | [symbol] |
| [symbol] | 17 | WGS | Paired-end | 100 | [symbol] | [symbol] | [symbol] |
| [symbol] | [symbol] | WGS | Paired-end | 100 | [symbol] | [symbol] | [symbol] |
| [symbol] | [symbol] | WGS | Paired-end | 100 | [symbol] | [symbol] | [symbol] |
| [symbol] | NA | WGS | Paired-end | 100 | [symbol] | [symbol] | [symbol] |
| [symbol] | 14 | WGS | Paired-end | 100 | [symbol] | [symbol] | [symbol] |
| [symbol] | 47 | WGS | Paired-end | 100 | [symbol] | [symbol] | [symbol] |
| [symbol] | NA | WGS | Paired-end | 100 | [symbol] | [symbol] | [symbol] |
| [symbol] | 2 | WGS | Paired-end | 100 | [symbol] | [symbol] | [symbol] |
| [symbol] | 13 | WGS | Paired-end | 100 | [symbol] | [symbol] | [symbol] |
| [symbol] | 0 | WGS | Paired-end | 100 | [symbol] | [symbol] | [symbol] |
| [symbol] | 12 | WGS | Paired-end | 100 | [symbol] | [symbol] | [symbol] |
| [symbol] | NA | WGS | Paired-end | 100 | [symbol] | [symbol] | [symbol] |

| | | Reads | Whole | Estimated | | | | |
|---|---|---|---|---|---|---|---|---|
| | Percent of | mapped to | genome | 8-oxo-dG | | | | |
| Patient ID | Q30 bases | genome | coverage | level | [symbol] | [symbol] | [symbol] | [symbol] |
| [symbol] | [symbol] | [symbol] | 2.0 | 1. [symbol] | 4.1 [symbol] 1 | 0.35 | 0.17 | 0.3 [symbol] |
| [symbol] | [symbol] | [symbol] | 2.2 | 1. [symbol] | 4.621 | 0. [symbol] 2 | 0. [symbol] 8 | 0.70 |
| [symbol] | [symbol] | [symbol] | 2.0 | 1.71 | 2.256 | 0. [symbol] | 0.1 [symbol] | 0.52 |
| [symbol] | [symbol] | [symbol] | 1. [symbol] | 1.43 | 2.269 | 0.25 | — | — |
| [symbol] | [symbol] | [symbol] | 2.1 | 1. [symbol] | 2.541 | 0. [symbol] | 0.3 [symbol] | 0.59 |
| [symbol] | [symbol] | [symbol] | 2.0 | 1.61 | 2.547 | 0.64 | 0.05 | 0.35 |
| [symbol] | [symbol] | [symbol] | 2.2 | 1.30 | 4.874 | 0. [symbol] | 0.12 | 0.35 |
| [symbol] | [symbol] | [symbol] | 1.8 | 1.31 | 2. [symbol] 3 | 0.76 | 0. [symbol] 1 | 0. [symbol] |
| [symbol] | [symbol] | [symbol] | 2.1 | 1.51 | 2.3 [symbol] | 0.25 | 0. [symbol] 5 | 0.15 |
| [symbol] | [symbol] | [symbol] | 1.5 | [symbol] | 2. [symbol] 3 | 0. [symbol] 4 | 0.16 | 0.35 |
| [symbol] | [symbol] | [symbol] | 2.1 | [symbol] | 2. [symbol] 2 | 0. [symbol] | 0. [symbol] | 0.57 |
| [symbol] | [symbol] | [symbol] | 1. [symbol] | 1.44 | 2.465 | 0.1 [symbol] | 0.2 [symbol] | 0.19 |
| [symbol] | [symbol] | [symbol] | 1.4 | 1.18 | 1.755 | 0.29 | — | — |
| [symbol] | [symbol] | [symbol] | 2.0 | 1.15 | 2. [symbol] | 0.40 | 0.25 | 0.3 [symbol] |
| [symbol] | [symbol] | [symbol] | 2. [symbol] | 1.1 [symbol] | 2.2 [symbol] 4 | 0.33 | 0.26 | 0.29 |
| [symbol] | [symbol] | [symbol] | 1. [symbol] | [symbol] | 2.231 | 0.7 [symbol] | 0.4 [symbol] | 0.81 |
| [symbol] | [symbol] | [symbol] | 2.0 | 1.22 | 2.427 | 0. [symbol] | 0.13 | 0.3 [symbol] |
| [symbol] | [symbol] | [symbol] | 2.2 | 1.15 | 2. [symbol] | 0.67 | 0.21 | 0.44 |
| [symbol] | [symbol] | [symbol] | 2.0 | 1.20 | 2. [symbol] 5 | 0.47 | 0.17 | 0.32 |
| [symbol] | [symbol] | [symbol] | 1.7 | 1.3 [symbol] | 1. [symbol] 55 | 0.22 | 0. [symbol] | 0.12 |
| [symbol] | [symbol] | [symbol] | 1. [symbol] | 1. [symbol] | [symbol] | 0. [symbol] | 1. [symbol] | 0. [symbol] |
| [symbol] | [symbol] | [symbol] | 2.1 | 1.73 | [symbol] | 0.32 | 0.4 [symbol] | 0.36 |
| [symbol] | [symbol] | [symbol] | 2.0 | 1.97 | [symbol] | 0.4 [symbol] | 0.27 | 0.37 |
| [symbol] | [symbol] | [symbol] | 1.9 | 1. [symbol] 1 | [symbol] | 0. [symbol] | — | — |
| [symbol] | [symbol] | [symbol] | 2.0 | 1.32 | [symbol] | 0.34 | — | — |
| [symbol] | [symbol] | [symbol] | 2.0 | 2. [symbol] 7 | [symbol] | 0.15 | 0.04 | 0.03 |
| [symbol] | [symbol] | [symbol] | 1. [symbol] | 1.50 | [symbol] | 0. [symbol] | 0.19 | 0.12 |
| [symbol] | [symbol] | [symbol] | 2.2 | 1.60 | [symbol] | 0.75 | — | — |
| [symbol] | [symbol] | [symbol] | 2.1 | 1.24 | [symbol] | 0.2 [symbol] | 0.26 | 0.35 |
| [symbol] | [symbol] | [symbol] | 2.0 | 1. [symbol] 1 | [symbol] | 0.1 [symbol] | — | — |
| [symbol] | [symbol] | [symbol] | 2.1 | 1. [symbol] | [symbol] | 0.21 | 0.14 | 0.17 |
| [symbol] | [symbol] | [symbol] | 2.1 | 2.0 [symbol] | [symbol] | 0.57 | — | — |
| [symbol] | [symbol] | [symbol] | 2.1 | 1.47 | [symbol] | 0.25 | 0.04 | 0.14 |
| [symbol] | [symbol] | [symbol] | 2.0 | [symbol] .42 | [symbol] | 0.35 | 0.29 | 0.32 |
| [symbol] | [symbol] | [symbol] | 2.0 | 1.79 | [symbol] | 0.3 [symbol] | 0.37 | 0.41 |
| [symbol] | [symbol] | [symbol] | 2.0 | 2.90 | [symbol] | 0. [symbol] | 0.53 | 0.45 |
| [symbol] | [symbol] | [symbol] | 2.2 | 1.34 | [symbol] | 0.64 | 0.31 | 0.4 [symbol] |
| [symbol] | [symbol] | [symbol] | 1.9 | 1. [symbol] | [symbol] | 0. [symbol] | 0.00 | 0. [symbol] |
| [symbol] | [symbol] | [symbol] | [symbol] .1 | 1.42 | [symbol] | 0. [symbol] | — | — |
| [symbol] | [symbol] | [symbol] | 2.0 | 1.75 | [symbol] | 0.7 [symbol] | 0.17 | 0.48 |
| [symbol] | [symbol] | [symbol] | 2.3 | 1.45 | [symbol] | 0. [symbol] | 0.32 | 0.34 |
| [symbol] | [symbol] | [symbol] | [symbol] .1 | 1. [symbol] | [symbol] | 0.7 [symbol] | — | — |
| [symbol] | [symbol] | [symbol] | 2.2 | 1.27 | [symbol] | 0.47 | 0.43 | 0.45 |
| [symbol] | [symbol] | [symbol] | 2.0 | 1.58 | [symbol] | 0.57 | 0.49 | 0.35 |
| [symbol] | [symbol] | [symbol] | 2.0 | 1.38 | [symbol] | 0. [symbol] | 0.43 | 0.3 [symbol] |
| [symbol] | [symbol] | [symbol] | 2.0 | 1.38 | [symbol] | 0. [symbol] | 0.17 | 0.34 |
| [symbol] | [symbol] | [symbol] | 1.9 | 1.47 | [symbol] | 0.67 | — | — |

| | Collection | | | | | | |
|---|---|---|---|---|---|---|---|
| | center/ | | Histological | Age at | | | |
| Patient ID | Consort [symbol] | Patient type | diagnosis | collection | Sex | Stage | Smoking status |
| CGPLLU703P | [symbol] | Lung cancer | Squamous cell | 62 | Male | IA | Current smoker |
| | | | carcinoma | | | | |
| CGPLLU705P | [symbol] | Lung cancer | Adenocarcinoma | 70 | Female | IB | Current smoker |
| CGPLLU707P | [symbol] | Benign | Benign | 69 | Female | NA | Former smoker |
| CGPLLU709P | [symbol] | Benign | Benign | 73 | Male | NA | Current smoker |
| CGPLLU710P | [symbol] | Lung cancer | Adenocarcinoma | 67 | Female | IA2 | Former smoker |
| CGPLLU712P | [symbol] | Lung cancer | Adenocarcinoma | 60 | Female | IB | Former smoker |
| CGPLLU713P | [symbol] | Lung cancer | Adenocarcinoma | 74 | Female | IA2 | Current smoker |
| CGPLLU714P | [symbol] | Benign | Benign | 64 | Female | NA | Former smoker |
| CGPLLU715P | [symbol] | Lung cancer | Adenocarcinoma | 82 | Male | IIA | Former smoker |
| CGPLLU718P | [symbol] | Lung cancer | Adenocarcinoma | 69 | Female | IB | Current smoker |

| | | Years | | | | | | |
|---|---|---|---|---|---|---|---|---|
| | | from | | | | Number of | Number of | |
| | Pack | quitting | Sequencing | | Read | sequenced | sequenced | Percent of |
| Patient ID | years | smoking | approach | Read type | length | fragments | reads | Q20 bases |
| CGPLLU703P | 23 | 0 | WGS | Paired-end | 100 | 33,574,122 | 67,148,244 | 95.69% |
| CGPLLU705P | 14.5 | 0 | WGS | Paired-end | 100 | 34,628,831 | 69,257,662 | 96.37% |
| CGPLLU707P | NA | 14 | WGS | Paired-end | 100 | 34,670,279 | 69,340,558 | 96.50% |
| CGPLLU709P | NA | 0 | WGS | Paired-end | 100 | 33,642,169 | 67,284,338 | 96.13% |
| CGPLLU710P | NA | 18 | WGS | Paired-end | 100 | 35,232,947 | 70,455,894 | 95.57% |
| CGPLLU712P | NA | 5 | WGS | Paired-end | 100 | 32,634,843 | 65,269,606 | 95.43% |
| CGPLLU713P | NA | 0 | WGS | Paired-end | 100 | 33,108,353 | 66,216,726 | 96.13% |
| CGPLLU714P | NA | NA | WGS | Paired-end | 100 | 37,889,844 | 75,779,688 | 96.21% |
| CGPLLU715P | 9 | 58 | WGS | Paired-end | 100 | 35,419,969 | 70,839,936 | 96.28% |
| CGPLLU718P | 45 | 0 | WGS | Paired-end | 100 | 15,463,476 | 30,926,956 | 95.66% |

| | | | | | Single | | | |
|---|---|---|---|---|---|---|---|---|
| | | Reads | Whole | Estimated | molecule | | | GEMINI/ |
| | Percent of | mapped to | genome | 8-oxo-dG | [symbol] | GEMINI | DELFI | DELFI |
| Patient ID | Q30 bases | genome | coverage | level | changes | score [symbol] | score | score |
| CGPLLU703P | 93.09% | 61,004,948 | 2.0 | 1.49 | 2.097 | 0.89 | 0.03 | 0.46 |
| CGPLLU705P | 93.79% | 64,451,339 | 2.1 | 1.56 | 2.620 | 0.92 | 0.72 | 0.82 |
| CGPLLU707P | 94.22% | 64,079,465 | 2.1 | 1.33 | 2.590 | 0.09 | 0.28 | 0.18 |
| CGPLLU709P | 93.46% | 62,532,585 | 2.0 | 1.22 | 3.560 | 0.45 | 0.17 | 0.31 |
| CGPLLU710P | 92.77% | 64,263,765 | 2.1 | 1.50 | 1.912 | 0.79 | 0.22 | 0.50 |
| CGPLLU712P | 93.91% | 60,637,256 | 2.0 | 1.60 | 2.414 | 0.25 | 0.14 | 0.20 |
| CGPLLU713P | 93.69% | 60,444,329 | 2.0 | 1.34 | 2.441 | 0.68 | 0.43 | 0.56 |
| CGPLLU714P | 93.56% | 70,849,258 | 2.3 | 1.30 | 3.913 | 0.48 | 0.09 | 0.29 |
| CGPLLU715P | 93.06% | 65,477,465 | 2.1 | 1.35 | 2.265 | 0.73 | — | — |
| CGPLLU718P | 93.04% | 28,344,668 | 0.9 | 1.24 | 1.792 | 0.99 | 0.25 | 0.62 |

[symbol]

indicates data missing or illegible when filed

SUPPLEMENTARY TABLE 7
Summary of SCLC vs. NSCLC analyses

| | | | Regional difference |
|---|---|---|---|
| | | | in single molecule |
| | | Lung cancer | C > A frequency (per |
| Patient ID | Cohort | subtype | Mb of evaluable bases) |
| CGPLLU283P | LUCAS | NSCLC | −4.50 |
| CGPLLU284P | LUCAS | NSCLC | 9.62 |
| CGPLLU290P | LUCAS | NSCLC | −0.89 |
| CGPLLU294P | LUCAS | NSCLC | −2.89 |
| CGPLLU302P | LUCAS | NSCLC | −2.16 |
| CGPLLU306P | LUCAS | NSCLC | −2.50 |
| CGPLLU307P | LUCAS | NSCLC | −3.67 |
| CGPLLU314P | LUCAS | NSCLC | −3.31 |
| CGPLLU316P | LUCAS | NSCLC | −0.41 |
| CGPLLU320P | LUCAS | NSCLC | −5.60 |
| CGPLLU321P | LUCAS | NSCLC | 0.32 |
| CGPLLU323P | LUCAS | NSCLC | 0.42 |
| CGPLLU325P | LUCAS | NSCLC | −0.79 |
| CGPLLU327P | LUCAS | SCLC | 8.49 |
| CGPLLU331P | LUCAS | NSCLC | 6.38 |
| CGPLLU333P | LUCAS | NSCLC | −2.98 |
| CGPLLU335P | LUCAS | NSCLC | 4.47 |
| CGPLLU336P | LUCAS | NSCLC | −2.72 |
| CGPLLU341P | LUCAS | NSCLC | −3.15 |
| CGPLLU343P | LUCAS | SCLC | 14.26 |
| CGPLLU344P | LUCAS | NSCLC | 2.14 |
| CGPLLU349P | LUCAS | NSCLC | −3.19 |
| CGPLLU369P | LUCAS | NSCLC | −3.62 |
| CGPLLU376P | LUCAS | NSCLC | −2.02 |
| CGPLLU376P | LUCAS | NSCLC | −2.30 |
| CGPLLU378P | LUCAS | NSCLC | −3.77 |
| CGPLLU384P | LUCAS | NSCLC | −3.54 |
| CGPLLU388P | LUCAS | NSCLC | −0.55 |
| CGPLLU397P | LUCAS | SCLC | −2.79 |
| CGPLLU398P | LUCAS | NSCLC | 0.34 |
| CGPLLU403P | LUCAS | NSCLC | −2.20 |
| CGPLLU404P | LUCAS | SCLC | 6.94 |
| CGPLLU410P | LUCAS | NSCLC | −3.02 |
| CGPLLU412P | LUCAS | NSCLC | 0.74 |
| CGPLLU414P | LUCAS | NSCLC | 1.52 |
| CGPLLU424P | LUCAS | NSCLC | 0.16 |
| CGPLLU426P | LUCAS | SCLC | 0.42 |
| CGPLLU435P | LUCAS | NSCLC | −2.99 |
| CGPLLU441P | LUCAS | NSCLC | −3.10 |
| CGPLLU443P | LUCAS | NSCLC | −3.06 |
| CGPLLU449P | LUCAS | NSCLC | −1.90 |
| CGPLLU452P | LUCAS | NSCLC | −1.17 |
| CGPLLU456P | LUCAS | NSCLC | −2.05 |
| CGPLLU457P | LUCAS | SCLC | 5.73 |
| CGPLLU458P | LUCAS | SCLC | 12.65 |
| CGPLLU467P | LUCAS | NSCLC | −2.39 |
| CGPLLU468P | LUCAS | NSCLC | −5.75 |
| CGPLLU469P | LUCAS | NSCLC | −6.37 |
| CGPLLU473P | LUCAS | SCLC | 0.15 |
| CGPLLU477P | LUCAS | NSCLC | −5.68 |
| CGPLLU480P | LUCAS | NSCLC | −1.73 |
| CGPLLU483P | LUCAS | NSCLC | −1.26 |
| CGPLLU488P | LUCAS | NSCLC | −0.86 |
| CGPLLU501P | LUCAS | NSCLC | −0.78 |
| CGPLLU504P | LUCAS | NSCLC | −0.20 |
| CGPLLU505P | LUCAS | NSCLC | 10.21 |
| CGPLLU506P | LUCAS | NSCLC | −4.73 |
| CGPLLU518P | LUCAS | NSCLC | 1.27 |
| CGPLLU526P | LUCAS | NSCLC | 10.07 |
| CGPLLU531P | LUCAS | SCLC | 3.50 |
| CGPLLU533P | LUCAS | NSCLC | −2.47 |
| CGPLLU538P | LUCAS | NSCLC | −1.87 |
| CGPLLU543P | LUCAS | NSCLC | 0.23 |
| CGPLLU544P | LUCAS | NSCLC | −1.01 |
| CGPLLU550P | LUCAS | NSCLC | −0.41 |
| CGPLLU558P | LUCAS | NSCLC | −0.12 |
| CGPLLU559P | LUCAS | NSCLC | −6.35 |
| CGPLLU565P | LUCAS | NSCLC | −4.28 |
| CGPLLU566P | LUCAS | NSCLC | −2.47 |
| CGPLLU569P | LUCAS | NSCLC | −3.19 |
| CGPLLU571P | LUCAS | NSCLC | −0.40 |
| CGPLLU578P | LUCAS | NSCLC | −3.22 |
| CGPLLU585P | LUCAS | NSCLC | −1.33 |
| CGPLLU587P | LUCAS | NSCLC | −0.30 |
| CGPLLU594P | LUCAS | SCLC | 4.72 |
| CGPLLU606P | LUCAS | NSCLC | −3.11 |
| CGPLLU607P | LUCAS | NSCLC | −2.94 |
| CGPLLU611P | LUCAS | NSCLC | −3.60 |
| CGPLLU616P | LUCAS | NSCLC | −2.34 |
| CGPLLU622P | LUCAS | NSCLC | −6.88 |
| CGPLLU626P | LUCAS | NSCLC | −7.10 |
| CGPLLU627P | LUCAS | NSCLC | −3.94 |
| CGPLLU633P | LUCAS | NSCLC | −5.13 |
| CGPLLU636P | LUCAS | NSCLC | 2.36 |
| CGPLLU637P | LUCAS | NSCLC | −2.61 |
| CGPLLU641P | Validation | SCLC | −2.10 |
| CGPLLU642P | Validation | NSCLC | −1.18 |
| CGPLLU644P | Validation | NSCLC | 0.62 |
| CGPLLU645A | Validation | NSCLC | −4.36 |
| CGPLLU646P | Validation | NSCLC | −5.50 |
| CGPLLU649P | Validation | SCLC | −0.97 |
| CGPLLU650P | Validation | NSCLC | −1.73 |
| CGPLLU658P | Validation | NSCLC | −1.29 |
| CGPLLU661P | Validation | SCLC | 7.42 |
| CGPLLU670P | Validation | NSCLC | −3.97 |
| CGPLLU672P_2 | Validation | NSCLC | −3.77 |
| CGPLLU675P | Validation | NSCLC | −5.83 |
| CGPLLU683P | Validation | NSCLC | −3.45 |
| CGPLLU684P | Validation | NSCLC | −2.16 |
| CGPLLU686P | Validation | NSCLC | −4.93 |
| CGPLLU688P | Validation | NSCLC | −3.52 |
| CGPLLU690P | Validation | NSCLC | −3.21 |
| CGPLLU693P | Validation | NSCLC | −2.07 |
| CGPLLU696P | Validation | NSCLC | −3.86 |
| CGPLLU697P | Validation | NSCLC | 1.72 |
| CGPLLU698P | Validation | NSCLC | −3.49 |
| CGPLLU703P | Validation | NSCLC | −0.69 |
| CGPLLU705P | Validation | NSCLC | −1.68 |
| CGPLLU710P | Validation | NSCLC | −0.53 |
| CGPLLU712P | Validation | NSCLC | 0.50 |
| CGPLLU713P | Validation | NSCLC | −2.98 |
| CGPLLU713P | Validation | NSCLC | −3.71 |

SUPPLEMENTARY TABLE 8
Summary of liver cohort and genomic analyses

| | | | | | | | | | | Percent | Percent | | Whole | | | |
|---|---|---|---|---|---|---|---|---|---|---|---|---|---|---|---|---|
| Patient | Patient | | | [symbol] | Sequencing | | Read | | | of Q20 | of Q30 | | genome | | | |
| ID | type | Age | Sex | stage | approach | Read type | length | [symbol] | [symbol] | bases | bases | [symbol] | coverage | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| | | | | | | | | | | Percent | Percent | | Whole | | | |
| Patient | Patient | | | [symbol] | Sequencing | | Read | | | of Q20 | of Q30 | | genome | | | |
| ID | type | Age | Sex | stage | approach | Read type | length | [symbol] | [symbol] | bases | bases | [symbol] | coverage | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Female | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |
| [symbol] | [symbol] | [symbol] | Male | [symbol] | WGS | Paired-end | 100 | [symbol] | [symbol] | [symbol] | [symbol] | [symbol] | [symbol] | [symbol] | [symbol] | [symbol] |

[symbol]

indicates data missing or illegible when filed

1. A method of determining the frequency of somatic mutations in a subject, comprising:
extracting cell-free DNA (cfDNA) from a subject's biological sample;
processing the cfDNA to generate genomic libraries from the extracted cfDNA;
sequencing individual cfDNA molecules to obtain mutation profiles;
scanning each sequenced cfDNA molecule for single nucleotide changes after removing common germline variants and/or unevaluable regions;
determining multiregional differences in mutation profiles;
quantifying multiregional differences in mutation profiles by one or more assays wherein the wherein the one or more assays comprise in silico dilution assays and/or downsampling assays; and
executing a machine learning model for determining the frequency of somatic mutations in the subject wherein the frequency of somatic mutations is defined as the number of variants per million evaluated positions across all the DNA molecules sequenced.

2. The method of claim 1, wherein the determining of genome-wide mutation and fragmentation profiles comprises identifying mutations in sequences of individual cfDNA molecules and changes in fragment lengths.

3. The method of claim 1, wherein the mutation profiles comprise mutation frequency and type of mutation across the subject's genome.

4. The method of claim 3, wherein the mutation profiles across the subject's genome are determined using non-overlapping bins ranging in size from at least about one thousand bases to at least about twenty million bases.

5. The method of claim 3, wherein the mutation profiles across the subject's genome are determined using non-overlapping bins ranging in size from at least about one thousand bases to at least about ten million bases.

6. The method of claim 3, wherein the mutation profiles across the subject's genome is determined using non-overlapping bins ranging in size from at least about one thousand bases to at least about five million bases.

7. The method of claim 3, wherein mutations for each sequenced molecule are determined after removing common germline variants, and unevaluable regions.

8. The method of claim 1, wherein the frequency of single molecule somatic mutations and type of mutation across the subject's genome is diagnostic of cancer as compared to the frequency of single molecule somatic mutations and type of mutation across a normal subject's genome.

9. The method of claim 1
where such analysis is performed in a subject from whom tumor tissue is unavailable.

10. A method of treating cancer in a subject, the method comprising:
extracting cell-free DNA (cfDNA) from a subject's biological sample;
processing the cfDNA to generate genomic libraries from the extracted cfDNA;
sequencing individual cfDNA molecules to obtain mutation profiles;
scanning each sequenced cfDNA molecule for single nucleotide changes after removing common germline variants and/or unevaluable regions;
determining multiregional differences in mutation profiles and determining the frequency of somatic mutations in the subject;
quantifying multiregional differences in mutation profiles by one or more assays wherein the wherein the one or more assays comprise in silico dilution assays and/or downsampling assays;
executing a machine learning model for determining the frequency of somatic mutations in the subject wherein the frequency of somatic mutations is defined as the number of variants per million evaluated positions across all the DNA molecules sequenced; and on the basis thereof;
administering a cancer treatment to the subject.

11. The method of claim 10, wherein the cancer treatment comprises: surgery, adjuvant chemotherapy, neoadjuvant chemotherapy, radiation therapy, hormone therapy, cytotoxic therapy, immunotherapy, adoptive T cell therapy, targeted therapy, and combinations thereof.

12. The method of claim 10, wherein the cancer comprises colorectal cancer, lung cancer, breast cancer, gastric cancers, pancreatic cancers, bile duct cancers, brain cancer or ovarian cancer.

13. The method of claim 12, wherein the lung cancer is small cell lung cancer (SCLC).

14. The method of claim 12, wherein the lung cancer is non-small cell lung cancer (NSCLC).

15. The method of claim 1, wherein subjects with cancer comprise altered mutational profiles associated with chromatin organization as compared to healthy individuals.

16. The method of claim 1, wherein genome-wide mutation and fragmentation profiles comprises identifying mutations in sequences of individual cfDNA molecules and changes in fragment lengths.

17. The method of claim 10, wherein the mutation profiles comprise mutation frequency and type of mutation across the subject's genome.

18-21. (canceled)

22. A method of determining regional frequency of mutations across a genome comprising:
extracting cell-free DNA (cfDNA) from a subject's biological sample;
processing the cfDNA to generate genomic libraries from the extracted cfDNA;
sequencing individual cfDNA molecules isolated from a subject,
scanning each sequenced cfDNA molecule for single nucleotide changes after removing common germline variants and/or unevaluable regions;
estimating mutation frequencies and types of mutations across the genome;
determining the mutation types and frequencies in genomic regions altered in cancer to mutation profiles and regions mutated in normal cfDNA to determine multiregional differences in mutation profiles;
quantifying multiregional differences in mutation profiles by one or more assays wherein the wherein the one or more assays comprise in silico dilution assays and/or downsampling assays; thereby,
executing a machine learning model for determining regional frequency of mutations across a genome.

23. The method of claim 22, wherein the estimation of mutation frequencies and types of mutations across the genome comprise using non-overlapping bins ranging in size from thousands to millions of bases.

24-30. (canceled)

31. A method of determining whether a subject is a responder to a treatment based on the outcome of performing a method of claim 1.

32. (canceled)
